SEQUENCE LISTING (5 sequences)

SEQ ID NO: 1 - DNA, 931 bp, Erwinia carotovora
atgaataatccgtctcttttaaaccatgctgttgagacaatggccgtaggttccaaaagctttgcgactgcatctaaacttttcgatgcaaaaacaagacggtcagtcctcatgttatacgcctggtgcagacactgtgacgatgttatcgatgatcagaccctgggattccaagcgaggcagcctgcactccagactccagaacaaagactaatgcagttagaaatgaagactagacaagcatacgcgggtagtcaaatgcacgaacctgcttttgctgcttttcaggaagtcgcaatggcacatgacatagctccagcttacgcgtttgatcacctcgagggattcgcaatggacgtcagggaggcacagtattcacaattggatgatacacttagatattgttatcatgttgctggggtggtgggactaatgatggcacaaattatgggagtgcgtgataatgctacattagaccgcgcttgcgatttgggccttgcattccaattgactaacatcgctcgtgatattgttgatgacgcgcacgctggtcggtgttatctaccagcttcttggctcgaacatgagggtcttaacaaagaaaattacgcagccccagagaacaggcaggccctttcaagaatcgctcgtcgactggttcaggaagcagaaccgtactatctttccgcaactgctggactcgctggacttcctcttaggtcggcttgggctatagctacagcgaagcaagtttatcgaaagattggtgtgaaggtagagcaagccggccaacaagcttgggatcagcgtcaatcaactacgacccctgagaagcttacgcttttgttggctgcaagcgggcaagccttgaccagtagaatgagagctcatcctcctaggcctgcccatctgtggcagagacccttgtgat

SEQ ID NO: 2 - DNA, 1256 bp, Zea mays
ggatccaccatggctattattttggttagagctgcttcacctggactttctgctgctgattctatttctcatcaaggtactttgcaatgttcaacacttttgaaaactaaaagaccagcagctagaagatggatgccttgttctttgttgggacttcatccttgggaagctggtagaccttctccagctgtttattcttctcttgctgttaacccagctggagaagctgttgtttcttcagaacagaaggtttacgatgttgttcttaagcaggctgctttgttgaagagacaacttagaactccagttttggatgcaagacctcaagatatggatatgcctagaaatggacttaaggaggcatatgatagatgtggagaaatctgtgaagaatacgctaaaacattttatttgggtactatgcttatgactgaggagagaagaagagctatttgggctatctacgtttggtgtagaagaactgatgagcttgttgatggaccaaacgctaactacatcactcctactgctcttgatagatgggagaaaagattggaggatctttttactggaagaccttacgatatgttggatgctgctctttcagatactatctcaagattccctatcgatatccaacctttcagagatatgatcgagggtatgagatctgatcttagaaagactagatacaacaacttcgatgaattgtacatgtactgttactatgttgctggtacagttggtttgatgtctgttcctgttatgggtatcgctactgagtctaaagctactacagaatctgtttattctgctgctcttgctttgggtattgctaatcaacttacaaacatccttagagatgttggagaagatgctagaaggggaagaatctaccttccacaggatgaacttgctcaagctgggttgtcagatgaggatatcttcaagggtgttgttactaacagatggagaaacttcatgaagagacagatcaagagagctagaatgttcttcgaggaagctgaaagaggtgttactgaattgtctcaagcatcaagatggcctgtttgggcttctcttcttttgtacagacaaatccttgatgagatcgaggctaacgattataacaacttcacaaagagagcttatgttggaaagggaaagaaactcttggctcttcctgttgcttatggaaaaagccttttgcttccttgttcattgagaaatggtcagacataattaattaaggatcc

SEQ ID NO: 3 - DNA, 2158 bp, Arabidopsis thaliana
aaaaatggcttcttctgcatttgctttcccctcttatataatcaccaaaggtggtttgtctactgactcatgtaagagtacttcacttagttcatccagatcattggttactgatttgcctagtccatgcctgaaacccaataacaacagtcattcaaatcgtcgagctaaggtatgtgcaagcctcgcggaaaagggagaatactattctaatcgccctccaacacctctcttagacacaatcaactaccctatacatatgaaaaatttatctgtaaaagagttgaagcaactatcggatgagctgagatccgatgtcattttcaacgtcagcaagaccgggggtcatcttggatctagtctaggcgttgtggagctgactgtcgctctccactacattttcaataccccgcaggataaaattttgtgggacgttgggcaccaatcatatcctcataagatccttacaggtagaagaggtaagatgccgaccatgagacaaactaacgggctttccggatttacaaagcggggagagagcgagcacgattgctttggaactggtcattcatcaacaactatcagtgccgggctcggtatggctgtgggtagagacttgaagggaaaaaataacaacgttgtggcagtaatcggtgatggtgcgatgacagccggccaggcctatgaagctatgaataatgccggatacttagacagcgatatgatcgtaatactcaacgacaacaaacaggtgtcccttccaacagcgactctagacggcccttcgccgccagttggcgctctatctagcgctctttctcgtctccaatcgaacccagcactcagagaactgagggaggtcgcaaagggaatgactaagcaaattggtggtcccatgcatcaattagctgctaaggtggatgagtatgcacgcggaatgatctcgggcacgggcagttctctgtttgaggagctggggctctattatattggaccagtggatggacacaacattgacgatctcgttgctatattaaaggaagttaagtctacgaggacaacaggtcctgttcttattcatgttgtaaccgaaaaggggcgtggatatccctatgcagaacgtgctgatgataagtatcatggagtcgtcaaatttgatccagcaactgggaggcagttcaaaaccaccaataaaactcagtcctacacgacgtattttgccgaagctctcgtggctgaagctgaggtagacaaagacgttgttgctatacatgcagctatgggaggaggaacaggtcttaacctgtttcaacgtagatttcctactcgatgtttcgatgtggggattgctgagcaacatgcggtcacgtttgccgctgggcttgcatgcgaaggtttaaaacctttctgtgcgatatactcttcttttatgcagagagcatacgatcaagtggtacatgatgttgatttgcaaaaacttccagttagattcgctatggatagggccggactcgttggagcagatgggccgactcactgcggcgcgtttgatgttacattcatggcatgtcttccaaatatgattgtgatggcaccaagcgatgaggcagatcttttcaatatggtggctacggccgttgcaatcgatgatagaccaagttgcttccgataccctcggggaaatggaataggcgttgcacttcctcctggaaacaagggagtccctattgaaataggtaaaggacgtatcttgaaagagggtgaaagggttgcacttctcggctacggttctgcagttcagtcttgtttgggagctgcggttatgctagaagaaagagggttaaatgtcacagttgcggacgccagattctgtaaaccgttggacagagcgcttattaggtcacttgctaagtctcacgaagttttgatcactgtggaagagggttccattggtggatttggttcgcacgtggtacagtttttggctttagatggtttgttggatggcaagcttaaatggcggcctatggtcctacctgataggtacattgatcatggtgctccggctgaccaacttgccgaggctggtcttatgccttcacacattgctgccaccgctttaaatttgatcggagctccacgagaagctctattctga

SEQ ID NO: 4 - DNA, 107 bp, Artificial Sequence
  Synthesized
aaatggccatgaaactgaatgccctcatgactcttcagtgcccaaaaaggaacatgtttacgagaattgcccctcctcaagcagggagagtgagatcaaaggtgtcc

SEQ ID NO: 5 - DNA, 868 bp, Solanum tuberosum
agctttaacgagatagaaaattataatactccgttttgttcattacttaacaaatgcaacagtatcttgtaccaaatcctctctcttttcaaacttttctatttggctgttgacagagtaatcaggatacaaaccacaagtatttaattgactcatccaccagatattatgatttatgaatcctcgaaaagcctatccattaagtcctcatctatggatatacttgacagtttcttcctatttgggtttttttttttcctgccaagtggaacggagacatgttatgttgtatacgggaatctcgttaaaaaaaaaaatacaataggaagaaatgtaacaaacattgaatgttgtttttaaccatccttccttttagcagtgtatcaattttgtaatagaaccatgcatctcaatcttaatactaaaaaatgcaacaaaattctagtggagggaccagtaccagtacattagatattattttttattactataataatattttaattaacacgagacataggaatgtcaagtggtagcggtaggagggagttggtttagtttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacactcactcacacagctcaacaagtggtaacttttactcatctcctccaattatttctgatttcatgcaggtcgactctagaaggcctg

(19) United States
(12) Patent Application Publication
(10) Pub. No.: US 2013/0219560 A1
(43) Pub. Date: Aug. 22, 2013

(54) REDUCTION OF POST-HARVEST PHYSIOLOGICAL DETERIORATION

(71) Applicant: Richard T. Sayre, Los Alamos, NM (US)

(72) Inventor: Richard T. Sayre, Los Alamos, NM (US)

(21) Appl. No.: 13/820,613

(22) PCT Filed: Sep. 2, 2011
(86) PCT No.: PCT/US11/50329

§ 371 Date: Mar. 20, 2013

Related U.S. Application Data
(60) Provisional application No. 61/379,727, filed on Sep. 2, 2010.

(51) Int. Cl. C12N 15/82

(52) U.S. Cl. CPC C12N 15/8243
USPC 800/287; 800/278; 800/298

(57) ABSTRACT

The invention provides methods for transgenically controlling physiological post-harvest deterioration (PPD) in plants. Among other aspects, the invention also provides transgenic plants produced by the methods and constructs for creating the plants. According to the present invention, PPD is controlled by expressing one or more transgenes which modulate ROS production or cyanogen levels. Examplary transgenes include alternative oxidase, ROS scavengers, carotenoid biosynthesis genes, and cyanogen metabolism.

### CROSS REFERENCE TO RELATED APPLICATIONS

This application claims the benefit of priority of U.S. provisional application No. 61/379,727, filed Sep. 2, 2010, the disclosure of which is incorporated by reference as if written herein in its entirety.

### BACKGROUND OF THE INVENTION

### Field of the Invention

The present invention relates to post harvest stability of plants.

Cassava roots are the major source of calories for subsistence farmers in sub-Saharan Africa and cassava ranks fifth globally among all crops directly consumed by humans. Cassava roots suffer, however, from rapid post-harvest physiological deterioration (PPD) within 24-48 hours of harvesting. This short shelf-life property is a constraint that limits its use and commercialization potential.

Sanchez et al. (Journal of the Science of Food and Agriculture; Volume 86 Issue 4, Pages 634-639) describe that PPD in selected cassava lines was inversely correlated with carotenoid content. However, Sanchez et al. do not teach genetic strategies for reducing PPD.

McKersie et al. (U.S. Pat. No. 6,518,486) describe the expression of a mitochondrial alternative oxidase in plants to increase the mass of the storage organ. However, McKersie et al. do not teach reducing PPD and do not teach expressing an alternative oxidase at sufficient levels to reduce PPD.

What are needed in the art are genetic strategies for reducing PPD.

### SUMMARY OF THE INVENTION

This invention provides a genetically modified plant comprising one or more gene constructs for decreasing post-harvest deterioration.

The invention also provides one or more constructs for creating genetically modified plants with decreased post-harvest deterioration.

The invention also provides a method for creating a genetically modified plant comprising transforming the plant with one or more constructs of the invention.

A genetically modified plant (e.g. cassava) of the present invention comprises one or more genes expressible by the host, wherein expression enhances post-harvest stability relative to a non-transformed host. The one or more genes are selected from those encoding AOX, cyanogen detoxification genes, and antioxidation products such as ROS scavengers and carotenoid biosynthesis genes.

In one embodiment, a plant of the present invention transgenically expresses AOX and one or more antioxidation products such as ROS scavengers and carotenoid biosynthesis genes

In one embodiment, a plant of the present invention transgenically expresses AOX and one or more cyanogen detoxification genes.

In one embodiment, a plant of the present invention transgenically expresses AOX, one or more cyanogen detoxification genes, and one or more antioxidation products such as ROS scavengers and carotenoid biosynthesis genes.

Optionally, the one or more carotenoid biosynthesis genes are selected from phytoene synthase (PSY), 1-deoxyxylulose-5-phosphate synthase (DXS), homogentisate phytyltransferase (HPT), geranylgeranyl reductase (GGR), Homogentisate geranylgeranyl transferase (HGGT),

Optionally the one or more ROS scavengers are selected from superoxide dismutase, catalase, ascorbate peroxidase, D-galacturonic acid reductase, γ-glutamylcysteine synthase, dehydroascorbate reductase, glutathione peroxidase, and glutathione reductase.

Optionally, the one or more cyanogen detoxification genes are selected from β-cyanoalanine synthase (β-CAS), Rhodanese, nitrilase 4 (NIT4), hydroxynitrile lyase (HNL), linamarase (e.g. vacuole targeted), and CYP79D1/D2 RNAi,

In one embodiment, a plant (e.g. cassava) of the present invention transgenically expresses AOX and phytoene synthase.

In one embodiment, a plant (e.g. cassava) of the present invention transgenically expresses AOX, phytoene synthase, and one or more ROS scavengers.

In one embodiment, a plant (e.g. cassava) of the present invention transgenically expresses AOX, phytoene synthase, and DXS.

Optionally, the plant is of the genus Manihot, for example M. walkerae, M. esculenta Crantz, M. esculenta ssp. Flabellifolia, M. esculenta sub spp peruviana, M. tristis., M. carthaginensis, M. brachyloba and M. fomentosa ed.

### BRIEF DESCRIPTION OF THE DRAWINGS

FIG. 1 depicts ROS scavengers of the present invention.

FIG. 2 depicts PPD in cassava roots.

FIG. 3 depicts ROS production in AOX overexpressing plants.

FIG. 4 depicts PPD in AOX over expressing plants

FIG. 5 depicts the structure of an examplary AOX.

FIG. 6 depicts ROS-induced fluorescence in transgenic low cyanide (CAB) cassava, high cyanide (wild type) cassava, and low cyanide supplemented with sodium cyanide. The chart shows quantitation of the fluorescence.

FIG. 7 depicts linamarin levels in the root of a transgenic plant with modulated cyanogen metabolism.

FIG. 8 depicts the expression level of cyanogen metabolism genes.

FIG. 9 depicts the expression level of a cyanogen metabolism gene.

FIG. 10 depicts the results of the field trial data of AOX2. AOX3 and AOX4 with respect to root development.

FIG. 11 depicts the results of room temperature storage for WT, AOX2, AOX3 and AOX4 for 5 days.

FIG. 12 depicts the results of room temperature storage for WT, AOX2, AOX3 and AOX4 for 10 days.

FIG. 13 depicts the results of refrigerated storage for WT, AOX2, AOX3 and AOX4 for 21 days.

### DETAILED DESCRIPTION OF THE INVENTION

As used here, the following definitions and abbreviations apply.

In order that the present disclosure may be more readily understood, certain terms are first defined. Additional definitions are set forth throughout the detailed description. As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The terms “about” or “approximately” means within an acceptable error range for the particular value as determined by one of ordinary skill in the art, which will depend in part on how the value is measured or determined, i.e., the limitations of the measurement system. For example, “about” can mean within 1 or 2 standard deviations, from the mean value. Alternatively, “about” can mean plus or minus a range of up to 20%, preferably up to 10%, more preferably up to 5%.

As used herein, the terms “cell,” “cells,” “cell line,” “host cell,” and “host cells,” are used interchangeably and, encompass animal cells and include plant, invertebrate, non-mammalian vertebrate, insect, algal, and mammalian cells. All such designations include cell populations and progeny. Thus, the terms “transformants” and “transfectants” include the primary subject cell and cell lines derived therefrom without regard for the number of transfers.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids can be defined where amino acids within a group exchange preferentially with each other, and therefore resemble each other most in their impact on the overall protein structure (Schulz, G. E. and R. H. Schirmer, Principles of Protein Structure, Springer-Verlag).

Examples of amino acid groups defined in this manner include: a “charged/polar group,” consisting of Glu, Asp, Asn, Gln, Lys, Arg and His; an “aromatic, or cyclic group,” consisting of Pro, Phe, Tyr and Trp; and an “aliphatic group” consisting of Gly, Ala, Val, Leu, Ile, Met, Ser, Thr and Cys.

Within each group, subgroups can also be identified, for example, the group of charged/polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gln. The aromatic or cyclic group can be sub-divided into the sub-groups consisting of the “nitrogen ring sub-group,” consisting of Pro, His and Trp; and the “phenyl sub-group” consisting of Phe and Tyr. The aliphatic group can be sub-divided into the sub-groups consisting of the “large aliphatic non-polar sub-group,” consisting of Val, Leu and Ile; the “aliphatic slightly-polar sub-group,” consisting of Met, Ser, Thr and Cys; and the “small-residue sub-group,” consisting of Gly and Ala.

Examples of conservative mutations include substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gln for Asn such that a free —NH₂ can be maintained.

“Derived from”, as it relates to proteins and genes, means that the protein or gene comprises the reference protein or gene, or is functionally and structurally related to the reference protein or gene. Similarly, a protein or gene is said to be derived from a reference organism when the protein or gene is derived from a protein or gene naturally expressed by the organism. According to the present invention, precise gene or protein sequences are not required and variants and fragments that retain the function of the reference protein or gene are also contemplated. For example, a protein or gene that is derived from a reference protein or gene can exhibit at least about any of: 80%, 85%, 90%, or 95% sequence identity to the reference protein or gene or to a fragment that retains the function of the reference gene or protein.

The phrase “DNA construct” as used herein refers to any DNA molecule in which two or more ordinarily distinct DNA sequences have been covalently linked. Examples of DNA constructs include but not limited to plasmids, cosmids, viruses, BACs (bacterial artificial chromosome), YACs (yeast artificial chromosome), plant minichromosomes, autonomously replicating sequences, phage, or linear or circular single-stranded or double-stranded DNA sequences, derived from any source, that are capable of genomic integration or autonomous replication. DNA constructs can be assembled by a variety of methods including but not limited to recombinant DNA techniques, DNA synthesis techniques, PCR (Polymerase Chain Reaction) techniques, or any combination of techniques.

“Enhanced trait” or “enhanced phenotype” as used herein refers to a measurable improvement in a trait of photosynthetic organism including, but not limited to, yield increase, including increased yield under non-stress conditions and increased yield under environmental stress conditions Many enhanced traits can affect “yield”, including without limitation, number of cells in a liquid culture of unicellular or multi cellular photosynthetic organism, increased efficiencies of light utilization by a photosynthetic organism, amount of biomass production by a photosynthetic organism, amount of bio fuel production by a photosynthetic organism, and amounts of nutraceuticals including but not limited to Agar, Alginate, Carrageenan, Omega fatty acids, Coenzyme Q10, Astaxanthin, and Beta-Carotene. Nutraceutical, a term combining the words “nutrition” and “pharmaceutical”, is a food or food product that provides health and medical benefits, including the prevention and treatment of disease. Such products may range from isolated nutrients, dietary supplements and specific diets to genetically engineered foods, herbal products, and processed foods such as cereals, soups, and beverages. Other enhanced trait include plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Other traits that can affect yield include, efficiency of germination (including germination in stressed conditions), growth rate (including growth rate in stressed conditions), ear number, seed number per ear, seed size, composition of seed (starch, oil, protein) and characteristics of seed fill.

“Examplary” (or “e.g.” or “by example”) means a non-limiting example.

“Extract” means a material derived from a photosynthetic host or plant part of the present invention. For example, an extract can be derived by purification or chemical alteration.

The term “expression” as used herein refers to transcription and/or translation of a nucleotide sequence within a host cell. The level of expression of a desired product in a host cell may be determined on the basis of either the amount of corresponding mRNA that is present in the cell, or the amount of the desired polypeptide encoded by the selected sequence. For example, mRNA transcribed from a selected sequence can be quantified by Northern blot hybridization, ribonuclease RNA protection, in situ hybridization to cellular RNA or by PCR. Proteins encoded by a selected sequence can be quantified by various methods including, but not limited to, e.g., ELISA, Western blotting, radioimmunoassays, immunoprecipitation, assaying for the biological activity of the protein, or by immunostaining of the protein followed by FACS analysis.

“Expression control sequences” are regulatory sequences of nucleic acids, such as promoters, leaders, enhancers, introns, recognition motifs for RNA, or DNA binding proteins, polyadenylation signals, terminators, internal ribosome entry sites (IRES) and the like, that have the ability to affect the transcription or translation of a coding sequence in a host cell. Exemplary expression control sequences are described in Goeddel; Gene Expression Technology: Methods in Enzymology 185, Academic Press, San Diego, Calif. (1990).

A “gene” is a sequence of nucleotides which code for a functional gene product. Generally, a gene product is a functional protein. However, a gene product can also be another type of molecule in a cell, such as RNA (e.g., a tRNA or an rRNA). A gene may also comprise regulatory (i.e., non-coding) sequences as well as coding sequences and introns. Exemplary regulatory sequences include promoters, enhancers and terminators. The transcribed region of the gene may also include untranslated regions including introns, a 5′-untranslated region (5′-UTR) and a 3′-untranslated region (3′-UTR).

The term “heterologous” refers to nucleic acids or proteins which has been introduced into a plant, or animal, or cell, or a nucleic acid molecule (such as chromosome, vector, or nucleic acid construct), that are derived from another source, or which are from the same source but are located in a different (i.e. non native) context.

The term “homology” describes a mathematically based comparison of sequence similarities which is used to identify genes or proteins with similar functions or motifs. The nucleic acid and protein sequences of the present invention can be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17):3389-3402. When utilizing BLAST and Gapped BLAST programs, the default parameters of the respective programs (e.g., XBLAST and BLAST) can be used.

The term “homologous” refers to the relationship between two proteins that possess a “common evolutionary origin”, including proteins from superfamilies (e.g., the immunoglobulin superfamily) in the same species of animal, as well as homologous proteins from different species of animal (for example, myosin light chain polypeptide, etc.; see Reeck et al., Cell, 50:667, 1987). Such proteins (and their encoding nucleic acids) have sequence homology, as reflected by their sequence similarity, whether in terms of percent identity or by the presence of specific residues or motifs and conserved positions.

As used herein, the term “increase” or the related terms “increased”, “enhance” or “enhanced” refers to a statistically significant increase. For the avoidance of doubt, the terms generally refer to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control value.

The term “isolated,” when used to describe a protein or nucleic acid, means that the material has been identified and separated and/or recovered from a component of its natural environment. Contaminant components of its natural environment are materials that would typically interfere with research, diagnostic or therapeutic uses for the protein or nucleic acid, and may include enzymes, hormones, and other proteinaceous or non-proteinaceous solutes. In some embodiments, the protein or nucleic acid will be purified to at least 95% homogeneity as assessed by SDS-PAGE under non-reducing or reducing conditions using Coomassie blue or, preferably, silver stain. Isolated protein includes protein in situ within recombinant cells, since at least one component of the protein of interest's natural environment will not be present. Ordinarily, however, isolated proteins and nucleic acids will be prepared by at least one purification step.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs.

Optimal alignment of sequences for comparison can be conducted, for example, by the local homology algorithm described in Smith & Waterman 1981, by the homology alignment algorithm described in Needleman & Wunsch 1970, by the search for similarity method described in Pearson & Lipman 1988, by computerized implementations of these algorithms (GAP, BESTFIT, PASTA, and TFASTA in the GCG Wisconsin Package, available from Accelrys, Inc., San Diego, Calif., United States of America), or by visual inspection. See generally, (Altschul, S. F. et al., J. Molec. Biol. 215: 403-410 (1990) and Altschul et al. Nuc. Acids Res. 25: 3389-3402 (1997)).

One example of an algorithm that is suitable for determining percent sequence identity and sequence similarity is the BLAST algorithm, which is described in (Altschul, S., et al., NCBI NLM NIH Bethesda, Md. 20894; & Altschul, S., et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold.

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always; 0) and N (penalty score for mismatching residues; always; 0). For amino acid sequences, a scoring matrix is used to calculate the cumulative score. Extension of the word hits in each direction are halted when the −27 cumulative alignment score falls off by the quantity X from its maximum achieved value, the cumulative score goes to zero or below due to the accumulation of one or more negative-scoring residue alignments, or the end of either sequence is reached. The BLAST algorithm parameters W. T. and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

The terms “operably linked”, “operatively linked,” or “operatively coupled” as used interchangeably herein, refer to the positioning of two or more nucleotide sequences or sequence elements in a manner which permits them to function in their intended manner. In some embodiments, a nucleic acid molecule according to the invention includes one or more DNA elements capable of opening chromatin and/or maintaining chromatin in an open state operably linked to a nucleotide sequence encoding a recombinant protein. In other embodiments, a nucleic acid molecule may additionally include one or more DNA or RNA nucleotide sequences including, but not limited to: (a) a nucleotide sequence capable of increasing translation; (b) a nucleotide sequence capable of increasing secretion of the recombinant protein outside a cell; (c) a nucleotide sequence capable of increasing the mRNA stability, and (d) a nucleotide sequence capable of binding a trans-acting factor to modulate transcription or translation, where such nucleotide sequences are operatively linked to a nucleotide sequence encoding a recombinant protein. Generally, but not necessarily, the nucleotide sequences that are operably linked are contiguous and, where necessary, in reading frame. However, although an operably linked DNA element capable of opening chromatin and/or maintaining chromatin in an open state is generally located upstream of a nucleotide sequence encoding a recombinant protein; it is not necessarily contiguous with it. Operable linking of various nucleotide sequences is accomplished by recombinant methods well known in the art, e.g. using PCR methodology, by ligation at suitable restrictions sites or by annealing. Synthetic oligonucleotide linkers or adaptors can be used in accord with conventional practice if suitable restriction sites are not present.

“PCD” means programmed cell death and refers generally to apoptotic mechanisms that lead to cell death.

The terms “polynucleotide,” “nucleotide sequence” and “nucleic acid” are used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. A nucleic acid molecule can take many different forms, e.g., a gene or gene fragment, one or more exons, one or more introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. As used herein, a polynucleotide includes not only naturally occurring bases such as A, T, U, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Prokaryotic promoters contain Shine-Dalgarno sequences in addition to the −10 and −35 consensus sequences.

A large number of promoters, including constitutive, inducible and repressible promoters, from a variety of different sources are well known in the art. Representative sources include for example, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters active in plants include, for example nopaline synthase (nos) promoter and octopine synthase (ocs) promoters carried on tumor-inducing plasmids of Agrobacterium tumefaciens and the caulimovirus promoters such as the Cauliflower Mosaic Virus (CaMV) 19S or 35S promoter (U.S. Pat. No. 5,352,605), CaMV 35S promoter with a duplicated enhancer (U.S. Pat. Nos. 5,164,316; 5,196,525; 5,322,938; 5,359,142; and 5,424,200), the Figwort Mosaic Virus (FMV) 35S promoter (U.S. Pat. No. 5,378,619), the cassava vein mosaic virus (U.S. Pat. No. 7,601,885). These promoters and numerous others have been used in the creation of constructs for transgene expression in plants or plant cells. Other useful promoters are described, for example, in U.S. Pat. Nos. 5,391,725; 5,428,147; 5,447,858; 5,608,144; 5,614,399; 5,633,441; 6,232,526; and 5,633,435, all of which are incorporated herein by reference.

As used herein a “photosynthetic organism” means an organism capable of performing photosynthetic reaction in presence of light belonging to kingdom “Plantae” that include familiar organisms such as trees, herbs, bushes, grasses, vines, ferns, mosses, and algae. Photosynthetic organisms can be unicellular, or multi cellular.

“Plant part” means any part of the plant less than the whole. For example, a plant part can be a specialized tissue or organ of the plant (e.g. seed, leaf, fruit, root, flower). In some embodiments of the present invention, a particular plant part does not contain the heterologous genes as taught herein while other plant parts do so contain.

“Plant product” means a product derived from a plant as the result of one or more processing steps. For example, a plant product can be an extract. Examples of cassava plant products include starch, tapioca, and other cassava plant products.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell. Methods for purification are well-known in the art. As used herein, the term “substantially free” is used operationally, in the context of analytical testing of the material. Preferably, purified material substantially free of contaminants is at least 50% pure; more preferably, at least 75% pure, and more preferably still at least 95% pure. Purity can be evaluated by chromatography, gel electrophoresis, immunoassay, composition analysis, biological assay, and other methods known in the art. The term “substantially pure” indicates the highest degree of purity, which can be achieved using conventional purification techniques known in the art.

“ROS” means reactive oxygen species, which are free radicals that contain the oxygen atom. They are highly reactive due to the presence of unpaired valence shell electrons. Examples of ROS's are oxygen ions and peroxides, superoxide (.O₂—), the hydroxyl radical (.OH), and hydrogen peroxide (H₂O₂),

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Terms used to describe sequence relationships between two or more polynucleotides or polypeptides include “reference sequence,” “comparison window,” “sequence identity,” “percentage of sequence identity” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology,” John Wiley & Sons Inc, 1994-1998, Chapter 15.

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes).

In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (1970, J. Mol. Biol. 48: 444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frame shift gap penalty of 5.

The percent identity between two amino acid or nucleotide sequences can be determined using the algorithm of E. Meyers and W. Miller (1989, Cabios, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997, Nucleic Acids Res, 25: 3389-3402). When utilizing BLAST and Gapped BLAST programs, the default parameters of the respective programs (e.g., XBLAST and NBLAST) can be used.

Similarly, in particular embodiments of the invention, two amino acid sequences are “substantially homologous” or “substantially similar” when greater than 90% of the amino acid residues are identical. Two sequences are functionally identical when greater than about 95% of the amino acid residues are similar. Preferably the similar or homologous polypeptide sequences are identified by alignment using, for example, the GCG (Genetics Computer Group, Version 7, Madison, Wis.) pileup program, or using any of the programs and algorithms described above. The program may use the local homology algorithm of Smith and Waterman with the default values: Gap creation penalty=−(1+1/k), k being the gap extension number, Average match=1, Average mismatch=−0.333.

The term “specific” in the context of “specific binding” is applicable to a situation in which one member of a specific binding pair will not show any significant binding to molecules other than its specific binding partner(s). The term is applicable, for example, to the situation where two complementary polynucleotide strands can anneal together, yet each single stranded polynucleotide exhibits little or no binding to other polynucleotide sequences under stringent hybridization conditions.

The term “regeneration” as used herein refers to any method of obtaining a whole plant from any one of a seed, a plant cell, a group of plant cells, plant callus tissue, or an excised piece of a plant.

As used herein, a “transgenic plant” is one whose genome has been altered by the incorporation of heterologous genetic material, e.g. by transformation as described herein. The term “transgenic plant” is used to refer to the plant produced from an original transformation event, or progeny from later generations or crosses of a transgenic plant, so long as the progeny contains the heterologous genetic material in its genome.

The term “transformation” or “transfection” refers to the transfer of one or more nucleic acid molecules into a host cell or organism. Methods of introducing nucleic acid molecules into host cells include, for instance, calcium phosphate transfection, DEAE-dextran mediated transfection, microinjection, cationic lipid-mediated transfection, electroporation, scrape loading, ballistic introduction or infection with viruses or other infectious agents.

“Transformed”, “transduced”, or “transgenic”, in the context of a cell, refers to a host cell or organism into which a recombinant or heterologous nucleic acid molecule (e.g., one or more DNA constructs or RNA, or siRNA counterparts) has been introduced. The nucleic acid molecule can be stably expressed (i.e. maintained in a functional form in the cell for longer than about three months) or non-stably maintained in a functional form in the cell for less than three months i.e. is transiently expressed. For example, “transformed,” “transformant,” and “transgenic” cells have been through the transformation process and contain foreign nucleic acid. The term “untransformed” refers to cells that have not been through the transformation process.

The term “vector” as used herein refers to a DNA or RNA molecule capable of replication in a host cell and/or to which another DNA or RNA segment can be operatively linked so as to bring about replication of the attached segment. A plasmid is an exemplary vector.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Buchanan et al., Biochemistry and Molecular Biology of Plants, Courier Companies, USA, 2000; Miki and Iyer, Plant Metabolism, 2^{nd} Ed. D. T. Dennis, D H Turpin, D D Lefebrve, D G Layzell (eds) Addison Wesly, Langgmans Ltd. London (1997); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

The publications discussed above are provided solely for their disclosure before the filing date of the present application. Nothing herein is to be construed as an admission that the invention is not entitled to antedate such disclosure by virtue of prior invention.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### Post-Harvest Physiological Deterioration (PPD)

The rapid development of post-harvest physiological deterioration in cassava is associated with mechanical damage which occurs during harvesting and handling operations. Tips are often broken off as the roots are pulled from the ground and severance from the plant necessarily creates a further wound. In most cases physiological deterioration develops from sites of tissue damage and is initially observed as blue-black discoloration of the vascular tissue which is often referred to as vascular streaking. Initial symptoms are rapidly followed by a more general discoloration of the storage parenchyma.

Tissue damage results in a cascade of wound responses that quickly result in the defense of the wounded tissue and the subsequent sealing of exposed tissue by regeneration of a protective barrier (periderm formation). Common wound responses directly involved in defense include lytic enzymes (glucanase and chitinase), protease inhibitor proteins, and hydroxyproline-rich glycoproteins production. Enzymes associated with the phenylpropanoid pathway, such as phenylalanine ammonia-lyase and chalcone synthase, lead to the biosynthesis of phenolics which can act directly as defense compounds (quinones, phytoalexins) or can form polymers, such as lignin, that render cell walls more resistant to water loss and attack from microbial enzymes.

Biochemical and molecular data show that the production and reactions of reactive oxygen species (ROS) are central to PPD. If oxygen is excluded from the root post-harvest then PPD can be substantially delayed. In developed countries, oxygen exclusion is achieved by waxing roots, but this strategy is too costly or unavailable for subsistence farmers in Africa. The accumulation of ROS during PPD is paralleled by an up-regulation of programmed cell death (PCD) and down-regulation of anti-PCD genes during PPD.

Without being bound by theory, it is hypothesized here that ROS production is associated with the poisoning of cytochrome C oxidase by cyanide that is generated following root excision. The subsequent over-reduction of mitochondrial complexes I and III leads to ROS generation. The present inventions employ molecular mechanisms for ROS inhibition and ROS scavenging in vasculature/laticifer tissues where cyanide generation is most intense following harvesting. Optionally, PPD is reduced by molecular mechanisms to limit PCD.

The present invention provides transgenic plants which exhibit reduced PPD. As used herein, the phrase “reduced PPD” means that the plant, or comestible portion thereof (e.g. cassava tuber) exhibits reduced propensity to incur PPD. The phrase “reduced PPD” is not intended to be limited to crops which have actually been harvested.

In some embodiments, a plant with reduced PPD is a plant in which the comestible (e.g. cassava tuber) exhibits a reduction in degree and/or a delay in onset after harvest of one or more of PPD symptoms listed in Table 1 (as compared to a non-transformed plant). Optionally, the reduction in degree of a symptom (e.g. % area discolored) is a reduction by at least about any of 40%, 70%, or 90%. Optionally, the delay in onset of a symptom (e.g. scopoletin autofluorescence) is a delay of at least about any of 3, 5, 10, 12, 15, 17, or 20 days. Optionally, the comestible (e.g. cassava root) is stored at low humidity, for example, less than 80%, 60%, 40%, or 20% humidity. Optionally, the reduction in degree of a symptom (e.g. % area discolored) is reduction of by at least about 70% (e.g. on day 5 post-harvest) and the delay in onset of a symptom (e.g. scopoletin autofluorescence) is a delay of at least about 3 days, and optionally, the comestible (e.g. cassava root) is stored in less than 80% humidity. Methods of quantifying such PPD symptoms are known in the art.

TABLE 1
PPD Symptoms

tissue disruption
discoloration
blue-black discoloration of xylem parenchyma (vascular
streaking)
general discoloration of the storage parenchyma
occlusions and/or tyloses in xylem parenchyma
scopoletin autofluorescence
changes associated with the plant's response to
wounding
Induced respiration, resulting in starch hydrolysis
dry weight of substantially unaffected tissue
suberization around wound sites

### AOX

In some embodiments, the invention provides a plant (e.g. cassava) that contains an AOX transgene that is functional in the plant. The AOX can be any AOX enzyme known in the art. AOX is an enzyme that diverts the flow of electrons through the electron transport chain from the phosphorylating cytochrome pathway (e.g. through cytochrome c) to the non-phosphorylating (alternative) pathway while catalyzing the oxidation of ubiquinol into ubiquinone and the reduction of oxygen to water.

Optionally, the AOX is any AOX set forth in Table 2. Optionally, the AOX exhibits a sequence identity of at least about any of 75%, 80%, 85%, 90%, or 95% to an AOX listed in Table 2, or an active fragment thereof. Optionally, the AOX is derived from any of the species set forth in Table 2.

Examplary AOX transgenes comprise one or more of the following features:

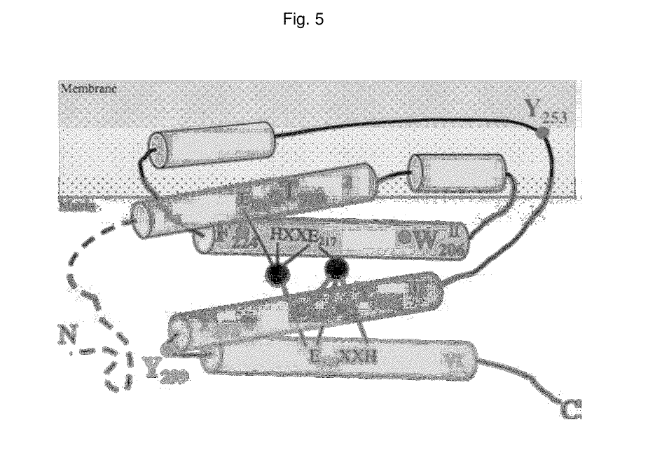

a. a quinol binding site;
b. a di-iron coordinating center (e.g. as shown in FIG. 5, in which iron atoms are represented by black circles);
c. a trans-membrane or membrane-interfacing segment (e.g. as shown in FIG. 5);
d. a four-helix bundle (e.g. as shown in FIG. 5);
e. one or more residues which are known to be conserved in examplary AOX's, for example, selected from the group consisting of: Tyr253, Tyr266, Tyr275, Tyr299, Trp206, Glu178, Glu268, Glu270, Thr179, His261, Arg262, His261/Arg262 dyad, Ser256, Gln242, Phe253, and Asp247; and
f. a MW of about 25 Kd to about 45 Kd.

The AOX can be derived from any organism, for example, a plant, fungus, protist, or lower invertebrate. Optionally, the plant is a higher plant (e.g. Arabidopsis), a monocot (e.g. nonthermogenic monocot), or a dicot (e.g. eudicot).

Optionally, the AOX is a cyanide insensitive AOX.

Optionally, the AOX transgene is a nuclear transgene.

Optionally, the AOX transgene is a mitochondrial transgene.

Optionally, the AOX transgene is an AOX1 (e.g. which is induced by stress stimuli in monocots and eudicots) or an AOX2 (e.g. constitutively or developmentally expressed in eudicots). Optionally, the AOX1 is an AOX1a, AOX1b, AOX1c, or AOX1d.

Optionally, the AOX is derived from an AOX from the genus Arum. Arum AOX enzymes that are especially useful according to the present invention are Arum-derived AOX enzymes that have a high catalytic activity.

Optionally, the AOX is derived from an AOX from the genus Zizania. Zizania AOX enzymes that are especially useful according to the present invention are Zizania-derived AOX enzymes that have a high affinity for oxygen.

Optionally, the AOX is derived from an AOX from Arabidopsis (AtAOX). The use of AtAOX provides one or more superior features. Plants transformed with an AtAOX can exhibit a surprisingly high capacity to inhibit PPD, for example, due to enhanced reduction of ROS in the transgenic plants.

A cassava comprising an AOX transgene of the present invention optionally exhibits lower reactive oxygen production.

Optionally, the AOX is operably linked to a comestible (e.g. root) specific promoter. Optionally, the AOX is operably linked to a patatin promoter.

Optionally, the AOX is operably linked to a terminator sequence (e.g. Nos terminator).

Optionally, the AOX is operably linked to a leader sequence (e.g. HSP70 leader).

Optionally, the AOX is operably linked to a terminator sequence (e.g. Nos terminator) and a terminator sequence (e.g. Nos terminator).

TABLE 2
AOX transgenes

| Accession | Entry name | Gene names | Organism |
|---|---|---|---|
| Q52RN3 | Q52RN3_ACTDE | | Actinidia deliciosa (Kiwi) |
| Q9Y711 | AOX_AJECA | AOX1 | Ajellomyces capsulata (Darling's disease fungus) (Histoplasma |
| | | | capsulatum) |
| C0NCS0 | C0NCS0_AJECG | HCBG_00916 | Ajellomyces capsulata (strain ATCC 26029/G186AR/H82/ |
| | | | RMSCC 2432) (Darling's disease fungus) (Histoplasma |
| | | | capsulatum) |
| C6HJJ6 | C6HJJ6_AJECH | HCDG_06377 | Ajellomyces capsulata (strain H143) (Darling's disease fungus) |
| | | | (Histoplasma capsulatum) |
| A6R263 | A6R263_AJECN | HCAG_03721 | Ajellomyces capsulata (strain NAm1/WU24) (Darling's disease |
| | | | fungus) (Histoplasma capsulatum) |
| C5GEN9 | C5GEN9_AJEDR | BDCG_02758 | Ajellomyces dermatitidis (strain ER-3) (Blastomyces |
| | | | dermatitidis) |
| C5JT97 | C5JT97_AJEDS | BDBG_05496 | Ajellomyces dermatitidis (strain SLH14081) (Blastomyces |
| | | | dermatitidis) |
| Q39219 | AOX1A_ARATH | AOX1A (AOX1) (At3g22370) | Arabidopsis thaliana (Mouse-ear cress) |
| | | (MCB17.11) | |
| O23913 | AOX1B_ARATH | AOX1B (At3g22360) (MCB17.10) | Arabidopsis thaliana (Mouse-ear cress) |
| O22048 | AOX1C_ARATH | AOX1C (At3g27620) (MGF10.3) | Arabidopsis thaliana (Mouse-ear cress) |
| O22049 | AOX2_ARATH | AOX2 (At5g64210) (MSJ1.5) | Arabidopsis thaliana (Mouse-ear cress) |
| Q8LEE7 | AOX3_ARATH | AOX3 (At1g32350) (F5D14.11) | Arabidopsis thaliana (Mouse-ear cress) |
| | | (F27G20_12) | |
| Q56X52 | AOX4_ARATH | AOX4 (IM) (PTOX) (At4g22260) | Arabidopsis thaliana (Mouse-ear cress) |
| | | (T10I14_90) | |
| Q08A65 | Q08A65_ARATH | | Arabidopsis thaliana (Mouse-ear cress) |
| Q9ZRT8 | Q9ZRT8_ARATH | hsr3 | Arabidopsis thaliana (Mouse-ear cress) |
| B9X258 | B9X258_9ARAE | AcoAOX1a | Arum concinnatum |
| B9X259 | B9X259_9ARAE | AcoAOX1b | Arum concinnatum |
| A1CCD7 | A1CCD7_ASPCL | ACLA_061560 | Aspergillus clavatus |
| A1CEG8 | A1CEG8_ASPCL | ACLA_089590 | Aspergillus clavatus |
| B8N494 | B8N494_ASPFN | AFLA_035070 | Aspergillus flavus (strain ATCC 200026/FGSC A1120/NRRL |
| | | | 3357/JCM 12722/SRRC 167) |
| B8NC42 | B8NC42_ASPFN | AFLA_038370 | Aspergillus flavus (strain ATCC 200026/FGSC A1120/NRRL |
| | | | 3357/JCM 12722/SRRC 167) |
| Q4WHK6 | Q4WHK6_ASPFU | AFUA_2G05060 | Aspergillus fumigatus (Sartorya fumigata) |
| Q8TFM2 | Q8TFM2_ASPFU | | Aspergillus fumigatus (Sartorya fumigata) |
| B0XVF7 | B0XVF7_ASPFC | AFUB_022090 | Aspergillus fumigatus (strain CEA10/CBS 144.89/FGSC A1163) |
| | | | (Sartorya fumigata) |
| O74180 | AOX_ASPNG | aox1 | Aspergillus niger |
| Q54AC8 | Q54AC8_ASPNG | aox1 | Aspergillus niger |
| A2QWD9 | A2QWD9_ASPNC | aox1 (An11g04810) | Aspergillus niger (strain CBS 513.88/FGSC A1513) |
| A2QXC9 | A2QXC9_ASPNC | An11g08460 | Aspergillus niger (strain CBS 513.88/FGSC A1513) |
| Q2U1I0 | Q2U1I0_ASPOR | AO090011000022 | Aspergillus oryzae |
| Q2ULQ6 | Q2ULQ6_ASPOR | AO090003000310 | Aspergillus oryzae |
| Q0CFU4 | Q0CFU4_ASPTN | ATEG_07440 | Aspergillus terreus (strain NIH 2624/FGSC A1156) |
| Q0CJY5 | Q0CJY5_ASPTN | ATEG_05999 | Aspergillus terreus (strain NIH 2624/FGSC A1156) |
| Q8X1N9 | AOX_BLUGR | | Blumeria graminis |
| Q8NJ59 | AOX_BOTFU | aox | Botryotinia fuckeliana (Noble rot fungus) (Botrytis cinerea) |
| A6RXS4 | A6RXS4_BOTFB | BC1G_05703 | Botryotinia fuckeliana (strain B05.10) (Noble rot fungus) |
| | | | (Botrytis cinerea) |
| O93853 | AOX1_CANAL | AOX1 (AOX1A) | Candida albicans (Yeast) |
| Q9UV71 | AOX2_CANAL | AOX2 (AOX1B) | Candida albicans (Yeast) |
| C4YDC2 | C4YDC2_CANAL | CAWG_00513 | Candida albicans (Yeast) |
| C4YDC3 | C4YDC3_CANAL | CAWG_00514 | Candida albicans (Yeast) |
| Q5APJ1 | Q5APJ1_CANAL | AOX1 (CaO19.12237) (CaO19.4774) | Candida albicans (Yeast) |
| Q5APJ2 | Q5APJ2_CANAL | AOX2 (CaO19.12236) | Candida albicans (Yeast) |
| Q5AQ35 | Q5AQ35_CANAL | AOX2 (CaO19.4773) | Candida albicans (Yeast) |
| B9W8T7 | B9W8T7_CANDC | CD36_08630 | Candida dubliniensis (strain CD36/CBS 7987/NCPF 3949/ |
| | | | NRRL Y-17841) (Yeast) |
| B9W8T8 | B9W8T8_CANDC | CD36_08640 | Candida dubliniensis (strain CD36/CBS 7987/NCPF 3949/ |
| | | | NRRL Y-17841) (Yeast) |
| Q564K1 | Q564K1_CANMA | Cm-AOX1a | Candida maltosa (Yeast) |
| Q564K2 | Q564K2_CANMA | Cm-AOX1b | Candida maltosa (Yeast) |
| C5MB28 | C5MB28_CANTT | CTRG_03270 | Candida tropicalis (strain ATCC MYA-3404/T1) (Yeast) |
| C5MB29 | C5MB29_CANTT | CTRG_03271 | Candida tropicalis (strain ATCC MYA-3404/T1) (Yeast) |
| Q6X812 | Q6X812_CAPAN | | Capsicum annuum (Bell pepper) |
| Q9FZ04 | Q9FZ04_CAPAN | PTOX | Capsicum annuum (Bell pepper) |
| O48519 | O48519_CATRO | | Catharanthus roseus (Madagascar periwinkle) (Vinca rosea) |
| Q9AYP1 | Q9AYP1_CATRO | | Catharanthus roseus (Madagascar periwinkle) (Vinca rosea) |
| Q2GZF3 | Q2GZF3_CHAGB | CHGG_05093 | Chaetomium globosum (Soil fungus) |
| Q2GZL4 | Q2GZL4_CHAGB | CHGG_05032 | Chaetomium globosum (Soil fungus) |
| Q1WLY7 | Q1WLY7_CHLIN | AOX1 | Chlamydomonas incerta |
| O65000 | O65000_CHLRE | AOX1 (CHLREDRAFT_129968) | Chlamydomonas reinhardtii |
| Q9FE26 | Q9FE26_CHLRE | AOX2 (CHLREDRAFT_77667) | Chlamydomonas reinhardtii |
| O48514 | O48514_CHLSW | | Chlamydomonas sp. (strain W80) |
| B3VA07 | B3VA07_CITSI | Aox1b | Citrus sinensis (Sweet orange) |
| B3VA08 | B3VA08_CITSI | Aox2 | Citrus sinensis (Sweet orange) |
| B3VA09 | B3VA09_CITSI | Aox1a | Citrus sinensis (Sweet orange) |
| C4Y1G0 | C4Y1G0_CLAL4 | CLUG_02042 | Clavispora lusitaniae (strain ATCC 42720) (Yeast) (Candida |
| | | | lusitaniae) |
| Q1E8R2 | Q1E8R2_COCIM | CIMG_01051 | Coccidioides immitis (Valley fever fungus) |
| C5PHD7 | C5PHD7_COCP7 | CPC735_053100 | Coccidioides posadasii (strain C735) (Valley fever fungus) |
| Q2WBI4 | Q2WBI4_COCNU | CLM | Cocos nucifera (Coconut) |
| Q0H3C8 | Q0H3C8_COFCA | PTOX | Coffea canephora (Robusta coffee) |
| A8NDS4 | A8NDS4_COPC7 | CC1G_10695 | Coprinopsis cinerea (strain Okayama-7/130/FGSC 9003) (Inky |
| | | | cap fungus) (Hormographiella aspergillata) |
| A8NQU0 | A8NQU0_COPC7 | CC1G_03463 | Coprinopsis cinerea (strain Okayama-7/130/FGSC 9003) (Inky |
| | | | cap fungus) (Hormographiella aspergillata) |
| A8QJP8 | A8QJP8_CRIPE | | Crinipellis perniciosa (Witches'-broom disease fungus) |
| | | | (Marasmius perniciosus) |
| Q84KA1 | Q84KA1_CROSA | | Crocus sativus (Saffron) |
| Q5KPU5 | Q5KPU5_CRYNE | CNA01500 (CNBA1450) | Cryptococcus neoformans (Filobasidiella neoformans) |
| Q8NKE2 | AOX_CRYNV | AOX1 | Cryptococcus neoformans var. grubii (Filobasidiella neoformans) |
| | | | var. grubii) |
| A1BQM5 | A1BQM5_CUCSA | | Cucumis sativus (Cucumber) |
| Q7Y1A3 | Q7Y1A3_CUCSA | | Cucumis sativus (Cucumber) |
| Q7Y1B3 | Q7Y1B3_CUCSA | aox2 | Cucumis sativus (Cucumber) |
| A9P3L0 | A9P3L0_CUCPE | | Cucurbita pepo (Vegetable marrow) (Summer squash) |
| B1P5D1 | B1P5D1_DAUCA | AOX1a | Daucus carota (Carrot) |
| B1P5D3 | B1P5D3_DAUCA | AOX2a | Daucus carota (Carrot) |
| B1P5D4 | B1P5D4_DAUCA | AOX2b | Daucus carota (Carrot) |
| Q6BVC1 | Q6BVC1_DEBHA | DEHA2C03828g | Debaryomyces hansenii (Yeast) (Torulaspora hansenii) |
| Q65YS0 | Q65YS0_9ARAE | DvAOX | Dracunculus vulgaris |
| Q9P959 | AOX_EMENI | alxA (aod-1) (AN2099) | Emericella nidulans (Aspergillus nidulans) |
| Q8J1Z2 | AOX_GELSS | aod-1 | Gelasinospora sp. (strain S23) |
| Q07185 | AOX1_SOYBN | AOX1 | Glycine max (Soybean) |
| Q41266 | AOX2_SOYBN | AOX2 | Glycine max (Soybean) |
| O03376 | AOX3_SOYBN | AOX3 | Glycine max (Soybean) |
| C6T8G8 | C6T8G8_SOYBN | | Glycine max (Soybean) |
| O82518 | O82518_SOYBN | Aox1 | Glycine max (Soybean) |
| Q41267 | Q41267_SOYBN | Aox3 | Glycine max (Soybean) |
| Q7XZQ0 | Q7XZQ0_SOYBN | Aox2a | Glycine max (Soybean) |
| Q7XZQ1 | Q7XZQ1_SOYBN | Aox2b | Glycine max (Soybean) |
| B3RH48 | B3RH48_GOSHI | AOX1 | Gossypium hirsutum (Upland cotton) (Gossypium mexicanum) |
| Q1A7V9 | Q1A7V9_GOSHI | AOX1 | Gossypium hirsutum (Upland cotton) (Gossypium mexicanum) |
| Q00912 | AOX_HANAN | AOX1 (ALX1) | Hansenula anomala (Yeast) (Candida pelliculosa) |
| B2Y052 | B2Y052_HYPPE | AOX2 | Hypericum perforatum (St. John's wort) |
| B2Y053 | B2Y053_HYPPE | AOX1a | Hypericum perforatum (St. John's wort) |
| B2Y054 | B2Y054_HYPPE | AOX1b | Hypericum perforatum (St. John's wort) |
| B2Y055 | B2Y055_HYPPE | AOX1c | Hypericum perforatum (St. John's wort) |
| B0CVV3 | B0CVV3_LACBS | LACBIDRAFT_309224 | Laccaria bicolor (strain S238N-H82) (Bicoloured deceiver) |
| | | | (Laccaria laccata var. bicolor) |
| Q0WX68 | Q0WX68_LACSA | AOX | Lactuca sativa (Garden lettuce) |
| A4HPV5 | A4HPV5_LEIBR | LbrM35_V2.4620 | Leishmania braziliensis |
| A5E1V7 | A5E1V7_LODEL | LELG_03594 | Lodderomyces elongisporus (Yeast) (Saccharomyces |
| | | | elongisporus) |
| O93788 | AOX_MAGGR | AOX1 (MGG_12936) | Magnaporthe grisea (Rice blast fungus) (Pyricularia grisea) |
| Q40294 | AOX1_MANIN | AOMI 1 | Mangifera indica (Mango) |
| Q94FU7 | Q94FU7_MANIN | | Mangifera indica (Mango) |
| Q94FU8 | Q94FU8_MANIN | | Mangifera indica (Mango) |
| Q94FU9 | Q94FU9_MANIN | | Mangifera indica (Mango) |
| Q94FV0 | Q94FV0_MANIN | | Mangifera indica (Mango) |
| Q2HTI6 | Q2HTI6_MEDTR | MtrDRAFT_AC150441g3v1 | Medicago truncatula (Barrel medic) |
| Q45N56 | Q45N56_METAN | | Metarhizium anisopliae |
| Q96UR9 | AOX_MONFR | AOX1 | Monilinia fructicola |
| C5FPX3 | C5FPX3_NANOT | MCYG_04745 | Nannizzia otae (strain CBS 113480) (Microsporum canis) |
| | | | (Arthroderma otae) |
| C0SUJ4 | C0SUJ4_NELNU | NnAOX1a | Nelumbo nucifera (Sacred lotus) |
| C0SUJ5 | C0SUJ5_NELNU | NnAOX1b | Nelumbo nucifera (Sacred lotus) |
| A1DFS4 | A1DFS4_NEOFI | NFIA_081770 | Neosartorya fischeri (strain ATCC 1020/DSM 3700/FGSC |
| | | | A1164/NRRL 181) (Aspergillus fischerianus) |
| Q01355 | AOX_NEUCR | and-1 (NCU07953) | Neurospora crassa |
| Q7S371 | Q7S371_NEUCR | NCU04874 (NCU04874.1) | Neurospora crassa |
| Q676U2 | Q676U2_9SOLA | AOX2 | Nicotiana attenuata |
| Q676U3 | Q676U3_9SOLA | AOX1 | Nicotiana attenuata |
| C1I1U5 | C1I1U5_NICGU | | Nicotiana glutinosa (Tobacco) |
| A0JCI0 | A0JCI0_TOBAC | AOX | Nicotiana tabacum (Common tobacco) |
| Q41224 | A0X1_TOBAC | AOX1 | Nicotiana tabacum (Common tobacco) |
| Q40578 | A0X2_TOBAC | AOX2 | Nicotiana tabacum (Common tobacco) |
| Q01HU3 | Q01HU3_ORYSA | B0403H10-OSIGBa0105A11.5 | Oryza sativa (Rice) |
| Q01IM2 | Q01IM2_ORYSA | OSIGBa0143N19.12 | Oryza sativa (Rice) |
| Q259P9 | Q259P9_ORYSA | H0818H01.2 (B0811B10.18) | Oryza sativa (Rice) |
| Q7GDL6 | Q7GDL6_ORYSA | Aox1(Ao1-1) (B0403H10- | Oryza sativa (Rice) |
| | | OSIGBa0105A11.4) | |
| Q8SBA9 | Q8SBA9_ORYSA | OSJNBa0030B02.13 | Oryza sativa (Rice) |
| A2X418 | A2X418_ORYSI | OsI_06950 | Oryza sativa subsp. indica (Rice) |
| A2X8M7 | A2X8M7_ORYSI | OsI_08589 | Oryza sativa subsp. indica (Rice) |
| A2XX54 | A2XX54_ORYSI | OsI_17254 | Oryza sativa subsp. indica (Rice) |
| A2XX55 | A2XX55_ORYSI | OsI_17255 | Oryza sativa subsp. indica (Rice) |
| B8ASV2 | B8ASV2_ORYSI | OsI_16922 | Oryza sativa subsp. indica (Rice) |
| B8AW16 | B8AW16_ORYSI | OsI_17850 | Oryza sativa subsp. indica (Rice) |
| B8AZ29 | B8AZ29_ORYSI | OsI_20294 | Oryza sativa subsp. indica (Rice) |
| B8B2W2 | B8B2W2_ORYSI | OsI_23112 | Oryza sativa subsp. indica (Rice) |
| B8B3F1 | B8B3F1_ORYSI | OsI_21855 | Oryza sativa subsp. indica (Rice) |
| A3A642 | A3A642_ORYSJ | OsJ_06456 | Oryza sativa subsp. japonica (Rice) |
| A3AAF8 | A3AAF8_ORYSJ | OsJ_08049 | Oryza sativa subsp. japonica (Rice) |
| A3AAF9 | A3AAF9_ORYSJ | OsJ_08050 | Oryza sativa subsp. japonica (Rice) |
| B9FKP7 | B9FKP7_ORYSJ | OsJ_18878 | Oryza sativa subsp. japonica (Rice) |
| B9FRS0 | B9FRS0_ORYSJ | OsJ_20292 | Oryza sativa subsp. japonica (Rice) |
| B9G7I5 | B9G7I5_ORYSJ | OsJ_30719 | Oryza sativa subsp. japonica (Rice) |
| B9GBB0 | B9GBB0_ORYSJ | OsJ_34333 | Oryza sativa subsp. japonica (Rice) |
| O82522 | O82522_ORYSJ | IM1 (OSJNBa0043A12.16) | Oryza sativa subsp. japonica (Rice) |
| | | (Os04g0668900) (OsJ_16557) | |
| O82766 | O82766_ORYSJ | AOX1b (OSJNBa0083N12.12) | Oryza sativa subsp. japonica (Rice) |
| | | (Os04g0600300) (OsJ_16033) | |
| O82807 | 082807_ORYSJ | AOX1a (OSJNBa0083N12.11) | Oryza sativa subsp. japonica (Rice) |
| | | (Os04g0600200) (OsJ_16032) | |
| Q33B30 | 033B30_ORYSJ | LOC_Os10g05620 | Oryza sativa subsp. japonica (Rice) |
| Q6EUL9 | Q6EUL9_ORYSJ | OJ1134_B09.10 | Oryza sativa subsp. japonica (Rice) |
| Q7XT33 | Q7XT33_ORYSJ | OSJNBa0010H02.19 | Oryza sativa subsp. japonica (Rice) |
| Q8W855 | Q8W855_ORYSJ | AOX1c (0J1111_E07.10) | Oryza sativa subsp. japonica (Rice) |
| | | (Os02g0700400) (P0459B01.39) | |
| A4S238 | A4S238_OSTLU | OSTLU_4970 (OSTLU_4977) | Ostreococcus lucimarinus (strain CCE9901) |
| B8XFS6 | B8XFS6_9EURO | alxA | Paecilomyces sp. J18 |
| C1GRD3 | C1GRD3_PARBA | PAAG_01078 | Paracoccidioides brasiliensis (strain ATCC MYA-826/Pb01) |
| C0S246 | C0S246_PARBP | PABG_01661 | Paracoccidioides brasiliensis (strain Pb03) |
| C1G911 | C1G911_PARBD | PADG_03747 | Paracoccidioides brasiliensis (strain Pb18) |
| Q6TBA7 | Q6TBA7_PENCH | aox | Penicillium chrysogenum (Penicillium notatum) |
| B6H0Q2 | B6H0Q2_PENCW | Pc12g10440 | Penicillium chrysogenum (strain ATCC 28089/DSM 1075/ |
| | | | Wisconsin 54-1255) (Penicillium notatum) |
| B6HCR7 | B6HCR7_PENCW | Pc18g06440 | Penicillium chrysogenum (strain ATCC 28089/DSM 1075/ |
| | | | Wisconsin 54-1255) (Penicillium notatum) |
| B6Q3L8 | B6Q3L8_PENMQ | PMAA_029240 | Penicillium marneffei (strain ATCC 18224/CBS 334.59/QM |
| | | | 7333) |
| Q6QUX0 | Q6QUX0_PETHY | | Petunia hybrida (Petunia) |
| Q6QUX1 | Q6QUX1_PETHY | | Petunia hybrida (Petunia) |
| Q6QUX2 | Q6QUX2_PETHY | | Petunia hybrida (Petunia) |
| Q6QUX3 | Q6QUX3_PETHY | | Petunia hybrida (Petunia) |
| Q6QUX4 | Q6QUX4_PETHY | | Petunia hybrida (Petunia) |
| Q0UYY3 | Q0UYY3_PHANO | SNOG_03031 | Phaeosphaeria nodorum (Glume blotch fungus) (Septoria |
| | | | nodorum) |
| Q3LFQ3 | Q3LFQ3_PHAVU | AOX | Phaseolus vulgaris (Kidney bean) (French bean) |
| Q65YQ8 | Q65YQ8_9ARAE | PsAOX | Philodendron bipinnatifidum |
| A9SJF9 | A9SJF9_PHYPA | PHYPADRAFT_130694 | Physcomitrella patens subsp. patens |
| A9T643 | A9T643_PHYPA | PHYPADRAFT_33582 | Physcomitrella patens subsp. patens |
| A9T8C5 | A9T8C5_PHYPA | PHYPADRAFT_89421 | Physcomitrella patens subsp. patens |
| A9NQ56 | A9NQ56_PICSI | | Picea sitchensis (Sitka spruce) |
| A9NRS5 | A9NRS5_PICSI | | Picea sitchensis (Sitka spruce) |
| A5DR92 | A5DR92_PICGU | PGUG_05793 | Pichia guilliermondii (Yeast) (Candida guilliermondii) |
| C4R4H1 | C4R4H1_PICPG | PAS_chr3_0408 | Pichia pastoris (strain GS115) (Yeast) |
| A4K8T8 | A4K8T8_PICPA | AOX | Pichia pastoris (Yeast) |
| Q9P414 | AOX_PICST | STO1 (AOX1) (PICST_67332) | Pichia stipitis (Yeast) |
| Q9C206 | AOX_PODAN | AOX1 (AOX) | Podospora anserina |
| B2ACQ1 | B2ACQ1_PODAN | | Podospora anserina |
| Q9P492 | Q9P492_PODAN | A0X | Podospora anserina |
| Q9M432 | Q9M432_9ROSI | aox1b | Populus tremula x Populus tremuloides |
| Q9SC31 | Q9SC31_9ROSI | aox1 | Populus tremula x Populus tremuloides |
| B9GZX6 | B9GZX6_POPTR | POPTRDRAFT_414439 | Populus trichocarpa (Western balsam poplar) (Populus |
| | | | balsamifera subsp. trichocarpa) |
| B9HZH9 | B9HZH9_POPTR | POPTRDRAFT_1093171 | Populus trichocarpa (Western balsam poplar) (Populus |
| | | | balsamifera subsp. trichocarpa) |
| B9I558 | B9I558_POPTR | POPTRDRAFT_891190 | Populus trichocarpa (Western balsam poplar) (Populus |
| | | | balsamifera subsp. trichocarpa) |
| B9I559 | B9I559_POPTR | POPTRDRAFT_422024 | Populus trichocarpa (Western balsam poplar) (Populus |
| | | | balsamifera subsp. trichocarpa) |
| B9IDX2 | B9IDX2_POPTR | POPTRDRAFT_732929 | Populus trichocarpa (Western balsam poplar) (Populus |
| | | | balsamifera subsp. trichocarpa) |
| B9N8H5 | B9N8H5_POPTR | POPTRDRAFT_1117798 | Populus trichocarpa (Western balsam poplar) (Populus |
| | | | balsamifera subsp. trichocarpa) |
| B2WNP7 | B2WNP7_PYRTR | PTRG_11607 | Pyrenophora tritici-repentis (strain Pt-1C-BFP) (Wheat tan spot |
| | | | fungus) (Drechslera tritici-repentis) |
| B9RXE2 | B9RXE2_RICCO | RCOM_0903090 | Ricinus communis ( Castor bean) |
| B9S038 | B9S038_RICCO | RCOM_1296790 | Ricinus communis ( Castor bean) |
| B9TN59 | B9TN59_RICCO | RCOM_1998260 | Ricinus communis ( Castor bean) |
| Q66PW9 | Q66PW9_SACOF | | Saccharum officinarum (Sugarcane) |
| Q66PX0 | Q66PX0_SACOF | | Saccharum officinarum (Sugarcane) |
| Q66PX1 | Q66PX1_SACOF | | Saccharum officinarum (Sugarcane) |
| Q66PX2 | Q66PX2_SACOF | | Saccharum officinarum (Sugarcane) |
| P22185 | AOX1_SAUGU | AOX1 | Sauromatum guttatum (Voodoo lily) (Sauromatum venosum) |
| A7EC44 | A7EC44_SCLS1 | SS1G_02882 | Sclerotinia sclerotiorum (strain ATCC 18683/1980/Ss-1) |
| | | | (White mold) (Whetzelinia sclerotiorum) |
| B0ZZ00 | B0ZZ00_SOLLC | | Solanum lycopersicum (Tomato) (Lycopersicon esculentum) |
| Q5F0I2 | Q5F0I2_SOLLC | | Solanum lycopersicum (Tomato) (Lycopersicon esculentum) |
| Q7XBG8 | Q7XBG8_SOLLC | | Solanum lycopersicum (Tomato) (Lycopersicon esculentum) |
| Q7XBG9 | Q7XBG9_SOLLC | | Solanum lycopersicum (Tomato) (Lycopersicon esculentum) |
| Q84V46 | Q84V46_SOLLC | | Solanum lycopersicum (Tomato) (Lycopersicon esculentum) |
| Q84V47 | Q84V47_SOLLC | | Solanum lycopersicum (Tomato) (Lycopersicon esculentum) |
| Q9FEC9 | Q9FEC9_SOLLC | PTOX | Solanum lycopersicum (Tomato) (Lycopersicon esculentum) |
| Q1XIH9 | Q1XIH9_SOLTU | AOX | Solanum tuberosum (Potato) |
| Q2XPM9 | Q2XPM9_SOLTU | | Solanum tuberosum (Potato) |
| C5Y0E2 | C5Y0E2_SORBI | Sb04g030820 | Sorghum bicolor (Sorghum) (Sorghum vulgare) |
| | | (SORBIDRAFT_04g030820) | |
| C5YF55 | C5YF55_SORBI | Sb06g027410 | Sorghum bicolor (Sorghum) (Sorghum vulgare) |
| | | (SORBIDRAFT_06g027410) | |
| C5YF56 | C5YF56_SORBI | Sb06g027420 | Sorghum bicolor (Sorghum) (Sorghum vulgare) |
| | | (SORBIDRAFT_06g027420) | |
| C5YF57 | C5YF57_SORBI | Sb06g027430 | Sorghum bicolor (Sorghum) (Sorghum vulgare) |
| | | (SORBIDRAFT_06g027430) | |
| Q5KSN9 | Q5KSN9_9ARAE | SrAOX | Symplocarpus renifolius |
| B8LT09 | B8LT09_TALSN | TSTA_069380 | Talaromyces stipitatus (strain ATCC 10500/CBS 375.48/QM |
| | | | 6759/NRRL 1006) (Penicillium stipitatum) |
| Q8S913 | Q8S913_WHEAT | Waox1c | Triticum aestivum (Wheat) |
| Q8S914 | Q85914_WHEAT | Waox1a | Triticum aestivum (Wheat) |
| Q9SW79 | Q9SW79_WHEAT | | Triticum aestivum (Wheat) |
| O96711 | O96711_9TRYP | | Trypanosoma brucei |
| Q38A00 | Q38A00_9TRYP | Tb10.6k15.0550 | Trypanosoma brucei |
| Q38AQ4 | Q38AQ4_9TRYP | Tb10.6k15.3640 | Trypanosoma brucei |
| Q26710 | AOX_TRYBB | AOX | Trypanosoma brucei brucei |
| Q4ZG96 | Q4ZG96_TRYBG | AOX | Trypanosoma brucei gambiense |
| Q1EPV4 | Q1EPV4_TRYBR | AOX | Trypanosoma brucei rhodesiense |
| Q4AEA1 | Q4AEA1_TRYCO | aox | Trypanosoma congolense |
| Q4AEA2 | Q4AEA2_TRYCO | aox | Trypanosoma congolense |
| Q4AEA3 | Q4AEA3_TRYCO | aox | Trypanosoma congolense |
| Q4DZM7 | Q4DZM7_TRYCR | aox (Tc00.1047053504147.180) | Trypanosoma cruzi |
| Q4AEA0 | Q4AEA0_9TRYP | aox | Trypanosoma evansi |
| Q76LX0 | Q76LX0_TRYVI | TVAOX | Trypanosoma vivax |
| C4JFI2 | C4JFI2_UNCRE | UREG_00996 | Uncinocarpus reesii (strain UAMH 1704) |
| Q4PAT9 | Q4PAT9_USTMA | UM02774.1 | Ustilago maydis (Smut fungus) |
| Q9P429 | AOX_VENIN | AOX1 | Venturia inaequalis (Apple scab fungus) |
| A2IBG3 | A2IBG3_VIGUN | Aox2a | Vigna unguiculata (Cowpea) |
| Q4F8G4 | Q4F8G4_VIGUN | | Vigna unguiculata (Cowpea) |
| Q93X12 | Q93X12_VIGUN | aox | Vigna unguiculata (Cowpea) |
| A5BPB4 | A5BPB4_VITVI | VITISV_018234 | Vitis vinifera (Grape) |
| A5BUW6 | A5BUW6_VITVI | VITISV_001908 | Vitis vinifera (Grape) |
| A7QIH9 | A7QIH9_VITVI | GSVIVT00000267001 | Vitis vinifera (Grape) |
| A7QQ22 | A7QQ22_VITVI | GSVIVT00003172001 | Vitis vinifera (Grape) |
| A7QQ23 | A7QQ23_VITVI | GSVIVT00003173001 | Vitis vinifera (Grape) |
| B2CKL9 | B2CKL9_VITVI | Aox2 | Vitis vinifera (Grape) |
| B6CGN7 | B6CGN7_VITVI | Aox2 | Vitis vinifera (Grape) |
| B6CGN8 | B6CGN8_VITVI | Aox1a | Vitis vinifera (Grape) |
| B6CGN9 | B6CGN9_VITVI | Aox1b | Vitis vinifera (Grape) |
| B6CGP0 | B6CGP0_VITVI | Aox1a | Vitis vinifera (Grape) |
| B6CGP1 | B6CGP1_VITVI | Aox1b | Vitis vinifera (Grape) |
| B6CGP2 | B6CGP2_VITVI | Aox1a | Vitis vinifera (Grape) |
| B6CGP3 | B6CGP3_VITVI | Aox1b | Vitis vinifera (Grape) |
| B6CGP4 | B6CGP4_VITVI | Aox1b | Vitis vinifera (Grape) |
| B6CGP5 | B6CGP5_VITVI | Aox2 | Vitis vinifera (Grape) |
| B6CGP7 | B6CGP7_VITVI | Aox1a | Vitis vinifera (Grape) |
| B6CGP8 | B6CGP8_VITVI | Aox1b | Vitis vinifera (Grape) |
| Q8J0I8 | AOX_YARLI | AOX (YALI0E00814g) | Yarrowia lipolytica (Candida lipolytica) |
| Q2UZR4 | Q2UZR4_YARLI | | Yarrowia lipolytica (Candida lipolytica) |
| Q2UZR5 | Q2UZR5_YARLI | | Yarrowia lipolytica (Candida lipolytica) |
| Q6C9M5 | Q6C9M5_YARLI | YALI0D09933g | Yarrowia lipolytica (Candida lipolytica) |
| B6SUK4 | B6SUK4_MAIZE | | Zea mays (Maize) |
| B6TMK6 | B6TMK6_MAIZE | | Zea mays (Maize) |
| O49161 | O49161_MAIZE | Aox | Zea mays (Maize) |
| Q8GT27 | Q8GT27_MAIZE | aox1 | Zea mays (Maize) |
| Q8GT70 | Q8GT70_MAIZE | aox3 | Zea mays (Maize) |
| Q8GT71 | Q8GT71_MAIZE | aox2 (Aox1a) | Zea mays (Maize) |

### Anti-PCD Transgenes

In some embodiments, the invention provides a cassava containing one or more anti-PCD transgenes functional in cassava. Examples of useful anti-PCD genes are Bcl-2, Bcl-xl, mcl-1, XIAP, crmA, Hsp10, 4F2 and pyridoxal kinase, PpBI-1 (e.g. as isolated from Phyllostachys praecox), and Ced-9 anti-apoptosis genes, together with the plant functional homologue, AtBAG4.

### Antioxidation Products

With the teachings provided herein, it is now evident that ROS mediate PPD and that cyanide-dependent inhibition of cytochrome C oxidase activity leads to copious ROS production. As taught herein, expression of an AOX transgene significantly reduces ROS production and PPD. Further surprisingly, however, such transgenic plants can still produce non-trivial levels of ROS. Accordingly, in one embodiment, the invention provides a plant (e.g. cassava) which overexpresses one or more antioxidation products alone or in combination with AOX or other transgene(s) taught herein. Optionally, the one or more antioxidation products are selected from ROS scavengers and carotenoid (or other isoprenoid) biosynthesis genes (referred to here simply as “carotenoid biosynthesis genes”).

### ROS Scavengers

In some embodiments, cassava (or other crop) contains one or more reactive oxygen species (ROS) scavengers (e.g. transgenes), that is, an agent that through an enzymatic or physicochemical property, results in the decrease in the level of ROS. While the ROS scavenger can be any ROS scavenger functional in cassava, examples are superoxide dismutase, catalase, ascorbate peroxidase, D-galacturonic acid reductase, γ-glutamylcysteine synthase, dehydroascorbate reductase, glutathione peroxidase, and glutathione reductase.

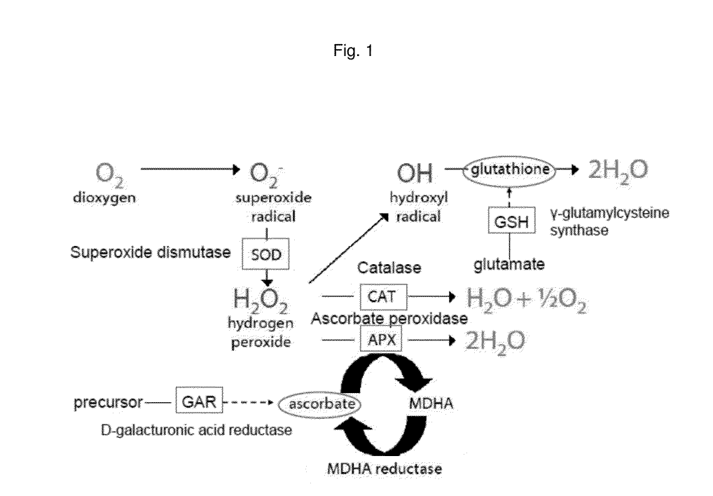

As depicted in FIG. 1, ROS scavengers act independently or in concert to reduce ROS's to species that are less prone to contribute to PPD.

Optionally, the ROS scavenger is operably linked to a comestible (e.g. root) specific promoter. Optionally, the ROS scavenger is operably linked to a patatin promoter.

Optionally, the ROS scavenger is operably linked to a leader sequence (e.g. HSP70 leader).

### Carotenoids

Some embodiments of the present invention provide a plant (e.g. cassava) containing a carotenoid biosynthesis gene. Carotenoids and isoprenoids (simply referred to herein as “carotenoids”) represent a widely distributed class of natural antioxidants and are synthesized by all plants, as well as some bacteria and fungi. The carotenoids are part of the larger isoprenoid biosynthesis pathway which, in addition to carotenoids, produces such compounds as chlorophyll and tocopherols, Vitamin E active agents. The carotenoid pathway in plants produces, for example, carotenes, such as α- and β-carotene, and lycopene, and xanthophylls, such as lutein.

### Phytoene Synthase

In some embodiments, cassava contains a phytoene synthase (PSY) transgene functional in cassava. The PSY can be any transferase enzyme that catalyzes the conversion of geranylgeranyl pyrophosphate to phytoene when expressed in cassava.

Optionally, the PSY is any PSY set forth in Table 3. Optionally, the PSY exhibits a sequence identity of at least about any of 75%, 80%, 85%, 90%, or 95% to a PSY listed in Table 3.

Examplary PSY transgenes comprise one or more of the following features:

g. a trans-Isoprenyl Diphosphate Synthase domain;
h. a large central cavity formed by mostly antiparallel alpha helices with two aspartate-rich regions (e.g. DXXXD) located on opposite walls;
i. an isoprenoid synthase fold;
j. MG2+ binding site;
k. active site lid residues.

Optionally, the PSY is a plant, bacterial, or fungal PSY.

Useful PSY transgenes can be isolated from any organism, such as Lycopersicon (e.g. L. esculentum), Mycoibacterium, Capsicum (e.g. C. annum) such as EC 2.5.1.1 and/or EC 2.5.1.32, Synechococcus, Erwinia (e.g. uredovora) such as 20D3, ATTC 19321, Narcissus (e.g. N. pseudonarcissus), Erwinia (e.g. E. herbicol), Sinapis (e.g. S. alba), Haematococcus (e.g. H. pluvialis), or maize:

Optionally, the PSY is a PSY1, PSY2, or PSY3.

Classes of PSY that are especially useful include those from maize that have high intrinsic activity.

Optionally, the PSY is derived from any of the species set forth in Table 3.

Optionally, the PSY gene sequence comprises Error! Reference source not found. or Error! Reference source not found., or derivative thereof. Optionally, the PSY protein sequence comprises the sequence encoded by Error! Reference source not found. or Error! Reference source not found., or derivative thereof.

Optionally, the PSY comprises a transit sequence. Optionally, the transit sequence is a plastid-transit sequence. Optionally, the plastid sequence is encoded by Error! Reference source not found., or derivative thereof.

Optionally, the PSY is operably linked to a comestible (e.g. root) specific promoter. Optionally, the PSY is operably linked to a patatin promoter.

TABLE 3
PSY Transgenes

| C5H5H0 | C5H5H0_MANES | Phytoene synthase (EC 2.5.1.32) | | Manihot esculenta (Cassava) (Manioc) |
|---|---|---|---|---|
| | | (Fragment) | | |
| Q5ISE0 | Q5ISE0_9MAGN | Phytoene synthase (EC 2.5.1.32) | Psy | Adonis aestivalis var. palaestina |
| | | (Fragment) | | |
| Q8KP35 | Q8KP35_AGRME | Phytoene synthase (EC 2.5.1.32) | crtB | Agromyces mediolanus (Corynebacterium |
| | | | | mediolanum) |
| C8WSG2 | C8WSG2_ALIAC | Phytoene synthase (EC 2.5.1.32) | Aaci_2441 | Alicyclobacillus acidocaldarius subsp. |
| | | | | acidocaldarius (strain ATCC 27009/DSM |
| | | | | 446/104-1A) (Bacillus acidocaldarius) |
| C8WSG3 | C8WSG3_ALIAC | Squalene synthase HpnC (EC | Aaci_2442 | Alicyclobacillus acidocaldarius subsp. |
| | | 2.5.1.32) | | acidocaldarius (strain ATCC 27009/DSM |
| | | | | 446/104-1A) (Bacillus acidocaldarius) |
| C8WUK7 | C8WUK7_ALIAC | Phytoene synthase (EC 2.5.1.32) | Aaci_2819 | Alicyclobacillus acidocaldarius subsp. |
| | | | | acidocaldarius (strain ATCC 27009/DSM |
| | | | | 446/104-1A) (Bacillus acidocaldarius) |
| Q3M3P5 | Q3M3P5_ANAVT | Phytoene synthase (EC 2.5.1.32) | Ava_4794 | Anabaena variabilis (strain ATCC 29413/ |
| | | | | PCC 7937) |
| P37271 | PSY_ARATH | Phytoene synthase, chloroplastic | PSY1 (PSY) | Arabidopsis thaliana (Mouse-ear cress) |
| | | (EC 2.5.1.32) | (At5g17230) | |
| | | | (MKP11.8) | |
| A2QM49 | A2QM49_ASPNC | Contig An07c0010, complete | An07g00800 | Aspergillus niger (strain CBS 513.88/FGSC |
| | | genome. (EC 2.5.1.32) | | A1513) |
| A2R1F4 | A2R1F4_ASPNC | Catalytic activity: heterotrimeric | An13g01040 | Aspergillus niger (strain CBS 513.88/FGSC |
| | | RABGGT of H. sapiens | | A1513) |
| | | (EC 2.5.1.32) | | |
| A2RBD5 | A2RBD5_ASPNC | Contig An18c0200, complete | An18g06340 | Aspergillus niger (strain CBS 513.88/FGSC |
| | | genome. (EC 2.5.1.32) | | A1513) |
| A1K6P5 | A1K6P5_AZOSB | Putative phytoene synthase (EC | crtB1 (azo1883) | Azoarcus sp. (strain BH72) |
| | | 2.5.1.32) | | |
| A1K6P6 | A1K6P6_AZOSB | Putative phytoene synthase (EC | crtB2 (azo1884) | Azoarcus sp. (strain BH72) |
| | | 2.5.1.32) | | |
| A8FBS9 | A8FBS9_BACP2 | Phytoene synthase (EC 2.5.1.32) | yisP (BPUM_1012) | Bacillus pumilus (strain SAFR-032) |
| A9IS32 | A9IS32_BART1 | Phytoene synthase (EC 2.5.1.32) | crtB (BT_0850) | Bartonella tribocorum (strain CIP 105476/ |
| | | | | IBS 506) |
| Q6MMB1 | Q6MMB1_BDEBA | Phytoene synthase (EC 2.5.1.32) | pys (Bd1725) | Bdellovibrio bacteriovorus |
| A5EQB0 | A5EQB0_BRASB | Phytoene synthase (EC 2.5.1.32) | crtB (BBta_6444) | Bradyrhizobium sp. (strain BTAi1/ATCC |
| | | | | BAA-1182) |
| Q13J25 | Q13J25_BURXL | Putative squalene/phytoene | Bxeno_B2946 | Burkholderia xenovorans (strain LB400) |
| | | synthase (EC 2.5.1.32) | (Bxe_B0010) | |
| Q13Z22 | Q13Z22_BURXL | Putative phytoene synthase (EC | Bxeno_A2129 | Burkholderia xenovorans (strain LB400) |
| | | 2.5.1.32) | (Bxe_A2303) | |
| B3U4U9 | B3U4U9_9BACT | Phytoene synthase (EC 2.5.1.32) | crtB | Candidatus Nitrospira defluvii |
| P37272 | PSY_CAPAN | Phytoene synthase, chloroplastic | PSY1 | Capsicum annuum (Bell pepper) |
| | | (EC 2.5.1.32) | | |
| Q6J214 | Q6J214_CHLRE | Chloroplast phytoene synthase | PSY (PSY1) | Chlamydomonas reinhardtii |
| | | (Phytoene synthase) (EC 2.5.1.32) | (CHLREDRAFT_59715) | |
| Q7NTS5 | Q7NTS5_CHRVO | Probable geranylgeranyl- | CV_2978 | Chromobacterium violaceum |
| | | diphosphate | | |
| | | geranylgeranyltransferase (EC | | |
| | | 2.5.1.32) | | |
| Q3C0I6 | Q3C0I6_CROSA | Phytoene synthase (EC 2.5.1.32) | psy | Crocus sativus (Saffron) |
| | | (Fragment) | | |
| C9Y0F0 | C9Y0F0_9ENTR | Phytoene synthase (EC 2.5.1.21) | crtB (Ctu_35440) | Cronobacter turicensis |
| | | (EC 2.5.1.32) | | |
| P49293 | PSY_CUCME | Phytoene synthase, chloroplastic | PSY | Cucumis melo (Muskmelon) |
| | | (EC 2.5.1.32) (MEL5) | | |
| B3RCD6 | B3RCD6_CUPTR | Phytoene synthase (EC 2.5.1.32) | crtB (RALTA_B1982) | Cupriavidus taiwanensis (strain R1/LMG |
| | | | | 19424) (Ralstonia taiwanensis (strain LMG |
| | | | | 19424)) |
| C7QU34 | C7QU34_CYAP0 | Phytoene synthase (EC 2.5.1.32) | Cyan8802_0309 | Cyanothece sp. (strain PCC 8802) |
| | | | | (Synechococcus sp. (strain RF-2)) |
| Q9SSU8 | PSY_DAUCA | Phytoene synthase, chloroplastic | PSY | Daucus carota (Carrot) |
| | | (EC 2.5.1.32) | | |
| Q2P9P0 | Q2P9P0_DAUCA | Phytoene synthase (EC 2.5.1.32) | psy | Daucus carota (Carrot) |
| | | (Fragment) | | |
| A8LQ02 | A8LQ02_DINSH | Phytoene synthase (EC 2.5.1.32) | crtB (Dshi_3509) | Dinoroseobacter shibae (strain DFL 12) |
| C7C5A3 | C7C5A3_9ENTR | Phytoene synthase crtB (EC | crtB | Enterobacter helveticus |
| | | 2.5.1.21) (EC 2.5.1.32) | | |
| C7C5F2 | C7C5F2_9ENTR | Phytoene synthase crtB (EC | crtB | Enterobacter pulveris |
| | | 2.5.1.21) (EC 2.5.1.32) | | |
| C7C533 | C7C533_9ENTR | Phytoene synthase crtB (EC | crtB | Enterobacter turicensis |
| | | 2.5.1.21) (EC 2.5.1.32) | | |
| P22872 | CRTB_ESCVU | Phytoene synthase (EC 2.5.1.32) | crtB | Escherichia vulneris |
| C4L3R8 | C4L3R8_EXISA | Phytoene synthase (EC 2.5.1.32) | EAT1b_2492 | Exiguobacterium sp. (strain ATCC BAA-1283/ |
| | | | | AT1b) |
| C6X6B5 | C6X6B5_FLAB3 | Phytoene synthase (EC 2.5.1.32) | FIC_01506 | Flavobacteriaceae bacterium (strain 3519- |
| | | | | 10) |
| A6GZK0 | A6GZK0_FLAPJ | Geranylgeranyl- | crtB (FP1450) | Flavobacterium psychrophilum (strain |
| | | diphosphategeranylgeranyltransferase | | JIP02/86/ATCC 49511) |
| | | (EC 2.5.1.32) | | |
| Q2JD82 | Q2JD82_FRASC | Phytoene synthase (EC 2.5.1.32) | Francci3_1383 | Frankia sp. (strain Ccl3) |
| C1AB42 | C1AB42_GEMAT | Phytoene synthase (EC 2.5.1.32) | pys (GAU_2406) | Gemmatimonas aurantiaca (strain T-27/ |
| | | | | DSM 14586/JCM 11422/NBRC 100505) |
| C0U9J1 | C0U9J1_9ACTO | Phytoene synthase (EC 2.5.1.32) | GobsDRAFT_37780 | Geodermatophilus obscurus DSM 43160 |
| Q0BQM3 | Q0BQM3_GRABC | Phytoene synthase (EC 2.5.1.32) | GbCGDNIH1_1981 | Granulibacter bethesdensis (strain ATCC |
| | | | | BAA-1260/CGDNIH1) |
| Q5V0N0 | Q5V0N0_HALMA | Phytoene synthase (EC 2.5.1.21) | crtB (rrnAC2069) | Haloarcula marismortui (Halobacterium |
| | | (EC 2.5.1.32) | | marismortui) |
| B0R5N1 | B0R5N1_HALS3 | Geranylgeranyl-diphosphate | crtB1 (OE3093R) | Halobacterium salinarum (strain ATCC |
| | | geranylgeranyltransferase | | 29341/DSM 671/R1) |
| | | (Phytoene synthase) (EC 2.5.1.32) | | |
| B0R649 | B0R649_HALS3 | Geranylgeranyl-diphosphate | crtB2 (OE3376F) | Halobacterium salinarum (strain ATCC |
| | | geranylgeranyltransferase | | 29341/DSM 671/R1) |
| | | (Phytoene synthase) (EC 2.5.1.32) | | |
| C1V579 | C1V579_9EURY | Phytoene synthase (EC 2.5.1.32) | HborDRAFT_2335 | Halogeometricum borinquense DSM 11551 |
| Q18GD2 | Q18GD2_HALWD | Geranylgeranyl-diphosphate | crtB (HQ2860A) | Haloquadratum walsbyi (strain DSM 16790) |
| | | geranylgeranyltransferase | | |
| | | (Phytoene synthase) (EC 2.5.1.32) | | |
| A1WXH0 | A1WXH0_HALHL | Phytoene synthase (EC 2.5.1.32) | Hhal_1618 | Halorhodospira halophila (strain DSM 244/ |
| | | | | SL1) (Ectothiorhodospira halophila (strain |
| | | | | DSM 244/SL1)) |
| Q9AVV8 | Q9AVV8_HELAN | Phytoene synthase (EC 2.5.1.32) | psy | Helianthus annuus (Common sunflower) |
| Q9FEY7 | Q9FEY7_HELAN | Phytoene synthase (EC 2.5.1.32) | psy | Helianthus annuus (Common sunflower) |
| Q28W53 | Q28W53_JANSC | Phytoene synthase (EC 2.5.1.32) | crtB (Jann_0142) | Jannaschia sp. (strain CCS1) |
| A6SY71 | A6SY71_JANMA | Phytoene synthase (EC 2.5.1.32) | crtb (mma_1528) | Janthinobacterium sp. (strain Marseille) |
| | | | | (Minibacterium massiliensis) |
| B2GHF5 | B2GHF5_KOCRD | Phytoene synthase (EC 2.5.1.32) | crtB (KRH_20840) | Kocuria rhizophila (strain ATCC 9341/DSM |
| | | | | 348/NBRC 103217/DC2201) |
| C1WPL9 | C1WPL9_9ACTO | Phytoene synthase (EC 2.5.1.32) | KflaDRAFT_3602 | Kribbella flavida DSM 17836 |
| C1D987 | C1D987_LARHH | Probable geranylgeranyl- | LHK_02043 | Laribacter hongkongensis (strain HLHK9) |
| | | diphosphate | | |
| | | geranylgeranyltransferase (EC | | |
| | | 2.5.1.32) | | |
| C2C1J2 | C2C1J2_LISGR | Possible phytoene synthase (EC | crtB | Listeria grayi DSM 20601 |
| | | 2.5.1.32) | (HMPREF0556_1550) | |
| C1XLJ4 | C1XLJ4_MEIRU | Phytoene synthase (EC 2.5.1.32) | MrubDRAFT_23010 | Meiothermus ruber DSM 1279 |
| C1XVR2 | C1XVR2_9DEIN | Phytoene synthase (EC 2.5.1.32) | MesilDRAFT_24320 | Meiothermus silvanus DSM 9946 |
| C6WVY4 | C6WVY4_METML | Squalene synthase HpnD (EC | Mmol_1177 | Methylotenera mobilis (strain JLW8/ATCC |
| | | 2.5.1.32) | | BAA-1282/DSM 17540) |
| C6XDN9 | C6XDN9_METSD | Squalene synthase HpnC (EC | Msip34_1419 | Methylovorus sp. (strain SIP3-4) |
| | | 2.5.1.32) | | (Methylotenera sp. (strain SIP3-4)) |
| C6XDP0 | C6XDP0_METSD | Squalene synthase HpnD (EC | Msip34_1420 | Methylovorus sp. (strain SIP3-4) |
| | | 2.5.1.32) | | (Methylotenera sp. (strain SIP3-4)) |
| P65861 | CRTB_MYCBO | Probable phytoene synthase (EC | crtB (phyA) | Mycobacterium bovis |
| | | 2.5.1.32) | (Mb3430c) | |
| A4T6X5 | A4T6X5_MYCGI | Phytoene synthase (EC 2.5.1.32) | Mflv_1846 | Mycobacterium gilvum (strain PYR-GCK) |
| | | | | (Mycobacterium flavescens (strain ATCC |
| | | | | 700033/PYR-GCK)) |
| A3Q7U1 | A3Q7U1_MYCSJ | Phytoene synthase (EC 2.5.1.32) | Mjls_5455 | Mycobacterium sp. (strain JLS) |
| A1UNE5 | A1UNE5_MYCSK | Phytoene synthase (EC 2.5.1.32) | Mkms_5164 | Mycobacterium sp. (strain KMS) |
| Q1B1Q4 | Q1B1Q4_MYCSS | Phytoene synthase (EC 2.5.1.32) | Mmcs_5076 | Mycobacterium sp. (strain MCS) |
| P65860 | CRTB_MYCTU | Probable phytoene synthase (EC | crtB (phyA) (Rv3397c) | Mycobacterium tuberculosis |
| | | 2.5.1.32) | (MT3505) | |
| | | | (MTCY78.31) | |
| A1T5F9 | A1T5F9_MYCVP | Phytoene synthase (EC 2.5.1.32) | Mvan_1579 | Mycobacterium vanbaalenii (strain DSM |
| | | | | 7251/PYR-1) |
| C8X6L3 | C8X6L3_9ACTO | Phytoene synthase (EC 2.5.1.32) | Namu_2501 | Nakamurella multipartita (strain ATCC |
| | | | | 700099/DSM 44233/JCM 9543/Y-104) |
| | | | | (Microsphaera multipartita) |
| P53797 | PSY_NARPS | Phytoene synthase, chloroplastic | PSY | Narcissus pseudonarcissus (Daffodil) |
| | | (EC 2.5.1.32) | | |
| Q3IN30 | Q3IN30_NATPD | Geranylgeranyl-diphosphate | crtB (NP4770A) | Natronomonas pharaonis (strain DSM 2160/ |
| | | geranylgeranyltransferase | | ATCC 35678) |
| | | (Phytoene synthase) (EC 2.5.1.32) | | |
| P37295 | PSY_NEUCR | Phytoene synthase (EC 2.5.1.32) | al-2 (B22I21.230) | Neurospora crassa |
| | | (Protein albino-2) | (NCU00585) | |
| Q078Y5 | Q078Y5_NICLS | Phytoene synthase (EC 2.5.1.32) | pys-1 | Nicotiana langsdorffii x Nicotiana sanderae |
| | | | | (Ornamental tobacco) |
| C1YR05 | C1YR05_NOCDA | Phytoene synthase (EC 2.5.1.32) | NdasDRAFT_3718 | Nocardiopsis dassonvillei subsp. |
| | | | | dassonvillei DSM 43111 |
| Q6ED64 | Q6ED64_ORYSI | Phytoene synthase 1 (EC 2.5.1.32) | | Oryza sativa subsp. indica (Rice) |
| P21683 | CRTB_PANAN | Phytoene synthase (EC 2.5.1.32) | crtB | Pantoea ananas (Erwinia uredovora) |
| P54975 | CRTB_PARSN | Phytoene synthase (EC 2.5.1.32) | crtB | Paracoccus sp. (strain N81106/MBIC |
| | | | | 01143) (Agrobacterium aurantiacum) |
| A3GH05 | A3GH05_PICST | Type II proteins | BET2.2 (PICST_37880) | Pichia stipitis (Yeast) |
| | | geranylgeranyltransferase beta | | |
| | | subunit (EC 2.5.1.32) | | |
| A3M018 | A3M018_PICST | Geranylgeranyltransferase beta | CDC43 (PICST_79863) | Pichia stipitis (Yeast) |
| | | subunit (EC 2.5.1.32) | | |
| A2BNT7 | A2BNT7_PROMS | Squalene and phytoene synthases | crtB (A9601_01601) | Prochlorococcus marinus (strain AS9601) |
| | | (EC 2.5.1.32) | | |
| A9BD01 | A9BD01_PROM4 | Squalene and phytoene synthase | crtB/pys | Prochlorococcus marinus (strain MIT 9211) |
| | | (EC 2.5.1.32) | (P9211_01581) | |
| A3PAL0 | A3PAL0_PROM0 | Squalene and phytoene synthase | crtB, pys | Prochlorococcus marinus (strain MIT 9301) |
| | | (EC 2.5.1.32) | (P9301_01621) | |
| A2CD34 | A2CD34_PROM3 | Squalene and phytoene synthase | crtB (P9303_26641) | Prochlorococcus marinus (strain MIT 9303) |
| | | (EC 2.5.1.32) | | |
| Q31D38 | Q31D38_PROM9 | Phytoene synthase (EC 2.5.1.32) | PMT9312_0145 | Prochlorococcus marinus (strain MIT 9312) |
| A2BUB9 | A2BUB9_PROM5 | Squalene and phytoene synthase | crtB (P9515_01711) | Prochlorococcus marinus (strain MIT 9515) |
| | | (EC 2.5.1.32) | | |
| A2BZW9 | A2BZW9_PROM1 | Squalene and phytoene synthases | crtB (NATL1_02151) | Prochlorococcus marinus (strain NATL1A) |
| | | (EC 2.5.1.32) | | |
| Q46HN1 | Q46HN1_PROMT | Phytoene synthase (EC 2.5.1.32) | PMN2A_1509 | Prochlorococcus marinus (strain NATL2A) |
| B6QY45 | B6QY45_9RHOB | Phytoene synthase protein (EC | crtB (PJE062_1370) | Pseudovibrio sp. JE062 |
| | | 2.5.1.32) | | |
| Q0KBM0 | Q0KBM0_RALEH | Squalene/phytoene synthase (EC | H16_A1466 | Ralstonia eutropha (strain ATCC 17699/ |
| | | 2.5.1.32) | | H16/DSM 428/Stanier 337) (Cupriavidus |
| | | | | necator (strain ATCC 17699/H16/DSM |
| | | | | 428/Stanier 337)) |
| A3RPK9 | A3RPK9_RALSO | Phytoene synthase (EC 2.5.1.32) | RRSL_04578 | Ralstonia solanacearum UW551 |
| Q2K959 | Q2K959_RHIEC | Phytoene synthase protein (EC | crtB (RHE_CH01834) | Rhizobium etli (strain CFN 42/ATCC |
| | | 2.5.1.32) | | 51251) |
| B3PXV1 | B3PXV1_RHIE6 | Phytoene synthase protein (EC | crtB | Rhizobium etli (strain CIAT 652) |
| | | 2.5.1.32) | (RHECIAT_CH0001920) | |
| P55350 | Y4AC_RHISN | Putative phytoene synthase (EC | NGR_a00440 (y4aC) | Rhizobium sp. (strain NGR234) |
| | | 2.5.1.32) | | |
| Q9UUQ6 | LCPS_RHIRA | Bifunctional enzyme CarRP | CARRP | Rhizomucor racemosus (Mucor |
| | | [Includes: Lycopene cyclase | | circinelloides f. lusitanicus) |
| | | (EC 1.—.—.—); Phytoene | | |
| | | synthase (EC 2.5.1.32)] | | |
| P17056 | CRTB_RHOCA | Phytoene synthase (EC 2.5.1.32) | crtB | Rhodobacter capsulatus |
| | | | | (Rhodopseudomonas capsulata) |
| P54905 | CRTB_RHOS4 | Phytoene synthase (EC 2.5.1.32) | crtB (RHOS4_18750) | Rhodobacter sphaeroides (strain ATCC |
| | | | (RSP_0270) | 17023/2.4.1/NCIB 8253/DSM 158) |
| A4WRB2 | A4WRB2_RHOS5 | Phytoene synthase (EC 2.5.1.32) | Rsph17025_1025 | Rhodobacter sphaeroides (strain ATCC |
| | | | | 17025/ATH 2.4.3) |
| A3PL01 | A3PL01_RHOS1 | Phytoene synthase (EC 2.5.1.32) | Rsph17029_1913 | Rhodobacter sphaeroides (strain ATCC |
| | | | | 17029/ATH 2.4.9) |
| C1A0Z6 | C1A0Z6_RHOE4 | Probable phytoene synthase (EC | crtB (RER_35730) | Rhodococcus erythropolis (strain PR4/ |
| | | 2.5.1.32) | | NBRC 100887) |
| C3JJ70 | C3JJ70_RHOER | Phytoene synthase (EC 2.5.1.32) | RHOER0001_6432 | Rhodococcus erythropolis SK121 |
| C1AU76 | C1AU76_RHOOB | Phytoene synthase (EC 2.5.1.32) | crtB (ROP_08370) | Rhodococcus opacus (strain B4) |
| Q07S16 | Q07S16_RHOP5 | Phytoene synthase (EC 2.5.1.32) | RPE_1316 | Rhodopseudomonas palustris (strain |
| | | | | BisA53) |
| Q219W2 | Q219W2_RHOPB | Phytoene synthase (EC 2.5.1.32) | RPC_1262 | Rhodopseudomonas palustris (strain |
| | | | | BisB18) |
| Q132K3 | Q132K3_RHOPS | Phytoene synthase (EC 2.5.1.32) | RPD_3765 | Rhodopseudomonas palustris (strain BisB5) |
| Q2ISV7 | Q2ISV7_RHOP2 | Phytoene synthase (EC 2.5.1.32) | RPB_4010 | Rhodopseudomonas palustris (strain HaA2) |
| Q2RX46 | Q2RX46_RHORT | Phytoene synthase (EC 2.5.1.32) | Rru_A0494 | Rhodospirillum rubrum (strain ATCC 11170/ |
| | | | | NCIB 8255) |
| Q1AXR7 | Q1AXR7_RUBXD | Phytoene synthase (EC 2.5.1.32) | Rxyl_0844 | Rubrobacter xylanophilus (strain DSM 9941/ |
| | | | | NBRC 16129) |
| A4FER9 | A4FER9_SACEN | Putative phytoene synthase (EC | crtB (SACE_3269) | Saccharopolyspora erythraea (strain NRRL |
| | | 2.5.1.32) | | 23338) |
| A4FFI8 | A4FFI8_SACEN | Phytoene synthase (EC 2.5.1.32) | crtB (SACE_3539) | Saccharopolyspora erythraea (strain NRRL |
| | | | | 23338) |
| A4FHS4 | A4FHS4_SACEN | Putative squalene/phytoene | hopD (SACE_4330) | Saccharopolyspora erythraea (strain NRRL |
| | | synthase (EC 2.5.1.32) | | 23338) |
| A4XD62 | A4XD62_SALTO | Phytoene synthase (EC 2.5.1.32) | Strop_4441 | Salinispora tropica (strain ATCC BAA-916/ |
| | | | | DSM 44818/CNB-440) |
| P08196 | PSY1_SOLLC | Phytoene synthase 1, chloroplastic | PSY1 (GTOM5) | Solanum lycopersicum (Tomato) |
| | | (EC 2.5.1.32) (Fruit-ripening- | (PTOM5) (TOM5) | (Lycopersicon esculentum) |
| | | specific protein pTOM5) | | |
| P37273 | PSY2_SOLLC | Phytoene synthase 2, chloroplastic | PSY2 | Solanum lycopersicum (Tomato) |
| | | (EC 2.5.1.32) (Fragment) | | (Lycopersicon esculentum) |
| Q2P9N9 | Q2P9N9_SOLLC | Phytoene synthase (EC 2.5.1.32) | psy | Solanum lycopersicum (Tomato) |
| | | (Fragment) | | (Lycopersicon esculentum) |
| O07333 | CRTY_SPIPL | Phytoene synthase (EC 2.5.1.32) | crtB (pys) | Spirulina platensis |
| B9DJ78 | B9DJ78_STACT | Similar to squalene synthase (EC | Sca_2274 | Staphylococcus carnosus (strain TM300) |
| | | 2.5.1.32) | | |
| Q9ACU1 | CRUP_STRCO | Bifunctional protein crtB/uppS | crtB/uppS3 (SCP1.212) | Streptomyces coelicolor |
| | | [Includes: Phytoene synthase (EC | | |
| | | 2.5.1.32); Undecaprenyl | | |
| | | pyrophosphate synthetase 3 (UPP | | |
| | | synthetase 3) (EC 2.5.1.31) (Di- | | |
| | | trans, poly-cis- | | |
| | | decaprenylcistransferase 3) | | |
| | | (Undecaprenyl diphosphate | | |
| | | synthase 3) (UDS 3)] | | |
| P54977 | CRTB_STRGR | Phytoene synthase (EC 2.5.1.32) | crtB (crtl) | Streptomyces griseus |
| C4EG11 | C4EG11_STRRS | Phytoene synthase (EC 2.5.1.32) | SrosDRAFT_56050 | Streptosporangium roseum DSM 43021 |
| P37269 | CRTB_SYNE7 | Phytoene synthase (EC 2.5.1.32) | crtB (pys) | Synechococcus elongatus (strain PCC 7942) |
| | | | (Synpcc7942_1984) | (Anacystis nidulans R2) |
| Q3B056 | Q3B056_SYNS9 | Phytoene synthase (EC 2.5.1.32) | Syncc9902_0299 | Synechococcus sp. (strain CC9902) |
| A5GQI3 | A5GQI3_SYNR3 | Phytoene synthase (EC 2.5.1.32) | crtB | Synechococcus sp. (strain RCC307) |
| | | | (SynRCC307_0239) | |
| A5GP30 | A5GP30_SYNPW | Phytoene synthase (EC 2.5.1.32) | crtB | Synechococcus sp. (strain WH7803) |
| | | | (SynWH7803_2269) | |
| D0CNA7 | D0CNA7_9SYNE | Phytoene synthase (EC 2.5.1.32) | SH8109_0768 | Synechococcus sp. WH 8109 |
| P37294 | CRTB_SYNY3 | Phytoene synthase (EC 2.5.1.32) | crtB (pys) (slr1255) | Synechocystis sp. (strain PCC 6803) |
| C7DFN4 | C7DFN4_9RHOB | Phytoene synthase (EC 2.5.1.32) | TR2A62_2730 | Thalassiobium sp. R2A62 |
| C4ZPQ1 | C4ZPQ1_THASP | Squalene synthase HpnC (EC | Tmz1t_2157 | Thauera sp. (strain MZ1T) |
| | | 2.5.1.32) | | |
| C4ZPQ2 | C4ZPQ2_THASP | Squalene synthase HpnD (EC | Tmz1t_2158 | Thauera sp. (strain MZ1T) |
| | | 2.5.1.32) | | |
| Q47KB3 | Q47KB3_THEFY | Phytoene synthase (EC 2.5.1.32) | Tfu_3076 | Thermobifida fusca (strain YX) |
| P37270 | CRTB_THET2 | Phytoene synthase (EC 2.5.1.32) | crtB (TT_P0057) | Thermus thermophilus (strain HB27/ATCC |
| | | | | BAA-163/DSM 7039) |
| Q10XJ4 | Q10XJ4_TRIEI | Phytoene synthase (EC 2.5.1.32) | Tery_4010 | Trichodesmium erythraeum (strain |
| | | | | IMS101) |
| A2T2K4 | A2T2K4_TRITU | Putative phytoene synthase 1-B1 | Psy1-B1 | Triticum turgidum subsp. durum (durum |
| | | (EC 2.5.1.32) (Fragment) | | wheat) |
| A2T2K5 | A2T2K5_TRITU | Phytoene synthase 1-B1 (EC | Psy1-B1 | Triticum turgidum subsp. durum (durum |
| | | 2.5.1.32) (Fragment) | | wheat) |
| A2T2K6 | A2T2K6_TRITU | Phytoene synthase 2-B1 (EC | Psy2-B1 | Triticum turgidum subsp. durum (durum |
| | | 2.5.1.32) (Fragment) | | wheat) |
| A2T2K7 | A2T2K7_TRITU | Phytoene synthase 2-B1 (EC | Psy2-B1 | Triticum turgidum subsp. durum (durum |
| | | 2.5.1.32) (Fragment) | | wheat) |
| A2T2K8 | A2T2K8_TRITU | Phytoene synthase 1-2 (EC | Psy1-2 | Triticum turgidum subsp. durum (durum |
| | | 2.5.1.32) (Fragment) | | wheat) |
| A2T2L0 | A2T2L0_TRITU | Phytoene synthase 2-2 (EC | Psy2-2 | Triticum turgidum subsp. durum (durum |
| | | 2.5.1.32) (Fragment) | | wheat) |
| C4N548 | C4N548_TRITU | Phytoene synthase 1 (EC 2.5.1.32) | | Triticum turgidum subsp. durum (durum |
| | | | | wheat) |
| C5CJS8 | C5CJS8_VARPS | Squalene synthase HpnD (EC | Vapar_2505 | Variovorax paradoxus (strain S110) |
| | | 2.5.1.32) | | |
| P49085 | PSY_MAIZE | Phytoene synthase, chloroplastic | Y1 | Zea mays (Maize) |
| | | (EC 2.5.1.32) | | |
| C8WB86 | C8WB86_ZYMMO | Squalene synthase HpnD (EC | Za10_0422 | Zymomonas mobilis subsp. mobilis (strain |
| | | 2.5.1.32) | | NCIB 11163) |

D-1-deoxyxylulose 5-phosphate synthase

In some embodiments, cassava (or other crop) contains a D-1-deoxyxylulose 5-phosphate synthase transgene (“DXS”) functional in cassava (EC 2.2.1.7). The DXS can be any enzyme that catalyzes the conversion of pyruvate and glyceraldehyde3-phosphate to 1-deoxyxyulose-5-phosphate. Optionally, the DXS is a plant or bacterial DSX.

Optionally, the DXS is any DXS set forth in Table 4. Optionally, the DXS exhibits a sequence identity of at least about any of 75%, 80%, 85%, 90%, or 95% to a DXS listed in Table 4.

Examplary DXS transgenes comprise one or more of the following features:

l. a thiamine pyrophosphate (TPP) binding domain;
m. a transketolase domain.

Useful DXS enzymes can be isolated from any organism such as Streptomyces, Escherichia coli, Bacillus subtilis, Synechocystis, Psueomonas (e.g. P. aeruginosa), Rhodabacter (e.g. R. capsulatus), or Arabidopsis.

Classes of DXS that are especially useful include those from plants that produce abundant terpenoids such as mints or conifers. These can be especially useful compared to others because of their high intrinsic activity.

Optionally, the DXS is derived from any of the species set forth in Table 4.

Optionally, the DXS gene sequence comprises Error! Reference source not found., or derivative thereof. Optionally, the DXS protein sequence comprises the sequence encoded by Error! Reference source not found., or derivative thereof.

Optionally, the DXS is operably linked to a comestible (e.g. root) specific promoter. Optionally, the DXS is operably linked to a patatin promoter.

TABLE 4
DXS Transgenes

| Accession | Entry name | Gene names | Organism |
|---|---|---|---|
| A4G1W9 | A4G1W9_HERAR | dxs (HEAR0279) | Herminiimonas arsenicoxydans |
| B0VF15 | B0VF15_9BACT | dxs (CLOAM0157) | Candidatus Cloacamonas acidaminovorans |
| B5S200 | B5S200_RALSO | dxs (RSMK01095) | Ralstonia solanacearum MolK2 |
| B5SFC9 | B5SFC9_RALSO | dxs (RSIPO_02012) | Ralstonia solanacearum IPO1609 |
| C4ILM0 | C4ILM0_CLOBU | CLP_0048 | Clostridium butyricum E4 str. BoNT E BL5262 |
| C7BYU0 | C7BYU0_HELPB | dxs (HELPY_0357) | Helicobacter pylori (strain B38) |
| C9X1Z1 | C9X1Z1_NEIME | dxs (NMV_2057) | Neisseria meningitidis 8013 |
| Q39UB1 | DXS1_GEOMG | dxs1 (Gmet_1934) | Geobacter metallireducens (strain GS-15/ATCC 53774/DSM |
| | | | 7210) |
| Q74FC3 | DXS1_GEOSL | dxs1 (GSU0686) | Geobacter sulfurreducens |
| Q28WA7 | DXS1_JANSC | dxs1 (Jann_0088) | Jannaschia sp. (strain CCS1) |
| Q9F1V2 | DXS1_KITGR | dxs1 (dxs) | Kitasatospora griseola (Streptomyces griseolosporeus) |
| Q2RYD6 | DXS1_RHORT | dxs1 (Rru_A0054) | Rhodospirillum rubrum (strain ATCC 11170/NCIB 8255) |
| Q3J1A8 | DXS1_RHOS4 | dxs1 (RHOS4_18580) | Rhodobacter sphaeroides (strain ATCC 17023/2.4.1/NCIB |
| | | (RSP_0254) | 8253/DSM 158) |
| Q16DV7 | DXS1_ROSDO | dxs1 (RD1_0101) | Roseobacter denitrificans (strain ATCC 33942/OCh 114) |
| | | | (Erythrobacter sp. (strain OCh 114)) (Roseobacter denitrificans) |
| Q82ML4 | DXS1_STRAW | dxs1 (SAV_1646) | Streptomyces avermitilis |
| Q9X7W3 | DXS1_STRCO | dxs1 (dxs) (SCO6768) | Streptomyces coelicolor |
| | | (SC6A5.17) | |
| Q5NN52 | DXS1_ZYMMO | dxs1 (ZMO1234) | Zymomonas mobilis |
| Q39RT4 | DXS2_GEOMG | dxs2 (Gmet_2822) | Geobacter metallireducens (strain GS-15/ATCC 53774/DSM |
| | | | 7210) |
| Q74CB0 | DXS2_GEOSL | dxs2 (GSU1764) | Geobacter sulfurreducens |
| Q28W25 | DXS2_JANSC | dxs2 (Jann_0170) | Jannaschia sp. (strain CCS1) |
| Q8VUR8 | DXS2_KITGR | dxs2 | Kitasatospora griseola (Streptomyces griseolosporeus) |
| Q2RR29 | DXS2_RHORT | dxs2 (Rru_A2619) | Rhodospirillum rubrum (strain ATCC 11170/NCIB 8255) |
| Q3IYR6 | DXS2_RHOS4 | dxs2 (RHOS4_27500) | Rhodobacter sphaeroides (strain ATCC 17023/2.4.1/NCIB |
| | | (RSP_1134) | 8253/DSM 158) |
| Q16CP0 | DXS2_ROSDO | dxs2 (RD1_0548) | Roseobacter denitrificans (strain ATCC 33942/OCh 114) |
| | | | (Erythrobacter sp. (strain OCh 114)) (Roseobacter denitrificans) |
| Q82KW8 | DXS2_STRAW | dxs2 (SAV_2244) | Streptomyces avermitilis |
| Q8CJP7 | DXS2_STRCO | dxs2 (SCO6013) (SC1C3.01) | Streptomyces coelicolor |
| | | (SC7B7.10) | |
| Q5NM38 | DXS2_ZYMMO | dxs2 (ZMO1598) | Zymomonas mobilis |
| B0C8J3 | DXS_ACAM1 | dxs (AM1_5186) | Acaryochloris marina (strain MBIC 11017) |
| A1TNR1 | DXS_ACIAC | dxs (Aave_2015) | Acidovorax avenae subsp. citrulli (strain AAC00-1) |
| Q6F7N5 | DXS_ACIAD | dxs (ACIAD3247) | Acinetobacter sp. (strain ADP1) |
| B0VQB8 | DXS_ACIBS | dxs (ABSDF0389) | Acinetobacter baumannii (strain SDF) |
| B0V710 | DXS_ACIBY | dxs (ABAYE0381) | Acinetobacter baumannii (strain AYE) |
| C1F3C4 | DXS_ACIC5 | dxs (ACP_2818) | Acidobacterium capsulatum (strain ATCC 51196/DSM 11244/ |
| | | | JCM 7670) |
| A1W4U9 | DXS_ACISJ | dxs (Ajs_1038) | Acidovorax sp. (strain JS42) |
| A3MYS9 | DXS_ACTP2 | dxs (APL_0207) | Actinobacillus pleuropneumoniae serotype 5b (strain L20) |
| B3H050 | DXS_ACTP7 | dxs (APP7_0210) | Actinobacillus pleuropneumoniae serotype 7 (strain AP76) |
| B0BSL0 | DXS_ACTPJ | dxs (APJL_0208) | Actinobacillus pleuropneumoniae serotype 3 (strain JL03) |
| A0KNF9 | DXS_AERHH | dxs (AHA_3321) | Aeromonas hydrophila subsp. hydrophila (strain ATCC 7966/ |
| | | | NCIB 9240) |
| A4SJP9 | DXS_AERS4 | dxs (ASA_0990) | Aeromonas salmonicida (strain A449) |
| B9JAL7 | DXS_AGRRK | dxs (Arad_1198) | Agrobacterium radiobacter (strain K84/ATCC BAA-868) |
| Q8UHD7 | DXS_AGRT5 | dxs (Atu0745) (AGR_C_1351) | Agrobacterium tumefaciens (strain C58/ATCC 33970) |
| B9JSL2 | DXS_AGRVS | dxs (Avi_0997) | Agrobacterium vitis (strain S4/ATCC BAA-846) (Rhizobium vitis |
| | | | (strain S4)) |
| Q0VMI4 | DXS_ALCBS | dxs (ABO_2166) | Alcanivorax borkumensis (strain SK2/ATCC 700651/DSM |
| | | | 11573) |
| Q0A8V7 | DXS_ALHEH | dxs (Mlg_1381) | Alkalilimnicola ehrlichei (strain MLHE-1) |
| B6EIA7 | DXS_ALISL | dxs (VSAL_I0933) | Aliivibrio salmonicida (strain LFI1238) (Vibrio salmonicida |
| | | | (strain LFI1238)) |
| A8MFI7 | DXS_ALKOO | dxs (Clos_1607) | Alkaliphilus oremlandii (strain OhILAs) (Clostridium oremlandii |
| | | | (strain OhILAs)) |
| B4RVY8 | DXS_ALTMD | dxs (MADE_01425) | Alteromonas macleodii (strain DSM 17117/Deep ecotype) |
| B8JFY1 | DXS_ANAD2 | dxs (A2cp1_1225) | Anaeromyxobacter dehalogenans (strain 2CP-1/ATCC BAA- |
| | | | 258) |
| Q2IPZ2 | DXS_ANADE | dxs (Adeh_1097) | Anaeromyxobacter dehalogenans (strain 2CP-C) |
| A7H9E8 | DXS_ANADF | dxs (Anae109_1136) | Anaeromyxobacter sp. (strain Fw109-5) |
| B4UHF5 | DXS_ANASK | dxs (AnaeK_1157) | Anaeromyxobacter sp. (strain K) |
| Q8YZ80 | DXS_ANASP | dxs (alr0599) | Anabaena sp. (strain PCC 7120) |
| Q3M4F6 | DXS_ANAVT | dxs (Ava_4532) | Anabaena variabilis (strain ATCC 29413/PCC 7937) |
| O67036 | DXS_AQUAE | dxs (aq_881) | Aquifex aeolicus |
| Q38854 | DXS_ARATH | CLA1 (DEF) (At4g15560) | Arabidopsis thaliana (Mouse-ear cress) |
| | | (dl3821w) | |
| A8EWN0 | DXS_ARCB4 | dxs (Abu_2139) | Arcobacter butzleri (strain RM4018) |
| A1R5N7 | DXS_ARTAT | dxs (AAur_1790) | Arthrobacter aurescens (strain TC1) |
| B8HH36 | DXS_ARTCA | dxs (Achl_1638) | Arthrobacter chlorophenolicus (strain A6/ATCC 700700/DSM |
| | | | 12829/JCM 12360) |
| A0JVG9 | DXS_ARTS2 | dxs (Arth_1645) | Arthrobacter sp. (strain FB24) |
| A8IBS1 | DXS_AZOC5 | dxs (AZC_3111) | Azorhizobium caulinodans (strain ATCC 43989/DSM 5975/ |
| | | | ORS 571) |
| B6YRV5 | DXS_AZOPC | dxs (CFPG_664) | Azobacteroides pseudotrichonymphae genomovar. CFP2 |
| A1K4R0 | DXS_AZOSB | dxs (azo1198) | Azoarcus sp. (strain BH72) |
| Q5P228 | DXS_AZOSE | dxs (AZOSEA25110) | Azoarcus sp. (strain EbN1) (Aromatoleum aromaticum (strain |
| | | (ebA4439) | EbN1)) |
| A7Z6J5 | DXS_BACA2 | dxs (RBAM_022600) | Bacillus amyloliquefaciens (strain FZB42) |
| C3P7V6 | DXS_BACAA | dxs (BAA_4418) | Bacillus anthracis (strain A0248) |
| C3LJV1 | DXS_BACAC | dxs (BAMEG_4436) | Bacillus anthracis (strain CDC 684/NRRL 3495) |
| Q81M54 | DXS_BACAN | dxs (BA_4400) (GBAA_4400) | Bacillus anthracis |
| | | (BAS4081) | |
| B7JM28 | DXS_BACC0 | dxs (BCAH820_4197) | Bacillus cereus (strain AH820) |
| Q731B7 | DXS_BACC1 | dxs (BCE_4249) | Bacillus cereus (strain ATCC 10987) |
| B7IXG8 | DXS_BACC2 | dxs (BCG9842_B0947) | Bacillus cereus (strain G9842) |
| B7HB48 | DXS_BACC4 | dxs (BCB4264_A4287) | Bacillus cereus (strain B4264) |
| B7HNU0 | DXS_BACC7 | dxs (BCAH187_A4307) | Bacillus cereus (strain AH187) |
| A7GSJ5 | DXS_BACCN | dxs (Bcer98_2870) | Bacillus cereus subsp. cytotoxis (strain NVH 391-98) |
| Q818R9 | DXS_BACCR | dxs (BC_4176) | Bacillus cereus (strain ATCC 14579/DSM 31) |
| Q635A7 | DXS_BACCZ | dxs (BCE33L3930) | Bacillus cereus (strain ZK/E33L) |
| Q5LH44 | DXS_BACFN | dxs (BF0796) | Bacteroides fragilis (strain ATCC 25285/NCTC 9343) |
| Q64Y02 | DXS_BACFR | dxs (BF0873) | Bacteroides fragilis |
| Q9K971 | DXS_BACHD | dxs (BH2779) | Bacillus halodurans |
| Q6HDY8 | DXS_BACHK | dxs (BT9727_3919) | Bacillus thuringiensis subsp. konkukian |
| Q65HJ2 | DXS_BACLD | dxs (BLi02598) (BL01523) | Bacillus licheniformis (strain DSM 13/ATCC 14580) |
| A8FF11 | DXS_BACP2 | dxs (BPUM_2159) | Bacillus pumilus (strain SAFR-032) |
| Q5WF63 | DXS_BACSK | dxs (ABC2462) | Bacillus clausii (strain KSM-K16) |
| P54523 | DXS_BACSU | dxs (yqiE) (BSU24270) | Bacillus subtilis |
| Q8A0C2 | DXS_BACTN | dxs (BT_4099) | Bacteroides thetaiotaomicron |
| A6L175 | DXS_BACV8 | dxs (BVU_1763) | Bacteroides vulgatus (strain ATCC 8482/DSM 1447/NCTC |
| | | | 11154) |
| A9VGD1 | DXS_BACWK | dxs (BcerKBAB4_4029) | Bacillus weihenstephanensis (strain KBAB4) |
| A1URW6 | DXS_BARBK | dxs (BARBAKC583_0400) | Bartonella bacilliformis (strain ATCC 35685/KC583) |
| Q6G4D1 | DXS_BARHE | dxs (BH04350) | Bartonella henselae (Rochalimaea henselae) |
| Q6G0D4 | DXS_BARQU | dxs (BQ03540) | Bartonella quintana (Rochalimaea quintana) |
| A9IQP2 | DXS_BART1 | dxs (BT_0649) | Bartonella tribocorum (strain CIP 105476/IBS 506) |
| Q1LTI9 | DXS_BAUCH | dxs (BCI_0275) | Baumannia cicadellinicola subsp. Homalodisca coagulata |
| B2IDK3 | DXS_BEII9 | dxs (Bind_1811) | Beijerinckia indica subsp. indica (strain ATCC 9039/DSM 1715/ |
| | | | NCIB 8712) |
| Q7VRH9 | DXS_BLOFL | dxs (Bfl238) | Blochmannia floridanus |
| Q493G7 | DXS_BLOPB | dxs (BPEN_244) | Blochmannia pennsylvanicus (strain BPEN) |
| Q2KZ15 | DXS_BORA1 | dxs (BAV2177) | Bordetella avium (strain 197N) |
| Q7WL37 | DXS_BORBR | dxs (BB1912) | Bordetella bronchiseptica (Alcaligenes bronchisepticus) |
| Q7W7Q0 | DXS_BORPA | dxs (BPP2464) | Bordetella parapertussis |
| A9ITB0 | DXS_BORPD | dxs (Bpet3060) | Bordetella petrii (strain ATCC BAA-461/DSM 12804/CCUG |
| | | | 43448) |
| Q7VV87 | DXS_BORPE | dxs (BP2798) | Bordetella pertussis |
| Q89RW1 | DXS_BRAJA | dxs (bII2651) | Bradyrhizobium japonicum |
| A5EEQ0 | DXS_BRASB | dxs (BBta_2479) | Bradyrhizobium sp. (strain BTAi1/ATCC BAA-1182) |
| A4YQ36 | DXS_BRASO | dxs (BRADO2161) | Bradyrhizobium sp. (strain ORS278) |
| C0ZC10 | DXS_BREBN | dxs (BBR47_23420) | Brevibacillus brevis (strain 47/JCM 6285/NBRC 100599) |
| B2S9T6 | DXS_BRUA1 | dxs (BAbS19_I04270) | Brucella abortus (strain S19) |
| Q2YMF0 | DXS_BRUA2 | dxs (BAB1_0462) | Brucella abortus (strain 2308) |
| Q57ET1 | DXS_BRUAB | dxs (BruAb1_0458) | Brucella abortus |
| A9M8W0 | DXS_BRUC2 | dxs (BCAN_A0440) | Brucella canis (strain ATCC 23365/NCTC 10854) |
| C0RHE3 | DXS_BRUMB | dxs (BMEA_A0469) | Brucella melitensis biotype 2 (strain ATCC 23457) |
| Q8YFM2 | DXS_BRUME | dxs (BMEI1498) | Brucella melitensis |
| A5VP09 | DXS_BRUO2 | dxs (BOV_0443) | Brucella ovis (strain ATCC 25840/63/290/NCTC 10512) |
| B0CKC0 | DXS_BRUSI | dxs (BSUIS_A0462) | Brucella suis (strain ATCC 23445/NCTC 10510) |
| Q8G292 | DXS_BRUSU | dxs (BR0436) | Brucella suis |
| B8D9P1 | DXS_BUCA5 | dxs (BUAP5A_457) | Buchnera aphidicola subsp. Acyrthosiphon pisum (strain 5A) |
| P57536 | DXS_BUCAI | dxs (BU464) | Buchnera aphidicola subsp. Acyrthosiphon pisum |
| | | | (Acyrthosiphon pisum symbiotic bacterium) |
| Q8K9A1 | DXS_BUCAP | dxs (BUsg_448) | Buchnera aphidicola subsp. Schizaphis graminum |
| B8D7Z3 | DXS_BUCAT | dxs (BUAPTUC7_458) | Buchnera aphidicola subsp. Acyrthosiphon pisum (strain Tuc7) |
| B1Z1G2 | DXS_BURA4 | dxs (BamMC406_3776) | Burkholderia ambifaria (strain MC40-6) |
| Q1BLY7 | DXS_BURCA | dxs (Bcen_4486) | Burkholderia cenocepacia (strain AU 1054) |
| B1K3S9 | DXS_BURCC | dxs (Bcenmc03_3648) | Burkholderia cenocepacia (strain MC0-3) |
| A0AYZ0 | DXS_BURCH | dxs (Bcen2424_3879) | Burkholderia cenocepacia (strain HI2424) |
| B4EN29 | DXS_BURCJ | dxs (BceJ2315_43660) | Burkholderia cepacia (strain J2315/LMG 16656) (Burkholderia |
| | | (BCAM0911) | cenocepacia (strain J2315)) |
| Q0BAL8 | DXS_BURCM | dxs (Bamb_3250) | Burkholderia ambifaria (strain ATCC BAA-244/AMMD) |
| | | | (Burkholderia cepacia (strain AMMD)) |
| Q62DU1 | DXS_BURMA | dxs (BMAA0330) | Burkholderia mallei (Pseudomonas mallei) |
| A3P7W4 | DXS_BURP0 | dxs (BURPS1106A_A2392) | Burkholderia pseudomallei (strain 1106a) |
| Q3JKA3 | DXS_BURP1 | dxs (BURPS1710b_A0842) | Burkholderia pseudomallei (strain 1710b) |
| A3NMF6 | DXS_BURP6 | dxs (BURPS668_A2534) | Burkholderia pseudomallei (strain 668) |
| B2JP68 | DXS_BURP8 | dxs (Bphy_3948) | Burkholderia phymatum (strain DSM 17167/STM815) |
| Q63JF4 | DXS_BURPS | dxs (BPSS1762) | Burkholderia pseudomallei (Pseudomonas pseudomallei) |
| Q393P4 | DXS_BURS3 | dxs (Bcep18194_B2211) | Burkholderia sp. (strain 383) (Burkholderia cepacia (strain |
| | | | ATCC 17760/NCIB 9086/R18194)) |
| Q2T7N5 | DXS_BURTA | dxs (BTH_II0614) | Burkholderia thailandensis (strain E264/ATCC 700388/DSM |
| | | | 13276/CIP 106301) |
| Q13RX1 | DXS_BURXL | dxs (Bxeno_B0200) | Burkholderia xenovorans (strain LB400) |
| | | (Bxe_B2827) | |
| A0RMN5 | DXS_CAMFF | dxs (CFF8240_0264) | Campylobacter fetus subsp. fetus (strain 82-40) |
| A7I2V7 | DXS_CAMHC | dxs (CHAB381_1297) | Campylobacter hominis (strain ATCC BAA-381/LMG 19568/ |
| | | | NCTC 13146/CH001A) |
| A8FKB0 | DXS_CAMJ8 | dxs (C8J_0298) | Campylobacter jejuni subsp. jejuni serotype O:6 (strain 81116/ |
| | | | NCTC 11828) |
| A7H552 | DXS_CAMJD | dxs (JJD26997_1642) | Campylobacter jejuni subsp. doylei (strain ATCC BAA-1458/ |
| | | | RM4099/269.97) |
| Q9PIH8 | DXS_CAMJE | dxs (Cj0321) | Campylobacter jejuni |
| A1VY40 | DXS_CAMJJ | dxs (CJJ81176_0343) | Campylobacter jejuni subsp. jejuni serotype O:23/36 (strain 81- |
| | | | 176) |
| Q5HWF0 | DXS_CAMJR | dxs (CJE0366) | Campylobacter jejuni (strain RM1221) |
| O78328 | DXS_CAPAN | TKT2 | Capsicum annuum (Bell pepper) |
| Q3AAN0 | DXS_CARHZ | dxs (CHY_1985) | Carboxydothermus hydrogenoformans (strain Z-2901/DSM |
| | | | 6008) |
| B8GXC4 | DXS_CAUCN | dxs (CCNA_02149) | Caulobacter crescentus (strain NA1000/CB15N) |
| Q9A6M5 | DXS_CAUCR | dxs (CC_2068) | Caulobacter crescentus (Caulobacter vibrioides) |
| B0T3X7 | DXS_CAUSK | dxs (Caul_3314) | Caulobacter sp. (strain K31) |
| B3PF22 | DXS_CELJU | dxs (CJA_3336) | Cellvibrio japonicus (strain Ueda107) |
| Q5L6H4 | DXS_CHLAB | dxs (CAB301) | Chlamydophila abortus |
| Q823V1 | DXS_CHLCV | dxs (CCA_00304) | Chlamydophila caviae |
| Q253R7 | DXS_CHLFF | dxs (CF0699) | Chlamydophila felis (strain Fe/C-56) |
| Q9PK62 | DXS_CHLMU | dxs (TC_0608) | Chlamydia muridarum |
| Q9Z6J9 | DXS_CHLPN | dxs (CPn_1060) (CP_0790) | Chlamydia pneumoniae (Chlamydophila pneumoniae) |
| | | (CpB1102) | |
| B0B7P9 | DXS_CHLT2 | dxs (CTL0585) | Chlamydia trachomatis (strain L2/434/Bu/ATCC VR-902B) |
| Q3KM28 | DXS_CHLTA | dxs (CTA_0359) | Chlamydia trachomatis (strain A/HAR-13/ATCC VR-571B) |
| B0BBW4 | DXS_CHLTB | dxs (CTLon_0582) | Chlamydia trachomatis (strain L2b/UCH-1/proctitis) |
| Q8KFI9 | DXS_CHLTE | dxs (CT0337) | Chlorobium tepidum |
| O84335 | DXS_CHLTR | dxs (CT_331) | Chlamydia trachomatis |
| Q1R1E5 | DXS_CHRSD | dxs (Csal_0099) | Chromohalobacter salexigens (strain DSM 3043/ATCC BAA- |
| | | | 138/NCIMB 13768) |
| Q7NUK5 | DXS_CHRVO | dxs (CV_2692) | Chromobacterium violaceum |
| A8AK34 | DXS_CITK8 | dxs (CKO_02741) | Citrobacter koseri (strain ATCC BAA-895/CDC 4225-83/ |
| | | | SGSC4696) |
| B0RC26 | DXS_CLAMS | dxs (CMS1644) | Clavibacter michiganensis subsp. sepedonicus (strain ATCC |
| | | | 33113/JCM 9667) |
| Q97HD5 | DXS_CLOAB | dxs (CA_C2077) | Clostridium acetobutylicum |
| A6LU48 | DXS_CLOB8 | dxs (Cbei_1706) | Clostridium beijerinckii (strain ATCC 51743/NCIMB 8052) |
| | | | (Clostridium acetobutylicum) |
| B2V4R3 | DXS_CLOBA | dxs (CLH_2166) | Clostridium botulinum (strain Alaska E43/Type E3) |
| B2TRM5 | DXS_CLOBB | dxs (CLL_A2401) | Clostridium botulinum (strain Eklund 17B/Type B) |
| Q18B68 | DXS_CLOD6 | dxs (CD1207) | Clostridium difficile (strain 630) |
| B9E104 | DXS_CLOK1 | dxs (CKR_1128) | Clostridium kluyveri (strain NBRC 12016) |
| A5N7J2 | DXS_CLOK5 | dxs (CKL_1231) | Clostridium kluyveri (strain ATCC 8527/DSM 555/NCIMB |
| | | | 10680) |
| A0Q0A4 | DXS_CLONN | dxs (NT01CX_1983) | Clostridium novyi (strain NT) |
| Q0TPD8 | DXS_CLOP1 | dxs (CPF_2073) | Clostridium perfringens (strain ATCC 13124/NCTC 8237/Type |
| | | | A) |
| Q8XJE1 | DXS_CLOPE | dxs (CPE1819) | Clostridium perfringens |
| A9KMB8 | DXS_CLOPH | dxs (Cphy_2511) | Clostridium phytofermentans (strain ATCC 700394/DSM |
| | | | 18823/ISDg) |
| Q0SS05 | DXS_CLOPS | dxs (CPR_1787) | Clostridium perfringens (strain SM101/Type A) |
| Q894H0 | DXS_CLOTE | dxs (CTC_01575) | Clostridium tetani |
| Q487D3 | DXS_COLP3 | dxs (CPS_1088) | Colwellia psychrerythraea (strain 34H/ATCC BAA-681) (Vibrio |
| | | | psychroerythus) |
| Q6NGV3 | DXS_CORDI | dxs (DIP1397) | Corynebacterium diphtheriae |
| Q8FPI2 | DXS_COREF | dxs (CE1796) | Corynebacterium efficiens |
| A4QEQ9 | DXS_CORGB | dxs (cgR_1731) | Corynebacterium glutamicum (strain R) |
| Q8NPB2 | DXS_CORGL | dxs (Cgl1902) (cg2083) | Corynebacterium glutamicum (Brevibacterium flavum) |
| Q4JVB5 | DXS_CORJK | dxs (jk1078) | Corynebacterium jeikeium (strain K411) |
| B3R5H4 | DXS_CUPTR | dxs (RALTA_A2235) | Cupriavidus taiwanensis (strain R1/LMG 19424) (Ralstonia |
| | | | taiwanensis (strain LMG 19424)) |
| B1WWM7 | DXS_CYAA5 | dxs (cce_1401) | Cyanothece sp. (strain ATCC 51142) |
| B8HWL8 | DXS_CYAP4 | dxs (Cyan7425_4130) | Cyanothece sp. (strain PCC 7425/ATCC 29141) |
| B7KAF7 | DXS_CYAP7 | dxs (PCC7424_4569) | Cyanothece sp. (strain PCC 7424) (Synechococcus sp. (strain |
| | | | ATCC 29155)) |
| B7JVJ6 | DXS_CYAP8 | dxs (PCC8801_0471) | Cyanothece sp. (strain PCC 8801) (Synechococcus sp. (strain |
| | | | PCC 8801/RF-1)) |
| Q11NY7 | DXS_CYTH3 | dxs (CHU_3643) | Cytophaga hutchinsonii (strain ATCC 33406/NCIMB 9469) |
| Q47BJ0 | DXS_DECAR | dxs (Daro_3061) | Dechloromonas aromatica (strain RCB) |
| Q3Z8G9 | DXS_DEHE1 | dxs (DET0745) | Dehalococcoides ethenogenes (strain 195) |
| A5FRB9 | DXS_DEHSB | dxs (DehaBAV1_0675) | Dehalococcoides sp. (strain BAV1) |
| Q3ZXC2 | DXS_DEHSC | dxs (cbdbA720) | Dehalococcoides sp. (strain CBDB1) |
| Q1IZP0 | DXS_DEIGD | dxs (Dgeo_0994) | Deinococcus geothermalis (strain DSM 11300) |
| Q9RUB5 | DXS_DEIRA | dxs (DR_1475) | Deinococcus radiodurans |
| B1I3J6 | DXS_DESAP | dxs (Daud_1027) | Desulforudis audaxviator (strain MP104C) |
| Q30Z99 | DXS_DESDG | dxs (Dde_2200) | Desulfovibrio desulfuricans (strain G20) |
| B8FQ45 | DXS_DESHD | dxs (Dhaf_3488) | Desulfitobacterium hafniense (strain DCB-2/DSM 10664) |
| Q24V05 | DXS_DESHY | dxs (DSY2348) | Desulfitobacterium hafniense (strain Y51) |
| Q6AJQ1 | DXS_DESPS | dxs (DP2700) | Desulfotalea psychrophila |
| Q72CD3 | DXS_DESVH | dxs (DVU_1350) | Desulfovibrio vulgaris (strain Hildenborough/ATCC 29579/ |
| | | | NCIMB 8303) |
| A1VE69 | DXS_DESVV | dxs (Dvul_1718) | Desulfovibrio vulgaris subsp. vulgaris (strain DP4) |
| B9MEU8 | DXS_DIAST | dxs (Dtpsy_0956) | Diaphorobacter sp. (strain TPSY) |
| B5YE06 | DXS_DICT6 | dxs (DICTH_0902) | Dictyoglomus thermophilum (strain ATCC 35947/DSM 3960/ |
| | | | H-6-12) |
| B8E247 | DXS_DICTD | dxs (Dtur_1044) | Dictyoglomus turgidum (strain Z-1310/DSM 6724) |
| A7ZIH3 | DXS_ECO24 | dxs (EcE24377A_0451) | Escherichia coli O139:H28 (strain E24377A/ETEC) |
| B7UJP3 | DXS_ECO27 | dxs (E2348_C_0355) | Escherichia coli O127:H6 (strain E2348/69/EPEC) |
| B7MD78 | DXS_ECO45 | dxs (ECS88_0415) | Escherichia coli O45:K1 (strain S88/ExPEC) |
| B7L654 | DXS_ECO55 | dxs (EC55989_0430) | Escherichia coli (strain 55989/EAEC) |
| Q8XE76 | DXS_ECO57 | dxs (Z0523) (ECs0474) | Escherichia coli O157:H7 |
| B5Z3S5 | DXS_ECO5E | dxs (ECH74115_0503) | Escherichia coli O157:H7 (strain EC4115/EHEC) |
| B7NJ77 | DXS_ECO7I | dxs (ECIAI39_0256) | Escherichia coli O7:K1 (strain IAI39/ExPEC) |
| B7MQD5 | DXS_ECO81 | dxs (ECED1_0443) | Escherichia coli O81 (strain ED1a) |
| B7M3Q9 | DXS_ECO8A | dxs (ECIAI1_0420) | Escherichia coli O8 (strain IAI1) |
| C4ZTH7 | DXS_ECOBW | dxs (BWG_0302) | Escherichia coli (strain BW2952) |
| B1XF08 | DXS_ECODH | dxs (ECDH10B_0376) | Escherichia coli (strain DH10B) |
| A7ZX72 | DXS_ECOHS | dxs (EcHS_A0491) | Escherichia coli O9:H4 (strain HS) |
| A1A890 | DXS_ECOK1 | dxs (Ecok1_03860) | Escherichia coli O1:K1/APEC |
| | | (APECO1_1590) | |
| Q0TKM1 | DXS_ECOL5 | dxs (ECP_0479) | Escherichia coli O6:K15:H31 (strain 536/UPEC) |
| Q8FKB9 | DXS_ECOL6 | dxs (c0531) | Escherichia coli O6 |
| B1J029 | DXS_ECOLC | dxs (EcolC_3213) | Escherichia coli (strain ATCC 8739/DSM 1576/Crooks) |
| P77488 | DXS_ECOLI | dxs (yajP) (b0420) (JW0410) | Escherichia coli (strain K12) |
| B7N8X3 | DXS_ECOLU | dxs (ECUMN_0459) | Escherichia coli O17:K52:H18 (strain UMN026/ExPEC) |
| B6HZM1 | DXS_ECOSE | dxs (ECSE_0442) | Escherichia coli (strain SE11) |
| B1LJH0 | DXS_ECOSM | dxs (EcSMS35_0456) | Escherichia coli (strain SMS-3-5/SECEC) |
| Q1RFC0 | DXS_ECOUT | dxs (UTI89_C0443) | Escherichia coli (strain UTI89/UPEC) |
| C5BCH9 | DXS_EDWI9 | dxs (NT01EI_1061) | Edwardsiella ictaluri (strain 93-146) |
| A4W791 | DXS_ENT38 | dxs (Ent638_0887) | Enterobacter sp. (strain 638) |
| A7MFG0 | DXS_ENTS8 | dxs (ESA_02882) | Enterobacter sakazakii (strain ATCC BAA-894) |
| Q6D844 | DXS_ERWCT | dxs (ECA1131) | Erwinia carotovora subsp. atroseptica (Pectobacterium |
| | | | atrosepticum) |
| B2VHS3 | DXS_ERWT9 | dxs (ETA_25270) | Erwinia tasmaniensis (strain DSM 17950/Et1/99) |
| Q2N6U5 | DXS_ERYLH | dxs (ELI_12520) | Erythrobacter litoralis (strain HTCC2594) |
| B7LMG7 | DXS_ESCF3 | dxs (EFER_2605) | Escherichia fergusonii (strain ATCC 35469/DSM 13698/CDC |
| | | | 0568-73) |
| B1YLQ5 | DXS_EXIS2 | dxs (Exig_0908) | Exiguobacterium sibiricum (strain DSM 17290/JCM 13490/ |
| | | | 255-15) |
| B0U0B3 | DXS_FRAP2 | dxs (Fphi_1718) | Francisella philomiragia subsp. philomiragia (strain ATCC |
| | | | 25017) |
| Q2JDD9 | DXS_FRASC | dxs (Francci3_1326) | Frankia sp. (strain Ccl3) |
| A8L0K9 | DXS_FRASN | dxs (Franean1_5184) | Frankia sp. (strain EAN1pec) |
| Q14HJ1 | DXS_FRAT1 | dxs (FTF1018c) | Francisella tularensis subsp. tularensis (strain FSC 198) |
| A7NCA4 | DXS_FRATF | dxs (FTA_1131) | Francisella tularensis subsp. holarctica (strain FTA) |
| Q2A3D3 | DXS_FRATH | dxs (FTL_1072) | Francisella tularensis subsp. holarctica (strain LVS) |
| B2SGK5 | DXS_FRATM | dxs (FTM_0932) | Francisella tularensis subsp. mediasiatica (strain FSC147) |
| A0Q6B9 | DXS_FRATN | dxs (FTN_0896) | Francisella tularensis subsp. novicida (strain U112) |
| Q0BLU9 | DXS_FRATO | dxs (FTH_1047) | Francisella tularensis subsp. holarctica (strain OSU18) |
| Q5NG39 | DXS_FRATT | dxs (FTT1018c) | Francisella tularensis subsp. tularensis |
| A4IXW5 | DXS_FRATW | dxs (FTW_0925) | Francisella tularensis subsp. tularensis (strain WY96-3418) |
| Q8R639 | DXS_FUSNN | dxs (FN1208) | Fusobacterium nucleatum subsp. nucleatum |
| Q75TB7 | DXS_GEOKA | dxs (GK2392) (GKC05) | Geobacillus kaustophilus |
| C5D467 | DXS_GEOSW | dxs (GWCH70_2319) | Geobacillus sp. (strain WCH70) |
| A4IQR7 | DXS_GEOTN | dxs (GTNG_2322) | Geobacillus thermodenitrificans (strain NG80-2) |
| Q7NP63 | DXS_GLOVI | dxs (gll0194) | Gloeobacter violaceus |
| Q5FUB1 | DXS_GLUOX | dxs (GOX0252) | Gluconobacter oxydans (Gluconobacter suboxydans) |
| Q7VNP7 | DXS_HAEDU | dxs (HD_0441) | Haemophilus ducreyi |
| Q4QKG6 | DXS_HAEI8 | dxs (NTHI1691) | Haemophilus influenzae (strain 86-028NP) |
| A5UC51 | DXS_HAEIE | dxs (CGSHiEE_04795) | Haemophilus influenzae (strain PittEE) |
| A5UEV6 | DXS_HAEIG | dxs (CGSHiGG_01080) | Haemophilus influenzae (strain PittGG) |
| P45205 | DXS_HAEIN | dxs (HI1439) | Haemophilus influenzae |
| B8F3A4 | DXS_HAEPS | dxs (HAPS_0107) | Haemophilus parasuis serovar 5 (strain SH0165) |
| Q0I3G1 | DXS_HAES1 | dxs (HS_0905) | Haemophilus somnus (strain 129Pt) (Histophilus somni (strain |
| | | | 129Pt)) |
| B0UUA4 | DXS_HAES2 | dxs (HSM_1383) | Haemophilus somnus (strain 2336) (Histophilus somni (strain |
| | | | 2336)) |
| Q2SA08 | DXS_HAHCH | dxs (HCH_05866) | Hahella chejuensis (strain KCTC 2396) |
| B8D2I3 | DXS_HALOH | dxs (Hore_06530) | Halothermothrix orenii (strain H 168/OCM 544/DSM 9562) |
| C4K6M7 | DXS_HAMD5 | dxs (HDEF_1608) | Hamiltonella defensa subsp. Acyrthosiphon pisum (strain 5AT) |
| Q7VIJ7 | DXS_HELHP | dxs (HH_0608) | Helicobacter hepaticus |
| B0TEJ5 | DXS_HELMI | dxs (Helmi_04700) | Heliobacterium modesticaldum (strain ATCC 51547/Ice1) |
| | | (HM1_0295) | |
| B5ZAB7 | DXS_HELPG | dxs (HPG27_331) | Helicobacter pylori (strain G27) |
| Q1CUF6 | DXS_HELPH | dxs (HPAG1_0349) | Helicobacter pylori (strain HPAG1) |
| Q9ZM94 | DXS_HELPJ | dxs (jhp_0328) | Helicobacter pylori J99 (Campylobacter pylori J99) |
| O25121 | DXS_HELPY | dxs (HP_0354) | Helicobacter pylori (Campylobacter pylori) |
| Q0C154 | DXS_HYPNA | dxs (HNE_1838) | Hyphomonas neptunium (strain ATCC 15444) |
| Q5QVE8 | DXS_IDILO | dxs (IL2138) | Idiomarina loihiensis |
| B5Y0X1 | DXS_KLEP3 | dxs (KPK_4312) | Klebsiella pneumoniae (strain 342) |
| A6T5F3 | DXS_KLEP7 | dxs (KPN78578_03630) | Klebsiella pneumoniae subsp. pneumoniae (strain ATCC |
| | | (KPN_00372) | 700721/MGH 78578) |
| B2GJ56 | DXS_KOCRD | dxs (KRH_14140) | Kocuria rhizophila (strain ATCC 9341/DSM 348/NBRC 103217/ |
| | | | DC2201) |
| C1DAW8 | DXS_LARHH | dxs (LHK_02324) | Laribacter hongkongensis (strain HLHK9) |
| Q1MRB3 | DXS_LAWIP | dxs (LI0408) | Lawsonia intracellularis (strain PHE/MN1-00) |
| Q6AFD5 | DXS_LEIXX | dxs (Lxx10450) | Leifsonia xyli subsp. xyli |
| Q04U59 | DXS_LEPBJ | dxs (LBJ_0917) | Leptospira borgpetersenii serovar Hardjo-bovis (strain JB197) |
| Q053M2 | DXS_LEPBL | dxs (LBL_0932) | Leptospira borgpetersenii serovar Hardjo-bovis (strain L550) |
| B1Y2X5 | DXS_LEPCP | dxs (Lcho_3373) | Leptothrix cholodnii (strain ATCC 51168/LMG 8142/SP-6) |
| | | | (Leptothrix discophora (strain SP-6)) |
| Q72U01 | DXS_LEPIC | dxs (LIC_10863) | Leptospira interrogans serogroup Icterohaemorrhagiae serovar |
| | | | copenhageni |
| Q8F153 | DXS_LEPIN | dxs (LA_3285) | Leptospira interrogans |
| Q92BZ0 | DXS_LISIN | dxs (lin1402) | Listeria innocua |
| C1L2S1 | DXS_LISMC | dxs (Lm4b_01374) | Listeria monocytogenes serotype 4b (strain Clip81459) |
| Q71ZV7 | DXS_LISMF | dxs (LMOf2365_1382) | Listeria monocytogenes serotype 4b (strain F2365) |
| Q8Y7C1 | DXS_LISMO | dxs (lmo1365) | Listeria monocytogenes |
| A0AIG6 | DXS_LISW6 | dxs (lwe1380) | Listeria welshimeri serovar 6b (strain ATCC 35897/DSM 20650/ |
| | | | SLCC5334) |
| B1HRX4 | DXS_LYSSC | dxs (Bsph_3509) | Lysinibacillus sphaericus (strain C3-41) |
| B9E6Q6 | DXS_MACCJ | dxs (MCCL_1167) | Macrococcus caseolyticus (strain JCSC5402) |
| Q2W367 | DXS_MAGSA | dxs (amb2904) | Magnetospirillum magneticum (strain AMB-1/ATCC 700264) |
| A0L6H3 | DXS_MAGSM | dxs (Mmc1_1048) | Magnetococcus sp. (strain MC-1) |
| Q65TP4 | DXS_MANSM | dxs (MS1059) | Mannheimia succiniciproducens (strain MBEL55E) |
| Q0ARE5 | DXS_MARMM | dxs (Mmar10_0849) | Maricaulis maris (strain MCS10) |
| A6VUE5 | DXS_MARMS | dxs (Mmwyl1_1145) | Marinomonas sp. (strain MWYL1) |
| Q11KE0 | DXS_MESSB | dxs (Meso_0735) | Mesorhizobium sp. (strain BNC1) |
| Q60AN1 | DXS_METCA | dxs (MCA0817) | Methylococcus capsulatus |
| Q1GZD7 | DXS_METFK | dxs (Mfla_2133) | Methylobacillus flagellatus (strain KT/ATCC 51484/DSM |
| | | | 6875) |
| B3DW88 | DXS_METI4 | dxs (Minf_1537) | Methylacidiphilum infernorum (isolate V4) (Methylokorus |
| | | | infernorum (strain V4)) |
| A2SJ46 | DXS_METPP | dxs (Mpe_A2631) | Methylibium petroleiphilum (strain PM1) |
| B0JL88 | DXS_MICAN | dxs (MAE_62650) | Microcystis aeruginosa (strain NIES-843) |
| Q2RIB9 | DXS_MOOTA | dxs (Moth_1511) | Moorella thermoacetica (strain ATCC 39073) |
| A0QIL6 | DXS_MYCA1 | dxs (MAV_3577) | Mycobacterium avium (strain 104) |
| B1MCU7 | DXS_MYCA9 | dxs (MAB_2990c) | Mycobacterium abscessus (strain ATCC 19977/DSM 44196) |
| P0A555 | DXS_MYCBO | dxs (Mb2701c) | Mycobacterium bovis |
| A1KM20 | DXS_MYCBP | dxs (BCG_2695c) | Mycobacterium bovis (strain BCG/Pasteur 1173P2) |
| C1AFE1 | DXS_MYCBT | dxs (JTY_2689) | Mycobacterium bovis (strain BCG/Tokyo 172/ATCC 35737/ |
| | | | TMC 1019) |
| A4TCS5 | DXS_MYCGI | dxs (Mflv_3923) | Mycobacterium gilvum (strain PYR-GCK) (Mycobacterium |
| | | | flavescens (strain ATCC 700033/PYR-GCK)) |
| B8ZQW9 | DXS_MYCLB | dxs (MLBr01038) | Mycobacterium leprae (strain Br4923) |
| Q50000 | DXS_MYCLE | dxs (tktB) (ML1038) | Mycobacterium leprae |
| Q73W57 | DXS_MYCPA | dxs (MAP_2803c) | Mycobacterium paratuberculosis |
| Q8EWX7 | DXS_MYCPE | dxs (MYPE730) | Mycoplasma penetrans |
| A0QW19 | DXS_MYCS2 | dxs (MSMEG_2776) | Mycobacterium smegmatis (strain ATCC 700084/mc(2)155) |
| A3PYK6 | DXS_MYCSJ | dxs (Mjls_2197) | Mycobacterium sp. (strain JLS) |
| A1UF44 | DXS_MYCSK | dxs (Mkms_2254) | Mycobacterium sp. (strain KMS) |
| Q1B9W8 | DXS_MYCSS | dxs (Mmcs_2208) | Mycobacterium sp. (strain MCS) |
| A5U634 | DXS_MYCTA | dxs (MRA_2710) | Mycobacterium tuberculosis (strain ATCC 25177/H37Ra) |
| P0A554 | DXS_MYCTU | dxs (Rv2682c) (MT2756) | Mycobacterium tuberculosis |
| | | (MTCY05A6.03c) | |
| A0PT40 | DXS_MYCUA | dxs (MUL_3319) | Mycobacterium ulcerans (strain Agy99) |
| A1T7Z0 | DXS_MYCVP | dxs (Mvan_2477) | Mycobacterium vanbaalenii (strain DSM 7251/PYR-1) |
| Q1D3G4 | DXS_MYXXD | dxs (MXAN_4643) | Myxococcus xanthus (strain DK 1622) |
| B2A526 | DXS_NATTJ | dxs (Nther_1694) | Natranaerobius thermophilus (strain ATCC BAA-1301/DSM |
| | | | 18059/JW/NM-WN-LF) |
| Q5FAI2 | DXS_NEIG1 | dxs (NGO0036) | Neisseria gonorrhoeae (strain ATCC 700825/FA 1090) |
| B4RNW6 | DXS_NEIG2 | dxs (NGK_0044) | Neisseria gonorrhoeae (strain NCCP11945) |
| A9M1G3 | DXS_NEIM0 | dxs (NMCC_0354) | Neisseria meningitidis serogroup C (strain 053442) |
| Q9JW13 | DXS_NEIMA | dxs (NMA0589) | Neisseria meningitidis serogroup A |
| Q9JXV7 | DXS_NEIMB | dxs (NMB1867) | Neisseria meningitidis serogroup B |
| A1KS32 | DXS_NEIMF | dxs (NMC0352) | Neisseria meningitidis serogroup C/serotype 2a (strain ATCC |
| | | | 700532/FAM18) |
| Q0AFY6 | DXS_NITEC | dxs (Neut_1501) | Nitrosomonas eutropha (strain C91) |
| Q82VD3 | DXS_NITEU | dxs (NE1161) | Nitrosomonas europaea |
| Q1QQ40 | DXS_NITHX | dxs (Nham_0778) | Nitrobacter hamburgensis (strain X14/DSM 10229) |
| Q2YCH7 | DXS_NITMU | dxs (Nmul_A0236) | Nitrosospira multiformis (strain ATCC 25196/NCIMB 11849) |
| Q3JAD1 | DXS_NITOC | dxs (Noc_1743) | Nitrosococcus oceani (strain ATCC 19707/NCIMB 11848) |
| A6Q1Z6 | DXS_NITSB | dxs (NIS_0391) | Nitratiruptor sp. (strain SB155-2) |
| Q3SUZ1 | DXS_NITWN | dxs (Nwi_0633) | Nitrobacter winogradskyi (strain Nb-255/ATCC 25391) |
| Q5YTA2 | DXS_NOCFA | dxs (NFA_37410) | Nocardia farcinica |
| A1SKM6 | DXS_NOCSJ | dxs (Noca_2859) | Nocardioides sp. (strain BAA-499/JS614) |
| B2J5P1 | DXS_NOSP7 | dxs (Npun_F5466) | Nostoc punctiforme (strain ATCC 29133/PCC 73102) |
| Q2GC13 | DXS_NOVAD | dxs (Saro_0161) | Novosphingobium aromaticivorans (strain DSM 12444) |
| A6WWC4 | DXS_OCHA4 | dxs (Oant_0547) | Ochrobactrum anthropi (strain ATCC 49188/DSM 6882/NCTC |
| | | | 12168) |
| O22567 | DXS_ORYSJ | CLA1 (Os05g0408900) | Oryza sativa subsp. japonica (Rice) |
| | | (LOC_Os05g33840) | |
| | | (OSJNBb0014K18.8) | |
| | | (P0040B10.17) | |
| A6LFB9 | DXS_PARD8 | dxs (BDI_2664) | Parabacteroides distasonis (strain ATCC 8503/DSM 20701/ |
| | | | NCTC 11152) |
| Q6MDK6 | DXS_PARUW | dxs (pc0619) | Protochlamydia amoebophila (strain UWE25) |
| P57848 | DXS_PASMU | dxs (PM0532) | Pasteurella multocida |
| C6DB37 | DXS_PECCP | dxs (PC1_1030) | Pectobacterium carotovorum subsp. carotovorum (strain PC1) |
| Q3A3Z6 | DXS_PELCD | dxs (Pcar_1667) | Pelobacter carbinolicus (strain DSM 2380/Gra Bd 1) |
| Q3B5P3 | DXS_PELLD | dxs (Plut_0450) | Pelodictyon luteolum (strain DSM 273) (Chlorobium luteolum |
| | | | (strain DSM 273)) |
| A5D2Z6 | DXS_PELTS | dxs (PTH_1196) | Pelotomaculum thermopropionicum (strain DSM 13744/JCM |
| | | | 10971/SI) |
| Q4FN07 | DXS_PELUB | dxs (SAR11_0611) | Pelagibacter ubique |
| B4RGW0 | DXS_PHEZH | dxs (PHZ_c0912) | Phenylobacterium zucineum (strain HLK1) |
| Q7N0J7 | DXS_PHOLL | dxs (plu3887) | Photorhabdus luminescens subsp. laumondii |
| Q6LU07 | DXS_PHOPR | dxs (PBPRA0805) | Photobacterium profundum (Photobacterium sp. (strain SS9)) |
| A1VMD7 | DXS_POLNA | dxs (Pnap_1501) | Polaromonas naphthalenivorans (strain CJ2) |
| Q12CQ9 | DXS_POLSJ | dxs (Bpro_1747) | Polaromonas sp. (strain JS666/ATCC BAA-500) |
| B2RMK4 | DXS_PORG3 | dxs (PGN_2081) | Porphyromonas gingivalis (strain ATCC 33277/DSM 20709/ |
| | | | JCM 12257) |
| Q7MSZ3 | DXS_PORGI | dxs (PG_2217) | Porphyromonas gingivalis (Bacteroides gingivalis) |
| Q6A8V3 | DXS_PROAC | dxs (PPA1062) | Propionibacterium acnes |
| A3PCV0 | DXS_PROM0 | dxs (P9301_09521) | Prochlorococcus marinus (strain MIT 9301) |
| A2C220 | DXS_PROM1 | dxs (NATL1_09721) | Prochlorococcus marinus (strain NATL1A) |
| A8G4R9 | DXS_PROM2 | dxs (P9215_09851) | Prochlorococcus marinus (strain MIT 9215) |
| A2C9X1 | DXS_PROM3 | dxs (P9303_15371) | Prochlorococcus marinus (strain MIT 9303) |
| A9BAC1 | DXS_PROM4 | dxs (P9211_08521) | Prochlorococcus marinus (strain MIT 9211) |
| A2BWN6 | DXS_PROM5 | dxs (P9515_09901) | Prochlorococcus marinus (strain MIT 9515) |
| Q31AZ2 | DXS_PROM9 | dxs (PMT9312_0893) | Prochlorococcus marinus (strain MIT 9312) |
| Q7VC14 | DXS_PROMA | dxs (Pro_0928) | Prochlorococcus marinus |
| B4EU31 | DXS_PROMH | dxs (PMI0094) | Proteus mirabilis (strain HI4320) |
| Q7V7Q3 | DXS_PROMM | dxs (PMT_0685) | Prochlorococcus marinus (strain MIT 9313) |
| Q7V1G6 | DXS_PROMP | dxs (PMM0907) | Prochlorococcus marinus subsp. pastoris (strain CCMP1986/ |
| | | | MED4) |
| A2BR27 | DXS_PROMS | dxs (A9601_09541) | Prochlorococcus marinus (strain AS9601) |
| Q46L36 | DXS_PROMT | dxs (PMN2A_0300) | Prochlorococcus marinus (strain NATL2A) |
| A4SDG1 | DXS_PROVI | dxs (Cvib_0498) | Prosthecochloris vibrioformis (strain DSM 265) (Chlorobium |
| | | | vibrioforme subsp. thiosulfatophilum (strain DSM 265)) |
| | | | (Chlorobium phaeovibrioides (strain DSM 265)) |
| Q48NX0 | DXS_PSE14 | dxs (PSPPH_0599) | Pseudomonas syringae pv. phaseolicola (strain 1448A/Race 6) |
| Q15W93 | DXS_PSEA6 | dxs (Patl_1319) | Pseudoalteromonas atlantica (strain T6c/BAA-1087) |
| A6V058 | DXS_PSEA7 | dxs (PSPA7_1057) | Pseudomonas aeruginosa (strain PA7) |
| B7V7R4 | DXS_PSEA8 | dxs (PLES_09321) | Pseudomonas aeruginosa (strain LESB58) |
| Q02SL1 | DXS_PSEAB | dxs (PA14_11550) | Pseudomonas aeruginosa (strain UCBPP-PA14) |
| Q9KGU7 | DXS_PSEAE | dxs (PA4044) | Pseudomonas aeruginosa |
| Q1IFL1 | DXS_PSEE4 | dxs (PSEEN0600) | Pseudomonas entomophila (strain L48) |
| Q4K5A5 | DXS_PSEF5 | dxs (PFL_5510) | Pseudomonas fluorescens (strain Pf-5/ATCC BAA-477) |
| C3K2R1 | DXS_PSEFS | dxs (PFLU_5462) | Pseudomonas fluorescens (strain SBW25) |
| Q3II09 | DXS_PSEHT | dxs (PSHAa2366) | Pseudoalteromonas haloplanktis (strain TAC 125) |
| A4XZ25 | DXS_PSEMY | dxs (Pmen_3844) | Pseudomonas mendocina (strain ymp) |
| A5VXW9 | DXS_PSEP1 | dxs (Pput_0561) | Pseudomonas putida (strain F1/ATCC 700007) |
| Q3K660 | DXS_PSEPF | dxs (Pfl01_5007) | Pseudomonas fluorescens (strain Pf0-1) |
| B0KL79 | DXS_PSEPG | dxs (PputGB1_0572) | Pseudomonas putida (strain GB-1) |
| Q88QG7 | DXS_PSEPK | dxs (PP_0527) | Pseudomonas putida (strain KT2440) |
| B1J3G4 | DXS_PSEPW | dxs (PputW619_0579) | Pseudomonas putida (strain W619) |
| Q889Q1 | DXS_PSESM | dxs (PSPTO_0698) | Pseudomonas syringae pv. tomato |
| Q4ZYU8 | DXS_PSEU2 | dxs (Psyr_0604) | Pseudomonas syringae pv. syringae (strain B728a) |
| A4VQS8 | DXS_PSEU5 | dxs (PST_3706) | Pseudomonas stutzeri (strain A1501) |
| Q4FV64 | DXS_PSYA2 | dxs (Psyc_0221) | Psychrobacter arcticus (strain DSM 17307/273-4) |
| Q1QE74 | DXS_PSYCK | dxs (Pcryo_0245) | Psychrobacter cryohalolentis (strain K5) |
| A1SWW6 | DXS_PSYIN | dxs (Ping_2240) | Psychromonas ingrahamii (strain 37) |
| Q0K860 | DXS_RALEH | dxs (H16_A2732) | Ralstonia eutropha (strain ATCC 17699/H16/DSM 428/ |
| | | | Stanier 337) (Cupriavidus necator (strain ATCC 17699/H16/ |
| | | | DSM 428/Stanier 337)) |
| Q474C2 | DXS_RALEJ | dxs (Reut_A0882) | Ralstonia eutropha (strain JMP134) (Alcaligenes eutrophus) |
| Q1LK34 | DXS_RALME | dxs (Rmet_2615) | Ralstonia metallidurans (strain CH34/ATCC 43123/DSM |
| | | | 2839) |
| B2U930 | DXS_RALPJ | dxs (Rpic_2426) | Ralstonia pickettii (strain 12J) |
| Q8XX95 | DXS_RALSO | dxs (RSc2221) (RS01378) | Ralstonia solanacearum (Pseudomonas solanacearum) |
| A9WRA9 | DXS_RENSM | dxs (RSal33209_2392) | Renibacterium salmoninarum (strain ATCC 33209/DSM 20767/ |
| | | | IFO 15589) |
| B3PS68 | DXS_RHIE6 | dxs (RHECIAT_CH0001005) | Rhizobium etli (strain CIAT 652) |
| Q2KBR2 | DXS_RHIEC | dxs (RHE_CH00913) | Rhizobium etli (strain CFN 42/ATCC 51251) |
| Q1MKN4 | DXS_RHIL3 | dxs (RL0973) | Rhizobium leguminosarum bv. viciae (strain 3841) |
| Q985Y3 | DXS_RHILO | dxs (mlr7474) | Rhizobium loti (Mesorhizobium loti) |
| B5ZS68 | DXS_RHILW | dxs (Rleg2_0564) | Rhizobium leguminosarum bv. trifolii (strain WSM2304) |
| Q92RJ1 | DXS_RHIME | dxs (R00880) (SMc00972) | Rhizobium meliloti (Sinorhizobium meliloti) |
| Q7UWB7 | DXS_RHOBA | dxs (RB2143) | Rhodopirellula baltica |
| P26242 | DXS_RHOCA | dxs | Rhodobacter capsulatus (Rhodopseudomonas capsulata) |
| B6IRB5 | DXS_RHOCS | dxs (RC1_0565) | Rhodospirillum centenum (strain ATCC 51521/SW) |
| C0ZYV9 | DXS_RHOE4 | dxs (RER_28360) | Rhodococcus erythropolis (strain PR4/NBRC 100887) |
| Q21UG7 | DXS_RHOFD | dxs (Rfer_2875) | Rhodoferax ferrireducens (strain DSM 15236/ATCC BAA-621/ |
| | | | T118) |
| Q2IRL7 | DXS_RHOP2 | dxs (RPB_4460) | Rhodopseudomonas palustris (strain HaA2) |
| Q07SR3 | DXS_RHOP5 | dxs (RPE_1067) | Rhodopseudomonas palustris (strain BisA53) |
| Q6NB76 | DXS_RHOPA | dxs (RPA0952) | Rhodopseudomonas palustris |
| Q21A74 | DXS_RHOPB | dxs (RPC_1149) | Rhodopseudomonas palustris (strain BisB18) |
| Q130G7 | DXS_RHOPS | dxs (RPD_4305) | Rhodopseudomonas palustris (strain BisB5) |
| B3QFY7 | DXS_RHOPT | dxs (Rpal_1022) | Rhodopseudomonas palustris (strain TIE-1) |
| Q0S1H1 | DXS_RHOSR | dxs (RHA1_ro06843) | Rhodococcus sp. (strain RHA1) |
| Q21F93 | DXS_SACD2 | dxs (Sde_3381) | Saccharophagus degradans (strain 2-40/ATCC 43961/DSM |
| | | | 17024) |
| A4FAQ5 | DXS_SACEN | dxs (SACE_1815) | Saccharopolyspora erythraea (strain NRRL_23338) |
| B5EXG3 | DXS_SALA4 | dxs (SeAg_B0461) | Salmonella agona (strain SL483) |
| A9MM42 | DXS_SALAR | dxs (SARI_02505) | Salmonella arizonae (strain ATCC BAA-731/CDC346-86/ |
| | | | RSK2980) |
| Q57SE2 | DXS_SALCH | dxs (SCH_0463) | Salmonella choleraesuis |
| B5FKS7 | DXS_SALDC | dxs (SeD_A0463) | Salmonella dublin (strain CT_02021853) |
| B5QTH0 | DXS_SALEP | dxs (SEN0404) | Salmonella enteritidis PT4 (strain P125109) |
| B5R6S3 | DXS_SALG2 | dxs (SG0433) | Salmonella gallinarum (strain 287/91/NCTC 13346) |
| B4T8R3 | DXS_SALHS | dxs (SeHA_C0524) | Salmonella heidelberg (strain SL476) |
| B4SWR4 | DXS_SALNS | dxs (SNSL254_A0469) | Salmonella newport (strain SL254) |
| Q5PFR6 | DXS_SALPA | dxs (SPA2301) | Salmonella paratyphi A |
| A9MX09 | DXS_SALPB | dxs (SPAB_03161) | Salmonella paratyphi B (strain ATCC BAA-1250/SPB7) |
| C0Q7U7 | DXS_SALPC | dxs (SPC_0434) | Salmonella paratyphi C (strain RKS4594) |
| B5BDB0 | DXS_SALPK | dxs (SSPA2143) | Salmonella paratyphi A (strain AKU_12601) |
| B4TMA2 | DXS_SALSV | dxs (SeSA_A0482) | Salmonella schwarzengrund (strain CVM19633) |
| Q8Z8X3 | DXS_SALTI | dxs (STY0461) (t2441) | Salmonella typhi |
| Q8ZRD1 | DXS_SALTY | dxs (STM0422) | Salmonella typhimurium |
| A8GAP2 | DXS_SERP5 | dxs (Spro_1078) | Serratia proteamaculans (strain 568) |
| A1S8D4 | DXS_SHEAM | dxs (Sama_2436) | Shewanella amazonensis (strain ATCC BAA-1098/SB2B) |
| B8EAU7 | DXS_SHEB2 | dxs (Sbal223_3003) | Shewanella baltica (strain OS223) |
| A3D2B2 | DXS_SHEB5 | dxs (Sbal_1357) | Shewanella baltica (strain OS155/ATCC BAA-1091) |
| A6WL04 | DXS_SHEB8 | dxs (Shew185_1343) | Shewanella baltica (strain OS185) |
| A9KTL5 | DXS_SHEB9 | dxs (Sbal195_1382) | Shewanella baltica (strain OS195) |
| Q12L26 | DXS_SHEDO | dxs (Sden_2571) | Shewanella denitrificans (strain OS217/ATCC BAA-1090/DSM |
| | | | 15013) |
| Q07ZD4 | DXS_SHEFN | dxs (Sfri_2790) | Shewanella frigidimarina (strain NCIMB 400) |
| B0TQ36 | DXS_SHEHH | dxs (Shal_3080) | Shewanella halifaxensis (strain HAW-EB4) |
| A3QGN9 | DXS_SHELP | dxs (Shew_2771) | Shewanella loihica (strain ATCC BAA-1088/PV-4) |
| Q8EGR9 | DXS_SHEON | dxs (SO_1525) | Shewanella oneidensis |
| A8H6X1 | DXS_SHEPA | dxs (Spea_2991) | Shewanella pealeana (strain ATCC 700345/ANG-SQ1) |
| A4Y4X0 | DXS_SHEPC | dxs (Sputcn32_1275) | Shewanella putrefaciens (strain CN-32/ATCC BAA-453) |
| B8CS19 | DXS_SHEPW | dxs (swp_3619) | Shewanella piezotolerans (strain WP3/JCM 13877) |
| A0KZA9 | DXS_SHESA | dxs (Shewana3_2901) | Shewanella sp. (strain ANA-3) |
| A8FYL0 | DXS_SHESH | dxs (Ssed_3329) | Shewanella sediminis (strain HAW-EB3) |
| Q0HGL5 | DXS_SHESM | dxs (Shewmr4_2731) | Shewanella sp. (strain MR-4) |
| Q0HSW6 | DXS_SHESR | dxs (Shewmr7_2804) | Shewanella sp. (strain MR-7) |
| A1RLV3 | DXS_SHESW | dxs (Sputw3181_2831) | Shewanella sp. (strain W3-18-1) |
| B1KQY8 | DXS_SHEWM | dxs (Swoo_3478) | Shewanella woodyi (strain ATCC 51908/MS32) |
| B2U4M3 | DXS_SHIB3 | dxs (SbBS512_E0341) | Shigella boydii serotype 18 (strain CDC 3083-94/BS512) |
| Q325I1 | DXS_SHIBS | dxs (SBO_0314) | Shigella boydii serotype 4 (strain Sb227) |
| Q32JH8 | DXS_SHIDS | dxs (SDY_0310) | Shigella dysenteriae serotype 1 (strain Sd197) |
| Q83SG2 | DXS_SHIFL | dxs (SF0357) (S0365) | Shigella flexneri |
| Q3Z4Y9 | DXS_SHISS | dxs (SSON_0397) | Shigella sonnei (strain Ss046) |
| Q5LX42 | DXS_SILPO | dxs (SPO0247) | Silicibacter pomeroyi |
| Q1GCG4 | DXS_SILST | dxs (TM1040_2920) | Silicibacter sp. (strain TM1040) |
| Q2NV94 | DXS_SODGM | dxs (SG0656) | Sodalis glossinidius (strain morsitans) |
| Q1GQK9 | DXS_SPHAL | dxs (Sala_2354) | Sphingopyxis alaskensis (Sphingomonas alaskensis) |
| A5V6A9 | DXS_SPHWW | dxs (Swit_1461) | Sphingomonas wittichii (strain RW1/DSM 6014/JCM 10273) |
| Q9RBN6 | DXS_STRC1 | dxs | Streptomyces sp. (strain CL190) |
| B1VWJ8 | DXS_STRGG | dxs (SGR_1495) | Streptomyces griseus subsp. griseus (strain JCM 4626/NBRC |
| | | | 13350) |
| B2FN57 | DXS_STRMK | dxs (Smlt3355) | Stenotrophomonas maltophilia (strain K279a) |
| Q30TC5 | DXS_SULDN | dxs (Suden_0475) | Sulfurimonas denitrificans (strain ATCC 33889/DSM 1251) |
| | | | (Thiomicrospira denitrificans (strain ATCC 33889/DSM 1251)) |
| Q67NB6 | DXS_SYMTH | dxs (STH1842) | Symbiobacterium thermophilum |
| Q2LUA7 | DXS_SYNAS | dxs (SYNAS_17860) | Syntrophus aciditrophicus (strain SB) |
| | | (SYN_02456) | |
| Q8GAA0 | DXS_SYNE7 | dxs (Synpcc7942_0430) | Synechococcus elongatus (strain PCC 7942) (Anacystis nidulans |
| | | (sel0040) | R2) |
| Q2JTX2 | DXS_SYNJA | dxs (CYA_1701) | Synechococcus sp. (strain JA-3-3Ab) (Cyanobacteria bacterium |
| | | | Yellowstone A-Prime) |
| Q2JK64 | DXS_SYNJB | dxs (CYB_1983) | Synechococcus sp. (strain JA-2-3B′a(2-13)) (Cyanobacteria |
| | | | bacterium Yellowstone B-Prime) |
| B1XKC5 | DXS_SYNP2 | dxs (SYNPCC7002_A1172) | Synechococcus sp. (strain ATCC 27264/PCC 7002/PR-6) |
| | | | (Agmenellum quadruplicatum) |
| Q9R6S7 | DXS_SYNP6 | dxs (syc1087_c) | Synechococcus sp. (strain ATCC 27144/PCC 6301/SAUG |
| | | | 1402/1) (Anacystis nidulans) |
| A5GL34 | DXS_SYNPW | dxs (SynWH7803_1223) | Synechococcus sp. (strain WH7803) |
| Q7U6P6 | DXS_SYNPX | dxs (SYNW1292) | Synechococcus sp. (strain WH8102) |
| A5GTT4 | DXS_SYNR3 | dxs (SynRCC307_1390) | Synechococcus sp. (strain RCC307) |
| Q0IAA6 | DXS_SYNS3 | dxs (sync_1410) | Synechococcus sp. (strain CC9311) |
| Q3AXZ4 | DXS_SYNS9 | dxs (Syncc9902_1069) | Synechococcus sp. (strain CC9902) |
| Q3AJP8 | DXS_SYNSC | dxs (Syncc9605_1430) | Synechococcus sp. (strain CC9605) |
| Q0AZE2 | DXS_SYNWW | dxs (Swol_0582) | Syntrophomonas wolfei subsp. wolfei (strain Goettingen) |
| P73067 | DXS_SYNY3 | dxs (sll1945) | Synechocystis sp. (strain PCC 6803) |
| Q8DL74 | DXS_THEEB | dxs (tll0623) | Thermosynechococcus elongatus (strain BP-1) |
| Q47NL9 | DXS_THEFY | dxs (Tfu_1917) | Thermobifida fusca (strain YX) |
| Q9X291 | DXS_THEMA | dxs (TM_1770) | Thermotoga maritima |
| A5ILK2 | DXS_THEP1 | dxs (Tpet_1058) | Thermotoga petrophila (strain RKU-1/ATCC BAA-488/DSM |
| | | | 13995) |
| B9L1L6 | DXS_THERP | dxs (trd_1276) | Thermomicrobium roseum (strain ATCC 27502/DSM 5159/P- |
| | | | 2) |
| B1LAQ3 | DXS_THESQ | dxs (TRQ2_1054) | Thermotoga sp. (strain RQ2) |
| Q72H81 | DXS_THET2 | dxs (TT_C1614) | Thermus thermophilus (strain HB27/ATCC BAA-163/DSM |
| | | | 7039) |
| Q5SMD7 | DXS_THET8 | dxs (TTHA0006) | Thermus thermophilus (strain HB8/ATCC 27634/DSM 579) |
| Q8RAC5 | DXS_THETN | dxs (TTE1298) | Thermoanaerobacter tengcongensis |
| Q3SKF1 | DXS_THIDA | dxs (Tbd_0879) | Thiobacillus denitrificans (strain ATCC 25259) |
| B8GN62 | DXS_THISH | dxs (Tgr7_0832) | Thioalkalivibrio sp. (strain HL-EbGR7) |
| Q73LF4 | DXS_TREDE | dxs (TDE_1910) | Treponema denticola |
| O83796 | DXS_TREPA | dxs (TP_0824) | Treponema pallidum |
| B2S462 | DXS_TREPS | dxs (TPASS_0824) | Treponema pallidum subsp. pallidum (strain SS14) |
| Q10ZY2 | DXS_TRIEI | dxs (Tery_3042) | Trichodesmium erythraeum (strain IMS101) |
| Q83I20 | DXS_TROW8 | dxs (TW280) | Tropheryma whipplei (strain TW08/27) (Whipple's bacillus) |
| Q83G46 | DXS_TROWT | dxs (TWT_484) | Tropheryma whipplei (strain Twist) (Whipple's bacillus) |
| A1WN06 | DXS_VEREI | dxs (Veis_3283) | Verminephrobacter eiseniae (strain EF01-2) |
| A5F331 | DXS_VIBC3 | dxs (VC0395_A0412) | Vibrio cholerae serotype O1 (strain ATCC 39541/Ogawa 395/ |
| | | | O395) |
| Q9KTL3 | DXS_VIBCH | dxs (VC_0889) | Vibrio cholerae |
| C3LTD9 | DXS_VIBCM | dxs (VCM66_0846) | Vibrio cholerae serotype O1 (strain M66-2) |
| Q5E6Z0 | DXS_VIBF1 | dxs (VF_0711) | Vibrio fischeri (strain ATCC 700601/ES114) |
| B5FBG6 | DXS_VIBFM | dxs (VFMJ11_0731) | Vibrio fischeri (strain MJ11) |
| A7MYC6 | DXS_VIBHB | dxs (VIBHAR_01173) | Vibrio harveyi (strain ATCC BAA-1116/BB120) |
| Q87RU0 | DXS_VIBPA | dxs (VP0686) | Vibrio parahaemolyticus |
| B7VJA1 | DXS_VIBSL | dxs (VS_2406) | Vibrio splendidus (strain LGP32) (Vibrio splendidus (strain |
| | | | Mel32)) |
| Q8DFA3 | DXS_VIBVU | dxs (VV1_0315) | Vibrio vulnificus |
| Q7MN49 | DXS_VIBVY | dxs (VV0868) | Vibrio vulnificus (strain YJ016) |
| Q8D357 | DXS_WIGBR | dxs (WIGBR1440) | Wigglesworthia glossinidia brevipalpis |
| Q7M7Z0 | DXS_WOLSU | dxs (WS1996) | Wolinella succinogenes |
| Q8PJG7 | DXS_XANAC | dxs (XAC2565) | Xanthomonas axonopodis pv. citri (Citrus canker) |
| Q3BRW8 | DXS_XANC5 | dxs (XCV2764) | Xanthomonas campestris pv. vesicatoria (strain 85-10) |
| Q4UW29 | DXS_XANC8 | dxs (XC_1678) | Xanthomonas campestris pv. campestris (strain 8004) |
| Q8P815 | DXS_XANCP | dxs (XCC2434) | Xanthomonas campestris pv. campestris |
| Q2P472 | DXS_XANOM | dxs (XOO1900) | Xanthomonas oryzae pv. oryzae (strain MAFF 311018) |
| B2SQV8 | DXS_XANOP | dxs (PXO_01171) | Xanthomonas oryzae pv. oryzae (strain PXO99A) |
| Q5H1A0 | DXS_XANOR | dxs (XOO2017) | Xanthomonas oryzae pv. oryzae |
| A7IPK6 | DXS_XANP2 | dxs (Xaut_4733) | Xanthobacter autotrophicus (strain ATCC BAA-1158/Py2) |
| B2I607 | DXS_XYLF2 | dxs (XfasM23_1378) | Xylella fastidiosa (strain M23) |
| Q9PB95 | DXS_XYLFA | dxs (XF_2249) | Xylella fastidiosa |
| B0U3E1 | DXS_XYLFM | dxs (Xfasm12_1447) | Xylella fastidiosa (strain M12) |
| Q87C03 | DXS_XYLFT | dxs (PD_1293) | Xylella fastidiosa (strain Temecula1/ATCC 700964) |
| A1JNR7 | DXS_YERE8 | dxs (YE3155) | Yersinia enterocolitica serotype O:8/biotype 1B (strain 8081) |
| A7FLE4 | DXS_YERP3 | dxs (YpslP31758_3112) | Yersinia pseudotuberculosis serotype O:1b (strain IP 31758) |
| Q1C4I9 | DXS_YERPA | dxs (YPA_2671) | Yersinia pestis bv. Antiqua (strain Antiqua) |
| B2K6T7 | DXS_YERPB | dxs (YPTS_0980) | Yersinia pseudotuberculosis serotype IB (strain PB1/+) |
| Q8ZC45 | DXS_YERPE | dxs (YPO3177) (y1008) | Yersinia pestis |
| | | (YP_0754) | |
| A9QZS3 | DXS_YERPG | dxs (YpAngola_A3074) | Yersinia pestis bv. Antiqua (strain Angola) |
| Q1CL87 | DXS_YERPN | dxs (YPN_0911) | Yersinia pestis bv. Antiqua (strain Nepal516) |
| | | (YP516_0987) | |
| A4TPG2 | DXS_YERPP | dxs (YPDSF_2812) | Yersinia pestis (strain Pestoides F) |
| Q66DV4 | DXS_YERPS | dxs (YPTB0939) | Yersinia pseudotuberculosis |
| B1JID8 | DXS_YERPY | dxs (YPK_3253) | Yersinia pseudotuberculosis serotype O:3 (strain YPIII) |
| O50408 | O50408_MYCTU | dxs2 (Rv3379c) | Mycobacterium tuberculosis |
| Q0RNZ8 | Q0RNZ8_FRAAA | dxs (FRAAL2088) | Frankia alni (strain ACN14a) |
| Q5MJZ4 | Q5MJZ4_ANTMA | DXPS | Antirrhinum majus (Garden snapdragon) |
| Q7TWL2 | Q7TWL2_MYCBO | dxs2 (Mb3413c) | Mycobacterium bovis |

### Homogentisate Phytyltransferase

In some embodiments, the invention provides a plant (e.g. cassava) that contains a homogentisate phytyltransferase (HPT) transgene that is functional in the plant. The HPT can be any HPT enzyme known in the art. HPT catalyzes the condensation of homogentisate (HGA) and phytyl diphosphate (PDP) to form 2-methyl-6-phytyl-1,4-benzoquinol (MPBQ). This is an initial step in the synthesis of tocopherols.

Optionally, the HPT is any HPT set forth in Table 5. Optionally, the HPT exhibits a sequence identity of at least about any of 75%, 80%, 85%, 90%, or 95% to an HPT listed in Table 5, or an active fragment thereof.

Examplary HPT transgenes comprise one or more of the following features:

n. a UbiA-like domain;
o. a transmembrane domain;
p. a plastid transit peptide;
q. MW of 35-55 kD (e.g. about 44 kD).

HPT activity can be calculated, for example, by adding radiolabeled HGA to a reaction mixture containing HPT and PDP, and measuring HGA consumption and/or incorporation of the radiolabel into MPBQ.

Optionally, the HPT is a plant or bacterial HPT.

Optionally, a plant HPT is a monocot, dicot, algal, or Arabidopsis HPT.

Optionally, a bacterial HPT is a cyanobacterial HPT.

Optionally, the HPT is derived from any of the species set forth in Table 5

Optionally, the HPT is operably linked to a comestible (e.g. root) specific promoter. Optionally, the HPT is operably linked to a patatin promoter.

TABLE 5
HPT Transgenes

| | | | |
|---|---|---|---|
| Q8VWJ1 | Q8VWJ1_ARATH | Homogentisate | Arabidopsis thaliana (Mouse-ear cress). |
| | | phytyltransferasePutative unch . . . | |
| B2M1Y0 | B2M1Y0_MANES | Homogentisate phytyltransferase | Manihot esculenta (Cassava) (Manioc). |
| B9ILX0 | B9ILX0_POPTR | Predicted protein | Populus trichocarpa (Western balsam poplar) |
| | | | (PI Populus balsamifera subsp. OS trichocarpa). |
| C7EZA1 | C7EZA1_MALDO | Homogentisate phytyltransferase | Malus domestica (Apple) (Pyrus malus). |
| B7X937 | B7X937_HEVBR | Homogentisate phytyl transferase | Hevea brasiliensis (Para rubber tree). |
| C6TBP2 | C6TBP2_SOYBN | Putative uncharacterized protein | Glycine max (Soybean). |
| Q1ACB4 | Q1ACB4_SOYBN | Homogentisate phytyltransferase VTE2-1 | Glycine max (Soybean). |
| A5C4H9 | A5C4H9_VITVI | Putative uncharacterized protein | Vitis vinifera (Grape). |
| D1H9L3 | D1H9L3_VITVI | Whole genome shotgun sequence of | Vitis vinifera (Grape). |
| | | line PN4002 . . . | |
| D2CZX9 | D2CZX9_LACSA | Homogentisate phytylprenyltransferase | Lactuca sativa (Garden lettuce). |
| Q5PT36 | Q5PT36_VITVI | Homogentisate geranylgeranyl | Vitis vinifera (Grape). |
| | | transferase | |
| B6CPP6 | B6CPP6_SESIN | Homogentisic acid phytyltransferase | Sesamum indicum (Oriental sesame) (Gingelly). |
| Q58FG4 | Q58FG4_SOYBN | Homogentisate phytylprenyltransferase | Glycine max (Soybean). |
| B6CPT3 | B6CPT3_9APIA | Homogentisic acid phytyltransferase | Angelica gigas. |
| B2Z8W9 | B2Z8W9_CORSA | Chloroplast homogentisate | Coriandrum sativum (Coriander). |
| | | phytyltransferase | |
| Q647J9 | Q647J9_MEDSA | Homogentisate phytylprenyltransferase | Medicago sativa (Alfalfa). |
| B9T664 | B9T664_RICCO | Bacteriochlorophyll synthase, putative | Ricinus communis (Castor bean). |
| Q1ACB5 | Q1ACB5_9MYRT | Homogentisate phytyltransferase VTE2-1 | Cuphea pulcherrima. |
| C0LTT9 | C0LTT9_LINUS | Homogentisate phytyltransferase | Linum usitatissimum (Flax) (Linseed). |
| B7FA90 | B7FA90_ORYSJ | cDNA, clone: J100057F06, full insert | Oryza sativa subsp. japonica (Rice). |
| | | sequence | |
| C5Z789 | C5Z789_SORBI | Putative uncharacterized protein | Sorghum bicolor (Sorghum) (Sorghum vulgare). |
| | | Sb10g026190 | |
| B6U7K6 | B6U7K6_MAIZE | Homogentisate geranylgeranyl | Zea mays (Maize). |
| | | transferasePuta . . . | |
| Q1ACB8 | Q1ACB8_MAIZE | Homogentisate phytyltransferase VTE2-1 | Zea mays (Maize). |
| Q1ACB7 | Q1ACB7_WHEAT | Homogentisate phytyltransferase VTE2-1 | Triticum aestivum (Wheat). |
| Q1ACB6 | Q1ACB6_ALLPO | Homogentisate phytyltransferase VTE2-1 | Allium porrum (Leek). |
| B9FQB7 | B9FQB7_ORYSJ | Putative uncharacterized protein | Oryza sativa subsp. japonica (Rice). |
| Q67U28 | Q67U28_ORYSJ | Putative uncharacterized protein | Oryza sativa subsp. japonica (Rice). |
| | | B1047G05.17 . . . | |
| A9SGV2 | A9SGV2_PHYPA | Predicted protein | Physcomitrella patens subsp. patens. |
| B1B5P5 | B1B5P5_9FABA | Flavonoid prenyltransferase | Sophora flavescens. |
| A9RVY9 | A9RVY9_PHYPA | Predicted protein | Physcomitrella patens subsp. patens. |
| B7X939 | B7X939_HEVBR | Homogentisate geranylgeranyl | Hevea brasiliensis (Para rubber tree). |
| | | transferase | |
| B6CPP7 | B6CPP7_9APIA | Homogentisate geranylgeranyl | Angelica gigas. |
| | | transferase | |
| D1HIV6 | D1HIV6_VITVI | Whole genome shotgun sequence of | Vitis vinifera (Grape). |
| | | line PN4002 . . . | |
| B9IL30 | B9IL30_POPTR | Predicted protein | Populus trichocarpa (Western balsam poplar) |
| | | | (Populus balsamifera subsp. OS trichocarpa). |
| Q7XB13 | Q7XB13_WHEAT | Homogentisic acid geranylgeranyl | Triticum aestivum (Wheat). |
| | | transferase | |
| A9RDP5 | A9RDP5_PHYPA | Predicted protein | Physcomitrella patens subsp. patens. |
| Q7XB14 | Q7XB14_HORVU | Homogentisic acid geranylgeranyl | Hordeum vulgare (Barley). |
| | | transferase | |
| B1B5P4 | B1B5P4_9FABA | Naringenin 8-dimethylallyltransferase | Sophora flavescens. |
| B1B3P3 | B1B3P3_9FABA | Naringenin 8-dimethylallyltransferase | Sophora flavescens. |
| Q7XB12 | Q7XB12_ORYSJ | Homogentisic acid geranylgeranyl | Oryza sativa subsp. japonica (Rice). |
| | | transferase | |
| B8B0R2 | B8B0R2_ORYSI | Putative uncharacterized protein | Oryza sativa subsp. indica (Rice). |
| A3BE29 | A3BE29_ORYSJ | Putative uncharacterized protein | Oryza sativa subsp. japonica (Rice). |
| C5Z6S0 | C5Z6S0_SORBI | Putative uncharacterized protein | Sorghum bicolor (Sorghum) (Sorghum vulgare). |
| | | Sb10g025475 | |
| B9A1Q4 | B9A1Q4_SOYBN | Pterocarpan 4-dimethylallyltransferase | Glycine max (Soybean). |
| Q0DAK7 | Q0DAK7_ORYSJ | Os06g0646900 protein | Oryza sativa subsp. japonica (Rice). |
| Q67W53 | Q67W53_ORYSJ | Putative homogentisic acid | Oryza sativa subsp. japonica (Rice). |
| | | geranylgeranyl tr . . . | |
| B1B5P3 | B1B5P3_9FABA | Flavonoid prenyltransferase | Sophora flavescens. |
| O64625 | O64625_ARATH | Putative uncharacterized protein | Arabidopsis thaliana (Mouse-ear cress). |
| | | At2g18950 | |
| C6TDB3 | C6TDB3_SOYBN | Putative uncharacterized protein | Glycine max (Soybean). |
| A8YN38 | A8YN38_MICAE | Genome sequencing data, contig C328 | Microcystis aeruginosa PCC 7806. |
| A5BU88 | A5BU88_VITVI | Putative uncharacterized protein | Vitis vinifera (Grape). |
| B0JR78 | B0JR78_MICAN | Homogentisate phytyltransferase | Microcystis aeruginosa (strain NIES-843). |
| B2J3Q6 | B2J3Q6_NOSP7 | UbiA prenyltransferase | Nostoc punctiforme (strain ATCC 29133/PCC |
| | | | 73102). |
| P73726 | P73726_SYNY3 | Slr1736 protein | Synechocystis sp. (strain PCC 6803). |
| B1X091 | B1X091_CYAA5 | Putative uncharacterized protein | Cyanothece sp. (strain ATCC 51142). |
| C1MHL9 | C1MHL9_9CHLO | Predicted protein | Micromonas pusilla CCMP1545. |
| B1XQG6 | B1XQG6_SYNP2 | Homogentisate geranylgeranyl | Synechococcus sp. (strain ATCC 27264/PCC |
| | | transferase | 7002/PR-6) (Agmenellum OS quadruplicatum). |
| A0Z9X9 | A0Z9X9_NODSP | Putative uncharacterized protein | Nodularia spumigena CCY9414. |
| Q3M7F7 | Q3M7F7_ANAVT | Homogentisate phytyltransferase | Anabaena variabilis (strain ATCC 29413/PCC |
| | | | 7937). |
| Q8YRJ8 | Q8YRJ8_ANASP | Alr3448 protein | Anabaena sp. (strain PCC 7120). |
| Q4C0Z2 | Q4C0Z2_CROWT | UbiA prenyltransferase | Crocosphaera watsonii WH 8501. |
| A3IY82 | A3IY82_9CHRO | Putative uncharacterized protein | Cyanothece sp. CCY0110. |
| A4RT35 | A4RT35_OSTLU | Homogentisate | Ostreococcus lucimarinus (strain CCE9901). |
| | | phytylprenyltransferase/homoge . . . | |
| C1EAI7 | C1EAI7_9CHLO | Predicted protein | Micromonas sp. RCC299. |
| B7K7Y8 | B7K7Y8_CYAP7 | UbiA prenyltransferase | Cyanothece sp. (strain PCC 7424) |
| | | | (Synechococcus sp. (strain ATCC 29155)). |
| A0YXJ4 | A0YXJ4_9CYAN | UbiA prenyltransferase | Lyngbya sp. PCC 8106. |
| Q2JTP7 | Q2JTP7_SYNJA | Prenyltransferase, UbiA family | Synechococcus sp. (strain JA-3-3Ab) |
| | | | (Cyanobacteria bacterium Yellowstone OS A- |
| | | | Prime). |
| B7JZF9 | B7JZF9_CYAP8 | UbiA prenyltransferase | Cyanothece sp. (strain PCC 8801) |
| | | | (Synechococcus sp. (strain PCC 8801/OS RF- |
| | | | 1)). |
| Q7NNX5 | Q7NNX5_GLOVI | GII0283 protein | Gloeobacter violaceus. |
| C7QPT1 | C7QPT1_CYAP0 | UbiA prenyltransferase | Cyanothece sp. (strain PCC 8802) |
| | | | (Synechococcus sp. (strain RF-2)). |
| B9YIB4 | B9YIB4_ANAAZ | UbiA prenyltransferase | ‘Nostoc azollae’ 0708. |
| A8J4W7 | A8J4W7_CHLRE | Predicted protein | Chlamydomonas reinhardtii. |
| Q0DAE9 | Q0DAE9_ORYSJ | Os06g0658900 protein | Oryza sativa subsp. japonica (Rice). |
| Q2JJI8 | Q2JJI8_SYNJB | Prenyltransferase, UbiA family | Synechococcus sp. (strain JA-2-3B′a(2-13)) |
| | | | (Cyanobacteria bacterium OS Yellowstone B- |
| | | | Prime). |
| B0BZ73 | B0BZ73_ACAM1 | Prenyltransferase, UbiA family | Acaryochloris marina (strain MBIC 11017). |
| B7G488 | B7G488_PHATR | Predicted protein | Phaeodactylum tricornutum CCAP 1055/1. |
| B5W0F0 | B5W0F0_SPIMA | UbiA prenyltransferase | Arthrospira maxima CS-328. |
| B4WKY1 | B4WKY1_9SYNE | Prenyltransferase, UbiA family | Synechococcus sp. PCC 7335. |
| B8HVT5 | B8HVT5_CYAP4 | UbiA prenyltransferase | Cyanothece sp. (strain PCC 7425/ATCC 29141). |
| Q10XV6 | Q10XV6_TRIEI | Homogentisate phytyltransferase | Trichodesmium erythraeum (strain IMS101). |
| C6TCV5 | C6TCV5_SOYBN | Putative uncharacterized protein | Glycine max (Soybean). |
| B9H9X9 | B9H9X9_POPTR | Predicted protein | Populus trichocarpa (Western balsam poplar) |
| | | | (Populus balsamifera subsp. OS trichocarpa). |
| B8G328 | B8G328_CHLAD | UbiA prenyltransferase | Chloroflexus aggregans (strain MD-66/DSM |
| | | | 9485). |
| B4FBU3 | B4FBU3_MAIZE | Putative uncharacterized protein | Zea mays (Maize). |
| Q1ACB3 | Q1ACB3_ARATH | Homogentisate phytyltransferase VTE2-2 | Arabidopsis thaliana (Mouse-ear cress). |
| Q1ACB2 | Q1ACB2_SOYBN | Homogentisate phytyltransferase VTE2-2 | Glycine max (Soybean). |
| D1I9Z9 | D1I9Z9_VITVI | Whole genome shotgun sequence of | Vitis vinifera (Grape). |
| | | line PN4002 . . . | |
| B9LDT9 | B9LDT9_CHLSY | UbiA prenyltransferase | Chloroflexus aurantiacus (strain ATCC 29364/ |
| | | | DSM 637/Y-400-fl). |
| A9WKI4 | A9WKI4_CHLAA | UbiA prenyltransferase | Chloroflexus aurantiacus (strain ATCC 29366/ |
| | | | DSM 635/J-10-fl). |
| A9SCS8 | A9SCS8_PHYPA | Predicted protein | Physcomitrella patens subsp. patens. |
| A1JHN0 | A1JHN0_CHLRE | Homogentisate prenyltransferase | Chlamydomonas reinhardtii. |
| Q017K0 | Q017K0_OSTTA | Putative tocopherol | Ostreococcus tauri. |
| | | polyprenyltransferase . . . | |
| C5XCE4 | C5XCE4_SORBI | Putative uncharacterized protein | Sorghum bicolor (Sorghum) (Sorghum vulgare). |
| | | Sb02g037370 | |
| Q0D576 | Q0D576_ORYSJ | Os07g0576000 proteinPutative | Oryza sativa subsp. japonica (Rice). |
| | | uncharacterized . . . | |
| B8B7R2 | B8B7R2_ORYSI | Putative uncharacterized protein | Oryza sativa subsp. indica (Rice). |
| A4RYL3 | A4RYL3_OSTLU | Predicted protein | Ostreococcus lucimarinus (strain CCE9901). |
| Q6ZLA8 | Q6ZLA8_ORYSJ | Putative tocopherol | Oryza sativa subsp. japonica (Rice). |
| | | polyprenyltransferase | |
| A8J261 | A8J261_CHLRE | Homogentisate solanesyltransferase | Chlamydomonas reinhardtii. |
| C1E4B5 | C1E4B5_9CHLO | Predicted protein | Micromonas sp. RCC299. |
| B8B0Z5 | B8B0Z5_ORYSI | Putative uncharacterized protein | Oryza sativa subsp. indica (Rice). |

### Geranylgeranyl Reductase

In some embodiments, the invention provides a plant (e.g. cassava) that contains a geranylgeranyl reductase (GGR) transgene that is functional in the plant. The GGR can be any GGR enzyme known in the art. GGR catalyzes the conversion of geranylgeranyl diphosphate to phytyl diphosphate. This is a step in the synthesis of tocopherols.

Optionally, the GGR is any GGR set forth in Table 6. Optionally, the GGR exhibits a sequence identity of at least about any of 75%, 80%, 85%, 90%, or 95% to a GGR listed in Table 6, or an active fragment thereof. Optionally, the GGR is derived from any of the species listed in Table 6

Examplary GGR transgenes comprise a Rossmann-fold NAD(P)H/NAD(P)(+) binding (NADB) domain.

Optionally, the GGR is a plant, bacterial, fungal, or animal GGR.

Optionally, the GGR is a monocot, dicot, or Arabidopsis GGR.

Optionally, the GGR is operably linked to a comestible (e.g. root) specific promoter. Optionally, the GGR is operably linked to a patatin promoter.

TABLE 6
GGR TransgenesP

| | | | |
|---|---|---|---|
| Q39108 | GGR_ARATH | Geranylgeranyl pyrophosphate | Arabidopsis thaliana (Mouse-ear cress). |
| | | synthase-relate . . . | |
| A5BEV9 | A5BEV9_VITVI | Putative uncharacterized protein | Vitis vinifera (Grape). |
| D1IWS2 | D1IWS2_VITVI | Whole genome shotgun sequence of | Vitis vinifera (Grape). |
| | | line PN4002 . . . | |
| B9HR16 | B9HR16_POPTR | Predicted protein | Populus trichocarpa (Western balsam poplar) |
| | | | (Populus balsamifera subsp. OS trichocarpa). |
| B9SUY3 | B9SUY3_RICCO | Geranyl geranyl pyrophosphate | Ricinus communis (Castor bean). |
| | | synthase, puta . . . | |
| C6T7T0 | C6T7T0_SOYBN | Putative uncharacterized protein | Glycine max |
| | | | (Soybean). |
| A9ZN21 | A9ZN21_HEVBR | Geranylgeranyl-diphosphate synthase | Hevea brasiliensis (Para rubber tree). |
| B9H1M5 | B9H1M5_POPTR | Predicted protein | Populus trichocarpa (Western balsam poplar) |
| | | | (Populus balsamifera subsp. OS trichocarpa). |
| Q0DYU0 | Q0DYU0_ORYSJ | Os02g0668100 protein | Oryza sativa subsp. japonica (Rice). |
| Q6ET88 | Q6ET88_ORYSJ | cDNA clone: 001-204-C07, full insert | Oryza sativa subsp. japonica (Rice). |
| | | sequence . . . | |
| A2X847 | A2X847_ORYSI | Putative uncharacterized protein | Oryza sativa subsp. indica (Rice). |
| C0PQN3 | C0PQN3_PICSI | Putative uncharacterized protein | Picea sitchensis (Sitka spruce). |
| A3A9Y5 | A3A9Y5_ORYSJ | Putative uncharacterized protein | Oryza sativa subsp. japonica (Rice). |
| B9GV66 | B9GV66_POPTR | Predicted protein | Populus trichocarpa (Western balsam poplar) |
| | | | (Populus balsamifera subsp. OS trichocarpa). |
| Q43133 | GGPPS_SINAL | Geranylgeranyl pyrophosphate | Sinapis alba (White mustard) (Brassica hirta). |
| | | synthase, chlor . . . | |
| P34802 | GGPP1_ARATH | Geranylgeranyl pyrophosphate | Arabidopsis thaliana (Mouse-ear cress). |
| | | synthase 1, chl . . . | |
| Q0WUL9 | Q0WUL9_ARATH | Geranylgeranyl pyrophosphate | Arabidopsis thaliana (Mouse-ear cress). |
| | | synthase | |
| Q9ZPM3 | Q9ZPM3_TAXCA | Geranylgeranyl diphosphate synthase | Taxus canadensis (Canadian yew). |
| Q6Q291 | Q6Q291_9CONI | Geranylgeranyl diphosphate synthase | Taxus x media. |
| Q2HYC6 | Q2HYC6_9CONI | Geranylgeranyl diphosphate synthase | Taxus wallichiana. |
| Q8LAW5 | Q8LAW5_ARATH | Geranylgeranyl pyrophosphate | Arabidopsis thaliana (Mouse-ear cress). |
| | | synthase | |
| B1A9K6 | B1A9K6_PICAB | Geranyl diphosphate synthase 2 | Picea abies (Norway spruce) (Picea excelsa). |
| A9T5D6 | A9T5D6_PHYPA | Predicted protein | Physcomitrella patens subsp. patens. |
| C0PRU8 | C0PRU8_PICSI | Putative uncharacterized protein | Picea sitchensis (Sitka spruce). |
| B1A9K9 | B1A9K9_PICAB | Geranylgeranyl diphosphate synthase 5 | Picea abies (Norway spruce) (Picea excelsa). |
| Q9FV47 | Q9FV47_TARER | GGDP synthase | Tagetes erecta (African marigold). |
| Q2VEY2 | Q2VEY2_DAUCA | Putative geranylgeranyl pyrophosphate | Daucus carota subsp. sativus. |
| | | syntha . . . | |
| A9RDQ7 | A9RDQ7_PHYPA | Predicted protein | Physcomitrella patens subsp. patens. |
| Q8W1R9 | Q8W1R9_ABIGR | Geranylgeranyl diphosphate synthase | Abies grandis (Grand fir). |
| D2IT07 | D2IT07_PICAB | Isoprenyl diphosphate synthase | Picea abies (Norway spruce) (Picea excelsa). |
| C0PT16 | C0PT16_PICSI | Putative uncharacterized protein | Picea sitchensis (Sitka spruce). |
| C5XCF6 | C5XCF6_SORBI | Putative uncharacterized protein | Sorghum bicolor (Sorghum) (Sorghum vulgare). |
| | | Sb02g037510 | |
| Q2HXJ9 | Q2HXJ9_CHRMO | Geranylgeranyl pyrophosphate | Chrysanthemum morifolium (Florist's daisy) |
| | | synthase | (Dendranthema grandiflorum). |
| A5AX87 | A5AX87_VITVI | Putative uncharacterized protein | Vitis vinifera (Grape). |
| Q8LKJ1 | Q8LKJ1_ABIGR | Geranyl diphosphate synthase | Abies grandis (Grand fir). |
| B1A9L0 | B1A9L0_PICAB | Geranylgeranyl diphosphate synthase 6 | Picea abies (Norway spruce) (Picea excelsa). |
| Q9SXZ6 | Q9SXZ6_DAUCA | GGPP synthase | Daucus carota (Carrot). |
| Q8LSC5 | Q8LSC5_9ROSI | Geranylgeranyl pyrophosphate | Cistus creticus. |
| | | synthase | |
| B9SGH7 | B9SGH7_RICCO | Geranyl geranyl pyrophosphate | Ricinus communis (Castor bean). |
| | | synthase, puta . . . | |
| B9SYX2 | B9SYX2_RICCO | Geranyl geranyl pyrophosphate | Ricinus communis (Castor bean). |
| | | synthase, puta . . . | |
| C4NAM8 | C4NAM8_HUMLU | Geranyl diphosphate synthase large | Humulus lupulus (European hop). |
| | | subunit | |
| C0PR55 | C0PR55_PICSI | Putative uncharacterized protein | Picea sitchensis (Sitka spruce). |
| B1N7F3 | B1N7F3_9SOLA | Geranylgeranyl pyrophosphate | Nicotiana attenuata. |
| | | synthase | |
| Q42698 | GGPPS_CATRO | Geranylgeranyl pyrophosphate | Catharanthus roseus (Madagascar periwinkle) |
| | | synthase, chlor . . . | (Vinca rosea). |
| Q5ISD7 | Q5ISD7_9MAGN | Geranylgeranyl diphosphate synthase | Adonis aestivalis var. palaestina. |
| Q1A7T0 | Q1A7T0_SOLLC | Geranylgeranyl pyrophosphate | Solanum lycopersicum (Tomato) (Lycopersicon |
| | | synthase 1 | esculentum). |
| Q6QLV1 | Q6QLV1_ANTMA | Geranyl diphosphate synthase large | Antirrhinum majus (Garden snapdragon). |
| | | subunit | |
| Q94ID7 | GGPPS_HEVBR | Geranylgeranyl pyrophosphate | Hevea brasiliensis (Para rubber tree). |
| | | synthase, chlor . . . | |
| Q9ZU77 | Q9ZU77_ARATH | Putative geranylgeranyl pyrophosphate | Arabidopsis thaliana (Mouse-ear cress). |
| | | syntha . . . | |
| Q64KQ5 | Q64KQ5_GINBI | Geranylgeranyl diphosphate synthase | Ginkgo biloba (Ginkgo). |
| A0MEM7 | A0MEM7_ARATH | Putative uncharacterized protein | Arabidopsis thaliana (Mouse-ear cress). |
| B9H374 | B9H374_POPTR | Predicted protein | Populus trichocarpa (Western balsam poplar) |
| | | | (Populus balsamifera subsp. OS trichocarpa). |
| Q9LUE1 | Q9LUE1_ARATH | Geranylgeranyl pyrophosphate | Arabidopsis thaliana (Mouse-ear cress). |
| | | synthaseAt3g145 . . . | |
| Q5ISD8 | Q5ISD8_9MAGN | Geranylgeranyl diphosphate synthase | Adonis aestivalis var. palaestina. |
| Q8LKJ2 | Q8LKJ2_ABIGR | Geranyl diphosphate synthase | Abies grandis (Grand fir). |
| Q5G1J1 | Q5G1J1_9LAMI | Geranyl diphosphate synthase | Picrorhiza kurrooa. |
| D0FZ25 | D0FZ25_9ASTE | Geranylgeranyl pyrophosphate | Ipomoea sp. Kenyan. |
| | | synthase | |
| Q9LUD9 | GGPP3_ARATH | Geranylgeranyl pyrophosphate | Arabidopsis thaliana (Mouse-ear cress). |
| | | synthase 3, chl . . . | |
| P80042 | GGPPS_CAPAN | Geranylgeranyl pyrophosphate | Capsicum annuum (Bell pepper). |
| | | synthase, chlor . . . | |
| Q1A7S9 | Q1A7S9_SOLLC | Geranylgeranyl pyrophosphate | Solanum lycopersicum (Tomato) (Lycopersicon |
| | | synthase 2 | esculentum). |
| Q9LHR4 | Q9LHR4_ARATH | Geranylgeranyl pyrophosphate | Arabidopsis thaliana (Mouse-ear cress). |
| | | synthaseAt3g320 . . . | |
| Q56Y72 | Q56Y72_ARATH | Geranylgeranyl pyrophosphate | Arabidopsis thaliana (Mouse-ear cress). |
| | | synthase | |
| Q5ISD9 | Q5ISD9_9MAGN | Geranylgeranyl diphosphate synthase | Adonis aestivalis var. palaestina. |
| A9ZN20 | A9ZN20_HEVBR | Geranylgeranyl-diphosphate synthase | Hevea brasiliensis (Para rubber tree). |
| Q9SSU0 | Q9SSU0_9ROSI | Geranylgeranyl pyrophosphate | Croton sublyratus. |
| | | synthase | |
| B9MXS6 | B9MXS6_POPTR | Predicted protein | Populus trichocarpa (Western balsam poplar) |
| | | | (Populus balsamifera subsp. OS trichocarpa). |
| Q2VEY3 | Q2VEY3_DAUCA | Putative geranylgeranyl pyrophosphate | Daucus carota subsp. sativus. |
| | | syntha . . . | |
| Q1W682 | Q1W682_STERE | Geranylgeranyl diphosphate synthase | Stevia rebaudiana (Stevia). |
| B9HEN2 | B9HEN2_POPTR | Predicted protein | Populus trichocarpa (Western balsam poplar) |
| | | | (Populus balsamifera subsp. OS trichocarpa). |
| C5G5X3 | C5G5X3_MAIZE | Putative geranylgeranyl pyrophosphate | Zea mays (Maize). |
| | | syntha . . . | |
| C1KH04 | C1KH04_ELAUM | Geranylgeranyl pyrophosphate | Elaeagnus umbellata (Autumn olive). |
| | | synthase | |
| B7TCA9 | B7TCA9_SALMI | Geranylgeranyl diphosphate synthase | Salvia miltiorrhiza (Chinese sage). |
| O81099 | O81099_HELAN | Geranylgeranyl pyrophosphate | Helianthus annuus (Common sunflower). |
| | | synthase | |
| Q9SXZ5 | Q9SXZ5_DAUCA | GGPP synthase | Daucus carota (Carrot). |
| Q8LSC4 | Q8LSC4_9ROSI | Geranylgeranyl pyrophosphate | Cistus creticus. |
| | | synthase | |
| A8JHU6 | A8JHU6_CHLRE | Geranylgeranyl diphosphate synthase | Chlamydomonas reinhardtii. |
| Q9LIA0 | Q9LIA0_ARATH | Geranylgeranyl pyrophosphate | Arabidopsis thaliana (Mouse-ear cress). |
| | | synthasePutativ . . . | |
| B9GV67 | B9GV67_POPTR | Predicted protein | Populus trichocarpa (Western balsam poplar) |
| | | | (Populus balsamifera subsp. OS trichocarpa). |
| Q8LKJ3 | Q8LKJ3_ABIGR | Geranyl diphosphate synthase | Abies grandis (Grand fir). |
| Q6R3F3 | Q6R3F3_9LAMI | Geranylgeranyl pyrophosphate | Plectranthus barbatus. |
| | | synthase | |
| B5W5L4 | B5W5L4_SPIMA | Polyprenyl synthetase | Arthrospira maxima CS-328. |
| A7XDE3 | A7XDE3_9LAMI | Geranyl pyrophosphate synthase large | Mentha haplocalyx var. piperascens. |
| | | subunit | |
| Q9SLG2 | GGPP4_ARATH | Geranylgeranyl pyrophosphate | Arabidopsis thaliana (Mouse-ear cress). |
| | | synthase 4 (GGP . . . | |
| A0MEM8 | A0MEM8_ARATH | Putative uncharacterized protein | Arabidopsis thaliana (Mouse-ear cress). |
| Q94IF0 | Q94IF0_EUCUL | Putative GGPP synthase | Eucommia ulmoides (Hardy rubber tree). |
| Q9SST9 | Q9SST9_SCODU | Geranylgeranyl pyrophosphate | Scoparia dulcis (Sweet broom). |
| | | synthase | |
| Q9SBR3 | Q9SBR3_MENPI | Geranyl diphosphate synthase large | Mentha piperita (Peppermint). |
| | | subunitGe . . . | |
| Q7XI92 | Q7XI92_ORYSJ | Os07g0580900 proteincDNA | Oryza sativa subsp. japonica (Rice). |
| | | clone: J023007O22, f . . . | |
| A2YN12 | A2YN12_ORYSI | Putative uncharacterized protein | Oryza sativa subsp. indica (Rice). |
| C5G5X4 | C5G5X4_MAIZE | Putative geranylgeranyl pyrophosphate | Zea mays (Maize). |
| | | syntha . . . | |
| P72683 | P72683_SYNY3 | Geranylgeranyl pyrophosphate | Synechocystis sp. (strain PCC 6803). |
| | | synthase | |
| O04046 | GGPP2_ARATH | Geranylgeranyl pyrophosphate | Arabidopsis thaliana (Mouse-ear cress). |
| | | synthase 2 (GGP . . . | |
| B6DVJ8 | B6DVJ8_ARATH | Geranylgeranyl pyrophosphate | Arabidopsis thaliana (Mouse-ear cress). |
| | | synthase 2 | |
| B4VH16 | B4VH16_9CYAN | Polyprenyl synthetase superfamily | Microcoleus chthonoplastes PCC 7420. |
| Q8DMU1 | Q8DMU1_THEEB | Geranylgeranyl pyrophosphate | Thermosynechococcus elongatus (strain BP-1). |
| | | synthase | |
| Q00Y25 | Q00Y25_OSTTA | Geranylgeranyl diphosphate synthase | Ostreococcus tauri. |
| | | (ISS) | |
| Q5N420 | Q5N420_SYNP6 | Geranylgeranyl pyrophosphate | Synechococcus sp. (strain ATCC 27144/PCC |
| | | synthase | 6301/SAUG 1402/1) (Anacystis OS nidulans). |
| Q31Q61 | Q31Q61_SYNE7 | Farnesyl-diphosphate synthase | Synechococcus elongatus (strain PCC 7942) |
| | | | (Anacystis nidulans R2). |
| Q9S5F1 | Q9S5F1_SYNEL | Geranylgeranyl diphosphate synthase | Synechococcus elongatus. |
| | | (SelGGPS) | |
| Q2JX96 | Q2JX96_SYNJA | Geranylgeranyl diphosphate synthase | Synechococcus sp. (strain JA-3-3Ab) |
| | | | (Cyanobacteria bacterium Yellowstone OS A- |
| | | | Prime). |

### Homogentisate Geranylgeranyl Transferase

In some embodiments, the invention provides a plant (e.g. cassava) that contains a Homogentisate geranylgeranyl transferase (HGGT) transgene that is functional in the plant. The HGGT can be any HGGT enzyme known in the art. HGGT catalyzes the conversion of homogentisic acid and geranylgeranyl diphosphateto 2-methyl-6-geranylgeranylplastoquinol. This is an initial step in the synthesis of tocotrienols.

Optionally, the HGGT is any HGGT set forth in Table 7. Optionally, the HGGT exhibits a sequence identity of at least about any of 75%, 80%, 85%, 90%, or 95% to an HGGT listed in Table 7, or an active fragment thereof. Optionally, the HGGT is derived from any of the species listed in Table 7.

Examplary HGGT transgenes comprise one or more of the following features:

r. UbiA-like domain; and
s. prenyltransferase/zinc ion binding domain.

Optionally, the HGGT is a plant, bacterial, or fungal HGGT.

Optionally, the HGGT is a monocot, dicot, or Arabidopsis HGGT.

Optionally, the HGGT is operably linked to a comestible (e.g. root) specific promoter. Optionally, the HGGT is operably linked to a patatin promoter.

TABLE 7
HGGT Transgenes

| | | | |
|---|---|---|---|
| B6U7K6 | B6U7K6_MAIZE | Homogentisate geranylgeranyl | Zea mays (Maize). |
| | | transferasePuta . . . | |
| Q1ACB8 | Q1ACB8_MAIZE | Homogentisate phytyltransferase VTE2-1 | Zea mays (Maize). |
| C5Z789 | C5Z789_SORBI | Putative uncharacterized protein | Sorghum bicolor (Sorghum) (Sorghum |
| | | Sb10g026190 | vulgare). |
| B7FA90 | B7FA90_ORYSJ | cDNA, clone: J100057F06, full insert | Oryza sativa subsp. japonica (Rice). |
| | | sequence | |
| Q1ACB7 | Q1ACB7_WHEAT | Homogentisate phytyltransferase VTE2-1 | Triticum aestivum (Wheat). |
| Q67U28 | Q67U28_ORYSJ | Putative uncharacterized protein | Oryza sativa subsp. japonica (Rice). |
| | | B1047G05.17 . . . | |
| B9FQB7 | B9FQB7_ORYSJ | Putative uncharacterized protein | Oryza sativa subsp. japonica (Rice). |
| A5C4H9 | A5C4H9_VITVI | Putative uncharacterized protein | Vitis vinifera (Grape). |
| D1H9L3 | D1H9L3_VITVI | Whole genome shotgun sequence of line | Vitis vinifera (Grape). |
| | | PN4002 . . . | |
| Q5PT36 | Q5PT36_VITVI | Homogentisate geranylgeranyl transferase | Vitis vinifera (Grape). |
| C7EZA1 | C7EZA1_MALDO | Homogentisate phytyltransferase | Malus domestica (Apple) (Pyrus malus). |
| Q1ACB4 | Q1ACB4_SOYBN | Homogentisate phytyltransferase VTE2-1 | Glycine max (Soybean). |
| C6TBP2 | C6TBP2_SOYBN | Putative uncharacterized protein | Glycine max (Soybean). |
| B6CPP6 | B6CPP6_SESIN | Homogentisic acid phytyltransferase | Sesamum indicum (Oriental sesame) |
| | | | (Gingelly). |
| Q647J9 | Q647J9_MEDSA | Homogentisate phytylprenyltransferase | Medicago sativa (Alfalfa). |
| B2M1Y0 | B2M1Y0_MANES | Homogentisate phytyltransferase | Manihot esculenta (Cassava) (Manioc). |
| Q58FG4 | Q58FG4_SOYBN | Homogentisate phytylprenyltransferase | Glycine max (Soybean). |
| B7X937 | B7X937_HEVBR | Homogentisate phytyl transferase | Hevea brasiliensis (Para rubber tree). |
| Q1ACB6 | Q1ACB6_ALLPO | Homogentisate phytyltransferase VTE2-1 | Allium porrum (Leek). |
| B2Z8W9 | B2Z8W9_CORSA | Chloroplast homogentisate | Coriandrum sativum (Coriander). |
| | | phytyltransferase | |
| Q1ACB5 | Q1ACB5_9MYRT | Homogentisate phytyltransferase VTE2-1 | Cuphea pulcherrima. |
| B6CPT3 | B6CPT3_9APIA | Homogentisic acid phytyltransferase | Angelica gigas. |
| Q8VWJ1 | Q8VWJ1_ARATH | Homogentisate phytyltransferasePutative | Arabidopsis thaliana (Mouse-ear cress). |
| | | unch . . . | |
| D2CZX9 | D2CZX9_LACSA | Homogentisate phytylprenyltransferase | Lactuca sativa (Garden lettuce). |
| B9ILX0 | B9ILX0_POPTR | Predicted protein | Populus trichocarpa (Western balsam |
| | | | poplar) (Populus balsamifera subsp. OS |
| | | | trichocarpa). |
| B9T664 | B9T664_RICCO | Bacteriochlorophyll synthase, putative | Ricinus communis (Castor bean). |
| C0LTT9 | C0LTT9_LINUS | Homogentisate phytyltransferase | Linum usitatissimum (Flax) (Linseed). |
| A9SGV2 | A9SGV2_PHYPA | Predicted protein | Physcomitrella patens subsp. patens. |
| A9RVY9 | A9RVY9_PHYPA | Predicted protein | Physcomitrella patens subsp. patens. |
| A9RDP5 | A9RDP5_PHYPA | Predicted protein | Physcomitrella patens subsp. patens. |
| B1B5P5 | B1B5P5_9FABA | Flavonoid prenyltransferase | Sophora flavescens. |
| B6CPP7 | B6CPP7_9APIA | Homogentisate geranylgeranyl transferase | Angelica gigas. |
| B7X939 | B7X939_HEVBR | Homogentisate geranylgeranyl transferase | Hevea brasiliensis (Para rubber tree). |
| D1HIV6 | D1HIV6_VITVI | Whole genome shotgun sequence of line | Vitis vinifera (Grape). |
| | | PN4002 . . . | |
| B9IL30 | B9IL30_POPTR | Predicted protein | Populus trichocarpa (Western balsam |
| | | | poplar) (Populus balsamifera subsp. OS |
| | | | trichocarpa). |
| B1B5P4 | B1B5P4_9FABA | Naringenin 8-dimethylallyltransferase | Sophora flavescens. |
| Q7XB13 | Q7XB13_WHEAT | Homogentisic acid geranylgeranyl | Triticum aestivum (Wheat). |
| | | transferase | |
| B1B3P3 | B1B3P3_9FABA | Naringenin 8-dimethylallyltransferase | Sophora flavescens. |
| Q7XB14 | Q7XB14_HORVU | Homogentisic acid geranylgeranyl | Hordeum vulgare (Barley). |
| | | transferase | |
| Q7XB12 | Q7XB12_ORYSJ | Homogentisic acid geranylgeranyl | Oryza sativa subsp. japonica (Rice). |
| | | transferase | |
| B8B0R2 | B8B0R2_ORYSI | Putative uncharacterized protein | Oryza sativa subsp. indica (Rice). |
| A3BE29 | A3BE29_ORYSJ | Putative uncharacterized protein | Oryza sativa subsp. japonica (Rice). |
| B9A1Q4 | B9A1Q4_SOYBN | Pterocarpan 4-dimethylallyltransferase | Glycine max (Soybean). |
| C5Z6S0 | C5Z6S0_SORBI | Putative uncharacterized protein | Sorghum bicolor (Sorghum) (Sorghum |
| | | Sb10g025475 | vulgare). |
| Q0DAK7 | Q0DAK7_ORYSJ | Os06g0646900 protein | Oryza sativa subsp. japonica (Rice). |
| Q67W53 | Q67W53_ORYSJ | Putative homogentisic acid geranylgeranyl | Oryza sativa subsp. japonica (Rice). |
| | | tr . . . | |
| B8B0Z5 | B8B0Z5_ORYSI | Putative uncharacterized protein | Oryza sativa subsp. indica (Rice). |
| B1B5P3 | B1B5P3_9FABA | Flavonoid prenyltransferase | Sophora flavescens. |
| C6TDB3 | C6TDB3_SOYBN | Putative uncharacterized protein | Glycine max (Soybean). |
| A5BU88 | A5BU88_VITVI | Putative uncharacterized protein | Vitis vinifera (Grape). |
| Q0DAE9 | Q0DAE9_ORYSJ | Os06g0658900 protein | Oryza sativa subsp. japonica (Rice). |
| C1MHL9 | C1MHL9_9CHLO | Predicted protein | Micromonas pusilla CCMP1545. |
| P73726 | P73726_SYNY3 | Slr1736 protein | Synechocystis sp. (strain PCC 6803). |
| B1XQG6 | B1XQG6_SYNP2 | Homogentisate geranylgeranyl transferase | Synechococcus sp. (strain ATCC 27264/ |
| | | | PCC 7002/PR-6) (Agmenellum OS |
| | | | quadruplicatum). |
| B2J3Q6 | B2J3Q6_NOSP7 | UbiA prenyltransferase | Nostoc punctiforme (strain ATCC 29133/ |
| | | | PCC 73102). |
| A8YN38 | A8YN38_MICAE | Genome sequencing data, contig C328 | Microcystis aeruginosa PCC 7806. |
| A8J4W7 | A8J4W7_CHLRE | Predicted protein | Chlamydomonas reinhardtii. |
| B1X091 | B1X091_CYAA5 | Putative uncharacterized protein | Cyanothece sp. (strain ATCC 51142). |
| B0JR78 | B0JR78_MICAN | Homogentisate phytyltransferase | Microcystis aeruginosa (strain NIES- |
| | | | 843). |
| Q3M7F7 | Q3M7F7_ANAVT | Homogentisate phytyltransferase | Anabaena variabilis (strain ATCC 29413/ |
| | | | PCC 7937). |
| B9YIB4 | B9YIB4_ANAAZ | UbiA prenyltransferase | ‘Nostoc azollae’ 0708. |
| A0Z9X9 | A0Z9X9_NODSP | Putative uncharacterized protein | Nodularia spumigena CCY9414. |
| Q7NNX5 | Q7NNX5_GLOVI | Gll0283 protein | Gloeobacter violaceus. |
| Q8YRJ8 | Q8YRJ8_ANASP | Alr3448 protein | Anabaena sp. (strain PCC 7120). |
| A3IY82 | A3IY82_9CHRO | Putative uncharacterized protein | Cyanothece sp. CCY0110. |
| B7JZF9 | B7JZF9_CYAP8 | UbiA prenyltransferase | Cyanothece sp. (strain PCC 8801) |
| | | | (Synechococcus sp. (strain PCC 8801/ |
| | | | OS RF-1)). |
| Q67U34 | Q67U34_ORYSJ | Putative uncharacterized protein | Oryza sativa subsp. japonica (Rice). |
| | | B1047G05.9P . . . | |
| C7QPT1 | C7QPT1_CYAP0 | UbiA prenyltransferase | Cyanothece sp. (strain PCC 8802) |
| | | | (Synechococcus sp. (strain RF-2)). |
| Q4C0Z2 | Q4C0Z2_CROWT | UbiA prenyltransferase | Crocosphaera watsonii WH 8501. |
| C1EAI7 | C1EAI7_9CHLO | Predicted protein | Micromonas sp. RCC299. |
| A4RT35 | A4RT35_OSTLU | Homogentisate | Ostreococcus lucimarinus (strain |
| | | phytylprenyltransferase/homoge . . . | CCE9901). |
| Q2JTP7 | Q2JTP7_SYNJA | Prenyltransferase, UbiA family | Synechococcus sp. (strain JA-3-3Ab) |
| | | | (Cyanobacteria bacterium Yellowstone |
| | | | OS A-Prime). |
| B7K7Y8 | B7K7Y8_CYAP7 | UbiA prenyltransferase | Cyanothece sp. (strain PCC 7424) |
| | | | (Synechococcus sp. (strain ATCC |
| | | | 29155)). |
| A0YXJ4 | A0YXJ4_9CYAN | UbiA prenyltransferase | Lyngbya sp. PCC 8106. |
| B0BZ73 | B0BZ73_ACAM1 | Prenyltransferase, UbiA family | Acaryochloris marina (strain MBIC |
| | | | 11017). |
| Q2JJI8 | Q2JJI8_SYNJB | Prenyltransferase, UbiA family | Synechococcus sp. (strain JA-2-3B′a(2- |
| | | | 13)) (Cyanobacteria bacterium OS |
| | | | Yellowstone B-Prime). |
| B5W0F0 | B5W0F0_SPIMA | UbiA prenyltransferase | Arthrospira maxima CS-328. |
| B4WKY1 | B4WKY1_9SYNE | Prenyltransferase, UbiA family | Synechococcus sp. PCC 7335. |
| Q10XV6 | Q10XV6_TRIEI | Homogentisate phytyltransferase | Trichodesmium erythraeum (strain |
| | | | IMS101). |
| B8HVT5 | B8HVT5_CYAP4 | UbiA prenyltransferase | Cyanothece sp. (strain PCC 7425/ |
| | | | ATCC 29141). |
| C6TCV5 | C6TCV5_SOYBN | Putative uncharacterized protein | Glycine max (Soybean). |
| O64625 | O64625_ARATH | Putative uncharacterized protein At2g18950 | Arabidopsis thaliana (Mouse-ear cress). |
| B7G488 | B7G488_PHATR | Predicted protein | Phaeodactylum tricornutum CCAP |
| | | | 1055/1. |
| B4FBU3 | B4FBU3_MAIZE | Putative uncharacterized protein | Zea mays (Maize). |
| B9H9X9 | B9H9X9_POPTR | Predicted protein | Populus trichocarpa (Western balsam |
| | | | poplar) (Populus balsamifera subsp. OS |
| | | | trichocarpa). |
| Q1ACB2 | Q1ACB2_SOYBN | Homogentisate phytyltransferase VTE2-2 | Glycine max (Soybean). |
| B8G328 | B8G328_CHLAD | UbiA prenyltransferase | Chloroflexus aggregans (strain MD-66/ |
| | | | DSM 9485). |
| Q017K0 | Q017K0_OSTTA | Putative tocopherol polyprenyltransferase | Ostreococcus tauri. |
| | | . . . | |
| C5XCE4 | C5XCE4_SORBI | Putative uncharacterized protein | Sorghum bicolor (Sorghum) (Sorghum |
| | | Sb02g037370 | vulgare). |
| Q0DAF2 | Q0DAF2_ORYSJ | Os06g0658300 protein | Oryza sativa subsp. japonica (Rice). |
| D1I9Z9 | D1I9Z9_VITVI | Whole genome shotgun sequence of line | Vitis vinifera (Grape). |
| | | PN4002 . . . | |
| A9SCS8 | A9SCS8_PHYPA | Predicted protein | Physcomitrella patens subsp. patens. |
| B9LDT9 | B9LDT9_CHLSY | UbiA prenyltransferase | Chloroflexus aurantiacus (strain ATCC |
| | | | 29364/DSM 637/Y-400-fl). |
| A9WKI4 | A9WKI4_CHLAA | UbiA prenyltransferase | Chloroflexus aurantiacus (strain ATCC |
| | | | 29366/DSM 635/J-10-fl). |
| Q0D576 | Q0D576_ORYSJ | Os07g0576000 proteinPutative | Oryza sativa subsp. japonica (Rice). |
| | | uncharacterized . . . | |
| B8B7R2 | B8B7R2_ORYSI | Putative uncharacterized protein | Oryza sativa subsp. indica (Rice). |
| Q1ACB3 | Q1ACB3_ARATH | Homogentisate phytyltransferase VTE2-2 | Arabidopsis thaliana (Mouse-ear cress). |
| A4RYL3 | A4RYL3_OSTLU | Predicted protein | Ostreococcus lucimarinus (strain |
| | | | CCE9901). |
| Q6ZLA8 | Q6ZLA8_ORYSJ | Putative tocopherol polyprenyltransferase | Oryza sativa subsp. japonica (Rice). |
| C1E4B5 | C1E4B5_9CHLO | Predicted protein | Micromonas sp. RCC299. |
| Q1ACB3 | Q1ACB3_ARATH | Homogentisate phytyltransferase VTE2-2 | Arabidopsis thaliana (Mouse-ear cress). |
| Q9LHM7 | Q9LHM7_ARATH | Dbj\|BAA17774.1 | Arabidopsis thaliana (Mouse-ear cress). |
| B9H9X9 | B9H9X9_POPTR | Predicted protein | Populus trichocarpa (Western balsam |
| | | | poplar) (Populus balsamifera subsp. OS |
| | | | trichocarpa). |
| D1I9Z9 | D1I9Z9_VITVI | Whole genome shotgun sequence of line | Vitis vinifera (Grape). |
| | | PN4002 . . . | |
| Q1ACB2 | Q1ACB2_SOYBN | Homogentisate phytyltransferase VTE2-2 | Glycine max (Soybean). |
| C5XCE4 | C5XCE4_SORBI | Putative uncharacterized protein | Sorghum bicolor (Sorghum) (Sorghum |
| | | Sb02g037370 | vulgare). |
| Q0D576 | Q0D576_ORYSJ | Os07g0576000 proteinPutative | Oryza sativa subsp. japonica (Rice). |
| | | uncharacterized . . . | |
| B8B7R2 | B8B7R2_ORYSI | Putative uncharacterized protein | Oryza sativa subsp. indica (Rice). |
| Q6ZLA8 | Q6ZLA8_ORYSJ | Putative tocopherol polyprenyltransferase | Oryza sativa subsp. japonica (Rice). |
| B6T6U8 | B6T6U8_MAIZE | Prenyltransferase/zinc ion binding protein | Zea mays (Maize). |
| B8A333 | B8A333_MAIZE | Putative uncharacterized protein | Zea mays (Maize). |
| B9T022 | B9T022_RICCO | Bacteriochlorophyll synthase, putative | Ricinus communis (Castor bean). |
| A9SCS8 | A9SCS8_PHYPA | Predicted protein | Physcomitrella patens subsp. patens. |
| A1JHN0 | A1JHN0_CHLRE | Homogentisate prenyltransferase | Chlamydomonas reinhardtii. |
| A8J261 | A8J261_CHLRE | Homogentisate solanesyltransferase | Chlamydomonas reinhardtii. |
| Q017K0 | Q017K0_OSTTA | Putative tocopherol polyprenyltransferase | Ostreococcus tauri. |
| | | . . . | |
| A4RYL3 | A4RYL3_OSTLU | Predicted protein | Ostreococcus lucimarinus (strain |
| | | | CCE9901). |
| C1MUF6 | C1MUF6_9CHLO | Predicted protein | Micromonas pusilla CCMP1545. |
| C1E4B5 | C1E4B5_9CHLO | Predicted protein | Micromonas sp. RCC299. |
| B8CAB5 | B8CAB5_THAPS | Tocopherol polyprenyltransferase-like | Thalassiosira pseudonana (Marine |
| | | protein | diatom). |
| B7FT51 | B7FT51_PHATR | Predicted protein | Phaeodactylum tricornutum CCAP |
| | | | 1055/1. |
| B1XQG6 | B1XQG6_SYNP2 | Homogentisate geranylgeranyl transferase | Synechococcus sp. (strain ATCC 27264/ |
| | | | PCC 7002/PR-6) (Agmenellum OS |
| | | | quadruplicatum). |
| B5W0F0 | B5W0F0_SPIMA | UbiA prenyltransferase | Arthrospira maxima CS-328. |
| B8HVT5 | B8HVT5_CYAP4 | UbiA prenyltransferase | Cyanothece sp. (strain PCC 7425/ |
| | | | ATCC 29141). |
| Q8YRJ8 | Q8YRJ8_ANASP | Alr3448 protein | Anabaena sp. (strain PCC 7120). |
| Q3M7F7 | Q3M7F7_ANAVT | Homogentisate phytyltransferase | Anabaena variabilis (strain ATCC 29413/ |
| | | | PCC 7937). |
| B7K7Y8 | B7K7Y8_CYAP7 | UbiA prenyltransferase | Cyanothece sp. (strain PCC 7424) |
| | | | (Synechococcus sp. (strain ATCC |
| | | | 29155)). |
| B7JZF9 | B7JZF9_CYAP8 | UbiA prenyltransferase | Cyanothece sp. (strain PCC 8801) |
| | | | (Synechococcus sp. (strain PCC 8801/ |
| | | | OS RF-1)). |
| C7QPT1 | C7QPT1_CYAP0 | UbiA prenyltransferase | Cyanothece sp. (strain PCC 8802) |
| | | | (Synechococcus sp. (strain RF-2)). |
| A0Z9X9 | A0Z9X9_NODSP | Putative uncharacterized protein | Nodularia spumigena CCY9414. |
| B1X091 | B1X091_CYAA5 | Putative uncharacterized protein | Cyanothece sp. (strain ATCC 51142). |
| Q7NNX5 | Q7NNX5_GLOVI | GII0283 protein | Gloeobacter violaceus. |
| Q2JTP7 | Q2JTP7_SYNJA | Prenyltransferase, UbiA family | Synechococcus sp. (strain JA-3-3Ab) |
| | | | (Cyanobacteria bacterium Yellowstone |
| | | | OS A-Prime). |
| P73726 | P73726_SYNY3 | Slr1736 protein | Synechocystis sp. (strain PCC 6803). |
| A3IY82 | A3IY82_9CHRO | Putative uncharacterized protein | Cyanothece sp. CCY0110. |
| B2J3Q6 | B2J3Q6_NOSP7 | UbiA prenyltransferase | Nostoc punctiforme (strain ATCC 29133/ |
| | | | PCC 73102). |
| B4WKY1 | B4WKY1_9SYNE | Prenyltransferase, UbiA family | Synechococcus sp. PCC 7335. |
| A0YXJ4 | A0YXJ4_9CYAN | UbiA prenyltransferase | Lyngbya sp. PCC 8106. |
| Q10XV6 | Q10XV6_TRIEI | Homogentisate phytyltransferase | Trichodesmium erythraeum (strain |
| | | | IMS101). |
| Q2JJI8 | Q2JJI8_SYNJB | Prenyltransferase, UbiA family | Synechococcus sp. (strain JA-2-3B'a(2- |
| | | | 13)) (Cyanobacteria bacterium OS |
| | | | Yellowstone B-Prime). |
| Q4C0Z2 | Q4C0Z2_CROWT | UbiA prenyltransferase | Crocosphaera watsonii WH 8501. |
| B9YIB4 | B9YIB4_ANAAZ | UbiA prenyltransferase | ‘Nostoc azollae’ 0708. |
| B0JR78 | B0JR78_MICAN | Homogentisate phytyltransferase | Microcystis aeruginosa (strain NIES- |
| | | | 843). |
| A8YN38 | A8YN38_MICAE | Genome sequencing data, contig C328 | Microcystis aeruginosa PCC 7806. |
| B6CPP7 | B6CPP7_9APIA | Homogentisate geranylgeranyl transferase | Angelica gigas. |
| Q1ACB5 | Q1ACB5_9MYRT | Homogentisate phytyltransferase VTE2-1 | Cuphea pulcherrima. |
| Q8VWJ1 | Q8VWJ1_ARATH | Homogentisate phytyltransferasePutative | Arabidopsis thaliana (Mouse-ear cress). |
| | | unch . . . | |
| D1H9L3 | D1H9L3_VITVI | Whole genome shotgun sequence of line | Vitis vinifera (Grape). |
| | | PN4002 . . . | |
| Q5PT36 | Q5PT36_VITVI | Homogentisate geranylgeranyl transferase | Vitis vinifera (Grape). |
| B0BZ73 | B0BZ73_ACAM1 | Prenyltransferase, UbiA family | Acaryochloris marina (strain MBIC |
| | | | 11017). |
| B2Z8W9 | B2Z8W9_CORSA | Chloroplast homogentisate | Coriandrum sativum (Coriander). |
| | | phytyltransferase | |
| A5C4H9 | A5C4H9_VITVI | Putative uncharacterized protein | Vitis vinifera (Grape). |
| A9RVY9 | A9RVY9_PHYPA | Predicted protein | Physcomitrella patens subsp. patens. |
| B6CPT3 | B6CPT3_9APIA | Homogentisic acid phytyltransferase | Angelica gigas. |
| A9SGV2 | A9SGV2_PHYPA | Predicted protein | Physcomitrella patens subsp. patens. |
| Q1ACB7 | Q1ACB7_WHEAT | Homogentisate phytyltransferase VTE2-1 | Triticum aestivum (Wheat). |
| B7FA90 | B7FA90_ORYSJ | cDNA, clone: J100057F06, full insert | Oryza sativa subsp. japonica (Rice). |
| | | sequence | |
| B6U7K6 | B6U7K6_MAIZE | Homogentisate geranylgeranyl | Zea mays (Maize). |
| | | transferasePuta . . . | |
| C5Z789 | C5Z789_SORBI | Putative uncharacterized protein | Sorghum bicolor (Sorghum) (Sorghum |
| | | Sb10g026190 | vulgare). |
| Q1ACB8 | Q1ACB8_MAIZE | Homogentisate phytyltransferase VTE2-1 | Zea mays (Maize). |
| B8B0R2 | B8B0R2_ORYSI | Putative uncharacterized protein | Oryza sativa subsp. indica (Rice). |
| Q7XB13 | Q7XB13_WHEAT | Homogentisic acid geranylgeranyl | Triticum aestivum (Wheat). |
| | | transferase | |
| Q1ACB4 | Q1ACB4_SOYBN | Homogentisate phytyltransferase VTE2-1 | Glycine max (Soybean). |
| Q7XB12 | Q7XB12_ORYSJ | Homogentisic acid geranylgeranyl | Oryza sativa subsp. japonica (Rice). |
| | | transferase | |
| A3BE29 | A3BE29_ORYSJ | Putative uncharacterized protein | Oryza sativa subsp. japonica (Rice). |
| B2M1Y0 | B2M1Y0_MANES | Homogentisate phytyltransferase | Manihot esculenta (Cassava) (Manioc). |
| C6TBP2 | C6TBP2_SOYBN | Putative uncharacterized protein | Glycine max (Soybean). |
| B6CPP6 | B6CPP6_SESIN | Homogentisic acid phytyltransferase | Sesamum indicum (Oriental sesame) |
| | | | Gingelly). |
| B9LDT9 | B9LDT9_CHLSY | UbiA prenyltransferase | Chloroflexus aurantiacus (strain ATCC |
| | | | 29364/DSM 637/Y-400-fl). |
| A9WKI4 | A9WKI4_CHLAA | UbiA prenyltransferase | Chloroflexus aurantiacus (strain ATCC |
| | | | 29366/DSM 635/J-10-fl). |
| Q647J9 | Q647J9_MEDSA | Homogentisate phytylprenyltransferase | Medicago sativa (Alfalfa). |
| Q1ACB6 | Q1ACB6_ALLPO | Homogentisate phytyltransferase VTE2-1 | Allium porrum (Leek). |
| A9RDP5 | A9RDP5_PHYPA | Predicted protein | Physcomitrella patens subsp. patens. |
| B7X937 | B7X937_HEVBR | Homogentisate phytyl transferase | Hevea brasiliensis (Para rubber tree). |
| Q7XB14 | Q7XB14_HORVU | Homogentisic acid geranylgeranyl | Hordeum vulgare (Barley). |
| | | transferase | |
| C7EZA1 | C7EZA1_MALDO | Homogentisate phytyltransferase | Malus domestica (Apple) (Pyrus malus). |
| D2CZX9 | D2CZX9_LACSA | Homogentisate phytylprenyltransferase | Lactuca sativa (Garden lettuce). |
| C5Z6S0 | C5Z6S0_SORBI | Putative uncharacterized protein | Sorghum bicolor (Sorghum) (Sorghum |
| | | Sb10g025475 | vulgare). |
| C0LTT9 | C0LTT9_LINUS | Homogentisate phytyltransferase | Linum usitatissimum (Flax) (Linseed). |
| B7X939 | B7X939_HEVBR | Homogentisate geranylgeranyl transferase | Hevea brasiliensis (Para rubber tree). |
| Q58FG4 | Q58FG4_SOYBN | Homogentisate phytylprenyltransferase | Glycine max (Soybean). |
| B8G328 | B8G328_CHLAD | UbiA prenyltransferase | Chloroflexus aggregans (strain MD-66/ |
| | | | DSM 9485). |
| B9FQB7 | B9FQB7_ORYSJ | Putative uncharacterized protein | Oryza sativa subsp. japonica (Rice). |
| C1MHL9 | C1MHL9_9CHLO | Predicted protein | Micromonas pusilla CCMP1545. |
| Q67U28 | Q67U28_ORYSJ | Putative uncharacterized protein | Oryza sativa subsp. japonica (Rice). |
| | | B1047G05.17 . . . | |
| Q0DAK7 | Q0DAK7_ORYSJ | Os06g0646900 protein | Oryza sativa subsp. japonica (Rice). |
| Q67W53 | Q67W53_ORYSJ | Putative homogentisic acid geranylgeranyl | Oryza sativa subsp. japonica (Rice). |
| | | tr . . . | |
| B9ILX0 | B9ILX0_POPTR | Predicted protein | Populus trichocarpa (Western balsam |
| | | | poplar) (Populus balsamifera subsp. OS |
| | | | trichocarpa). |
| B4W4M5 | B4W4M5_9CYAN | Prenyltransferase, UbiA family | Microcoleus chthonoplastes PCC 7420. |
| B7G488 | B7G488_PHATR | Predicted protein | Phaeodactylum tricornutum CCAP |
| | | | 1055/1. |
| D1HIV6 | D1HIV6_VITVI | Whole genome shotgun sequence of line | Vitis vinifera (Grape). |
| | | PN4002 . . . | |
| B9T664 | B9T664_RICCO | Bacteriochlorophyll synthase, putative | Ricinus communis (Castor bean). |
| B9IL30 | B9IL30_POPTR | Predicted protein | Populus trichocarpa (Western balsam |
| | | | poplar) (Populus balsamifera subsp. OS |
| | | | trichocarpa). |
| B1B5P5 | B1B5P5_9FABA | Flavonoid prenyltransferase | Sophora flavescens. |
| A4RT35 | A4RT35_OSTLU | Homogentisate | Ostreococcus lucimarinus (strain |
| | | phytylprenyltransferase/homoge . . . | CCE9901). |
| B1B5P4 | B1B5P4_9FABA | Naringenin 8-dimethylallyltransferase | Sophora flavescens. |
| B1B3P3 | B1B3P3_9FABA | Naringenin 8-dimethylallyltransferase | Sophora flavescens. |
| C1EAI7 | C1EAI7_9CHLO | Predicted protein | Micromonas sp. RCC299. |
| B9A1Q4 | B9A1Q4_SOYBN | Pterocarpan 4-dimethylallyltransferase | Glycine max (Soybean). |
| A8J4W7 | A8J4W7_CHLRE | Predicted protein | Chlamydomonas reinhardtii. |

### Cyanogen Detoxification

As taught herein, the production of ROS is associated with cyanide levels in comestibles (e.g. cassava roots). Expression of AOX significantly reduces ROS production and PPD. Further surprising, however, is that such transgenic plants can still produce non-trivial levels of ROS. Without being bound by theory, the present inventors believe that the expression of cyanogen detoxification genes reduces PPD by reducing cytochrome-toxic levels of cyanide. Accordingly, in one embodiment, the invention provides a plant (e.g. cassava) which overexpresses one or more cyanogen detoxification genes alone or in combination with AOX or other transgene(s) taught herein (e.g. antioxidation products). Optionally, the one or more cyanogen detoxification genes are selected from cyanogen metabolizing enzymes and cyanogen biosynthesis inhibitors (e.g. cyanogen biosynthesis-targeted RNAi).

### Cyanogen Metabolism

In some embodiments, the invention provides a plant (e.g. cassava) that contains a cyanogen metabolism transgene (e.g. enzyme). The cyanogen metabolism gene can be any gene (enzyme) that reduces cyanide levels in a harvested and/or processed comestible (e.g. cassava root) when overexpressed. Examples of such are well known in the art. With the teachings provided herein, one can now select cyanogen metabolism transgenes for expression in order to reduce cyanide-induced ROS and PPD production.

### β-Cyanoalanine Synthase

In some embodiments, the invention provides a plant (e.g. cassava) that contains a β-cyanoalanine synthase (β-CAS) transgene. The β-CAS can be any β-CAS transgene that is functional in the plant. The β-CAS can be any β-CAS enzyme known in the art. β-CAS catalyzes the conversion of β-cyano-alanine from cystein and cyanide. Without being bound by theory, the present inventors believe that β-CAS provides a key step in cyanide/nitrogen assimilation to amino acids.

Optionally, the β-CAS is any β-CAS set forth in Table 8. Optionally, the β-CAS exhibits a sequence identity of at least about any of 75%, 80%, 85%, 90%, or 95% to a β-CAS listed in Table 8, or an active fragment thereof. Optionally, the β-CAS is derived from any of the species listed in Table 8.

Examplary β-CAS transgenes comprise one or more of the following features:

t. cystathione beta synthase-like domain;
u. tryptophan synthase beta II-like domain.

Optionally, the β-CAS is a plant, bacterial, or fungal β-CAS.

Optionally, the β-CAS is a monocot, dicot, cassava, or Arabidopsis β-CAS.

Optionally, the β-CAS is operably linked to a comestible (e.g. root) specific promoter. Optionally, the β-CAS is operably linked to a patatin promoter.

TABLE 8
β-CAS Transgenes

| | | | |
|---|---|---|---|
| B0FTX3 | B0FTX3_MANES | Cysteine synthase (EC 2.5.1.47) | Manihot esculenta (Cassava) (Manioc). |
| Q5VLJ4 | Q5VLJ4_HEVBR | Cysteine synthase (EC 2.5.1.47) | Hevea brasiliensis (Para rubber tree). |
| Q5VLJ3 | Q5VLJ3_HEVBR | Cysteine synthase (EC 2.5.1.47) | Hevea brasiliensis (Para rubber tree). |
| B9GQA5 | B9GQA5_POPTR | Cysteine synthase (EC 2.5.1.47) | Populus trichocarpa (Western balsam poplar) (Populus |
| | | | balsamifera subsp. OS trichocarpa). |
| A9PGL6 | A9PGL6_POPTR | Cysteine synthase (EC 2.5.1.47) | Populus trichocarpa (Western balsam poplar) (Populus |
| | | | balsamifera subsp. OS trichocarpa). |
| A5AEP0 | A5AEP0_VITVI | Cysteine synthase (EC 2.5.1.47) | Vitis vinifera (Grape). |
| D1J462 | D1J462_VITVI | Cysteine synthase (EC 2.5.1.47) | Vitis vinifera (Grape). |
| B9I8L3 | B9I8L3_POPTR | Cysteine synthase (EC 2.5.1.47) | Populus trichocarpa (Western balsam poplar) (Populus |
| | | | balsamifera subsp. OS trichocarpa). |
| Q7Y256 | Q7Y256_BETVE | Cysteine synthase (EC 2.5.1.47) | Betula verrucosa (White birch) (Betula pendula). |
| A5YT86 | A5YT86_SOYBN | Cysteine synthase (EC 2.5.1.47) | Glycine max (Soybean). |
| B9RDU1 | B9RDU1_RICCO | Cysteine synthase (EC 2.5.1.47) | Ricinus communis (Castor bean). |
| Q1KLZ2 | Q1KLZ2_MALDO | Cysteine synthase (EC 2.5.1.47) | Malus domestica (Apple) (Pyrus malus). |
| Q1KLZ1 | Q1KLZ1_MALDO | Cysteine synthase (EC 2.5.1.47) | Malus domestica (Apple) (Pyrus malus). |
| B9RZ17 | B9RZ17_RICCO | Cysteine synthase (EC 2.5.1.47) | Ricinus communis (Castor bean). |
| Q76MX2 | Q76MX2_SOLTU | Cysteine synthase (EC 2.5.1.47) | Solanum tuberosum (Potato). |
| Q9S757 | Q9S757_ARATH | Cysteine synthase (EC 2.5.1.47) | Arabidopsis thaliana (Mouse-ear cress). |
| Q9FS29 | Q9FS29_SOLTU | Cysteine synthase (EC 2.5.1.47) | Solanum tuberosum (Potato). |
| Q43153 | Q43153_SPIOL | Cysteine synthase (EC 2.5.1.47) | Spinacia oleracea (Spinach). |
| Q5UJF9 | Q5UJF9_ORYSI | Cysteine synthase (EC 2.5.1.47) | Oryza sativa subsp. indica (Rice). |
| Q7XS58 | Q7XS58_ORYSJ | Cysteine synthase (EC 2.5.1.47) | Oryza sativa subsp. japonica (Rice). |
| C5YC80 | C5YC80_SORBI | Cysteine synthase (EC 2.5.1.47) | Sorghum bicolor (Sorghum) (Sorghum vulgare). |
| C0PCX2 | C0PCX2_MAIZE | Cysteine synthase (EC 2.5.1.47) | Zea mays (Maize). |
| B6TBZ1 | B6TBZ1_MAIZE | Cysteine synthase (EC 2.5.1.47) | Zea mays (Maize). |
| A3AQX8 | A3AQX8_ORYSJ | Cysteine synthase (EC 2.5.1.47) | Oryza sativa subsp. japonica (Rice). |
| B8LQB8 | B8LQB8_PICSI | Cysteine synthase (EC 2.5.1.47) | Picea sitchensis (Sitka spruce). |
| A9NR69 | A9NR69_PICSI | Cysteine synthase (EC 2.5.1.47) | Picea sitchensis (Sitka spruce). |
| A9RMD3 | A9RMD3_PHYPA | Cysteine synthase (EC 2.5.1.47) | Physcomitrella patens subsp. patens. |
| A9SEU2 | A9SEU2_PHYPA | Cysteine synthase (EC 2.5.1.47) | Physcomitrella patens subsp. patens. |
| B9SFU8 | B9SFU8_RICCO | Cysteine synthase (EC 2.5.1.47) | Ricinus communis (Castor bean). |
| Q3LAG6 | Q3LAG6_TOBAC | Cysteine synthase (EC 2.5.1.47) | Nicotiana tabacum (Common tobacco). |
| Q3L197 | Q3L197_ALLSA | Cysteine synthase (EC 2.5.1.47) | Allium sativum (Garlic). |
| P32260 | CYSKP_SPIOL | Cysteine synthase, | Spinacia oleracea (Spinach). |
| | | chloroplastic/chromoplast . . . | |
| Q9XEA8 | CYSK2_ORYSJ | Cysteine synthase (CSase) (EC | Oryza sativa subsp. japonica (Rice). |
| | | 2.5.1.47) (O . . . | |
| Q10CX6 | Q10CX6_ORYSJ | Cysteine synthase (EC 2.5.1.47) | Oryza sativa subsp. japonica (Rice). |
| B9FBT5 | B9FBT5_ORYSJ | Putative uncharacterized protein | Oryza sativa subsp. japonica (Rice). |
| B8AJV7 | B8AJV7_ORYSI | Putative uncharacterized protein | Oryza sativa subsp. indica (Rice). |
| A9NS10 | A9NS10_PICSI | Cysteine synthase (EC 2.5.1.47) | Picea sitchensis (Sitka spruce). |
| O81155 | CYSKP_SOLTU | Cysteine synthase, | Solanum tuberosum (Potato). |
| | | chloroplastic/chromoplast . . . | |
| B5U9V0 | B5U9V0_SPIOL | Cysteine synthase (EC 2.5.1.47) | Spinacia oleracea (Spinach). |
| A9NRJ4 | A9NRJ4_PICSI | Cysteine synthase (EC 2.5.1.47) | Picea sitchensis (Sitka spruce). |
| Q9FS26 | Q9FS26_SOLTU | Cysteine synthase (EC 2.5.1.47) | Solanum tuberosum (Potato). |
| D1HU72 | D1HU72_VITVI | Cysteine synthase (EC 2.5.1.47) | Vitis vinifera (Grape). |
| B8A367 | B8A367_MAIZE | Cysteine synthase (EC 2.5.1.47) | Zea mays (Maize). |
| A5AFH5 | A5AFH5_VITVI | Cysteine synthase (EC 2.5.1.47) | Vitis vinifera (Grape). |
| C5XFP1 | C5XFP1_SORBI | Cysteine synthase (EC 2.5.1.47) | Sorghum bicolor (Sorghum) (Sorghum vulgare). |
| B4FR08 | B4FR08_MAIZE | Cysteine synthase (EC 2.5.1.47) | Zea mays (Maize). |
| A3RM03 | A3RM03_SOYBN | Cysteine synthase (EC 2.5.1.47) | Glycine max (Soybean). |
| C6TMX6 | C6TMX6_SOYBN | Cysteine synthase (EC 2.5.1.47) | Glycine max (Soybean). |
| B9RTR4 | B9RTR4_RICCO | Cysteine synthase (EC 2.5.1.47) | Ricinus communis (Castor bean). |
| O23733 | CYSK1_BRAJU | Cysteine synthase (CSase) (EC | Brassica juncea (Leaf mustard) (Indian mustard). |
| | | 2.5.1.47) (O . . . | |
| Q5JNB0 | Q5JNB0_ORYSJ | Cysteine synthase (EC 2.5.1.47) | Oryza sativa subsp. japonica (Rice). |
| D0V0B2 | D0V0B2_BRARC | Cysteine synthase (EC 2.5.1.47) | Brassica rapa subsp. chinensis (Pak-choi). |
| Q00834 | CYSK_SPIOL | Cysteine synthase (EC 2.5.1.47) | Spinacia oleracea (Spinach). |
| | | (O-acetylse . . . | |
| B5U9U9 | B5U9U9_SPIOL | Cysteine synthase (EC 2.5.1.47) | Spinacia oleracea (Spinach). |
| Q2QLX5 | Q2QLX5_ORYSJ | Cysteine synthase (EC 2.5.1.47) | Oryza sativa subsp. japonica (Rice). |
| C6TDJ4 | C6TDJ4_SOYBN | Cysteine synthase (EC 2.5.1.47) | Glycine max (Soybean). |
| A9PJI4 | A9PJI4_9ROSI | Cysteine synthase (EC 2.5.1.47) | Populus trichocarpa X Populus deltoides. |
| A2ZMY2 | A2ZMY2_ORYSI | Cysteine synthase (EC 2.5.1.47) | Oryza sativa subsp. indica (Rice). |
| Q43725 | CYSKM_ARATH | Cysteine synthase, | Arabidopsis thaliana (Mouse-ear cress). |
| | | mitochondrial (EC 2.5.1.4 . . . | |
| Q3EAH3 | Q3EAH3_ARATH | Cysteine synthase (EC 2.5.1.47) | Arabidopsis thaliana (Mouse-ear cress). |
| Q0WWQ5 | Q0WWQ5_ARATH | Cysteine synthase (EC 2.5.1.47) | Arabidopsis thaliana (Mouse-ear cress). |
| C6TIP9 | C6TIP9_SOYBN | Cysteine synthase (EC 2.5.1.47) | Glycine max (Soybean). |
| B9RET4 | B9RET4_RICCO | Cysteine synthase (EC 2.5.1.47) | Ricinus communis (Castor bean). |
| B9P570 | B9P570_POPTR | Cysteine synthase (EC 2.5.1.47) | Populus trichocarpa (Western balsam poplar) (Populus |
| | | | balsamifera subsp. OS trichocarpa). |
| Q8W1A0 | Q8W1A0_SOYBN | Cysteine synthase (EC 2.5.1.47) | Glycine max (Soybean). |
| Q9MAZ2 | Q9MAZ2_ALLTU | Cysteine synthase (EC 2.5.1.47) | Allium tuberosum (Garlic chives). |
| Q6STL6 | Q6STL6_NICPL | Cysteine synthase (EC 2.5.1.47) | Nicotiana plumbaginifolia (Leadwort-leaved tobacco). |
| Q3LAG5 | Q3LAG5_TOBAC | Cysteine synthase (EC 2.5.1.47) | Nicotiana tabacum (Common tobacco). |
| B8A377 | B8A377_MAIZE | Cysteine synthase (EC 2.5.1.47) | Zea mays (Maize). |
| B9MZH9 | B9MZH9_POPTR | Cysteine synthase (EC 2.5.1.47) | Populus trichocarpa (Western balsam poplar) (Populus |
| | | | balsamifera subsp. OS trichocarpa). |
| A9Y098 | A9Y098_SESIN | Cysteine synthase (EC 2.5.1.47) | Sesamum indicum (Oriental sesame) (Gingelly). |
| D0V0B3 | D0V0B3_BRARC | Cysteine synthase (EC 2.5.1.47) | Brassica rapa subsp. chinensis (Pak-choi). |
| O23735 | CYSK2_BRAJU | Cysteine synthase (CSase) (EC | Brassica juncea (Leaf mustard) (Indian mustard). |
| | | 2.5.1.47) (O . . . | |
| P47998 | CYSK1_ARATH | Cysteine synthase (EC 2.5.1.47) | Arabidopsis thaliana (Mouse-ear cress). |
| | | (O-acetylse . . . | |
| Q00XK0 | Q00XK0_OSTTA | Cysteine synthase (EC 2.5.1.47) | Ostreococcus tauri. |
| B7FKU7 | B7FKU7_MEDTR | Cysteine synthase (EC 2.5.1.47) | Medicago truncatula (Barrel medic). |
| B9N5X9 | B9N5X9_POPTR | Cysteine synthase (EC 2.5.1.47) | Populus trichocarpa (Western balsam poplar) (Populus |
| | | | balsamifera subsp. OS trichocarpa). |
| Q9FS27 | Q9FS27_SOLTU | Cytosolic cysteine synthase | Solanum tuberosum (Potato). |
| A3CJM0 | A3CJM0_ORYSJ | Cysteine synthase (EC 2.5.1.47) | Oryza sativa subsp. japonica (Rice). |
| Q0WLF5 | Q0WLF5_ARATH | Cysteine synthase (EC 2.5.1.47) | Arabidopsis thaliana (Mouse-ear cress). |
| Q43317 | CYSK_CITLA | Cysteine synthase (CSase) (EC | Citrullus lanatus (Watermelon) (Citrullus vulgaris). |
| | | 2.5.1.47) (O . . . | |
| A8ISA9 | A8ISA9_CHLRE | Cysteine synthase (EC 2.5.1.47) | Chlamydomonas reinhardtii. |
| A5HKN4 | A5HKN4_GLYSO | Cysteine synthase (EC 2.5.1.47) | Glycine soja (Wild soybean). |
| P80608 | CYSK_MAIZE | Cysteine synthase (CSase) (EC | Zea mays (Maize). |
| | | 2.5.1.47) (O . . . | |
| A9PA30 | A9PA30_POPTR | Cysteine synthase (EC 2.5.1.47) | Populus trichocarpa (Western balsam poplar) (Populus |
| | | | balsamifera subsp. OS trichocarpa). |
| Q9XEA6 | CYSK1_ORYSJ | Cysteine synthase (CSase) (EC | Oryza sativa subsp. japonica (Rice). |
| | | 2.5.1.47) (O . . . | |
| Q3L196 | Q3L196_ALLSA | Cysteine synthase (EC 2.5.1.47) | Allium sativum (Garlic). |
| Q0ILS7 | Q0ILS7_ORYSJ | Cysteine synthase (EC 2.5.1.47) | Oryza sativa subsp. japonica (Rice). |
| A8ISB0 | A8ISB0_CHLRE | Cysteine synthase (EC 2.5.1.47) | Chlamydomonas reinhardtii. |
| A3RM04 | A3RM04_SOYBN | Cysteine synthase (EC 2.5.1.47) | Glycine max (Soybean). |
| P31300 | CYSKP_CAPAN | Cysteine synthase, | Capsicum annuum (Bell pepper). |
| | | chloroplastic/chromoplast . . . | |
| O81154 | CYSK_SOLTU | Cysteine synthase (EC 2.5.1.47) | Solanum tuberosum (Potato). |
| | | (O-acetylse . . . | |
| A5YT88 | A5YT88_SOYBN | Cysteine synthase (EC 2.5.1.47) | Glycine max (Soybean). |
| B9HJY5 | B9HJY5_POPTR | Cysteine synthase (EC 2.5.1.47) | Populus trichocarpa (Western balsam poplar) (Populus |
| | | | balsamifera subsp. OS trichocarpa). |
| P38076 | CYSK_WHEAT | Cysteine synthase (EC 2.5.1.47) | Triticum aestivum (Wheat). |
| | | (O-acetylse . . . | |
| O81523 | O81523_CHLRE | Cysteine synthase (EC 2.5.1.47) | Chlamydomonas reinhardtii. |
| A4S621 | A4S621_OSTLU | Cysteine synthase (EC 2.5.1.47) | Ostreococcus lucimarinus (strain CCE9901). |
| D1ILH3 | D1ILH3_VITVI | Cysteine synthase (EC 2.5.1.47) | Vitis vinifera (Grape). |
| B2Z452 | B2Z452_9CARY | Cysteine synthase (EC 2.5.1.47) | Knorringia sibirica. |
| A5BDL2 | A5BDL2_VITVI | Cysteine synthase (EC 2.5.1.47) | Vitis vinifera (Grape). |

### Nitrilase 4

In some embodiments, the invention provides a plant (e.g. cassava) that contains a nitrilase 4 (NIT4). The NIT4 can be any NIT4 transgene that is functional in the plant. NIT4 catalyzes the conversion of β-cyano-alanine from cysteine and cyanide. Without being bound by theory, the present inventors believe that NIT4 provides a key step in cyanide/nitrogen assimilation to amino acids.

Optionally, the NIT4 is any NIT4 set forth in Table 9. Optionally, the NIT4 exhibits a sequence identity of at least about any of 75%, 80%, 85%, 90%, or 95% to a NIT4 listed in Table 9, or an active fragment thereof.

Examplary NIT4 transgenes comprise one or more of the following features:

v. a nitrolase (e.g. nitrolase-I) domain; and
w. an amidohydrolase domain.

The NIT4 can be any NIT4 enzyme known in the art. Optionally, the NIT4 is a plant, bacterial, or fungal NIT4.

Optionally, the NIT4 is a monocot, dicot, cassava, or Arabidopsis NIT4.

Optionally, the NIT4 is operably linked to a comestible (e.g. root) specific promoter. Optionally, the NIT4 is operably linked to a patatin promoter.

Optionally, the NIT is derived from any of the species listed in Table 9.

TABLE 9
NIT4 Transgenes

| | | | |
|---|---|---|---|
| P46011 | NRL4_ARATH | Bifunctional nitrilase/nitrile | Arabidopsis thaliana (Mouse-ear cress). |
| | | hydratase NIT . . . | |
| A3QYW4 | A3QYW4_BRACM | Nitrilase 4 | Brassica campestris (Field mustard). |
| B9MYU3 | B9MYU3_POPTR | Nitrilase 1 | Populus trichocarpa (Western balsam poplar) |
| | | | (Populus balsamifera subsp. OS trichocarpa). |
| B9SCY8 | B9SCY8_RICCO | Nitrilase, putative | Ricinus communis (Castor bean). |
| Q42965 | NRL4A_TOBAC | Bifunctional nitrilase/nitrile | Nicotiana tabacum (Common tobacco). |
| | | hydratase NIT . . . | |
| C6T972 | C6T972_SOYBN | Putative uncharacterized protein | Glycine max (Soybean). |
| Q42966 | NRL4B_TOBAC | Bifunctional nitrilase/nitrile | Nicotiana tabacum (Common tobacco). |
| | | hydratase NIT . . . | |
| Q5QGZ8 | Q5QGZ8_LUPAN | Nitrilase 4A | Lupinus angustifolius (Narrow-leaved blue lupin). |
| Q3LRV4 | Q3LRV4_LUPAN | Nitrilase 4B | Lupinus angustifolius (Narrow-leaved blue lupin). |
| B9SCY5 | B9SCY5_RICCO | Nitrilase, putative | Ricinus communis (Castor bean). |
| B9SCY6 | B9SCY6_RICCO | Nitrilase, putative | Ricinus communis (Castor bean). |
| Q6H849 | NRL4_ORYSJ | Bifunctional nitrilase/nitrile | Oryza sativa subsp. japonica (Rice). |
| | | hydratase NIT . . . | |
| B7EVI5 | B7EVI5_ORYSJ | cDNA clone: 001-020-F10, full | Oryza sativa subsp. japonica (Rice). |
| | | insert sequence . . . | |
| A2X7K6 | A2X7K6_ORYSI | Putative uncharacterized protein | Oryza sativa subsp. indica (Rice). |
| B9SCY7 | B9SCY7_RICCO | Nitrilase, putative | Ricinus communis (Castor bean). |
| Q6YDN0 | Q6YDN0_MAIZE | Nitrilase 4Putative uncharacterized | Zea mays (Maize). |
| | | proteinN . . . | |
| C5XY71 | C5XY71_SORBI | Putative uncharacterized protein | Sorghum bicolor (Sorghum) (Sorghum vulgare). |
| | | Sb04g026950 | |
| A4ULE0 | A4ULE0_MAIZE | Nitrilase 1 | Zea mays (Maize). |
| Q6YDN1 | Q6YDN1_MAIZE | Nitrilase 2 | Zea mays (Maize). |
| B6TVQ5 | B6TVQ5_MAIZE | Nitrilase 4 | Zea mays (Maize). |
| B4FQE2 | B4FQE2_MAIZE | Putative uncharacterized protein | Zea mays (Maize). |
| A4ULE1 | A4ULE1_MAIZE | Nitrilase 2 | Zea mays (Maize). |
| C5XY70 | C5XY70_SORBI | Putative uncharacterized protein | Sorghum bicolor (Sorghum) (Sorghum vulgare). |
| | | Sb04g026930 | |
| B8LLB3 | B8LLB3_PICSI | Putative uncharacterized protein | Picea sitchensis (Sitka spruce). |
| Q6H851 | Q6H851_ORYSJ | Os02g0635000 proteincDNA | Oryza sativa subsp. japonica (Rice). |
| | | clone: 001-020-C07, . . . | |
| A9T599 | A9T599_PHYPA | Predicted protein | Physcomitrella patens subsp. patens. |
| D1IVN2 | D1IVN2_VITVI | Whole genome shotgun sequence | Vitis vinifera (Grape). |
| | | of line PN4002 . . . | |
| D1HQ22 | D1HQ22_VITVI | Whole genome shotgun sequence | Vitis vinifera (Grape). |
| | | of line PN4002 . . . | |
| O04907 | O04907_ARATH | Nitrilase 2 | Arabidopsis thaliana (Mouse-ear cress). |
| D1IVN7 | D1IVN7_VITVI | Whole genome shotgun sequence | Vitis vinifera (Grape). |
| | | of line PN4002 . . . | |
| P32962 | NRL2_ARATH | Nitrilase 2 (EC 3.5.5.1) | Arabidopsis thaliana (Mouse-ear cress). |
| Q1LYZ1 | Q1LYZ1_ARATH | At3g44300Nitrilase 2 | Arabidopsis thaliana (Mouse-ear cress). |
| P46010 | NRL3_ARATH | Nitrilase 3 (EC 3.5.5.1) | Arabidopsis thaliana (Mouse-ear cress). |
| A5BPZ6 | A5BPZ6_VITVI | Putative uncharacterized protein | Vitis vinifera (Grape). |
| D1IVN0 | D1IVN0_VITVI | Whole genome shotgun sequence | Vitis vinifera (Grape). |
| | | of line PN4002 . . . | |
| A5B4Q5 | A5B4Q5_VITVI | Putative uncharacterized protein | Vitis vinifera (Grape). |
| Q8LAZ4 | Q8LAZ4_ARATH | Nitrilase 3 | Arabidopsis thaliana (Mouse-ear cress). |
| P32961 | NRL1_ARATH | Nitrilase 1 (EC 3.5.5.1) | Arabidopsis thaliana (Mouse-ear cress). |
| C0SVD5 | C0SVD5_ARATH | Putative uncharacterized protein | Arabidopsis thaliana (Mouse-ear cress). |
| | | At3g44310 | |
| D1IVP2 | D1IVP2_VITVI | Whole genome shotgun sequence | Vitis vinifera (Grape). |
| | | of line PN4002 . . . | |
| D1IVN8 | D1IVN8_VITVI | Whole genome shotgun sequence | Vitis vinifera (Grape). |
| | | of line PN4002 . . . | |
| Q944K7 | Q944K7_ARATH | AT3g44310/T10D17_100 | Arabidopsis thaliana (Mouse-ear cress). |
| D1IVN6 | D1IVN6_VITVI | Whole genome shotgun sequence | Vitis vinifera (Grape). |
| | | of line PN4002 . . . | |
| B5U8Z2 | B5U8Z2_BRARP | Putative nitrilase | Brassica rapa subsp. pekinensis (Chinese cabbage). |
| A3QYW3 | A3QYW3_BRACM | Nitrilase 2 | Brassica campestris(Field mustard). |
| D1IVM9 | D1IVM9_VITVI | Whole genome shotgun sequence | Vitis vinifera (Grape). |
| | | of line PN4002 . . . | |
| B9F194 | B9F194_ORYSJ | Putative uncharacterized protein | Oryza sativa subsp. japonica (Rice). |
| Q94JL5 | Q94JL5_BRANA | Nitrilase-like protein | Brassica napus (Rape). |
| A3QYW2 | A3QYW2_BRACM | Nitrilase 1 | Brassica campestris (Field mustard). |
| B5U8Z3 | B5U8Z3_BRARP | Putative nitrilase | Brassica rapa subsp. pekinensis (Chinese cabbage). |
| D1IVM6 | D1IVM6_VITVI | Whole genome shotgun sequence | Vitis vinifera (Grape). |
| | | of line PN4002 . . . | |
| D1C8L7 | D1C8L7_SPHTD | Nitrilase/cyanide hydratase and | Sphaerobacter thermophilus (strain DSM 20745/S |
| | | apolipoprote . . . | 6022). |
| A5B7G9 | A5B7G9_VITVI | Putative uncharacterized protein | Vitis vinifera (Grape). |
| B5U8Z4 | B5U8Z4_BRARP | Putative nitrilase | Brassica rapa subsp. pekinensis (Chinese cabbage). |
| A0LKP2 | A0LKP2_SYNFM | Nitrilase/cyanide hydratase and | Syntrophobacter fumaroxidans (strain DSM 10017/ |
| | | apolipoprote . . . | MPOB). |
| A7IFM1 | A7IFM1_XANP2 | Nitrilase/cyanide hydratase and | Xanthobacter autotrophicus (strain ATCC BAA-1158/ |
| | | apolipoprote . . . | Py2). |
| B9IIQ6 | B9IIQ6_POPTR | Nitrilase 3 | Populus trichocarpa (Western balsam poplar) |
| | | | (Populus balsamifera subsp. OS trichocarpa). |
| B9HBW3 | B9HBW3_POPTR | Nitrilase 2 | Populus trichocarpa (Western balsam poplar) |
| | | | (Populus balsamifera subsp. OS trichocarpa). |
| Q7WNC4 | Q7WNC4_BORBR | Nitrilase | Bordetella bronchiseptica (Alcaligenes |
| | | | bronchisepticus). |
| D0DDB0 | D0DDB0_9RHOB | Nitrilase 2 | Citreicella sp. SE45. |
| C5YCH6 | C5YCH6_SORBI | Putative uncharacterized protein | Sorghum bicolor (Sorghum) (Sorghum vulgare). |
| | | Sb06g023120 | |
| D2R9H8 | D2R9H8_9PLAN | Nitrilase/cyanide hydratase and | Pirellula staleyi DSM 6068. |
| | | apolipoprote . . . | |
| B9SWZ9 | B9SWZ9_RICCO | Nitrilase, putative | Ricinus communis (Castor bean). |
| A6T0X3 | A6T0X3_JANMA | Nitrilase | Janthinobacterium sp. (strain Marseille) |
| | | | (Minibacterium massiliensis). |
| Q89PT3 | Q89PT3_BRAJA | Nitrilase | Bradyrhizobium japonicum. |
| C5TGS3 | C5TGS3_ZYMMO | Nitrilase/cyanide hydratase and | Zymomonas mobilis subsp. mobilis ATCC 10988. |
| | | apolipoprote . . . | |
| C3UJS9 | C3UJS9_ARAAL | Putative nitrilase/cyanide | Arabis alpina (Alpine rock-cress). |
| | | hydratase and apo . . . | |
| C8WFJ2 | C8WFJ2_ZYMMN | Nitrilase/cyanide hydratase and | Zymomonas mobilis subsp. mobilis (strain NCIB |
| | | apolipoprote . . . | 11163). |
| Q5NN79 | Q5NN79_ZYMMO | Nitrilase/cyanide hydratase and | Zymomonas mobilis. |
| | | apolipoprote . . . | |
| C6QQS3 | C6QQS3_9BACI | Nitrilase/cyanide hydratase and | Geobacillus sp. Y4.1MC1. |
| | | apolipoprote . . . | |
| Q8LFU8 | Q8LFU8_ARATH | Nitrilase 1 | Arabidopsis thaliana (Mouse-ear cress). |
| Q0PIV8 | Q0PIV8_9BACI | Nitrilase | Geobacillus pallidus. |
| B0T9J3 | B0T9J3_CAUSK | Nitrilase/cyanide hydratase and | Caulobacter sp. (strain K31). |
| | | apolipoprote . . . | |
| D2MB94 | D2MB94_RHOPA | Nitrilase/cyanide hydratase and | Rhodopseudomonas palustris DX-1. |
| | | apolipoprote . . . | |
| B3QKN9 | B3QKN9_RHOPT | Nitrilase/cyanide hydratase and | Rhodopseudomonas palustris (strain TIE-1). |
| | | apolipoprote . . . | |
| Q6N284 | Q6N284_RHOPA | Putative nitrilase | Rhodopseudomonas palustris. |
| Q23384 | Q23384_CAEEL | Protein ZK1058.6, confirmed by | Caenorhabditis elegans. |
| | | transcript ev . . . | |
| C0Z5P2 | C0Z5P2_BREBN | Probable nitrilase | Brevibacillus brevis (strain 47/JCM 6285/NBRC |
| | | | 100599). |
| Q183S8 | Q183S8_CLOD6 | Nitrilase (Carbon-nitrogen | Clostridium difficile (strain 630). |
| | | hydrolase) | |
| A1R1P2 | A1R1P2_ARTAT | Putative nitrilase | Arthrobacter aurescens (strain TC1). |
| C9YQ68 | C9YQ68_CLODR | Nitrilase (Carbon-nitrogen | Clostridium difficile (strain R20291). |
| | | hydrolase) | |
| C9XPE9 | C9XPE9_CLODC | Nitrilase (Carbon-nitrogen | Clostridium difficile (strain CD196). |
| | | hydrolase) | |
| C6C4P7 | C6C4P7_DICDC | Nitrilase/cyanide hydratase and | Dickeya dadantii (strain Ech703). |
| | | apolipoprote . . . | |
| B9E2U6 | B9E2U6_CLOK1 | Putative uncharacterized protein | Clostridium kluyveri (strain NBRC 12016). |
| A5MYU1 | A5MYU1_CLOK5 | Predicted nitrilase | Clostridium kluyveri (strain ATCC 8527/DSM 555/ |
| | | | NCIMB 10680). |
| C8Q5J0 | C8Q5J0_9ENTR | Nitrilase/cyanide hydratase and | Pantoea sp. At-9b. |
| | | apolipoprote . . . | |
| B2A133 | B2A133_NATTJ | Nitrilase/cyanide hydratase and | Natranaerobius thermophilus (strain ATCC BAA- |
| | | apolipoprote . . . | 1301/DSM 18059/OS JW/NM-WN-LF). |
| Q4KCL8 | Q4KCL8_PSEF5 | Nitrilase family protein | Pseudomonas fluorescens (strain Pf-5/ATCC BAA- |
| | | | 477). |
| B1YTF2 | B1YTF2_BURA4 | Nitrilase/cyanide hydratase and | Burkholderia ambifaria (strain MC40-6). |
| | | apolipoprote . . . | |
| B5U8Z5 | B5U8Z5_BRARP | Putative nitrilase | Brassica rapa subsp. pekinensis (Chinese cabbage). |
| Q1I7X1 | Q1I7X1_PSEE4 | Nitrilase | Pseudomonas entomophila (strain L48). |
| A4JBM5 | A4JBM5_BURVG | Nitrilase/cyanide hydratase and | Burkholderia vietnamiensis (strain G4/LMG 22486) |
| | | apolipoprote . . . | (Burkholderia cepacia OS (strain R1808)). |
| Q3KD43 | Q3KD43_PSEPF | Nitrilase | Pseudomonas fluorescens (strain Pf0-1). |
| Q0BI69 | Q0BI69_BURCM | Nitrilase/cyanide hydratase and | Burkholderia ambifaria (strain ATCC BAA-244/ |
| | | apolipoprote . . . | AMMD) (Burkholderia cepacia OS (strain AMMD)). |
| D1SVE1 | D1SVE1_9BURK | Nitrilase/cyanide hydratase and | Acidovorax avenae subsp. avenae ATCC 19860. |
| | | apolipoprote . . . | |
| B1FFB0 | B1FFB0_9BURK | Nitrilase/cyanide hydratase and | Burkholderia ambifaria IOP40-10. |
| | | apolipoprote . . . | |
| A2VSU1 | A2VSU1_9BURK | Putative uncharacterized protein | Burkholderia cenocepacia PC184. |
| Q39JH5 | Q39JH5_BURS3 | Nitrilase/cyanide hydratase and | Burkholderia sp. (strain 383) (Burkholderia cepacia |
| | | apolipoprote . . . | (strain ATCC 17760/OS NCIB 9086/R18194)). |
| B1TG61 | B1TG61_9BURK | Nitrilase/cyanide hydratase and | Burkholderia ambifaria MEX-5. |
| | | apolipoprote . . . | |
| A6V5Q2 | A6V5Q2_PSEA7 | Nitrilase 4 | Pseudomonas aeruginosa (strain PA7). |

### Linamarase

In some embodiments, the invention provides a plant (e.g. cassava) that contains a linamarase. The linamarase can be any linamarase transgene that is functional in the plant. Linamarase (or beta-D-glucosidase) catalyzes the conversion of cyanogens (e.g. linamarin) into acetone cyanohydrin Without being bound by theory, the present inventors believe that linamarase turns over cyanogens such as linamarin to provide cyanide for amino acid synthesis enzymes (e.g. β-CAS and/or NIT4).

Optionally, the linamarase is any linamarase set forth in Table 10.

Optionally, the linamarase exhibits a sequence identity of at least about any of 75%, 80%, 85%, 90%, or 95% to a linamarase listed in Table 10, or an active fragment thereof.

Examplary linamarase transgenes comprise one or more of the following features:

x. a glycosyl hydrolase family 1 domain; and
y. generalized β-glucosidases with broad substrate specificity.

The linamarase can be any linamarase enzyme known in the art. Optionally, the linamarase is a plant, bacterial, or fungal linamarase.

Optionally, the linamarase is a monocot, dicot, cassava, or Arabidopsis linamarase.

Optionally, the linamarase is operably linked to a comestible (e.g. root) specific promoter. Optionally, the linamarase is operably linked to a patatin promoter.

Optionally, the linamarase is targeted to the vacuole or cytoplasm.

Optionally, the linamarase is fused with a targeting sequence (e.g. vacuolar-targeting sequence). Optionally, the linamarase lacks a native transit sequence (e.g. N-terminal cell wall transit sequence). Optionally, the linamarase lacks a cell wall transit sequence and comprises a vacuolar-targeting sequence.

Without being bound by theory, the present inventors believe that cyanogens such as linamarin, are synthesized in the leaves of cassava and transported symplastically to the roots where and stored in vacuoles but that the majority of stored linamarin is sequestered from linamarase, which is localized to the cell wall and laticifers. Surprisingly, overexpressing linamarase and/or expressing targeted linamarin as a gene fusion with a targeting sequence (e.g. vacuolar) localizes linamarase to the microenvironment of cyanogens, and reduces ROS production and PPD caused by cyanide poisoning.

TABLE 10
Linamarase Transgenes

| | | | |
|---|---|---|---|
| Q41172 | Q41172_MANES | Linamarase | Manihot esculenta (Cassava) (Manioc). |
| O24524 | O24524_MANES | Linamarase | Manihot esculenta (Cassava) (Manioc). |
| Q84L69 | Q84L69_HEVBR | P66 protein | Hevea brasiliensis (Para rubber tree). |
| A1E2C0 | A1E2C0_HEVBR | Beta glucosidase | Hevea brasiliensis (Para rubber tree). |
| Q40283 | Q40283_MANES | Beta glucosidase | Manihot esculenta (Cassava) (Manioc). |
| B9MZ87 | B9MZ87_POPTR | Predicted protein | Populus trichocarpa (Western balsam poplar) (Populus |
| | | | balsamifera subsp. OS trichocarpa). |
| B9S3R9 | B9S3R9_RICCO | Beta-glucosidase, putative | Ricinus communis (Castor bean). |
| B2ZUU1 | B2ZUU1_LOTJA | Beta-glucosidase D2 | Lotus japonicus. |
| B9N6U4 | B9N6U4_POPTR | Predicted protein | Populus trichocarpa (Western balsam poplar) (Populus |
| | | | balsamifera subsp. OS trichocarpa). |
| B9N6U2 | B9N6U2_POPTR | Predicted protein | Populus trichocarpa (Western balsam poplar) (Populus |
| | | | balsamifera subsp. OS trichocarpa). |
| A5C932 | A5C932_VITVI | Putative uncharacterized protein | Vitis vinifera (Grape). |
| D1HUK5 | D1HUK5_VITVI | Whole genome shotgun sequence of | Vitis vinifera (Grape). |
| | | line PN4002 . . . | |
| Q01KB2 | Q01KB2_ORYSA | OSIGBa0135C13.7 protein | Oryza sativa (Rice). |
| B8AVF0 | B8AVF0_ORYSI | Putative uncharacterized protein | Oryza sativa subsp. indica (Rice). |
| Q7XKV4 | BGL12_ORYSJ | Beta-glucosidase 12 (Os4bglu12) | Oryza sativa subsp. japonica (Rice). |
| | | (EC 3.2.1.2 . . . | |
| D1HUJ5 | D1HUJ5_VITVI | Whole genome shotgun sequence of | Vitis vinifera (Grape). |
| | | line PN4002 . . . | |
| C5YAD5 | C5YAD5_SORBI | Putative uncharacterized protein | Sorghum bicolor (Sorghum) (Sorghum vulgare). |
| | | Sb06g019840 | |
| B9N6U5 | B9N6U5_POPTR | Predicted protein | Populus trichocarpa (Western balsam poplar) (Populus |
| | | | balsamifera subsp. OS trichocarpa). |
| Q945G7 | Q945G7_PRUSE | Amygdalin hydrolase isoform AH I | Prunus serotina (Black cherry). |
| Q40984 | Q40984_PRUSE | Amygdalin hydrolase isoform AH I | Prunus serotina (Black cherry). |
| D1HUL0 | D1HUL0_VITVI | Whole genome shotgun sequence of | Vitis vinifera (Grape). |
| | | line PN4002 . . . | |
| B9NC20 | B9NC20_POPTR | Predicted protein | Populus trichocarpa (Western balsam poplar) (Populus |
| | | | balsamifera subsp. OS trichocarpa). |
| B9N6U3 | B9N6U3_POPTR | Predicted protein | Populus trichocarpa (Western balsam poplar) (Populus |
| | | | balsamifera subsp. OS trichocarpa). |
| B2ZUU0 | B2ZUU0_LOTJA | Beta-glucosidase D4 | Lotus japonicus. |
| Q01IX2 | Q01IX2_ORYSA | OSIGBa0106G07.1 protein | Oryza sativa (Rice). |
| Q7XKV2 | BGL13_ORYSJ | Beta-glucosidase 13 (Os4bglu13) | Oryza sativa subsp. japonica (Rice). |
| | | (EC 3.2.1.2 . . . | |
| B9GTS5 | B9GTS5_POPTR | Predicted protein | Populus trichocarpa (Western balsam poplar) (Populus |
| | | | balsamifera subsp. OS trichocarpa). |
| B2ZUU2 | B2ZUU2_LOTJA | Beta-glucosidase D7 | Lotus japonicus. |
| B9GEP0 | B9GEP0_POPTR | Predicted protein | Populus trichocarpa (Western balsam poplar) (Populus |
| | | | balsamifera subsp. OS trichocarpa). |
| A8C6P5 | A8C6P5_TRIRP | Beta-glucosidase-like protein | Trifolium repens (Creeping white clover). |
| D1H5J2 | D1H5J2_VITVI | Whole genome shotgun sequence of | Vitis vinifera (Grape). |
| | | line PN4002 . . . | |
| B9REG9 | B9REG9_RICCO | Beta-glucosidase, putative | Ricinus communis (Castor bean). |
| B9GEP1 | B9GEP1_POPTR | Predicted protein | Populus trichocarpa (Western balsam poplar) (Populus |
| | | | balsamifera subsp. OS trichocarpa). |
| B8B155 | B8B155_ORYSI | Putative uncharacterized protein | Oryza sativa subsp. indica (Rice). |
| Q01KB3 | Q01KB3_ORYSA | OSIGBa0135C13.6 protein | Oryza sativa (Rice). |
| A9Z0X2 | A9Z0X2_LEUGL | Glycosylhydrolase 1 | Leucaena glauca (White popinac) (Leucaena |
| | | | leucocephala). |
| Q7XKV5 | BGL11_ORYSJ | Beta-glucosidase 11 (Os4bglu11) | Oryza sativa subsp. japonica (Rice). |
| | | (EC 3.2.1.2 . . . | |
| Q5Z9Z0 | BGL24_ORYSJ | Beta-glucosidase 24 (Os6bglu24) | Oryza sativa subsp. japonica (Rice). |
| | | (EC 3.2.1.2 . . . | |
| Q945G5 | Q945G5_PRUSE | Prunasin hydrolase isoform PH I | Prunus serotina (Black cherry). |
| Q43073 | Q43073_PRUSE | Prunasin hydrolase isoform PH I | Prunus serotina (Black cherry). |
| A8TVQ9 | A8TVQ9_MEDTR | Beta-glucosidase G3 | Medicago truncatula (Barrel medic). |
| B9GMA6 | B9GMA6_POPTR | Predicted protein | Populus trichocarpa (Western balsam poplar) (Populus |
| | | | balsamifera subsp. OS trichocarpa). |
| B9H2X5 | B9H2X5_POPTR | Predicted protein | Populus trichocarpa (Western balsam poplar) (Populus |
| | | | balsamifera subsp. OS trichocarpa). |
| B1B611 | B1B611_ROSHC | Beta-glucosidase | Rosa hybrid cultivar. |
| Q945I4 | Q945I4_PRUSE | Prunasin hydrolase isoform PH C | Prunus serotina (Black cherry). |
| Q8W594 | Q8W594_PRUSE | Prunasin hydrolase isoform PH C | Prunus serotina (Black cherry). |
| Q8W1W7 | Q8W1W7_PRUSE | Prunasin hydrolase isoform PH B | Prunus serotina (Black cherry). |
| Q43014 | Q43014_PRUAV | Beta-glucosidase | Prunus avium (Cherry). |
| Q945N9 | Q945N9_PRUSE | Prunasin hydrolase isoform PH B | Prunus serotina (Black cherry). |
| A8C6N7 | A8C6N7_9FABA | Cyanogenic beta-glucosidase | Trifolium nigrescens subsp. petrisavii. |
| A8C6N9 | A8C6N9_9FABA | Cyanogenic beta-glucosidase | Trifolium nigrescens subsp. petrisavii. |
| Q84YK7 | BGL27_ORYSJ | Beta-glucosidase 27 (Os8bglu27) | Oryza sativa subsp. japonica (Rice). |
| | | (EC 3.2.1.2 . . . | |
| B8BCB0 | B8BCB0_ORYSI | Putative uncharacterized protein | Oryza sativa subsp. indica (Rice). |
| A8TVQ5 | A8TVQ5_MEDTR | Beta-glucosidase G2 | Medicago truncatula (Barrel medic). |
| A8C6M3 | A8C6M3_TRIRP | Cyanogenic beta-glucosidase | Trifolium repens (Creeping white clover). |
| A8C6J3 | A8C6J3_TRIRP | Cyanogenic beta-glucosidase | Trifolium repens (Creeping white clover). |
| A8C6G0 | A8C6G0_TRIRP | Cyanogenic beta-glucosidase | Trifolium repens (Creeping white clover). |
| B9RI71 | B9RI71_RICCO | Beta-glucosidase, putative | Ricinus communis (Castor bean). |
| B7FLM5 | B7FLM5_MEDTR | Putative uncharacterized protein | Medicago truncatula (Barrel medic). |
| A8C6L1 | A8C6L1_TRIRP | Cyanogenic beta-glucosidase | Trifolium repens (Creeping white clover). |
| A8C6P2 | A8C6P2_9FABA | Cyanogenic beta-glucosidase | Trifolium isthmocarpum. |
| A8C6K7 | A8C6K7_TRIRP | Cyanogenic beta-glucosidase | Trifolium repens (Creeping white clover). |
| A8C6N4 | A8C6N4_9FABA | Cyanogenic beta-glucosidase | Trifolium nigrescens subsp. petrisavii. |
| A8C6H2 | A8C6H2_TRIRP | Cyanogenic beta-glucosidase | Trifolium repens (Creeping white clover). |
| Q9M5X4 | Q9M5X4_PRUSE | Putative prunasin hydrolase isoform | Prunus serotina (Black cherry). |
| | | PH-L1 | |
| Q945G6 | Q945G6_PRUSE | Putative prunasin hydrolase | Prunus serotina (Black cherry). |
| Q945I3 | Q945I3_PRUSE | Prunasin hydrolase isoform PH A | Prunus serotina (Black cherry). |
| Q7X9A9 | Q7X9A9_CAMSI | Beta-primeverosidase | Camellia sinensis (Tea). |
| Q9M5X5 | Q9M5X5_PRUSE | Prunasin hydrolase isoform PHA | Prunus serotina (Black cherry). |
| B8BCW5 | B8BCW5_ORYSI | Putative uncharacterized protein | Oryza sativa subsp. indica (Rice). |
| Q01KA9 | Q01KA9_ORYSA | OSIGBa0135C13.2 protein | Oryza sativa (Rice). |
| A2SY66 | A2SY66_VICAN | Vicianin hydrolase | Vicia angustifolia (Common vetch). |
| B9GEM1 | B9GEM1_POPTR | Predicted protein | Populus trichocarpa (Western balsam poplar) (Populus |
| | | | balsamifera subsp. OS trichocarpa). |
| Q7F9K4 | BGL10_ORYSJ | Beta-glucosidase 10 (Os4bglu10) | Oryza sativa subsp. japonica (Rice). |
| | | (EC 3.2.1.2 . . . | |
| D1HUV6 | D1HUV6_VITVI | Whole genome shotgun sequence of | Vitis vinifera (Grape). |
| | | line PN4002 . . . | |
| Q01KB4 | Q01KB4_ORYSA | OSIGBa0135C13.5 protein | Oryza sativa (Rice). |
| B8AVE8 | B8AVE8_ORYSI | Putative uncharacterized protein | Oryza sativa subsp. indica (Rice). |
| Q0J0N4 | BGL30_ORYSJ | Beta-glucosidase 30 (Os9bglu30) | Oryza sativa subsp. japonica (Rice). |
| | | (EC 3.2.1.2 . . . | |
| A2Z2L2 | A2Z2L2_ORYSI | Putative uncharacterized protein | Oryza sativa subsp. indica (Rice). |
| C5YAE1 | C5YAE1_SORBI | Putative uncharacterized protein | Sorghum bicolor (Sorghum) (Sorghum vulgare). |
| | | Sb06g019880 | |
| Q14QP8 | Q14QP8_CAMSI | Beta-glucosidase-like protein | Camellia sinensis (Tea). |
| D1HUJ3 | D1HUJ3_VITVI | Whole genome shotgun sequence of | Vitis vinifera (Grape). |
| | | line PN4002 . . . | |
| Q9SPK3 | Q9SPK3_9FABA | Dalcochinin 8′-O-beta-glucoside beta- | Dalbergia cochinchinensis. |
| | | glucosi . . . | |
| B9S3R8 | B9S3R8_RICCO | Beta-glucosidase, putative | Ricinus communis (Castor bean). |
| Q5UB04 | Q5UB04_9FABA | Beta-glycosidase | Dalbergia nigrescens. |
| Q08IT7 | Q08IT7_SOYBN | Isoflavone conjugate-specific beta- | Glycine max (Soybean). |
| | | glucosida . . . | |
| D1HUK1 | D1HUK1_VITVI | Whole genome shotgun sequence of | Vitis vinifera (Grape). |
| | | line PN4002 . . . | |
| B9N6G0 | B9N6G0_POPTR | Predicted protein | Populus trichocarpa (Western balsam poplar) (Populus |
| | | | balsamifera subsp. OS trichocarpa). |
| D1HUJ2 | D1HUJ2_VITVI | Whole genome shotgun sequence of | Vitis vinifera (Grape). |
| | | line PN4002 . . . | |
| B9N6G1 | B9N6G1_POPTR | Predicted protein | Populus trichocarpa (Western balsam poplar) (Populus |
| | | | balsamifera subsp. OS trichocarpa). |
| B8AVF1 | B8AVF1_ORYSI | Putative uncharacterized protein | Oryza sativa subsp. indica (Rice). |
| D1HUK2 | D1HUK2_VITVI | Whole genome shotgun sequence of | Vitis vinifera (Grape). |
| | | line PN4002 . . . | |
| C5YAD8 | C5YAD8_SORBI | Putative uncharacterized protein | Sorghum bicolor (Sorghum) (Sorghum vulgare). |
| | | Sb06g019860 | |
| Q9M1C9 | BGL30_ARATH | Beta-glucosidase 30 (AtBGLU30) | Arabidopsis thaliana (Mouse-ear cress). |
| | | (EC 3.2.1.21 . . . | |
| Q9M1D0 | BGL16_ARATH | Beta-glucosidase 16 (AtBGLU16) | Arabidopsis thaliana (Mouse-ear cress). |
| | | (EC 3.2.1.21 . . . | |
| Q700B1 | Q700B1_CICAR | Non-cyanogenic beta-glucosidase | Cicer arietinum (Chickpea) (Garbanzo). |
| A3C053 | BGL29_ORYSJ | Beta-glucosidase 29 (Os9bglu29) | Oryza sativa subsp. japonica (Rice). |
| | | (EC 3.2.1.2 . . . | |
| A3RF67 | A3RF67_9FABA | Beta-glycosidase | Dalbergia nigrescens. |
| B9RI70 | B9RI70_RICCO | Beta-glucosidase, putative | Ricinus communis (Castor bean). |
| C5YAD7 | C5YAD7_SORBI | Putative uncharacterized protein | Sorghum bicolor (Sorghum) (Sorghum vulgare). |
| | | Sb06g019850 | |

### Hydroxynitrile Lyase (HNL)

In some embodiments, the invention provides a plant (e.g. cassava) that contains a nitrilase 4 (HNL). The HNL can be any HNL transgene that is functional in the plant. HNL catalyzes the synthesis of cyanohydrins (a hydroxynitriles) from carbonyl compounds in the presence of a cyanide donor, for example, catalyzing the conversion of acyanohydrin to HCN plus the corresponding aldehyde or ketone. Without being bound by theory, the present inventors believe that HNL provides cyanide detoxification converting cyanide to a form that's removed during comestible (e.g. cassava root) processing.

Optionally, the HNL is an HNL-A or HNL-B.

Optionally, the HNL is any HNL set forth in Table 11 or Table 12.

Optionally, the HNL exhibits a sequence identity of at least about any of 75%, 80%, 85%, 90%, or 95% to an HNL listed in Table 11 or Table 12, or an active fragment thereof.

Examplary HNL transgenes comprise one or more of the following features:

z. An α/β hydrolase domain;
aa. a catalytic triad made of Ser80, His235 and Asp207, or conserved variant thereof; and
bb. an oxyanion hole.

The HNL can be any HNL enzyme known in the art. Optionally, the HNL is a plant, bacterial, or fungal HNL.

Optionally, the HNL is a monocot, dicot, cassava, or Arabidopsis HNL.

Optionally, the HNL is operably linked to a comestible (e.g. root) specific promoter. Optionally, the HNL is operably linked to a patatin promoter.

TABLE 11
HNL-A Transgenes

| | | | |
|---|---|---|---|
| Q5S2C5 | Q5S2C5_MANES | Alpha-hydroxynitrile lyase | Manihot esculenta (Cassava) (Manioc). |
| P52705 | HNL_MANES | Hydroxynitrilase (EC 4.1.2.37) ((S)-acetone . . . | Manihot esculenta (Cassava) (Manioc). |
| P52704 | HNL_HEVBR | Hydroxynitrilase (EC 4.1.2.37) ((S)-acetone . . . | Hevea brasiliensis (Para rubber tree). |
| O49897 | O49897_MANES | Alpha-hydroxynitrile lyase | Manihot esculenta (Cassava) (Manioc). |
| D1MX83 | D1MX83_9ROSI | (S) | Baliospermum montanum. |
| D1MX82 | D1MX82_9ROSI | (S) | Baliospermum montanum. |
| D1MX81 | D1MX81_9ROSI | (S) | Baliospermum montanum. |
| D1MX80 | D1MX80_9ROSI | (S) | Baliospermum montanum. |
| D1MX73 | D1MX73_9ROSI | (S) | Baliospermum montanum. |
| D1MX77 | D1MX77_9ROSI | (S) | Baliospermum montanum. |
| D1MX74 | D1MX74_9ROSI | (S) | Baliospermum montanum. |
| D1MX76 | D1MX76_9ROSI | (S) | Baliospermum montanum. |
| D1MX78 | D1MX78_9ROSI | (S) | Baliospermum montanum. |
| D1MX79 | D1MX79_9ROSI | (S) | Baliospermum montanum. |
| Q9LFT6 | Q9LFT6_ARATH | Alpha-hydroxynitrile lyase-like proteinAT5g1 . . . | Arabidopsis thaliana (Mouse-ear cress). |
| Q94AI5 | Q94AI5_ARATH | Putative alpha-hydroxynitrile lyase | Arabidopsis thaliana (Mouse-ear cress). |
| Q93Z57 | Q93Z57_ARATH | AT5g10300/F18D22_70 | Arabidopsis thaliana (Mouse-ear cress). |
| B9S8M5 | B9S8M5_RICCO | Polyneuridine-aldehyde esterase, putative | Ricinus communis (Castor bean). |
| O49893 | O49893_MANES | Alpha-hydroxynitrile lyase | Manihot esculenta (Cassava) (Manioc). |
| B9HE92 | B9HE92_POPTR | Predicted protein | Populus trichocarpa (Western balsam poplar) |
| | | | (Populus balsamifera subsp. OS trichocarpa). |
| B9HEE0 | B9HEE0_POPTR | Predicted protein | Populus trichocarpa (Western balsam poplar) |
| | | | (Populus balsamifera subsp. OS trichocarpa). |
| C6TB10 | C6TB10_SOYBN | Putative uncharacterized protein | Glycine max (Soybean). |
| Q6RYA0 | Q6RYA0_TOBAC | Salicylic acid-binding protein 2 | Nicotiana tabacum (Common tobacco). |
| Q94G63 | Q94G63_CITSI | Ethylene-induced esterase | Citrus sinensis (Sweet orange). |
| Q9SE93 | PNAE_RAUSE | Polyneuridine-aldehyde esterase (EC | Rauvolfia serpentina (Serpentwood) |
| | | 3.1.1.78 . . . | (Devilpepper). |
| D1HBL9 | D1HBL9_VITVI | Whole genome shotgun sequence of line | Vitis vinifera (Grape). |
| | | PN4002 . . . | |
| B9H6C4 | B9H6C4_POPTR | Predicted protein | Populus trichocarpa (Western balsam poplar) |
| | | | (Populus balsamifera subsp. OS trichocarpa). |
| B9H6C2 | B9H6C2_POPTR | Predicted protein | Populus trichocarpa (Western balsam poplar) |
| | | | (Populus balsamifera subsp. OS trichocarpa). |
| A5A7N4 | A5A7N4_GENTR | Alpha/beta hydrolase fold superfamily | Gentiana triflora var. japonica. |
| A5A7N5 | A5A7N5_GENTR | Alpha/beta hydrolase fold superfamily | Gentiana triflora var. japonica. |
| B9P9I8 | B9P9I8_POPTR | Predicted protein | Populus trichocarpa (Western balsam poplar) |
| | | | (Populus balsamifera subsp. OS trichocarpa). |
| C6TJI9 | C6TJI9_SOYBN | Putative uncharacterized protein | Glycine max (Soybean). |
| C6T4P4 | C6T4P4_SOYBN | Putative uncharacterized protein | Glycine max (Soybean). |
| B7FIX6 | B7FIX6_MEDTR | Putative uncharacterized protein | Medicago truncatula (Barrel medic). |
| C6TAV4 | C6TAV4_SOYBN | Putative uncharacterized protein | Glycine max (Soybean). |
| A2WVV5 | A2WVV5_ORYSI | Putative uncharacterized protein | Oryza sativa subsp. indica (Rice). |
| Q8S125 | Q8S125_ORYSJ | Os01g0787600 proteincDNA clone: 006-205- | Oryza sativa subsp. japonica (Rice). |
| | | H08, . . . | |
| O80475 | O80475_ARATH | Putative acetone-cyanohydrin lyase | Arabidopsis thaliana (Mouse-ear cress). |
| Q5XLS1 | Q5XLS1_CATRO | Protein S | Catharanthus roseus (Madagascar periwinkle) |
| | | | (Vinca rosea). |
| O80477 | O80477_ARATH | Putative acetone-cyanohydrin lyaseAt2g23610 | Arabidopsis thaliana (Mouse-ear cress). |
| Q8LCI0 | Q8LCI0_ARATH | Putative acetone-cyanohydrin lyase | Arabidopsis thaliana (Mouse-ear cress). |
| O80476 | O80476_ARATH | Putative acetone-cyanohydrin | Arabidopsis thaliana (Mouse-ear cress). |
| | | lyaseAt2g23600/ . . . | |
| O80471 | O80471_ARATH | Putative acetone-cyanohydrin lyase | Arabidopsis thaliana (Mouse-ear cress). |
| Q84W83 | Q84W83_ARATH | Putative acetone-cyanohydrin lyase | Arabidopsis thaliana (Mouse-ear cress). |
| B9S8M7 | B9S8M7_RICCO | Polyneuridine-aldehyde esterase, putative | Ricinus communis (Castor bean). |
| Q8S8S9 | Q8S8S9_ARATH | Putative acetone-cyanohydrin lyaseAt2g23620 | Arabidopsis thaliana (Mouse-ear cress). |
| C6TM24 | C6TM24_SOYBN | Putative uncharacterized protein | Glycine max (Soybean). |
| Q84WR3 | Q84WR3_ARATH | Putative acetone-cyanohydrin lyase | Arabidopsis thaliana (Mouse-ear cress). |
| C5XMB9 | C5XMB9_SORBI | Putative uncharacterized protein Sb03g036770 | Sorghum bicolor (Sorghum) (Sorghum vulgare). |
| B9HYX2 | B9HYX2_POPTR | Predicted protein | Populus trichocarpa (Western balsam poplar) |
| | | | (Populus balsamifera subsp. OS trichocarpa). |
| B5M1Z2 | B5M1Z2_RHEAU | Ethylene esterase-like protein | Rheum australe (Himalayan rhubarb) (Rheum |
| | | | emodi). |
| B9S8N3 | B9S8N3_RICCO | Polyneuridine-aldehyde esterase, putative | Ricinus communis (Castor bean). |
| B9NGA7 | B9NGA7_POPTR | Predicted protein | Populus trichocarpa (Western balsam poplar) |
| | | | (Populus balsamifera subsp. OS trichocarpa). |
| Q9SCT0 | Q9SCT0_ARATH | Putative uncharacterized protein T20E23_40 | Arabidopsis thaliana (Mouse-ear cress). |
| Q8S9K8 | Q8S9K8_ARATH | AT3g50440/T20E23_40At3g50440/T20E23_40 | Arabidopsis thaliana (Mouse-ear cress). |
| O23171 | O23171_ARATH | Hydroxynitrile lyase like proteinPutative un . . . | Arabidopsis thaliana (Mouse-ear cress). |
| B9HEE6 | B9HEE6_POPTR | Predicted protein | Populus trichocarpa (Western balsam poplar) |
| | | | (Populus balsamifera subsp. OS trichocarpa). |
| B9HEE1 | B9HEE1_POPTR | Predicted protein | Populus trichocarpa (Western balsam poplar) |
| | | | (Populus balsamifera subsp. OS trichocarpa). |
| B9H6C5 | B9H6C5_POPTR | Predicted protein | Populus trichocarpa (Western balsam poplar) |
| | | | (Populus balsamifera subsp. OS trichocarpa). |
| O80474 | O80474_ARATH | Putative acetone-cyanohydrin lyaseAt2g23580 | Arabidopsis thaliana (Mouse-ear cress). |
| B9HEE5 | B9HEE5_POPTR | Predicted protein | Populus trichocarpa (Western balsam poplar) |
| | | | (Populus balsamifera subsp. OS trichocarpa). |
| B9HEE2 | B9HEE2_POPTR | Predicted protein | Populus trichocarpa (Western balsam poplar) |
| | | | (Populus balsamifera subsp. OS trichocarpa). |
| D1IGG9 | D1IGG9_VITVI | Whole genome shotgun sequence of line | Vitis vinifera (Grape). |
| | | PN4002 . . . | |
| A5BJI4 | A5BJI4_VITVI | Putative uncharacterized protein | Vitis vinifera (Grape). |
| D1IGH5 | D1IGH5_VITVI | Whole genome shotgun sequence of line | Vitis vinifera (Grape). |
| | | PN4002 . . . | |
| D1IGG6 | D1IGG6_VITVI | Whole genome shotgun sequence of line | Vitis vinifera (Grape). |
| | | PN4002 . . . | |
| D1HBM0 | D1HBM0_VITVI | Whole genome shotgun sequence of line | Vitis vinifera (Grape). |
| | | PN4002 . . . | |
| D1IGI2 | D1IGI2_VITVI | Whole genome shotgun sequence of line | Vitis vinifera (Grape). |
| | | PN4002 . . . | |
| B9HRV8 | B9HRV8_POPTR | Predicted protein | Populus trichocarpa (Western balsam poplar) |
| | | | (Populus balsamifera subsp. OS trichocarpa). |
| Q6ED34 | Q6ED34_SOLLC | Methylesterase | Solanum lycopersicum (Tomato) (Lycopersicon |
| | | | esculentum). |
| Q56SE1 | Q56SE1_SOLTU | Methyl jasmonate esterase | Solanum tuberosum (Potato). |
| D1HBM1 | D1HBM1_VITVI | Whole genome shotgun sequence of line | Vitis vinifera (Grape). |
| | | PN4002 . . . | |
| O80472 | O80472_ARATH | Putative acetone-cyanohydrin lyaseAt2g23560 | Arabidopsis thaliana (Mouse-ear cress). |
| B9HRV7 | B9HRV7_POPTR | Predicted protein | Populus trichocarpa (Western balsam poplar) |
| | | | (Populus balsamifera subsp. OS trichocarpa). |
| D2CKY0 | D2CKY0_9SOLA | Methyl jasmonate esterase | Nicotiana attenuata. |
| Q4ABP0 | Q4ABP0_BRARP | 80A08_27 | Brassica rapa subsp. pekinensis (Chinese |
| | | | cabbage). |
| Q0JG99 | PIR7B_ORYSJ | Esterase PIR7B (EC 3.1.—.—) | Oryza sativa subsp. japonica (Rice). |
| A2WYS7 | PIR7B_ORYSI | Esterase PIR7B (EC 3.1.—.—) | Oryza sativa subsp. indica (Rice). |
| B9HEE3 | B9HEE3_POPTR | Predicted protein | Populus trichocarpa (Western balsam poplar) |
| | | | (Populus balsamifera subsp. OS trichocarpa). |
| A9P066 | A9P066_PICSI | Putative uncharacterized protein | Picea sitchensis (Sitka spruce). |
| D1HBL8 | D1HBL8_VITVI | Whole genome shotgun sequence of line | Vitis vinifera (Grape). |
| | | PN4002 . . . | |
| A2ZYJ7 | A2ZYJ7_ORYSJ | Putative uncharacterized protein | Oryza sativa subsp. japonica (Rice). |
| Q0JLY5 | Q0JLY5_ORYSJ | Os01g0557200 protein | Oryza sativa subsp. japonica (Rice). |
| B8LPI5 | B8LPI5_PICSI | Putative uncharacterized protein | Picea sitchensis (Sitka spruce). |
| C5XH46 | C5XH46_SORBI | Putative uncharacterized protein Sb03g045110 | Sorghum bicolor (Sorghum) (Sorghum vulgare). |
| Q3EBV1 | Q3EBV1_ARATH | Putative uncharacterized protein At2g23550.2 | Arabidopsis thaliana (Mouse-ear cress). |
| B9S8N0 | B9S8N0_RICCO | Polyneuridine-aldehyde esterase, putative | Ricinus communis (Castor bean). |
| C5XMZ5 | C5XMZ5_SORBI | Putative uncharacterized protein Sb03g024830 | Sorghum bicolor (Sorghum) (Sorghum vulgare). |
| C5XH43 | C5XH43_SORBI | Putative uncharacterized protein Sb03g045090 | Sorghum bicolor (Sorghum) (Sorghum vulgare). |
| C5XH42 | C5XH42_SORBI | Putative uncharacterized protein Sb03g045080 | Sorghum bicolor (Sorghum) (Sorghum vulgare). |
| Q0JG98 | PIR7A_ORYSJ | Probable esterase PIR7A (EC 3.1.—.—) | Oryza sativa subsp. japonica (Rice). |
| A2WYS8 | PIR7A_ORYSI | Probable esterase PIR7A (EC 3.1.—.—) | Oryza sativa subsp. indica (Rice). |
| B8A8T5 | B8A8T5_ORYSI | Putative uncharacterized protein | Oryza sativa subsp. indica (Rice). |
| Q8S0U8 | Q8S0U8_ORYSJ | Putative uncharacterized proteinPutative sal . . . | Oryza sativa subsp. japonica (Rice). |
| A2WRC2 | A2WRC2_ORYSI | Putative uncharacterized protein | Oryza sativa subsp. indica (Rice). |
| Q5ZCR3 | Q5ZCR3_ORYSJ | Os01g0355800 proteinPutative salicylic acid- . . . | Oryza sativa subsp. japonica (Rice). |
| A5BHZ4 | A5BHZ4_VITVI | Putative uncharacterized protein | Vitis vinifera (Grape). |
| C5XH41 | C5XH41_SORBI | Putative uncharacterized protein Sb03g045070 | Sorghum bicolor (Sorghum) (Sorghum vulgare). |
| D1HBM2 | D1HBM2_VITVI | Whole genome shotgun sequence of line | Vitis vinifera (Grape). |
| | | PN4002 . . . | |
| Q8S0V0 | Q8S0V0_ORYSJ | Os01g0557100 proteinPutative | Oryza sativa subsp. japonica (Rice). |
| | | uncharacterized . . . | |

TABLE 12
HNL-B Transgenes

| | | | |
|---|---|---|---|
| P52705 | HNL_MANES | Hydroxynitrilase (EC 4.1.2.37) ((S)-acetone . . . | Manihot esculenta (Cassava) (Manioc). |
| Q5S2C5 | Q5S2C5_MANES | Alpha-hydroxynitrile lyase | Manihot esculenta (Cassava) (Manioc). |
| P52704 | HNL_HEVBR | Hydroxynitrilase (EC 4.1.2.37) ((S)-acetone . . . | Hevea brasiliensis (Para rubber tree). |
| O49897 | O49897_MANES | Alpha-hydroxynitrile lyase | Manihot esculenta (Cassava) (Manioc). |
| D1MX83 | D1MX83_9ROSI | (S) | Baliospermum montanum. |
| D1MX82 | D1MX82_9ROSI | (S) | Baliospermum montanum. |
| D1MX81 | D1MX81_9ROSI | (S) | Baliospermum montanum. |
| D1MX80 | D1MX80_9ROSI | (S) | Baliospermum montanum. |
| D1MX73 | D1MX73_9ROSI | (S) | Baliospermum montanum. |
| D1MX77 | D1MX77_9ROSI | (S) | Baliospermum montanum. |
| D1MX74 | D1MX74_9ROSI | (S) | Baliospermum montanum. |
| D1MX76 | D1MX76_9ROSI | (S) | Baliospermum montanum. |
| D1MX78 | D1MX78_9ROSI | (S) | Baliospermum montanum. |
| D1MX79 | D1MX79_9ROSI | (S) | Baliospermum montanum. |
| Q9LFT6 | Q9LFT6_ARATH | Alpha-hydroxynitrile lyase-like proteinAT5g1 . . . | Arabidopsis thaliana (Mouse-ear cress). |
| Q94AI5 | Q94AI5_ARATH | Putative alpha-hydroxynitrile lyase | Arabidopsis thaliana (Mouse-ear cress). |
| Q93Z57 | Q93Z57_ARATH | AT5g10300/F18D22_70 | Arabidopsis thaliana (Mouse-ear cress). |
| O49893 | O49893_MANES | Alpha-hydroxynitrile lyase | Manihot esculenta (Cassava) (Manioc). |
| B9S8M5 | B9S8M5_RICCO | Polyneuridine-aldehyde esterase, putative | Ricinus communis (Castor bean). |
| C6TB10 | C6TB10_SOYBN | Putative uncharacterized protein | Glycine max (Soybean). |
| B9HE92 | B9HE92_POPTR | Predicted protein | Populus trichocarpa (Western balsam poplar) |
| | | | (Populus balsamifera subsp. OS trichocarpa). |
| B9HEE0 | B9HEE0_POPTR | Predicted protein | Populus trichocarpa (Western balsam poplar) |
| | | | (Populus balsamifera subsp. OS trichocarpa). |
| Q94G63 | Q94G63_CITSI | Ethylene-induced esterase | Citrus sinensis (Sweet orange). |
| Q9SE93 | PNAE_RAUSE | Polyneuridine-aldehyde esterase (EC | Rauvolfia serpentina (Serpentwood) |
| | | 3.1.1.78 . . . | (Devilpepper). |
| Q6RYA0 | Q6RYA0_TOBAC | Salicylic acid-binding protein 2 | Nicotiana tabacum (Common tobacco). |
| B9H6C4 | B9H6C4_POPTR | Predicted protein | Populus trichocarpa (Western balsam poplar) |
| | | | (Populus balsamifera subsp. OS trichocarpa). |
| D1HBL9 | D1HBL9_VITVI | Whole genome shotgun sequence of line | Vitis vinifera (Grape). |
| | | PN4002 . . . | |
| B9H6C2 | B9H6C2_POPTR | Predicted protein | Populus trichocarpa (Western balsam poplar) |
| | | | (Populus balsamifera subsp. OS trichocarpa). |
| A5A7N4 | A5A7N4_GENTR | Alpha/beta hydrolase fold superfamily | Gentiana triflora var. japonica. |
| B9P9I8 | B9P9I8_POPTR | Predicted protein | Populus trichocarpa (Western balsam poplar) |
| | | | (Populus balsamifera subsp. OS trichocarpa). |
| A5A7N5 | A5A7N5_GENTR | Alpha/beta hydrolase fold superfamily | Gentiana triflora var. japonica. |
| A2WVV5 | A2WVV5_ORYSI | Putative uncharacterized protein | Oryza sativa subsp. indica (Rice). |
| Q8S125 | Q8S125_ORYSJ | Os01g0787600 proteincDNA clone: 006-205- | Oryza sativa subsp. japonica (Rice). |
| | | H08, . . . | |
| C6TJI9 | C6TJI9_SOYBN | Putative uncharacterized protein | Glycine max (Soybean). |
| C6T4P4 | C6T4P4_SOYBN | Putative uncharacterized protein | Glycine max (Soybean). |
| B7FIX6 | B7FIX6_MEDTR | Putative uncharacterized protein | Medicago truncatula (Barrel medic). |
| C6TAV4 | C6TAV4_SOYBN | Putative uncharacterized protein | Glycine max (Soybean). |
| O80475 | O80475_ARATH | Putative acetone-cyanohydrin lyase | Arabidopsis thaliana (Mouse-ear cress). |
| Q5XLS1 | Q5XLS1_CATRO | Protein S | Catharanthus roseus (Madagascar periwinkle) |
| | | | (Vinca rosea). |
| O80477 | O80477_ARATH | Putative acetone-cyanohydrin lyaseAt2g23610 | Arabidopsis thaliana (Mouse-ear cress). |
| Q8LCI0 | Q8LCI0_ARATH | Putative acetone-cyanohydrin lyase | Arabidopsis thaliana (Mouse-ear cress). |
| O80476 | O80476_ARATH | Putative acetone-cyanohydrin | Arabidopsis thaliana (Mouse-ear cress). |
| | | lyaseAt2g23600/ . . . | |
| O80471 | O80471_ARATH | Putative acetone-cyanohydrin lyase | Arabidopsis thaliana (Mouse-ear cress). |
| B9S8M7 | B9S8M7_RICCO | Polyneuridine-aldehyde esterase, putative | Ricinus communis (Castor bean). |
| Q84W83 | Q84W83_ARATH | Putative acetone-cyanohydrin lyase | Arabidopsis thaliana (Mouse-ear cress). |
| C6TM24 | C6TM24_SOYBN | Putative uncharacterized protein | Glycine max (Soybean). |
| Q8S8S9 | Q8S8S9_ARATH | Putative acetone-cyanohydrin lyaseAt2g23620 | Arabidopsis thaliana (Mouse-ear cress). |
| Q84WR3 | Q84WR3_ARATH | Putative acetone-cyanohydrin lyase | Arabidopsis thaliana (Mouse-ear cress). |
| C5XMB9 | C5XMB9_SORBI | Putative uncharacterized protein Sb03g036770 | Sorghum bicolor (Sorghum) (Sorghum vulgare). |
| B9HYX2 | B9HYX2_POPTR | Predicted protein | Populus trichocarpa (Western balsam poplar) |
| | | | (Populus balsamifera subsp. OS trichocarpa). |
| B9S8N3 | B9S8N3_RICCO | Polyneuridine-aldehyde esterase, putative | Ricinus communis (Castor bean). |
| B9NGA7 | B9NGA7_POPTR | Predicted protein | Populus trichocarpa (Western balsam poplar) |
| | | | (Populus balsamifera subsp. OS trichocarpa). |
| B5M1Z2 | B5M1Z2_RHEAU | Ethylene esterase-like protein | Rheum australe (Himalayan rhubarb) (Rheum |
| | | | emodi). |
| B9HEE6 | B9HEE6_POPTR | Predicted protein | Populus trichocarpa (Western balsam poplar) |
| | | | (Populus balsamifera subsp. OS trichocarpa). |
| B9HEE1 | B9HEE1_POPTR | Predicted protein | Populus trichocarpa (Western balsam poplar) |
| | | | (Populus balsamifera subsp. OS trichocarpa). |
| O23171 | O23171_ARATH | Hydroxynitrile lyase like proteinPutative un . . . | Arabidopsis thaliana (Mouse-ear cress). |
| Q9SCT0 | Q9SCT0_ARATH | Putative uncharacterized protein T20E23_40 | Arabidopsis thaliana (Mouse-ear cress). |
| Q8S9K8 | Q8S9K8_ARATH | AT3g50440/T20E23_40At3g50440/T20E23_40 | Arabidopsis thaliana (Mouse-ear cress). |
| B9HEE5 | B9HEE5_POPTR | Predicted protein | Populus trichocarpa (Western balsam poplar) |
| | | | (Populus balsamifera subsp. OS trichocarpa). |
| B9H6C5 | B9H6C5_POPTR | Predicted protein | Populus trichocarpa (Western balsam poplar) |
| | | | (Populus balsamifera subsp. OS trichocarpa). |
| O80474 | O80474_ARATH | Putative acetone-cyanohydrin lyaseAt2g23580 | Arabidopsis thaliana (Mouse-ear cress). |
| B9HEE2 | B9HEE2_POPTR | Predicted protein | Populus trichocarpa (Western balsam poplar) |
| | | | (Populus balsamifera subsp. OS trichocarpa). |
| D1IGG9 | D1IGG9_VITVI | Whole genome shotgun sequence of line | Vitis vinifera (Grape). |
| | | PN4002 . . . | |
| A5BJI4 | A5BJI4_VITVI | Putative uncharacterized protein | Vitis vinifera (Grape). |
| D1IGH5 | D1IGH5_VITVI | Whole genome shotgun sequence of line | Vitis vinifera (Grape). |
| | | PN4002 . . . | |
| D1IGG6 | D1IGG6_VITVI | Whole genome shotgun sequence of line | Vitis vinifera (Grape). |
| | | PN4002 . . . | |
| D1IGI2 | D1IG12_VITVI | Whole genome shotgun sequence of line | Vitis vinifera (Grape). |
| | | PN4002 . . . | |
| D1HBM0 | D1HBM0_VITVI | Whole genome shotgun sequence of line | Vitis vinifera (Grape). |
| | | PN4002 . . . | |
| D1HBM1 | D1HBM1_VITVI | Whole genome shotgun sequence of line | Vitis vinifera (Grape). |
| | | PN4002 . . . | |
| B9HRV8 | B9HRV8_POPTR | Predicted protein | Populus trichocarpa (Western balsam poplar) |
| | | | (Populus balsamifera subsp. OS trichocarpa). |
| Q6ED34 | Q6ED34_SOLLC | Methylesterase | Solanum lycopersicum (Tomato) (Lycopersicon |
| | | | esculentum). |
| Q56SE1 | Q56SE1_SOLTU | Methyl jasmonate esterase | Solanum tuberosum (Potato). |
| Q0JG99 | PIR7B_ORYSJ | Esterase PIR7B (EC 3.1.—.—) | Oryza sativa subsp. japonica (Rice). |
| A2WYS7 | PIR7B_ORYSI | Esterase PIR7B (EC 3.1.—.—) | Oryza sativa subsp. indica (Rice). |
| B9HRV7 | B9HRV7_POPTR | Predicted protein | Populus trichocarpa (Western balsam poplar) |
| | | | (Populus balsamifera subsp. OS trichocarpa). |
| O80472 | O80472_ARATH | Putative acetone-cyanohydrin lyaseAt2g23560 | Arabidopsis thaliana (Mouse-ear cress). |
| Q4ABP0 | Q4ABP0_BRARP | 80A08_27 | Brassica rapa subsp. pekinensis (Chinese |
| | | | cabbage). |
| D2CKY0 | D2CKY0_9SOLA | Methyl jasmonate esterase | Nicotiana attenuata. |
| A9P066 | A9P066_PICSI | Putative uncharacterized protein | Picea sitchensis (Sitka spruce). |
| A2ZYJ7 | A2ZYJ7_ORYSJ | Putative uncharacterized protein | Oryza sativa subsp. japonica (Rice). |
| B9HEE3 | B9HEE3_POPTR | Predicted protein | Populus trichocarpa (Western balsam poplar) |
| | | | (Populus balsamifera subsp. OS trichocarpa). |
| Q0JLY5 | Q0JLY5_ORYSJ | Os01g0557200 protein | Oryza sativa subsp. japonica (Rice). |
| B8LPI5 | B8LPI5_PICSI | Putative uncharacterized protein | Picea sitchensis (Sitka spruce). |
| D1HBL8 | D1HBL8_VITVI | Whole genome shotgun sequence of line | Vitis vinifera (Grape). |
| | | PN4002 . . . | |
| C5XH46 | C5XH46_SORBI | Putative uncharacterized protein Sb03g045110 | Sorghum bicolor (Sorghum) (Sorghum vulgare). |
| B9S8N0 | B9S8N0_RICCO | Polyneuridine-aldehyde esterase, putative | Ricinus communis (Castor bean). |
| Q3EBV1 | Q3EBV1_ARATH | Putative uncharacterized protein At2g23550.2 | Arabidopsis thaliana (Mouse-ear cress). |
| C5XMZ5 | C5XMZ5_SORBI | Putative uncharacterized protein Sb03g024830 | Sorghum bicolor (Sorghum) (Sorghum vulgare). |
| C5XH42 | C5XH42_SORBI | Putative uncharacterized protein Sb03g045080 | Sorghum bicolor (Sorghum) (Sorghum vulgare). |
| Q8S0U8 | Q8S0U8_ORYSJ | Putative uncharacterized proteinPutative sal . . . | Oryza sativa subsp. japonica (Rice). |
| C5XH43 | C5XH43_SORBI | Putative uncharacterized protein Sb03g045090 | Sorghum bicolor (Sorghum) (Sorghum vulgare). |
| A2WRC2 | A2WRC2_ORYSI | Putative uncharacterized protein | Oryza sativa subsp. indica (Rice). |
| Q0JG98 | PIR7A_ORYSJ | Probable esterase PIR7A (EC 3.1.—.—) | Oryza sativa subsp. japonica (Rice). |
| A2WYS8 | PIR7A_ORYSI | Probable esterase PIR7A (EC 3.1.—.—) | Oryza sativa subsp. indica (Rice). |
| B8A8T5 | B8A8T5_ORYSI | Putative uncharacterized protein | Oryza sativa subsp. indica (Rice). |
| Q5ZCR3 | Q5ZCR3_ORYSJ | Os01g0355800 proteinPutative salicylic acid- . . . | Oryza sativa subsp. japonica (Rice). |
| A5BHZ4 | A5BHZ4_VITVI | Putative uncharacterized protein | Vitis vinifera (Grape). |
| C5XH41 | C5XH41_SORBI | Putative uncharacterized protein Sb03g045070 | Sorghum bicolor (Sorghum) (Sorghum vulgare). |
| D1HBM2 | D1HBM2_VITVI | Whole genome shotgun sequence of line | Vitis vinifera (Grape). |
| | | PN4002 . . . | |
| Q8S0V0 | Q8S0V0_ORYSJ | Os01g0557100 proteinPutative | Oryza sativa subsp. japonica (Rice). |
| | | uncharacterized . . . | |

### Cyanogen Biosynthesis Inhibition

In some embodiments, the invention provides a plant (e.g. cassava) that contains transgenic inhibitor of a cyanogen biosynthesis gene (e.g. enzyme). Optionally, the inhibitor is an RNAi agent. RNAi agents include, for example, antisense RNA, dsRNA, sRNA, miRNA, shRNA, and other nucleic acids containing a segment complementary to a target sequence, and capable of inhibiting or reducing expression of the target cyanogen biosynthesis gene. Surprisingly, inhibition (e.g. by RNAi) of cyanogen biosynthesis genes such as CYP79D1 and CYP79D2 is capable of reducing cyanide levels in comestibles (e.g. cassava roots) such that ROS production and PPD are reduced.

### CYP79D1/D2 Inhibition

In some embodiments, the invention provides a plant (e.g. cassava) that contains a CYP79D1 and/or CYP79/D2 (CYP79D1/D2) inhibitor, for example, an RNAi agent. The CYP79D1/D2 inhibitor can be any product that inhibits the activity of CYP79D1/D2. CYP79D1 and CYP79D2 are P450 enzymes which catalyze the conversion of valine to its oxime, the first dedicated step in linamarin synthesis.

Optionally, the RNAi agent comprises a sequence which targets (e.g. is complementary to) a sequence which encodes a peptide listed in Table 13 and/or Table 14.

Optionally, the RNAi agent comprises a sequence which targets (e.g. is complementary to) a cassava CYP79D1 and/or a CYP79/D2 sequence (e.g. native sequence).

Optionally, the RNAi agent is operably linked to a leaf-specific promoter. Optionally, the leaf-specific promoter is a Cab1 promoter.

TABLE 13
CYP79D1 Targets

| | | | |
|---|---|---|---|
| Q5MD53 | Q5MD53_MANES | N-hydroxylating cytochrome | Manihot esculenta (Cassava) (Manioc). |
| | | P450 CYP79D1 | |
| Q9M7B8 | Q9M7B8_MANES | N-hydroxylating cytochrome | Manihot esculenta (Cassava) (Manioc). |
| | | P450 | |
| Q9M7B7 | Q9M7B7_MANES | N-hydroxylating cytochrome | Manihot esculenta (Cassava) (Manioc). |
| | | P450 | |
| Q5MD54 | Q5MD54_MANES | N-hydroxylating cytochrome | Manihot esculenta (Cassava) (Manioc). |
| | | P450 CYP79D2 | |
| B9I6Y3 | B9I6Y3_POPTR | Cytochrome P450 | Populus trichocarpa (Western balsam poplar) (Populus |
| | | | balsamifera subsp. OS trichocarpa). |
| B9I6X7 | B9I6X7_POPTR | Cytochrome P450 | Populus trichocarpa (Western balsam poplar) (Populus |
| | | | balsamifera subsp. OS trichocarpa). |
| B9NH49 | B9NH49_POPTR | Cytochrome P450 | Populus trichocarpa (Western balsam poplar) (Populus |
| | | | balsamifera subsp. OS trichocarpa). |
| B9I6Y2 | B9I6Y2_POPTR | Cytochrome P450 | Populus trichocarpa (Western balsam poplar) (Populus |
| | | | balsamifera subsp. OS trichocarpa). |
| B9H2J6 | B9H2J6_POPTR | Cytochrome P450 | Populus trichocarpa (Western balsam poplar) (Populus |
| | | | balsamifera subsp. OS trichocarpa). |
| Q43135 | C79A1_SORBI | Tyrosine N-monooxygenase | Sorghum bicolor (Sorghum) (Sorghum vulgare). |
| | | (EC 1.14.13.41) (C . . . | |
| C5WSW6 | C5WSW6_SORBI | Putative uncharacterized | Sorghum bicolor (Sorghum) (Sorghum vulgare). |
| | | protein Sb01g001200 | |
| Q6J540 | Q6J540_LOTJA | Cytochrome P450 | Lotus japonicus. |
| Q6J541 | Q6J541_LOTJA | Cytochrome P450 | Lotus japonicus. |
| O81346 | C79B2_ARATH | Tryptophan N-hydroxylase 1 | Arabidopsis thaliana (Mouse-ear cress). |
| | | (EC 1.14.13.n2) . . . | |
| B2Y2W4 | B2Y2W4_TRIRP | Cytochrome P450 | Trifolium repens (Creeping white clover). |
| B2Y2W3 | B2Y2W3_TRIRP | Cytochrome P450 | Trifolium repens (Creeping white clover). |
| B2Y2T7 | B2Y2T7_TRIRP | Cytochrome P450 | Trifolium repens (Creeping white clover). |
| B2Y2T9 | B2Y2T9_TRIRP | Cytochrome P450 | Trifolium repens (Creeping white clover). |
| B2Y2X3 | B2Y2X3_9FABA | Cytochrome P450 | Trifolium isthmocarpum. |
| B2Y2U5 | B2Y2U5_TRIRP | Cytochrome P450 | Trifolium repens (Creeping white clover). |
| B2Y2U2 | B2Y2U2_TRIRP | Cytochrome P450 | Trifolium repens (Creeping white clover). |
| O81345 | C79B1_SINAL | Cytochrome P450 79B1 (EC | Sinapis alba (White mustard) (Brassica hirta). |
| | | 1.14.—.—) | |
| B2Y2W2 | B2Y2W2_TRIRP | Cytochrome P450 | Trifolium repens (Creeping white clover). |
| B2Y2T4 | B2Y2T4_TRIRP | Cytochrome P450 | Trifolium repens (Creeping white clover). |
| B2Y2X1 | B2Y2X1_9FABA | Cytochrome P450 | Trifolium nigrescens subsp. petrisavii. |
| B2Y2U8 | B2Y2U8_TRIRP | Cytochrome P450 | Trifolium repens (Creeping white clover). |
| B2Y2T3 | B2Y2T3_TRIRP | Cytochrome P450 | Trifolium repens (Creeping white clover). |
| B2Y2X4 | B2Y2X4_9FABA | Cytochrome P450 | Trifolium isthmocarpum. |
| B2Y2X2 | B2Y2X2_9FABA | Cytochrome P450 | Trifolium nigrescens subsp. petrisavii. |
| Q8GZQ1 | Q8GZQ1_BRANA | Cytochrome P450 | Brassica napus (Rape). |
| C5H9N5 | C5H9N5_BRARP | Cytochrome P450 79b2 | Brassica rapa subsp. pekinensis (Chinese cabbage). |
| A5ASK6 | A5ASK6_VITVI | Putative uncharacterized | Vitis vinifera (Grape). |
| | | protein | |
| Q1WBS7 | Q1WBS7_9POAL | Cytochrome P450 | Bambusa ventricosa. |
| C5H9N4 | C5H9N4_BRARP | Cytochrome P450 79b2 | Brassica rapa subsp. pekinensis (Chinese cabbage). |
| B2Y2W9 | B2Y2W9_9FABA | Cytochrome P450 | Trifolium nigrescens subsp. petrisavii. |
| B2Y2X0 | B2Y2X0_9FABA | Cytochrome P450 | Trifolium nigrescens subsp. petrisavii. |
| Q501D8 | C79B3_ARATH | Tryptophan N-hydroxylase 2 | Arabidopsis thaliana (Mouse-ear cress). |
| | | (EC 1.14.13.n2) . . . | |
| A5BQ78 | A5BQ78_VITVI | Putative uncharacterized | Vitis vinifera (Grape). |
| | | protein | |
| C5Z517 | C5Z517_SORBI | Putative uncharacterized | Sorghum bicolor (Sorghum) (Sorghum vulgare). |
| | | protein Sb10g022470 | |
| C5H9N6 | C5H9N6_BRARP | Cytochrome P450 79b3 | Brassica rapa subsp. pekinensis (Chinese cabbage). |
| Q9FLC8 | C79A2_ARATH | Phenylalanine N- | Arabidopsis thaliana (Mouse-ear cress). |
| | | hydroxylase (EC 1.14.13.n1) . . . | |
| B9VQX4 | B9VQX4_HORVD | Cytochrome P450 | Hordeum vulgare var. distichum (Two-rowed barley). |
| B8XX43 | B8XX43_HORVD | Cytochrome P450 | Hordeum vulgare var. distichum (Two-rowed barley). |
| C5H9N7 | C5H9N7_BRARP | Cytochrome P450 79a2 | Brassica rapa subsp. pekinensis (Chinese cabbage). |
| D1HW49 | D1HW49_VITVI | Whole genome shotgun | Vitis vinifera (Grape). |
| | | sequence of line PN4002 . . . | |
| C5WV73 | C5WV73_SORBI | Putative uncharacterized | Sorghum bicolor (Sorghum) (Sorghum vulgare). |
| | | protein Sb01g016480 | |
| C5WV72 | C5WV72_SORBI | Putative uncharacterized | Sorghum bicolor (Sorghum) (Sorghum vulgare). |
| | | protein Sb01g016470 | |
| C5WV67 | C5WV67_SORBI | Putative uncharacterized | Sorghum bicolor (Sorghum) (Sorghum vulgare). |
| | | protein Sb01g016460 | |
| B9T7E4 | B9T7E4_RICCO | Cytochrome P450, putative | Ricinus communis (Castor bean). |
| Q9AY90 | Q9AY90_ORYSA | Putative cytochrome p450tyr | Oryza sativa (Rice). |
| Q10HZ6 | Q10HZ6_ORYSJ | Os03g0570100 | Oryza sativa subsp. japonica (Rice). |
| | | proteincDNA clone: 002-117- | |
| | | A08, . . . | |
| Q0JF25 | Q0JF25_ORYSJ | Os04g0171800 | Oryza sativa subsp. japonica (Rice). |
| | | proteincDNA | |
| | | clone: J023074N24, f . . . | |
| Q5H9W6 | Q5H9W6_ORYSA | B1168G10.4 protein | Oryza sativa (Rice). |
| A3ARM1 | A3ARM1_ORYSJ | Putative uncharacterized | Oryza sativa subsp. japonica (Rice). |
| | | protein | |
| C5Y4T6 | C5Y4T6_SORBI | Putative uncharacterized | Sorghum bicolor (Sorghum) (Sorghum vulgare). |
| | | protein Sb05g022010 | |
| D1HW43 | D1HW43_VITVI | Whole genome shotgun | Vitis vinifera (Grape). |
| | | sequence of line PN4002 . . . | |
| B6SYQ1 | B6SYQ1_MAIZE | Cytochrome P450 | Zea mays (Maize). |
| | | CYP79A33 | |
| Q9M7B9 | Q9M7B9_9LILI | Cytochrome P450 CYP79E2 | Triglochin maritima. |
| Q9M7C0 | Q9M7C0_9LILI | Cytochrome P450 CYP79E1 | Triglochin maritima. |
| C5YFX2 | C5YFX2_SORBI | Putative uncharacterized | Sorghum bicolor (Sorghum) (Sorghum vulgare). |
| | | protein Sb06g015920 | |
| C5Y4U5 | C5Y4U5_SORBI | Putative uncharacterized | Sorghum bicolor (Sorghum) (Sorghum vulgare). |
| | | protein Sb05g022070 | |
| Q01MC1 | Q01MC1_ORYSA | OSIGBa0114I04.1 protein | Oryza sativa (Rice). |
| D1HW45 | D1HW45_VITVI | Whole genome shotgun | Vitis vinifera (Grape). |
| | | sequence of line PN4002 . . . | |
| B8AEG6 | B8AEG6_ORYSI | Putative uncharacterized | Oryza sativa subsp. indica (Rice). |
| | | protein | |
| D1HW44 | D1HW44_VITVI | Whole genome shotgun | Vitis vinifera (Grape). |
| | | sequence of line PN4002 . . . | |
| Q949U1 | C79F1_ARATH | Dihomomethionine N- | Arabidopsis thaliana (Mouse-ear cress). |
| | | hydroxylase (EC 1.14.13.n . . . | |
| Q9FUY7 | C79F2_ARATH | Hexahomomethionine N- | Arabidopsis thaliana (Mouse-ear cress). |
| | | hydroxylase (EC 1.14.13 . . . | |
| A5B623 | A5B623_VITVI | Putative uncharacterized | Vitis vinifera (Grape). |
| | | protein | |
| Q9LQB7 | Q9LQB7_ARATH | Cytochrome P450, | Arabidopsis thaliana (Mouse-ear cress). |
| | | putativeF19C14.12 protein | |
| Q0JF26 | Q0JF26_ORYSJ | Os04g0171600 | Oryza sativa subsp. japonica (Rice). |
| | | proteincDNA | |
| | | clone: J023119K20, f . . . | |
| B2D2I0 | B2D2I0_BRAOL | CYP79F1 | Brassica oleracea (Wild cabbage). |
| C5H9N3 | C5H9N3_BRARP | Cytochrome P450 79f1 | Brassica rapa subsp. pekinensis (Chinese cabbage). |
| A3AJP9 | A3AJP9_ORYSJ | Putative uncharacterized | Oryza sativa subsp. japonica (Rice). |
| | | protein | |
| Q5H9W8 | Q5H9W8_ORYSA | B1168G10.2 protein | Oryza sativa (Rice). |
| B9FE23 | B9FE23_ORYSJ | Putative uncharacterized | Oryza sativa subsp. japonica (Rice). |
| | | protein | |
| B8AR68 | B8AR68_ORYSI | Putative uncharacterized | Oryza sativa subsp. indica (Rice). |
| | | protein | |
| B8A7X1 | B8A7X1_ORYSI | Putative uncharacterized | Oryza sativa subsp. indica (Rice). |
| | | protein | |
| B9SBS2 | B9SBS2_RICCO | Cytochrome P450, putative | Ricinus communis (Castor bean). |
| B9SBR9 | B9SBR9_RICCO | Cytochrome P450, putative | Ricinus communis (Castor bean). |
| B9SBR6 | B9SBR6_RICCO | Cytochrome P450, putative | Ricinus communis (Castor bean). |
| Q949U1-2 | Q949U1-2 | Dihomomethionine N- | Arabidopsis thaliana (Mouse-ear cress). |
| | | hydroxylase (EC 1.14.13.n . . . | |
| O64514 | O64514_ARATH | YUP8H12R.1 protein | Arabidopsis thaliana (Mouse-ear cress). |
| D1HW48 | D1HW48_VITVI | Whole genome shotgun | Vitis vinifera (Grape). |
| | | sequence of line PN4002 . . . | |
| B5AXG1 | B5AXG1_BRARC | Cytochrome P450 CYP79A2 | Brassica rapa subsp. chinensis (Pak-choi). |
| B9SBG5 | B9SBG5_RICCO | Flavonoid 3-hydroxylase, | Ricinus communis (Castor bean). |
| | | putative | |
| D1HT71 | D1HT71_VITVI | Whole genome shotgun | Vitis vinifera (Grape). |
| | | sequence of line PN4002 . . . | |
| Q9LNJ4 | Q9LNJ4_ARATH | Putative cytochrome | Arabidopsis thaliana (Mouse-ear cress). |
| | | P450At1g01280 | |
| B9GR20 | B9GR20_POPTR | Cytochrome P450 | Populus trichocarpa (Western balsam poplar) (Populus |
| | | | balsamifera subsp. OS trichocarpa). |
| A5B291 | A5B291_VITVI | Putative uncharacterized | Vitis vinifera (Grape). |
| | | protein | |
| Q8W0R8 | Q8W0R8_SORBI | Putative uncharacterized | Sorghum bicolor (Sorghum) (Sorghum vulgare). |
| | | protein Sb07g002610 . . . | |
| Q9MBF5 | Q9MBF5_PETHY | Cytochrome P450 | Petunia hybrida (Petunia). |
| B6TC85 | B6TC85_MAIZE | Flavonoid 3- | Zea mays (Maize). |
| | | monooxygenase | |
| C0PLY5 | C0PLY5_MAIZE | Putative uncharacterized | Zea mays (Maize). |
| | | protein | |
| D1J4M9 | D1J4M9_VITVI | Whole genome shotgun | Vitis vinifera (Grape). |
| | | sequence of line PN4002 . . . | |
| A5BYM3 | A5BYM3_VITVI | Putative uncharacterized | Vitis vinifera (Grape). |
| | | protein | |
| Q0JF20 | Q0JF20_ORYSJ | Os04g0174100 protein | Oryza sativa subsp. japonica (Rice). |
| Q7EZR4 | Q7EZR4_ORYSJ | Os08g0131100 | Oryza sativa subsp. japonica (Rice). |
| | | proteinPutative cytochrome | |
| | | P450 . . . | |
| A3BPC5 | A3BPC5_ORYSJ | Putative uncharacterized | Oryza sativa subsp. japonica (Rice). |
| | | protein | |
| A2YQX7 | A2YQX7_ORYSI | Putative uncharacterized | Oryza sativa subsp. indica (Rice). |
| | | protein | |
| C5IGQ3 | C5IGQ3_MALDO | Flavonoid 3′ hydroxylase | Malus domestica (Apple) (Pyrus malus). |

TABLE 14
CYP79D2 Targets

| | | | |
|---|---|---|---|
| Q5MD54 | Q5MD54_MANES | N-hydroxylating cytochrome P450 CYP79D2 | Manihot esculenta (Cassava) (Manioc). |
| Q9M7B7 | Q9M7B7_MANES | N-hydroxylating cytochrome P450 | Manihot esculenta (Cassava) (Manioc). |
| Q9M7B8 | Q9M7B8_MANES | N-hydroxylating cytochrome P450 | Manihot esculenta (Cassava) (Manioc). |
| Q5MD53 | Q5MD53_MANES | N-hydroxylating cytochrome P450 CYP79D1 | Manihot esculenta (Cassava) (Manioc). |
| Q43135 | C79A1_SORBI | Tyrosine N-monooxygenase (EC | Sorghum bicolor (Sorghum) (Sorghum |
| | | 1.14.13.41) (C . . . | vulgare). |
| C5WSW6 | C5WSW6_SORBI | Putative uncharacterized protein | Sorghum bicolor (Sorghum) (Sorghum |
| | | Sb01g001200 | vulgare). |
| B9I6Y3 | B9I6Y3_POPTR | Cytochrome P450 | Populus trichocarpa (Western balsam poplar) |
| | | | (Populus balsamifera subsp. OS trichocarpa). |
| B9I6X7 | B9I6X7_POPTR | Cytochrome P450 | Populus trichocarpa (Western balsam poplar) |
| | | | (Populus balsamifera subsp. OS trichocarpa). |
| B9NH49 | B9NH49_POPTR | Cytochrome P450 | Populus trichocarpa (Western balsam poplar) |
| | | | (Populus balsamifera subsp. OS trichocarpa). |
| B9I6Y2 | B9I6Y2_POPTR | Cytochrome P450 | Populus trichocarpa (Western balsam poplar) |
| | | | (Populus balsamifera subsp. OS trichocarpa). |
| Q6J540 | Q6J540_LOTJA | Cytochrome P450 | Lotus japonicus. |
| Q6J541 | Q6J541_LOTJA | Cytochrome P450 | Lotus japonicus. |
| B9H2J6 | B9H2J6_POPTR | Cytochrome P450 | Populus trichocarpa (Western balsam poplar) |
| | | | (Populus balsamifera subsp. OS trichocarpa). |
| B2Y2W4 | B2Y2W4_TRIRP | Cytochrome P450 | Trifolium repens (Creeping white clover). |
| B2Y2X1 | B2Y2X1_9FABA | Cytochrome P450 | Trifolium nigrescens subsp. petrisavii. |
| B2Y2W3 | B2Y2W3_TRIRP | Cytochrome P450 | Trifolium repens (Creeping white clover). |
| B2Y2T7 | B2Y2T7_TRIRP | Cytochrome P450 | Trifolium repens (Creeping white clover). |
| B2Y2X3 | B2Y2X3_9FABA | Cytochrome P450 | Trifolium isthmocarpum. |
| B2Y2U2 | B2Y2U2_TRIRP | Cytochrome P450 | Trifolium repens (Creeping white clover). |
| B2Y2T9 | B2Y2T9_TRIRP | Cytochrome P450 | Trifolium repens (Creeping white clover). |
| O81346 | C79B2_ARATH | Tryptophan N-hydroxylase 1 (EC | Arabidopsis thaliana (Mouse-ear cress). |
| | | 1.14.13.n2) . . . | |
| B2Y2W2 | B2Y2W2_TRIRP | Cytochrome P450 | Trifolium repens (Creeping white clover). |
| B2Y2U5 | B2Y2U5_TRIRP | Cytochrome P450 | Trifolium repens (Creeping white clover). |
| B2Y2X2 | B2Y2X2_9FABA | Cytochrome P450 | Trifolium nigrescens subsp. petrisavii. |
| B2Y2T4 | B2Y2T4_TRIRP | Cytochrome P450 | Trifolium repens (Creeping white clover). |
| B2Y2T3 | B2Y2T3_TRIRP | Cytochrome P450 | Trifolium repens (Creeping white clover). |
| B2Y2U8 | B2Y2U8_TRIRP | Cytochrome P450 | Trifolium repens (Creeping white clover). |
| B2Y2X4 | B2Y2X4_9FABA | Cytochrome P450 | Trifolium isthmocarpum. |
| Q8GZQ1 | Q8GZQ1_BRANA | Cytochrome P450 | Brassica napus (Rape). |
| A5ASK6 | A5ASK6_VITVI | Putative uncharacterized protein | Vitis vinifera (Grape). |
| O81345 | C79B1_SINAL | Cytochrome P450 79B1 (EC 1.14.—.—) | Sinapis alba (White mustard) (Brassica hirta). |
| C5H9N4 | C5H9N4_BRARP | Cytochrome P450 79b2 | Brassica rapa subsp. pekinensis (Chinese |
| | | | cabbage). |
| B2Y2W9 | B2Y2W9_9FABA | Cytochrome P450 | Trifolium nigrescens subsp. petrisavii. |
| B2Y2X0 | B2Y2X0_9FABA | Cytochrome P450 | Trifolium nigrescens subsp. petrisavii. |
| A5BQ78 | A5BQ78_VITVI | Putative uncharacterized protein | Vitis vinifera (Grape). |
| C5H9N5 | C5H9N5_BRARP | Cytochrome P450 79b2 | Brassica rapa subsp. pekinensis (Chinese |
| | | | cabbage). |
| C5Z517 | C5Z517_SORBI | Putative uncharacterized protein | Sorghum bicolor (Sorghum) (Sorghum |
| | | Sb10g022470 | vulgare). |
| Q1WBS7 | Q1WBS7_9POAL | Cytochrome P450 | Bambusa ventricosa. |
| Q501D8 | C79B3_ARATH | Tryptophan N-hydroxylase 2 (EC | Arabidopsis thaliana (Mouse-ear cress). |
| | | 1.14.13.n2) . . . | |
| Q9FLC8 | C79A2_ARATH | Phenylalanine N-hydroxylase (EC | Arabidopsis thaliana (Mouse-ear cress). |
| | | 1.14.13.n1) . . . | |
| C5H9N6 | C5H9N6_BRARP | Cytochrome P450 79b3 | Brassica rapa subsp. pekinensis (Chinese |
| | | | cabbage). |
| B8XX43 | B8XX43_HORVD | Cytochrome P450 | Hordeum vulgare var. distichum (Two-rowed |
| | | | barley). |
| B9VQX4 | B9VQX4_HORVD | Cytochrome P450 | Hordeum vulgare var. distichum (Two-rowed |
| | | | barley). |
| C5H9N7 | C5H9N7_BRARP | Cytochrome P450 79a2 | Brassica rapa subsp. pekinensis (Chinese |
| | | | cabbage). |
| D1HW49 | D1HW49_VITVI | Whole genome shotgun sequence of line | Vitis vinifera (Grape). |
| | | PN4002 . . . | |
| C5WV73 | C5WV73_SORBI | Putative uncharacterized protein | Sorghum bicolor (Sorghum) (Sorghum |
| | | Sb01g016480 | vulgare). |
| C5WV67 | C5WV67_SORBI | Putative uncharacterized protein | Sorghum bicolor (Sorghum) (Sorghum |
| | | Sb01g016460 | vulgare). |
| C5WV72 | C5WV72_SORBI | Putative uncharacterized protein | Sorghum bicolor (Sorghum) (Sorghum |
| | | Sb01g016470 | vulgare). |
| Q5H9W6 | Q5H9W6_ORYSA | B1168G10.4 protein | Oryza sativa (Rice). |
| Q0JF25 | Q0JF25_ORYSJ | Os04g0171800 proteincDNA | Oryza sativa subsp. japonica (Rice). |
| | | clone: J023074N24, f . . . | |
| A3ARM1 | A3ARM1_ORYSJ | Putative uncharacterized protein | Oryza sativa subsp. japonica (Rice). |
| C5Y4T6 | C5Y4T6_SORBI | Putative uncharacterized protein | Sorghum bicolor (Sorghum) (Sorghum |
| | | Sb05g022010 | vulgare). |
| Q9AY90 | Q9AY90_ORYSA | Putative cytochrome p450tyr | Oryza sativa (Rice). |
| Q10HZ6 | Q10HZ6_ORYSJ | Os03g0570100 proteincDNA clone: 002-117- | Oryza sativa subsp. japonica (Rice). |
| | | A08, . . . | |
| B6SYQ1 | B6SYQ1_MAIZE | Cytochrome P450 CYP79A33 | Zea mays (Maize). |
| B9T7E4 | B9T7E4_RICCO | Cytochrome P450, putative | Ricinus communis (Castor bean). |
| D1HW43 | D1HW43_VITVI | Whole genome shotgun sequence of line | Vitis vinifera (Grape). |
| | | PN4002 . . . | |
| Q9M7C0 | Q9M7C0_9LILI | Cytochrome P450 CYP79E1 | Triglochin maritima. |
| Q9M7B9 | Q9M7B9_9LILI | Cytochrome P450 CYP79E2 | Triglochin maritima. |
| C5YFX2 | C5YFX2_SORBI | Putative uncharacterized protein | Sorghum bicolor (Sorghum) (Sorghum |
| | | Sb06g015920 | vulgare). |
| C5Y4U5 | C5Y4U5_SORBI | Putative uncharacterized protein | Sorghum bicolor (Sorghum) (Sorghum |
| | | Sb05g022070 | vulgare). |
| Q01MC1 | Q01MC1_ORYSA | OSIGBa0114I04.1 protein | Oryza sativa (Rice). |
| D1HW45 | D1HW45_VITVI | Whole genome shotgun sequence of line | Vitis vinifera (Grape). |
| | | PN4002 . . . | |
| B8AEG6 | B8AEG6_ORYSI | Putative uncharacterized protein | Oryza sativa subsp. indica (Rice). |
| D1HW44 | D1HW44_VITVI | Whole genome shotgun sequence of line | Vitis vinifera (Grape). |
| | | PN4002 . . . | |
| Q949U1 | C79F1_ARATH | Dihomomethionine N-hydroxylase (EC | Arabidopsis thaliana (Mouse-ear cress). |
| | | 1.14.13.n . . . | |
| A5B623 | A5B623_VITVI | Putative uncharacterized protein | Vitis vinifera (Grape). |
| Q9FUY7 | C79F2_ARATH | Hexahomomethionine N-hydroxylase (EC | Arabidopsis thaliana (Mouse-ear cress). |
| | | 1.14.13 . . . | |
| Q0JF26 | Q0JF26_ORYSJ | Os04g0171600 proteincDNA | Oryza sativa subsp. japonica (Rice). |
| | | clone: J023119K20, f . . . | |
| C5H9N3 | C5H9N3_BRARP | Cytochrome P450 79f1 | Brassica rapa subsp. pekinensis (Chinese |
| | | | cabbage). |
| B2D2I0 | B2D2I0_BRAOL | CYP79F1 | Brassica oleracea (Wild cabbage). |
| Q9LQB7 | Q9LQB7_ARATH | Cytochrome P450, putativeF19C14.12 | Arabidopsis thaliana (Mouse-ear cress). |
| | | protein | |
| Q5H9W8 | Q5H9W8_ORYSA | B1168G10.2 protein | Oryza sativa (Rice). |
| A3AJP9 | A3AJP9_ORYSJ | Putative uncharacterized protein | Oryza sativa subsp. japonica (Rice). |
| B9FE23 | B9FE23_ORYSJ | Putative uncharacterized protein | Oryza sativa subsp. japonica (Rice). |
| B8AR68 | B8AR68_ORYSI | Putative uncharacterized protein | Oryza sativa subsp. indica (Rice). |
| B8A7X1 | B8A7X1_ORYSI | Putative uncharacterized protein | Oryza sativa subsp. indica (Rice). |
| B9SBS2 | B9SBS2_RICCO | Cytochrome P450, putative | Ricinus communis (Castor bean). |
| B9SBR9 | B9SBR9_RICCO | Cytochrome P450, putative | Ricinus communis (Castor bean). |
| Q949U1-2 | Q949U1-2 | Dihomomethionine N-hydroxylase (EC | Arabidopsis thaliana (Mouse-ear cress). |
| | | 1.14.13.n . . . | |
| B9SBR6 | B9SBR6_RICCO | Cytochrome P450, putative | Ricinus communis (Castor bean). |
| D1HW48 | D1HW48_VITVI | Whole genome shotgun sequence of line | Vitis vinifera (Grape). |
| | | PN4002 . . . | |
| O64514 | O64514_ARATH | YUP8H12R.1 protein | Arabidopsis thaliana (Mouse-ear cress). |
| B5AXG1 | B5AXG1_BRARC | Cytochrome P450 CYP79A2 | Brassica rapa subsp. chinensis (Pak-choi). |
| B9SBG5 | B9SBG5_RICCO | Flavonoid 3-hydroxylase, putative | Ricinus communis (Castor bean). |
| B9GR20 | B9GR20_POPTR | Cytochrome P450 | Populus trichocarpa (Western balsam poplar) |
| | | | (Populus balsamifera subsp. OS trichocarpa). |
| D1HT71 | D1HT71_VITVI | Whole genome shotgun sequence of line | Vitis vinifera (Grape). |
| | | PN4002 . . . | |
| Q9LNJ4 | Q9LNJ4_ARATH | Putative cytochrome P450At1g01280 | Arabidopsis thaliana (Mouse-ear cress). |
| A5B291 | A5B291_VITVI | Putative uncharacterized protein | Vitis vinifera (Grape). |
| Q8W0R8 | Q8W0R8_SORBI | Putative uncharacterized protein | Sorghum bicolor (Sorghum) (Sorghum |
| | | Sb07g002610 . . . | vulgare). |
| B6TC85 | B6TC85_MAIZE | Flavonoid 3-monooxygenase | Zea mays (Maize). |
| Q9MBF5 | Q9MBF5_PETHY | Cytochrome P450 | Petunia hybrida (Petunia). |
| C0PLY5 | C0PLY5_MAIZE | Putative uncharacterized protein | Zea mays (Maize). |
| D1J4M9 | D1J4M9_VITVI | Whole genome shotgun sequence of line | Vitis vinifera (Grape). |
| | | PN4002 . . . | |
| Q0JF20 | Q0JF20_ORYSJ | Os04g0174100 protein | Oryza sativa subsp. japonica (Rice). |
| A5BYM3 | A5BYM3_VITVI | Putative uncharacterized protein | Vitis vinifera (Grape). |
| B8LLA5 | B8LLA5_PICSI | Putative uncharacterized protein | Picea sitchensis (Sitka spruce). |
| Q69P77 | Q69P77_ORYSJ | Os09g0441100 proteincDNA clone: 002-130- | Oryza sativa subsp. japonica (Rice). |
| | | E07, . . . | |
| Q7EZR4 | Q7EZR4_ORYSJ | Os08g0131100 proteinPutative cytochrome | Oryza sativa subsp. japonica (Rice). |
| | | P450 . . . | |
| A3BPC5 | A3BPC5_ORYSJ | Putative uncharacterized protein | Oryza sativa subsp. japonica (Rice). |

In one aspect the DNA constructs and expression vectors for the transgenes of the present invention are operatively coupled to an expression control sequences, and transcriptional terminator for efficient expression in the plant of interest.

In one aspect of any of these expression vectors, and DNA constructs the nucleic acid encoding the transgene of the present invention is codon optimized for expression in the plant of interest.

In some embodiments, the DNA constructs and expression vectors of the invention further comprise polynucleotide sequences encoding one or more of the following elements i) a selectable marker gene to enable antibiotic selection, ii) a screenable marker gene to enable visual identification of transformed cells, and iii) T-element DNA sequences to enable Agrobacterium tumefaciens mediated transformation. In some embodiments the expression vector comprises a vector backbone selected from pBin, pCAMBIA, pCGN, EHA105 and pZP212.

Those of skill in the art will appreciate that the foregoing descriptions of expression cassettes represents only illustrative examples of expression cassettes that could be readily constructed, and is not intended to represent an exhaustive list of all possible DNA constructs or expression cassettes that could be constructed.

Moreover expression vectors suitable for use in expressing the claimed DNA constructs in plants, and methods for their construction are generally well known, and need not be limited. These techniques, including techniques for nucleic acid manipulation of genes such as subcloning a subject promoter, or nucleic acid sequences encoding a gene of interest into expression vectors, labeling probes, DNA hybridization, and the like, and are described generally in Sambrook, et al., Molecular Cloning—A Laboratory Manual (2nd Ed.), Vol. 1-3, Cold Spring Harbor Laboratory, Cold Spring Harbor, N.Y., 1989, which is incorporated herein by reference. For instance, various procedures, such as PCR, or site directed mutagenesis can be used to introduce a restriction site at the start codon of a heterologous gene of interest. Heterologous DNA sequences are then linked to a suitable expression control sequences such that the expression of the gene of interest are regulated (operatively coupled) by the promoter.

DNA constructs comprising an expression cassette for the gene of interest can then be inserted into a variety of expression vectors. Such vectors include expression vectors that are useful in the transformation of plant cells. Many other such vectors useful in the transformation of plant cells can be constructed by the use of recombinant DNA techniques well known to those of skill in the art as described above.

Exemplary expression vectors for expression in protoplasts or plant tissues include pUC 18/19 or pUC 118/119 (GIBCO BRL, Inc., MD); pBluescript SK (+/−) and pBluescript KS (+/−) (STRATAGENE, La Jolla, Calif.); pT7Blue T-vector (NOVAGEN, Inc., WI); pGEM-3Z/4Z (PROMEGA Inc., Madison, Wis.), and the like vectors, such as is described herein

Exemplary vectors for expression using Agrobacterium tumefaciens-mediated plant transformation include for example, pBin 19 (CLONETECH), Frisch et al, Plant Mol. Biol., 27:405-409, 1995; pCAMBIA 1200 and pCAMBIA 1201 (Center for the Application of Molecular Biology to International Agriculture, Canberra, Australia); pGA482, An et al, EMBO J., 4:277-284, 1985; pCGN1547, (CALGENE Inc.) McBride et al, Plant Mol. Biol., 14:269-276, 1990, pZP212 (Hajdukiewicz et al., Plant. Mol. Biol. 25 989-994), EHA105 and the like vectors, such as is described herein.

DNA constructs will typically include expression control sequences comprising promoters to drive expression of the transgene of interest within the organism. Promoters may provide ubiquitous, cell type specific, constitutive promoter or inducible promoter expression. Basal promoters in plants typically comprise canonical regions associated with the initiation of transcription, such as CAAT and TATA boxes. The TATA box element is usually located approximately 20 to 35 nucleotides upstream of the initiation site of transcription. The CAAT box element is usually located approximately 40 to 200 nucleotides upstream of the start site of transcription. The location of these basal promoter elements result in the synthesis of an RNA transcript comprising nucleotides upstream of the translational ATG start site. The region of RNA upstream of the ATG is commonly referred to as a 5′ untranslated region or 5′ UTR. It is possible to use standard molecular biology techniques to make combinations of basal promoters, that is, regions comprising sequences from the CAAT box to the translational start site, with other upstream promoter elements to enhance or otherwise alter promoter activity or specificity.

In some aspects promoters may be altered to contain “enhancer DNA” to assist in elevating gene expression. As is known in the art certain DNA elements can be used to enhance the transcription of DNA. These enhancers often are found 5′ to the start of transcription in a promoter that functions in eukaryotic cells, but can often be inserted upstream (5′) or downstream (3′) to the coding sequence. In some instances, these 5′ enhancer DNA elements are introns. Among the introns that are particularly useful as enhancer DNA are the 5′ introns from the rice actin 1 gene (see U.S. Pat. No. 5,641,876), the rice actin 2 gene, the maize alcohol dehydrogenase gene, the maize heat shock protein 70 gene (U.S. Pat. No. 5,593,874), the maize shrunken 1 gene, the light sensitive 1 gene of Solanum tuberosum, the maize Ubi1 promoter/intron/tobacco etch virus mRNA leader sequence, and the heat shock protein 70 gene of Petunia hybrida (U.S. Pat. No. 5,659,122).

In one embodiment, a DNA construct is useful to transform a host with a transgene of the present invention (e.g. AOX) operably linked to a promoter. Such a DNA construct can further comprise other genetic elements such as promoters, terminators, elements to facilitate cloning, etc. Optionally, the promoter is constitutive, tissue-specific, cell-type specific, developmentally regulated, One skilled in the art will readily appreciate methods to operably link a desired promoter to the transgene, for example, as described in “Current Protocols in Molecular Biology” (Copyright© 2007 by John Wiley and Sons, Inc); “Plant Gene Transfer and Expression Protocols (Methods in Molecular Biology)” by Heddwyn Jones et al.; 1995 Humana Press Inc.; and “Plant Genomics: Methods and Protocols (Methods in Molecular Biology)” by Gustafson et al.; 2009 Humana Press Inc.

The DNA construct can be any vector capable of transforming cells of plants to express an exogenous gene. Non-limiting examples include plasmids, viruses or other suitable replicons, and Agrobacterium vectors. Other vectors, for example, are described by as described in “Current Protocols in Molecular Biology” (Copyright© 2007 by John Wiley and Sons, Inc); “Agrobacterium Protocols (Methods in Molecular Biology)” by Gartland et al.; 1995 Humana Press Inc.; “Agrobacterium Protocols Volumes 1 and 2 (Methods in Molecular Biology)” by Kan Wang 2006 Humana Press Inc.; and “Plant Gene Transfer and Expression Protocols (Methods in Molecular Biology)” by Heddwyn Jones et al.; 1995 Humana Press Inc.

In some embodiments, the DNA construct is a vector that allows integration in a host cell genome, including chromosomal genomes and plastid genomes, for example, by homologous recombination. Such vectors are well known in the art and provide an expression cassette flanked by DNA sequences which are homologous to DNA sequences of a plastid genome of the plant.

In some embodiments, the methods and constructs are useful for expressing a transgene in mitochondria.

In some embodiments, the methods and DNA constructs are useful for expressing a transgene is a plastid. In some embodiments, the plastid is selected from a chloroplast, chromoplasts, amyloplast, proplastid, leucoplasts and etioplasts. Optionally, the plastid is a chloroplast.

In some embodiments, vectors are capable of plastid transformation such as, for example, for chloroplast transformation. Such vectors include plastid transcription vectors such as pUC, pBR322, pBLUESCRIPT, pGEM, and all others identified by Daniel in U.S. Pat. No. 5,693,507 and U.S. Pat. No. 5,932,479, each of which is hereby incorporated by reference. Included are also vectors whose flanking sequences are located outside of the embroidered repeat of the chloroplast genome.

The present invention also contemplates the use of universal vectors described in WO 99/10513 which was published on Mar. 4, 1999, and application Ser. No. 09/079,640 which was filed on can 15, 1998, wherein both of said references are incorporated in their entirety.

The present invention also contemplates the use of basic pLD vectors, developed for chloroplast transformation (Daniell et al., 1998; Daniell et al., 2001b; De Cosa et al., 2001; Guda et al., 2000; Kota et al., 1999). Optionally present vectors use the SD 5′ sequence (Daniell et al., 2001b; Degray et al., 2001; Kota et al., 1999) for high levels of polynucleotide transcription in chloroplasts (e.g. 3-21% of total soluble leaf protein).

It should be noted that the DNA constructs described herein are illustrative examples and vectors can be constructed with different promoters such as was described in U.S. patent application Ser. No. 09/079,640, different selectable markers such as those described in U.S. patent application Ser. No. 09/807,722, and different flanking sequences suitable for integration into a variety of plant plastid genomes. Other vectors, for example, are described by as described in “Current Protocols in Molecular Biology” (Copyright© 2007 by John Wiley and Sons, Inc).

In one embodiment, a DNA construct is constructed to enhance expression in the host, part thereof. Examples of such construction are well known in the art, for example, codon optimization, gene fusions, and non-translated sequences.

Optionally, a DNA construct is constructed such that a transgene (e.g. AOX) is fused to a targeting sequence. Examples of such are well known in the art, for example, plastid targeting sequences, mitochondrial targeting sequences, vacuole targeting sequences, and the like. Optionally, a vector is constructed such that a transgene (e.g. AOX) is fused to a mitochondrial-targeting signal sequence to provide localization upon translation, as described, for example, in “Plant Gene Transfer and Expression Protocols (Methods in Molecular Biology)” by Heddwyn Jones et al.; 1995 Humana Press Inc.

Optionally, a plastid targeting sequence is encoded by Error! Reference source not found., or derivative thereof.

Optionally, a DNA construct is constructed with one or more non-translated elements which enhance expression in the host. Such elements are well known in the art, for example, leader sequences and terminator sequences.

Optionally, a DNA construct is constructed such that a transgene (e.g. AOX) is operably linked to a leader sequence (e.g. HSP70 leader). Examples of useful leader sequences are described, for example, in U.S. Pat. No. 5,362,865.

Optionally, a DNA construct is constructed such that a transgene (e.g. AOX) is operably linked to a terminator sequence (e.g. Nos terminator).

Optionally, a DNA construct is constructed such that a transgene (e.g. AOX) is operably linked to a leader sequence and a terminator sequence.

Optionally, DNA construct comprise a selectable marker or screenable marker, useful, e.g., for identifying desired transformation events.

### Promoters

A transgene of the present invention (e.g. AOX and/or antioxidation genes) is operably linked to a promoter functional in the host plant (e.g. cassava). The skilled artisan will readily recognize now that promoter selection can be made based upon the localization of the transgene (e.g. nuclear, plastid, or mitochondria), the host (e.g. cassava), the tissue specificity (e.g. constitutive or tissue-specific), and the expression level desired. In addition, for coexpression of transgenes (e.g. AOX and PSY), the transgenes can be operably linked to the same promoter (e.g. patatin) or to different promoters.

Optionally, the promoter is a sequence that is homologous to a host promoter (e.g. a cassava promoter is transformed into a cassava). Optionally, the promoter is endogenous to the host (e.g. not inserted by transformation).

Optionally, the promoter is constitutive. Optionally, the promoter is tissue-specific. Optionally, a tissue-specific promoter is a root-specific promoter. Optionally, the tissue specific promoter is a leaf-specific promoter (e.g. Cab1 promoter).

Optionally, the tissue-specific promoter is comestible-specific (e.g. tissue specific). Comestible portions (plant parts which are generally regarded as food for animals such as humans) of plants (e.g. cyanogenic crops) are known in the art (e.g. the root of a cassava or fruit of a mango). With the teachings provided herein, the skilled artisan can now select an appropriate comestible-specific promoter, depending on the plant to be transformed. (e.g. specific to a tissue that is harvested for food).

Optionally, the tissue- or comestible-specific promoter is a fruit-specific promoter.

Optionally, a root-specific promoter is selected from the group consisting of: a patatin promoter (e.g. B33 described in U.S. Pat. No. 5,723,757), an isoflavone synthase promoter (e.g. ifs1 or isf2 described in U.S. Pat. No. 7,196,247), a granular bound starch synthase (GBSS) promoter, a sporamin promoter (e.g. as described in U.S. Pat. No. 7,041,815), and a sugar beet promoter (e.g. as described in U.S. Pat. No. 6,248,936).

Optionally, the root-specific promoter is a GBSS promoter comprising Error! Reference source not found., or derivative thereof.

### Transformation

The skilled artisan will recognize that plants can be transformed according to the present invention by using any useful method.

Useful methods include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, and U.S. Pat. No. 5,464,765, each specifically incorporated herein by reference), by Agrobacterium-mediated transformation (U.S. Pat. No. 5,591,616 and U.S. Pat. No. 5,563,055; each specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference), lipofection, viral methods, and other methods known in the art.

In one embodiment, transformation comprises Agrobacterium-mediated transfer, for example, as described below.

Agrobacterium-mediated transfer is a system that is widely applicable for introducing genes into plant. The use of Agrobacterium-mediated plant integrating vectors to introduce DNA into plant cells is well known in the art. See, for example, the methods described by Fraley et al. (1985), Rogers et al. (1987) and U.S. Pat. No. 5,563,055, specifically incorporated herein by reference in its entirety.

Agrobacterium-mediated transformation is efficient in dicotyledonous plants and advances in Agrobacterium-mediated transformation techniques have now made the technique applicable to nearly all monocotyledonous plants. For example, Agrobacterium-mediated transformation techniques have now been applied to rice (Hiei et al, 1997; Zhang et al., 1997; U.S. Pat. No. 5,591,616, specifically incorporated herein by reference in its entirety), wheat (McCormac et al., 1998), barley (Tingay et al., 1997; McCormac et al., 1998), and maize (Ishida et al., 1996; U.S. Pat. No. 5,981,840).

Modern Agrobacterium transformation vectors are capable of replication in E. coli as well as Agrobacterium, allowing for convenient manipulations as described (Klee et al, 1985). Moreover, recent technological advances in vectors for Agrobacterium-mediated gene transfer have improved the arrangement of genes and restriction sites in the vectors to facilitate the construction of vectors capable of expressing various polypeptide encoding genes. The vectors described (Rogers et al., 1987) have convenient multi-linker regions flanked by a promoter and a polyadenylation site for direct expression of inserted polypeptide encoding genes and are suitable for present purposes. In addition, Agrobacterium containing both armed and disarmed Ti genes can be used for the transformations.

A number of wild-type and disarmed strains of Agrobacterium tumefaciens and Agrobacterium rhizogenes harboring Ti or Ri plasmids can be used for gene transfer into plants. Optionally, the Agrobacterium hosts contain disarmed Ti and Ri plasmids that do not contain the oncogenes which cause tumorigenesis or rhizogenesis, respectively, which are used as the vectors and contain the genes of interest that are subsequently introduced into plants. Optional strains include but are not limited to Agrobacterium tumefaciens strain AGL1, C58, a nopaline-type strain that is used to mediate the transfer of DNA into a plant cell, octopine-type strains such as LBA4404 or succinamopine-type strains e.g., EHA101 or EHA105. The use of these strains for plant transformation has been reported and the methods are familiar to those of skill in the art.

Those of skill in the art are aware of the typical steps in the plant transformation process. The Agrobacterium can be prepared, for example, by inoculating a liquid such as Luria Burtani (LB) media directly from a glycerol stock or streaking the Agrobacterium onto a solidified media from a glycerol stock, allowing the bacteria to grow under the appropriate selective conditions, generally from about 26.degree. C.-30.degree. C., optionally about 28.degree. C., and taking a single colony from the plate and inoculating a liquid culture medium containing the selective agents. Alternatively, for example, a loopful or slurry of Agrobacterium can be taken from the plate and resuspended in liquid and used for inoculation. Those of skill in the art are familiar with procedures for growth and suitable culture conditions for Agrobacterium as well as subsequent inoculation procedures. The density of the Agrobacterium culture used for inoculation and the ratio of Agrobacterium cells to explant can vary from one system to the next, and therefore optimization of these parameters for any transformation method is expected.

Optionally, an Agrobacterium culture is inoculated from a streaked plate or glycerol stock and is grown overnight, and the bacterial cells are washed and resuspended in a culture medium suitable for inoculation of the explant. Suitable inoculation media for the present invention include, but are not limited 1/2 MSPL (2.2 g/L GIBCO (Carlsbad, Calif.) MS salts, 2 mg/L glycine, 0.5 g/L niacin, 0.5 g/L L-pyridoxine-HCl, 0.1 mg/L thiamine, 115 g/L L-proline, 26 g/L D-glucose, 68.5 g/L sucrose, pH 5.4) or 1/2 MS VI (2.2 g/L GIBCO (Carlsbad, Calif.) MS salts, 2 mg/L glycine, 0.5 g/L niacin, 0.5 g/L L-pyridoxine-HCl, 0.1 mg/L thiamine, 115 g/L L-proline, 10 g/L D-glucose, and 10 g/L sucrose, pH 5.4). The inoculation media can be supplemented with a growth inhibiting agent (PCT Publication WO 01/09302). The range and concentration of the growth inhibition agent can vary and depends of the agent and plant system. Growth inhibiting agents including, but not limited to, silver nitrate, silver thiosulfate, or carbenicillin are the preferred growth inhibition agents. The growth inhibiting agent is added in the amount necessary to achieve the desired effect. Silver nitrate is optionally used in the inoculation media at a concentration of about 1 μM (micromolar) to 1 mM (millimolar), or 5.mu.M-100 .mu.M. The concentration of carbenicillin used in the inoculation media is about 5 mg/L to 100 mg/L, or about 50 mg/L. A compound which induces Agrobacterium virulence genes such as acetosyringone can also be added to the inoculation medium. In one embodiment, the Agrobacterium used for inoculation are pre-induced in a medium such as a buffered media with appropriate salts containing acetosyringone, a carbohydrate, and selective antibiotics. In a preferred embodiment, the Agrobacterium cultures used for transformation are pre-induced by culturing at about 28.degree. C. in AB-glucose minimal medium (Chilton et al., 1974; Lichtenstein and Draper, 1986) supplemented with acetosyringone at about 200 .mu.M and glucose at about 2%. The concentration of selective antibiotics for Agrobacterium in the pre-induction medium is about half the concentration normally used in selection. The density of the Agrobacterium cells used is about 10.sup.7-10¹⁰ cfu/ml of Agrobacterium. Prior to inoculation the Agrobacterium can be washed in a suitable media such as 1/2 MS.

The next stage of the transformation process is the inoculation. In this stage the explants and Agrobacterium cell suspensions are mixed together. The mixture of Agrobacterium and explant(s) can also occur prior to or after a wounding step. By wounding as used herein is meant any method to disrupt the plant cells thereby allowing the Agrobacterium to interact with the plant cells. Those of skill in the art are aware of the numerous methods for wounding. These methods would include, but are not limited to, particle bombardment of plant tissues, sonicating, vacuum infiltrating, shearing, piercing, poking, cutting, or tearing plant tissues with a scalpel, needle or other device. The duration and condition of the inoculation and Agrobacterium cell density will vary depending on the plant transformation system. The inoculation is generally performed at a temperature of about 15.degree. C.-30.degree. C., optionally 23.degree. C.-28.degree. C. from less than one minute to about 3 hours. The inoculation can also be done using a vacuum infiltration system.

After inoculation, any excess Agrobacterium suspension can be removed and the Agrobacterium and target plant material are co-cultured. The co-culture refers to the time post-inoculation and prior to transfer to a delay or selection medium. Any number of plant tissue culture media can be used for the co-culture step. For the present invention, a reduced salt media such as half-strength MS-based co-culture media is used and the media lacks complex media additives including but not limited to undefined additives such as casein hydrosylate, and B5 vitamins and organic additives. Plant tissues after inoculation with Agrobacterium can be cultured in a liquid media. Optionally, plant tissues after inoculation with Agrobacterium are cultured on a semi-solid culture medium solidified with a gelling agent such as agarose, such as a low EEO agarose. The co-culture duration is from about one hour to 72 hours, or less than 36 hours, or about 6 hours to 35 hours. The co-culture media can contain one or more Agrobacterium growth inhibiting agent(s) or combination of growth inhibiting agents such as silver nitrate, silver thiosulfate, or carbenicillin. The concentration of silver nitrate or silver thiosulfate is optionally about 1 .mu.M to 1 mM, optionally about 5 .mu.M to 100 .mu.M, even optionally about 10 .mu.M to 50 .mu.M, most optionally about 20 .mu.M. The concentration of carbenicillin in the co-culture medium is optionally about 5 mg/L to 100 mg/L optionally 10 mg/L to 50 mg/L, even optionally about 50 mg/L. The co-culture is typically performed for about one to three days optionally for less than 24 hours at a temperature of about 18.degree. C.-30.degree. C., optionally about 23.degree. C.-25.degree. C. The co-culture can be performed in the light or in light-limiting conditions. Optionally, the co-culture is performed in light-limiting conditions. By light-limiting conditions as used herein is meant any conditions which limit light during the co-culture period including but not limited to covering a culture dish containing the plant/Agrobacterium mixture with a cloth, foil, or placing the culture dishes in a black bag, or placing the cultured cells in a dark room. Lighting conditions can be optimized for each plant system as is known to those of skill in the art.

After co-culture with Agrobacterium, the explants can be placed directly onto selective media. The explants can be sub-cultured onto selective media in successive steps or stages. For example, the first selective media can contain a low amount of selective agent, and the next sub-culture can contain a higher concentration of selective agent or vice versa. The explants can also be placed directly on a fixed concentration of selective agent. Alternatively, after co-culture with Agrobacterium, the explants can be placed on media without the selective agent. Those of skill in the art are aware of the numerous modifications in selective regimes, media, and growth conditions that can be varied depending on the plant system and the selective agent. In the preferred embodiment, after incubation on non-selective media containing the antibiotics to inhibit Agrobacterium growth without selective agents, the explants are cultured on selective growth media. Typical selective agents include but are not limited to antibiotics such as geneticin (G418), kanamycin, paromomycin, herbicides such as glyphosate or phosephinothericine, or other growth inhibitory compounds such as amino acid analogues, e.g., 5 methyltryptophan. Additional appropriate media components can be added to the selection or delay medium to inhibit Agrobacterium growth. Such media components can include, but are not limited to antibiotics such as carbenicillin or cefotaxime.

After the co-culture step, and optionally before the explants are placed on selective or delay media, cells can be analyzed for efficiency of DNA delivery by a transient assay that can be used to detect the presence of one or more gene(s) contained on the transformation vector, including, but not limited to a screenable marker gene such as the gene that codes for .beta.-glucuronidase (GUS). The total number of blue spots (indicating GUS expression) for a selected number of explants is used as a positive correlation of DNA transfer efficiency. The efficiency of T-DNA delivery and the effect of various culture condition manipulations on T-DNA delivery can be tested in transient analyses as described. A reduction in the T-DNA transfer process can result in a decrease in copy number and complexity of integration as complex integration patterns can originate from co-integration of separate T-DNAs (DeNeve et al., 1997). The effect of culture conditions of the target tissue can be tested by transient analyses and optionally, in stably transformed plants. Any number of methods are suitable for plant analyses, including but not limited to, histochemical assays, biological assays, and molecular analyses.

After effecting delivery of exogenous DNA to recipient cells, the next steps generally concern identifying the transformed cells for further culturing and plant regeneration. As mentioned herein, in order to improve the ability to identify transformants, one can desire to employ a selectable or screenable marker gene as, or in addition to, the expressible gene of interest. In this case, one would then generally assay the potentially transformed cell population by exposing the cells to a selective agent or agents, or one would screen the cells for the desired marker gene trait.

Other useful Agrobacterium methods include transformation of other cassava tissues capable of regenerating into complete plants.

In one embodiment a transgenic cassava plant (or other plant) is produced via organogenesis, somatic embryogenesis, and/or friable embryogenic callus.

Useful callus-based methods and other methods are described, for example, by Sudarmonowati et al. (“Factors affecting friable embryogenic callus in several plant species”; JOURNAL of BIOTECHNOLOGY RESEARCH in TROPICAL REGION, Vol. 2, No. 2, October 2009) and Hankuoa et al. (“Production of the first transgenic cassava in Africa via direct shoot organogenesis from friable embryogenic calli and germination of maturing somatic embryos”; African Journal of Biotechnology Vol. 5 (19), pp. 1700-1712, 2 Oct. 2006).

In one embodiment, a callus is produced by adding tyrosine to culture medium to stimulate production of a callus.

Transformation can also be accomplished by use of the vectors and constructs discussed below.

Transformation can be stable or transient, integrated or non-integrated.

### Host Plants

With the present invention, it is now possible to use AOX and antioxidation products to control PPD in plants. Optionally, the host plant is of the genus Manihot, for example M. walkerae, M. esculenta Crantz, M. esculenta ssp. Flabellifolia, M. esculenta sub spp peruviana, M. tristis., M. carthaginensis, M. brachyloba and M. fomentosa ed.

As taught herein, certain embodiments of the present invention (e.g. expression of AOX) are especially useful in plants which contain high levels of cyanogenic glycosides in a comestible thereof (i.e. cyanogenic crops). In one embodiment, the host plant is a cyanogenic crop. Optionally, the cyanogenic crop is a crop that normally undergoes rapid PPD. Numerous examples of cyanogenic crops are known in the art. Optionally, the cyanogenic crop is selected from the group consisting of cassava, sorghum, barley, cherry, apricot, plum, peach, mango, and lima bean.

Among other aspects, the present invention also contemplates a plant product or a plant part of a genetically modified plant taught herein.

### Examplary Plants

Table 15 lists examplary plants of the present invention which comprise combinations of transgenes taught herein.

In one embodiment, a host plant (e.g. cassava or other Manihot) is a plant selected from the group consisting of Plants 1-37, as listed in Table 15.

Optionally, one or more of the genes (e.g. all, or all excluding any CYP79D1/D2 RNAi) are operably linked to a comestible-specific promoter (e.g. root or fruit specific promoter).

Optionally, one or more of the genes (e.g. all, or all excluding any CYP79D1/D2 RNAi) are operably linked to root-specific promoter (e.g. patatin).

Optionally, one or more of the genes (e.g. all, or all excluding any CYP79D1/D2 RNAi) are operably linked to a fruit-specific promoter.

Optionally, the plant is a cyanogenic crop. Optionally, the cyanogenic crop is selected from the group consisting of cassava, other crops such as sorghum, barley, cherry, apricot, plum, peach, mango, and lima bean. Optionally, the plant is a cyanogenic crop and one or more of the genes (e.g. all, or all excluding any CYP79D1/D2 RNAi) are operably linked to a comestible-specific promoter. Optionally, the cyanogenic crop (e.g. cassava) exhibits reduced PPD.

Optionally, the plant comprises any optional feature of transgenic plants taught herein.

TABLE 15
Examplary Plants

| | | | | ROS | | | | CYP79D1/ | | | | | |
|---|---|---|---|---|---|---|---|---|---|---|---|---|---|
| Plant | AOX | PSY | DXS | scavenger(s) | HPT | HGGT | GGR | D2 RNAi | linamarase | β-CAS | NIT4 | HNL | anti-PCD |
| 1 | x | x | | | | | | | | | | | |
| 2 | x | | x | | | | | | | | | | |
| 3 | x | | | | | | | x | | | | | |
| 4 | x | | | x | | | | | | | | | |
| 5 | x | | | | | | | | | | | | x |
| 6 | x | x | x | | | | | | | | | | |
| 7 | x | | | | | | | | | | | | |
| 8 | x | | | | | | | | x | | | | |
| 9 | x | x | | x | | | | | | | | | |
| 10 | x | | x | x | | | | | | | | | |
| 11 | x | | | x | | | | x | | | | | |
| 12 | x | | | x | | | | | | | | | x |
| 13 | x | | | x | | | | | | | | | |
| 14 | x | x | x | x | | | | | | | | | |
| 15 | x | | | x | | | | | | | | | |
| 16 | x | | | | | | | | | | | x | |
| 17 | x | | | x | | | | | x | | | | |
| 18 | x | x | x | | x | x | | | | | | | |
| 19 | x | | x | | | x | | | | | | | |
| 20 | x | x | x | | x | | | | | | | | |
| 21 | x | x | x | | | | | | | | | | |
| 22 | x | x | | | | x | | | | | | | |
| 23 | x | | | | x | | x | | | | | | |
| 24 | x | | | | | | | | | x | x | | |
| 25 | x | | | | | | | | x | x | x | | |
| 26 | x | | | | | | | | x | x | | | |
| 27 | x | x | | | | | x | | | | | | |
| 28 | x | | | | | | | | x | | x | | |
| 29 | | x | x | | x | x | | | | | | | |
| 30 | | | x | | | x | | | | | | | |
| 31 | | x | x | | x | | | | | | | | |
| 32 | | x | x | | | | | | | | | | |
| 33 | | x | | | | x | | | | | | | |
| 34 | | | | | x | | x | | | | | | |
| 35 | | | | | | | | | | x | x | | |
| 36 | | | | | | | | | x | x | x | | |
| 37 | | | | | | | | | x | x | | | |
| 38 | | | | | | | | | x | | x | | |
| 39 | | x | | | | | x | | | | | | |

The citations provided herein are hereby incorporated by reference for the cited subject matter.

### EXAMPLES

### Example 1

### Non-Transgenic Cassava

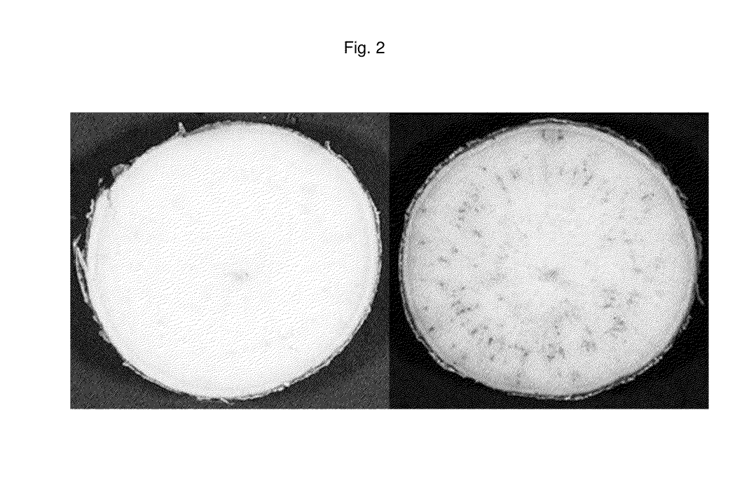

Once cassava is harvested, its starchy storage roots must be consumed or processed within 24 hours or they will deteriorate, becoming unpalatable and unmarketable. Roots deteriorate rapidly after harvest as a result of complex biochemical changes following harvest- and processing-induced wounding. Cassava roots generally start to deteriorate 24 to 48 hours after harvest (FIG. 2).

### Example 2

### ROS in Wounded, Cyanide-Free Cassava

Although cassava is a major source of carbohydrates for over 600 million people, the roots contain potentially toxic levels cyanogenic glucosides, primarily (95%) linamarin. The first dedicated step in linamarin synthesis is catalyzed by two similar P450 enzymes, CYP79D1 and CYP79D2. Antisense knock down of CYP79D1 and CYP79D2 under the control of the Arabidopsis CAB1 promoter produced transgenic cassava with cyanide-free roots.

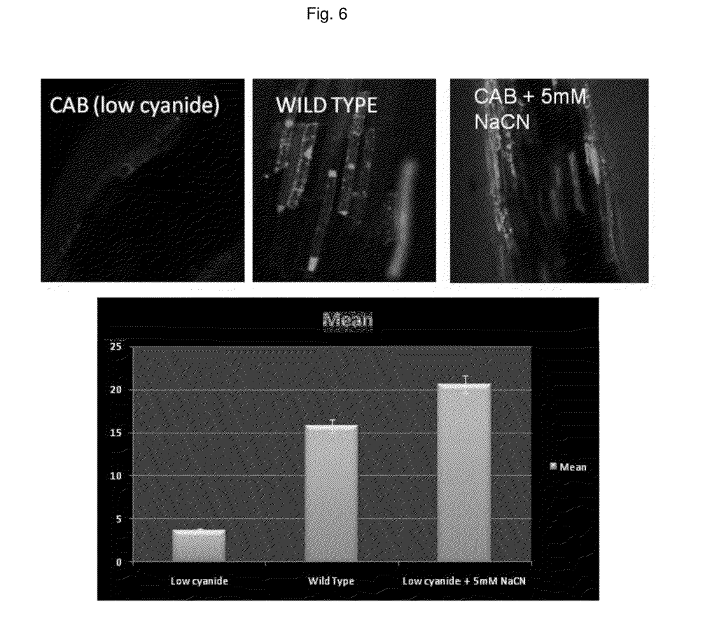

ROS production in the cyanide-free CAB transgenic line was significantly reduced compared to wild-type cassava (in situ detecting using fluorescent dye CM-H₂DCFDA, as well as 3,3 diaminobenzidine). Supplementing the cyanide-free roots with cyanide (5 mM NaNC) restored ROS production to wild type levels (FIG. 6).

Although these data support a link between cyanide and ROS levels, significant fluorescence was still seen in the cyanide-free roots. This ROS production presumably arose from plasma membrane NADPH oxidase activity (cyanide insensitive). However, inhibition of the plasma membrane NADPH oxidase does not affect the production of ROS (data not shown). These data support the hypothesis taught here in which the source of the ROS is mitochondrial, where cyanide inhibits cytochrome oxidase in the mitochondrial respiratory chain.

### Example 3

### Expression of AOX Reduces PPD and ROS Production

Mitochondrial alternative oxidase (AtAOX1a) was over-expressed in cassava. Without being bound by theory, the inventors believe that AOX provides an escape valve for electrons in cyanide poisoned cassava mitochondria. Unlike cytochrome C oxidase, AOX is cyanide insensitive.

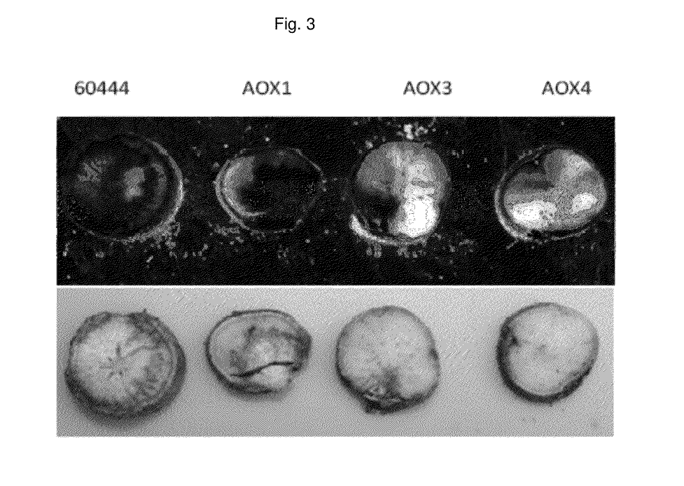

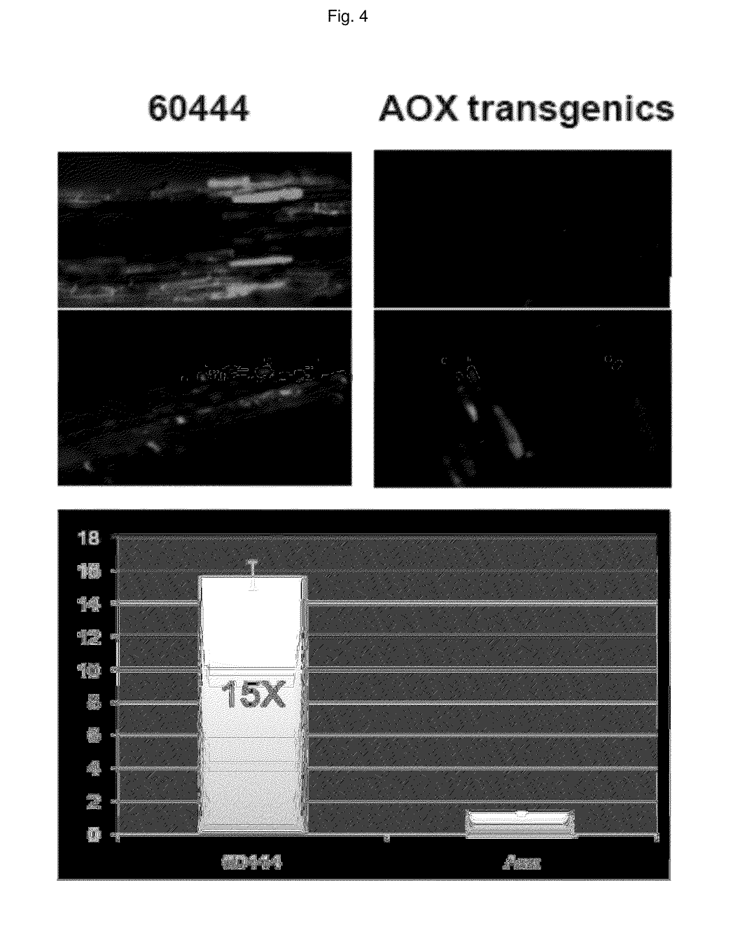

Surprisingly, transgenic plants over-expressing AOX had substantially reduced ROS levels that were undetectable in some cases (FIG. 4). Even further surprising was that mature (6 month old) transgenic plants expressing the highest AOX levels (FIG. 3, plants designated AOX3 and AOX4) had substantially reduced PPD symptoms one week after harvest. The only apparent undesirable phenotype in high AOX expressing plants was vasculature discoloration, representative of the earliest events in PPD.

The wild-type (60444) and AOX1 plants lacked scopoletin fluorescence at six days after harvest indicating a more progressed PPD status compared to the AOX3 and AOX4 transgenics which expressed the highest AOX levels. These results support the reduction of PPD (e.g. delaying tissue disruption and discoloration) by over expressing an AOX gene at sufficient levels, but indicate that additional strategies need to be explored to reduce early symptoms that lead to vascular discoloration and accumulation of scopoletin.

### Example 4

### Over-Expression of AOX in Comestibles

As detailed in Example 3, the level of PPD and ROS production is negatively correlated with AOX expression level. Further demonstrated was that PPD can be substantially reduced (e.g. delaying tissue disruption and discoloration) by expressing AOX at a sufficient level.

To enhance the level of transgenic AOX expression in a comestible (e.g. cassava root), multiple strategies were pursued, including comestible-specific expression, codon-optimization, the use of leader sequences and terminator sequences, and the inclusion of introns in the exon sequences.

As the tuberous roots are the primary comestible of cassava, the patatin promoter was selected as a comestible-specific promoter to drive AOX expression in cassava roots. In addition, the Arabidopsis AOX1a gene was codon-optimized for expression in cassava (e.g. by site-directed mutagenesis).

The HSP70 leader sequence was selected as a leader sequence. The Nos terminator was selected as a terminator. The final construct was inserted into a pBI121-based binary vector 3D.

The final plasmid was introduced in to Agrobacterium strain LBA4404 for transformation into cassava cultivar TMS 60444. Briefly, a number of apical leaves were obtained and placed on somatic embryo induction medium. Once somatic embryos were obtained, they were matured and used for transformation.

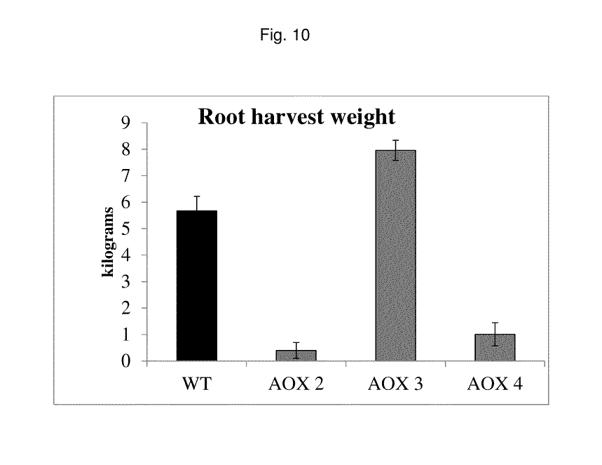

Field trial data showed that AOX2 and AOX4 had very poor root development, while bt comparison AOX3 had higher root yield than wild type, an intermediate AOX activity. (FIG. 10)

Transgenic cassava expressing the highest level of AOX showed no signs of PPD after two weeks, in contrast to wild-type plants which showed symptoms in three days. Wild-type and transgenic cassava expressing AOX were harvested after one year of growth in the field. The storage roots were removed from the plant and divided in to three sets. In one set the wild-type and storage roots where sliced to remove the proximal and distal ends so that only a 16 cm section remained. One end of the root was covered with plastic wrap, the other end was left exposed to the environment, the roots were kept at 80% humidity in a growth chamber. After 5 days the roots were analyzed for PPD development. The second set of roots was treated similarly to the first set but the roots were analyzed after 10 days for PPD development. The third set of roots was left unaltered in the cold room for 21 days after which time the roots were analyzed for PPD development. The quantification of PPD was done using ImageJ software. The comparison between the wild-type roots and those from transgenic lines expressing AOX was done in each set, the value of wild-type PPD was taken as 100%.

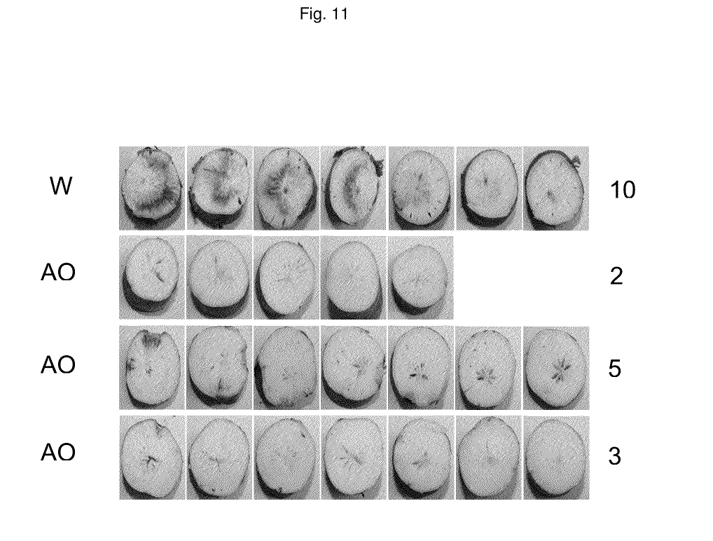

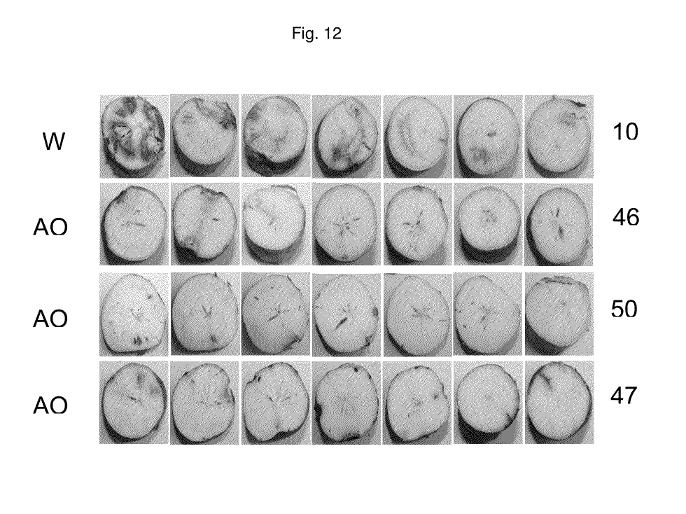

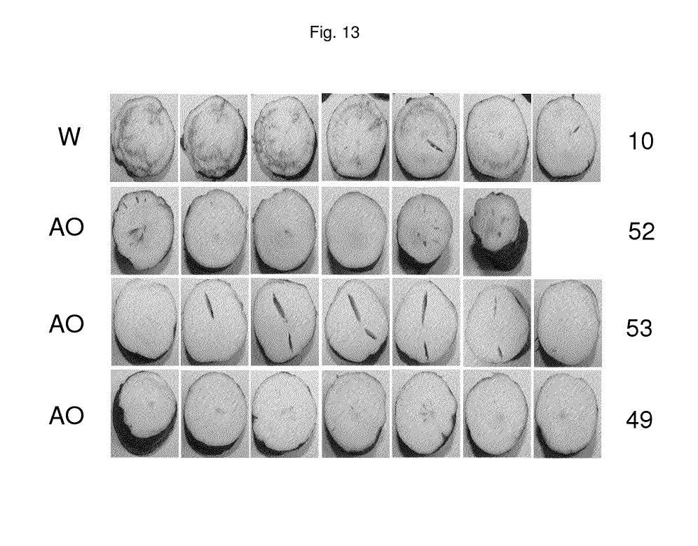

The results of room temperature storage at 5 days are shown in FIG. 11. The results of room temperature storage at 10 days are shown in FIG. 12. The results of refrigerated storage at 21 days are shown in FIG. 13.

Although the present invention was demonstrated using cassava as the comestible, the invention is not limited to any particular crop. For example, in addition to cassava, other crops such as sorghum, barley, cherry, apricot, plum, peach, mango, and lima bean are all known to contain high levels of cyanogenic glycosides and undergo some degree of PPD. With the teachings provided herein, the skilled artisan can now reduce PPD in any plant (e.g. cyanogenic crops) by overexpressing AOX in a comestible of the plant. For any given crop, the skilled artisan will readily appreciate the appropriate comestible in which to overexpress AOX. Although comestible-specific expression may not be required to achieve sufficient expression levels for a given crop, methods for such targeted expression are well known in the art (e.g. using a fruit-specific promoter in a mango plant).

### Example 5

### Expression of PSY

PSY was overexpressed in cassava. The crtB gene from Erwenia was selected as the PSY. The patatin promoter was chosen as a root- and comestible-specific promoter. 25 transgenic lines were produced. Total amounts of carotenoids based on spectrophotometric measurement ranged from approximately 20 to 52 μg/g dry weight in transgenic plants versus approximately 2 μg/g dry weight in roots from control plants. Surprisingly, these results indicate that a 10- to 20-fold increase in carotenoid content of cassava storage roots is possible by expression of crtB. This supports the expression of PSY to reduce ROS levels and PPD in comestibles such as cassava roots. Plants having high β-carotene levels (>30 ppm dry weight) were observed to have extended shelf life out to 4 weeks before notable discoloration of the vasculature.

### Example 6

### Expression of ROS Scavengers

Several ROS scavengers were expressed in cassava to reduce ROS production and PPD. The ROS scavengers selected were Ascor. perox., CuZn SOD, GSH synthase, and D-galacturonic acid reductase.

Several lines were produced, each with the ROS scavengers under the control of a root-specific promoter (patatin) or the PX3 (MecPX3) promoter for expression in vascular tissues where discoloration is observed first during PPD.

### Example 7

### Expression of DXS

DXS is overexpressed in cassava. The DXS gene from Arabidopsis is selected as the DXS. The patatin promoter is selected as a root- and comestible-specific promoter. Plants expressing the highest levels of DXS have no detectable ROS production after wounding and cyanide production. In addition the shelf life is extended to one week.

### Example 8

### Coexpression of AOX and PSY

AOX and PSY were coexpressed in cassava. The Arabidopsis AOX1a gene was selected as the AOX. The crtB gene from Erwenia was selected as the PSY. The patatin promoter was selected as a root- and comestible-specific promoter. Plants expressing the highest levels of AOX had no detectable ROS production after wounding and cyanide production. In addition the shelf life was extended to one week.

### Example 9

### Coexpression of PSY and DXS

PSY and DSX were coexpressed in cassava. The crtB gene from Erwenia was selected as the PSY. The Arabidopsis AtDSX gene was selected as the DSX. The patatin promoter was selected as a root- and comestible-specific promoter. Transgenic plants derived from this example demonstrate remarkable PPD reduction and extended shelf life.

### Example 10

### Coexpression of PSY and GGR

PSY and GGR were coexpressed in cassava. The crtB gene from Erwenia was selected as the PSY. The Arabidopsis AtGGR gene was selected as the GGR. The patatin promoter was selected as a root- and comestible-specific promoter. Co-expression of DXS with psy resulted in a 3 fold higher level of B-carotene and an equivalent level of vitamin e as psy single gene transgenic plants. Transgenic plants derived from this example demonstrate remarkable PPD reduction and extended shelf life.

### Example 11

### Coexpression of HPT and GGR

HPT and GGR are coexpressed in cassava. The Arabidopsis AtHPT gene is selected as the HPT. The Arabidopsis AtGGR gene is selected as the GGR. The patatin promoter was selected as a root- and comestible-specific promoter. Transgenic plants derived from this example demonstrate remarkable PPD reduction and extended shelf life.

### Example 12

### Coexpression of DSX and HPT

DSX and HPT and are coexpressed in cassava. The Arabidopsis AtHPT gene is selected as the HPT. The Arabidopsis AtDSX gene is selected as the DSX. The patatin promoter is selected as a root- and comestible-specific promoter. Transgenic plants derived from this example demonstrate remarkable PPD reduction and extended shelf life.

### Example 13

### Coexpression of DSX and HGGT

DSX and HGGT are coexpressed in cassava. The Arabidopsis AtHPT gene is selected as the HPT. The patatin promoter is selected as a root- and comestible-specific promoter. Transgenic plants derived from this example demonstrate remarkable PPD reduction and extended shelf life.

### Example 14

### Coexpression of AOX and CYP79D1/D2 RNAi

In view of the reduced ROS production exhibited by the CYP79D1/D2 RNAi transgenic lines, and the reduced ROS and PPD exhibited by the AOX transgenic lines, the transgenes are coexpressed in cassava to further reduce PPD. Transgenic plants derived from this example demonstrate remarkable PPD reduction and extended shelf life.

### Example 15

### Coexpression of PSY, DXS, and HPT

PSY, DSX, and HPT are coexpressed in cassava. The crtB gene from Erwenia is selected as the PSY. The Arabidopsis AtDSX gene is selected as the DSX. The Arabidopsis AtHPT gene was selected as the HPT. The patatin promoter is selected as a root- and comestible-specific promoter. Transgenic plants derived from this example demonstrate remarkable PPD reduction and extended shelf life.

### Example 16

### Coexpression of PSY, DXS, HPT, and GGR

PSY, DSX, HPT, and GGR are coexpressed in cassava. The crtB gene from Erwenia is selected as the PSY. The Arabidopsis AtDSX gene is selected as the DSX. The Arabidopsis AtHPT gene is selected as the HPT. The Arabidopsis AtGGR gene is selected as the GGR. The patatin promoter is selected as a root- and comestible-specific promoter. Transgenic plants derived from this example demonstrate remarkable PPD reduction and extended shelf life.

### Example 17

### Coexpression of PSY, DXS, HPT, and HGGT

PSY, DSX, HPT, and HGGT are coexpressed in cassava. The crtB gene from Erwenia is selected as the PSY. The Arabidopsis AtDSX gene is selected as the DSX. The Arabidopsis AtHPT gene is selected as the HPT. The patatin promoter is selected as a root- and comestible-specific promoter.

### Example 18

### Detection of Cyanogen Metabolizing Gene Expression

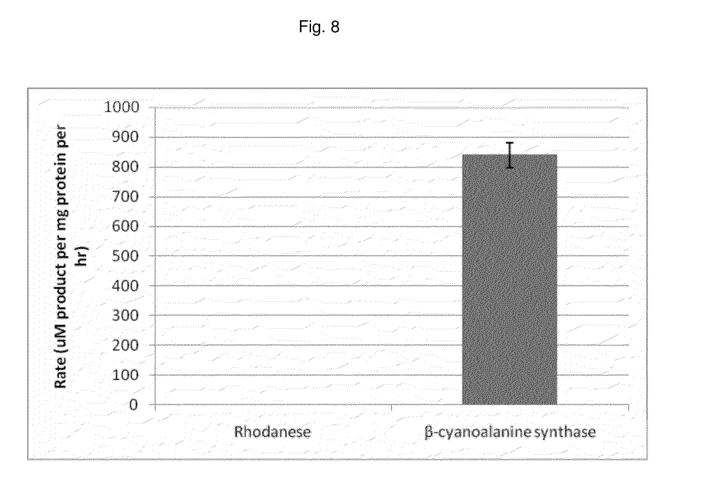

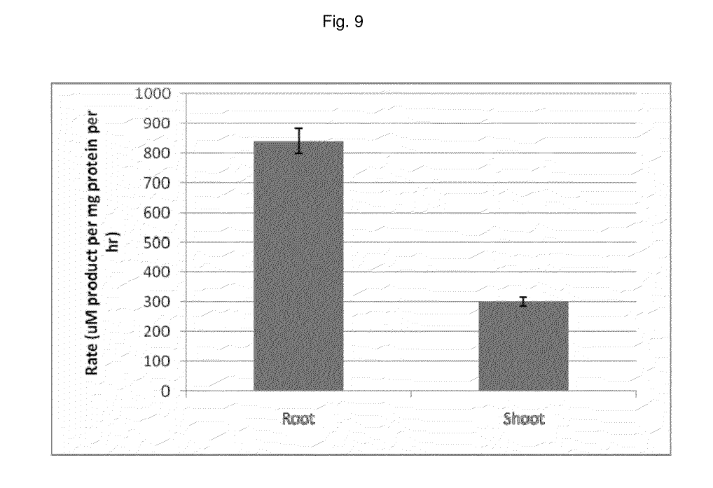

Expression of cyanogen metabolizing genes was detected in cassava. The sulfurtransferase rhodanese, which is involved in cyanide detoxification as thiocyantes in humans has no detectable activity in cassava roots; however, β-cyanoalanine synthase (β-CAS), involved in cyanide assimilation into amino acids, showed significant expression in roots (FIG. 8). β-CAS showed 3 times more activity in cassava roots than in leaves (FIG. 9).

This data indicates that β-CAS and not rhodanese, is the key cyanide detoxifying enzyme in cassava roots (it has higher root rates than shoots rate even against low root protein). Rhodanese is barely detectable in cassava roots.

These data suggests that cyanogenic glucosides are transportable forms of reduced nitrogen in plants such as cassava. With the teachings provided herein, this supports the expression of cyanogen metabolizing genes (e.g. β-CAS, NIT4, linamarase, HNL) to reduce ROS formation and PPD.

### Example 19

### Expression of Linamarase

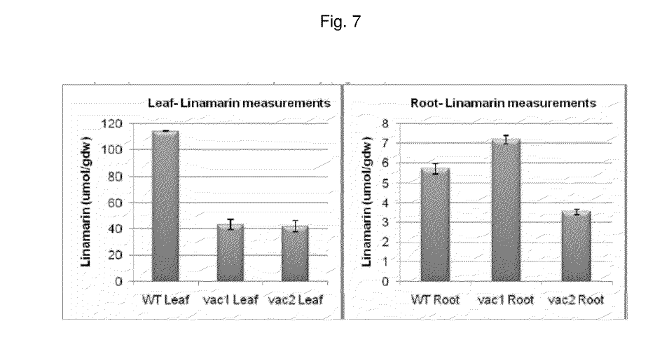

A ΔN-terminal linamarase was fused to either a vacuolar targeting sequence or a cytoplasmic targeting sequence and expressed in cassava. The patatin promoter was selected as a root- and comestible-specific promoter. Transgenic plants from two independent events (vac1 and vac2) were assayed for cyanogen (linamarin) levels in the leaves and roots. The results are shown in FIG. 7. Linamarin levels in leaves, roots and stems were measured by GC-MS. The plants expressing linamarase in the vacuole demonstrated a remarkable reduction in leaf linamarin levels (e.g. reduced by 35-36% in the transgenics relative to WT [FIG. 7]). Linamarin levels in roots ranged from a 22% increase to a 41% decrease in vac-1 and vac-2, respectively (FIG. 7).

### Example 20

### Coexpression of β-CAS and NIT4

β-CAS and NIT4 were coexpressed in cassava. The patatin promoter was selected as a root- and comestible-specific promoter. Transgenic plants derived from this example demonstrate remarkable PPD reduction and extended shelf life.

### Example 21

### Expression of HNL

HNL was expressed in cassava. The cassava HNL gene was selected as the HNL The patatin promoter was selected as a root- and comestible-specific promoter. Transgenic plants derived from this example demonstrate remarkable PPD reduction and extended shelf life.

### Example 22

### Detection of Reactive Oxygen Species

The following methods were used to detect ROS in wild-type and transgenic cassava.

Spectrofluorometry.

Intracellular production of ROS is measured by using 2′,7′-dichlorofluorescein diacetate which is converted to the membrane-impermeant polar derivative H₂DCF by esterases when it is taken up by the cell. H₂DCF is nonfluorescent but is rapidly oxidized to the highly fluorescent DCF by intracellular H₂O₂ and other peroxides. Fluorescence is measured by using a Hitachi F2000 fluorescence spectrophotometer (Tokyo) with excitation and emission wavelengths set at 488 nm and 520 nm, respectively.

Laser-Scanning Confocal Microscopy.

Laser-Scanning confocal microscopy is performed on cells loaded with H₂DCF-DA (15 μM) and Mitotracker Red (0.5 μM; Molecular Probes), a dye that is specifically taken up by metabolically active mitochondria. DCF is excited at 488 nm and detected through a 530/30-nm bandpass filter. Mitotracker Red is excited at 568 and detected through a >665-nm long-pass filter. Data is collected by a dedicated instrument computer and stored on the hard drive.

### Example 23

### PPD Quantification

Surprisingly, transgenic plants of the present invention exhibit reduced PPD. Among the various PPD symptoms taught herein, PPD can be quantified by measuring blue-black discoloration of xylem parenchyma (vascular streaking), for example, using the following method:

The central sections of the root are used for PPD quantification. Measurements are made individually for each root. PPD is determined, generally using the method of Wheatley et al. (Post-harvest deterioration of cassava 2 roots, in Cassava: Research, Production and Utilization, Ed by Cock J H and 3 Reyes J A. UNDP-CIAT, Cali, Colombia, pp 655-671 (1985)). For example, prepared roots are stored for a waiting period (e.g. 3, 5, 7, 14 days). Roots are kept in a controlled environment chamber at 25° C. and 60-80% relative humidity before PPD quantification. The proximal and distal root ends are removed and covered with clingfilm. After the waiting period, seven 2-cm thick transversal slices are cut along the root, starting at the proximal end. A score between 1 and 10 is assigned to each slice, corresponding to the percentage of the cut surface showing discoloration (1=10%, 2=20%, etc). The mean PPD score for each root is calculated by averaging the scores of the seven slices.

I claim:

1. A method for conferring resistance to physiological post-harvest deterioration (PPD) in a plant comprising transforming the plant with a gene encoding an alternative oxidase (AOX), wherein:
a. the gene encoding the AOX is operably linked to a promoter;
b. if the plant is grown and harvested for its comestible, a sufficient level of AOX accumulates in the comestible to reduce PPD; and
c. optionally, the plant is cassava and the comestible is a cassava tuber.

2. The method of claim 1, wherein the promoter is a comestible-specific promoter.

3. The method of claim 2, wherein the comestible-specific promoter is a root- or tuber-specific promoter selected from the group consisting of a patatin promoter, an iso flavones synthase promoter, a granular bound starch synthase promoter, a sporamin promoter, and a sugar beet storage promoter.

4. The method of claim 1, wherein the AOX is an AOX derived from a higher plant, optionally wherein the higher plant is Arabidopsis.

5. The method of claim 1, wherein the AOX is an AOX1, optionally wherein the AOX1 is an AOX1A.

6. The method of claim 5, wherein the AOX is derived from Arabidopsis.

7. The method of any of claims 1-6, further comprising transforming the plant with one or more genes encoding antioxidation products, wherein the one or more genes encoding antioxidation products are:
a. selected from the group consisting of:
i. one or more carotenoid biosynthesis genes, and
ii. one or more ROS scavengers; and
b. operably linked to a promoter, optionally a comestible-specific promoter.

8. The method of claim 7, wherein the one or more carotenoid biosynthesis genes are selected from the group consisting of phytoene synthase (PSY), 1-deoxyxylulose-5-phosphate synthase (DXS), geranylgeranyl reductase (GGR), homogentisate phytyltransferase (HPT), and combinations thereof.

9. The method of claim 8, wherein the one or more carotenoid biosynthesis genes comprise a phytoene synthase.

10. The method of claim 9, wherein the phytoene synthase is derived from a bacterium optionally, wherein the bacterium is Erwinia.

11. The method of claim 8, wherein the one or more carotenoid biosynthesis genes comprise a DXS.

12. The method of claim 11, wherein the DXS is derived from Arabidopsis.

13. The method of claim 8, wherein the one or more carotenoid biosynthesis genes comprise a phytoene synthase and a DXS.

14. The method of claim 7, wherein the one or more ROS scavengers are selected from the group consisting of a superoxide dismutase, a catalase, an ascorbate peroxidase, a D-galacturonic acid reductase, a γ-glutamylcysteine synthase, a dehydroascorbate reductase, a glutathione peroxidase, and a glutathione reductase.

15. The method of any of claims 1-6, further comprising transforming the plant with one or more genes encoding cyanogen detoxifying products.

16. The method of claim 15, wherein the cyanogen detoxifying products are selected from the group consisting of cyanogen metabolism genes and cyanogen biosynthesis inhibitors.

17. The method of claim 16, wherein the cyanogen metabolism genes are cyanogen metabolizing enzymes selected from the group consisting of β-cyanoalanine synthase (β-CAS), Rhodanese, nitrilase 4 (NIT4), linamarase, and hydroxynitrile lyase (HNL).

18. The method of claim 16 wherein the cyanogen biosynthesis inhibitors is an RNAi agent which targets at least one of CYP79D1 and CYP79D2.

19. A genetically modified plant produced by the method of any of claims 1-6.

20. A genetically modified plant produced by the method of any of claim 7.

21. A genetically modified plant produced by the method of any of claim 9.

22. A genetically modified plant produced by the method of any of claim 11.

23. A genetically modified plant produced by the method of any of claim 13.

24. A genetically modified plant produced by the method of any of claim 15.

25. The genetically modified plant of any of claims 19-24, wherein the plant exhibits reduced PPD.

26. The genetically modified plant of claim 25, wherein the reduced PPD comprises one or more reduced PPD symptoms selected from:
a. tissue disruption;
b. vascular streaking;
c. general discoloration of the storage parenchyma;
d. occlusions and/or tyloses in xylem parenchyma;
e. scopoletin autofluorescence;
f. changes associated with the plant's response to wounding; and
g. suberization around wound sites.

27. A plant product derived from a genetically modified plant according to any of claims 19-26.

28. A plant part derived from a genetically modified plant according to any of claims 19-26.
